(19) United States
(12) Patent Application Publication
(10) Pub. No.: US 2010/0136537 A1
(43) Pub. Date: Jun. 3, 2010

(54) PHOTORECEPTOR PRECURSOR CELLS

(75) Inventors: Anand Swaroop, Ann Arbor, MI (US); Masayuki Akimoto, Kyoto (JP); Alan Mears, Ottawa (CA); Hong Cheng, Ann Arbor, MI (US); Edwin C.T. Oh, Baltimore, MD (US)

Correspondence Address: Casimir Jones, S.C., 2275 DEMING WAY, SUITE 310, MIDDLETON, WI, 53562, US

(73) Assignee: THE REGENTS OF THE UNIVERSITY OF MICHIGAN, Ann Arbor, MI (US)

(21) Appl. No.: 12/445,031

(22) PCT Filed: Oct. 10, 2007
(86) PCT No.: PCT/US07/80975

§ 371 Date: Jan. 5, 2010

Related U.S. Application Data
(60) Provisional application No. 60/850,471, filed on Oct. 10, 2006.
(60) Provisional application No. 60/881,527, filed on Jan. 19, 2007.

(51) Int. Cl. C12Q 1/68; C12N 5/07; C12N 15/00

(52) U.S. Cl. 435/6; 435/354; 435/440

(57) ABSTRACT

The present invention relates to photoreceptor cells. In particular, the present invention provides photoreceptor cells comprising heterologous nucleic acid sequences and transgenic animals comprising the same. The present invention also provides photoreceptor precursor cells (e.g., rod photoreceptor precursor cells), and methods of identifying, characterizing, isolating and utilizing the same. Compositions and methods of the present invention find use in, among other things, research, clinical, diagnostic, drug discovery, and therapeutic applications.

The present invention claims priority to U.S. Provisional Patent Application Ser. No. 60/850,471 filed Oct. 10, 2006, and U.S. Provisional Patent Application Ser. No. 60/881,527 filed Jan. 19, 2007, each of which is herein incorporated by reference in its entirety.

This invention was made with government support under Contract Nos. EY11115, EY014259, EY013934, DK020572 and EY007003 awarded by the National Institutes of Health. The government has certain rights in the invention.

### FIELD OF THE INVENTION

The present invention relates to photoreceptor cells. In particular, the present invention provides photoreceptor cells comprising heterologous nucleic acid sequences and transgenic animals comprising the same. The present invention also provides photoreceptor precursor cells (e.g., rod photoreceptor precursor cells), and methods of identifying, characterizing, isolating and utilizing the same. Compositions and methods of the present invention find use in, among other things, research, clinical, diagnostic, drug discovery, and therapeutic applications.

### BACKGROUND OF THE INVENTION

An overwhelming majority of the world's population will experience some degree of vision loss in their lifetime. Vision loss affects virtually all people regardless of age, race, economic or social status, or geographical location. Ocular-related disorders, while often not life threatening, necessitate life-style changes that jeopardize the independence of the afflicted individual. Vision impairment can result from a host of disorders, (e.g., diabetic retinopathies, proliferative retinopathies, retinal detachment, toxic retinopathies), diseases (e.g., retinal vascular diseases and/or retinal degeneration), aging, and other events (e.g., injury).

Photoreceptor loss (e.g., caused by a disorder, disease, aging, genetic predisposition, or injury) causes irreversible blindness. Cell transplantation was initially thought to be a feasible type of central nervous system repair. For example, photoreceptor degeneration initially leaves the inner retinal circuitry intact and new photoreceptors only need to make a single, short synaptic connection to contribute to the retinotopic map. However, there has been little to no success transplanting cells (e.g., brain or retina derived stem cells) into mature, adult retina resulting in the integration of the cells and synaptic connections.

Given the prevalence of ocular-related disorders, there exists a need for a better understanding of photoreceptor development (e.g., of the developmental stages of photoreceptor cells) and function (e.g., characterization and identification of cells capable of forming synaptic connections with the retina), and identification of photoreceptor cells (e.g., precursor cells) that may be used for research and/or clinical (e.g., therapeutic) applications.

### SUMMARY OF THE INVENTION

The present invention relates to photoreceptor cells. In particular, the present invention provides photoreceptor cells comprising heterologous nucleic acid sequences and transgenic animals comprising the same. The present invention also provides photoreceptor precursor cells (e.g., rod photoreceptor precursor cells), and methods of identifying, characterizing, isolating and utilizing the same. Compositions and methods of the present invention find use in, among other things, research, clinical, diagnostic, drug discovery, and therapeutic applications.

Accordingly, in some embodiments, the present invention provides a composition comprising a purified or isolated photoreceptor precursor cell. In some embodiments, the cell expresses a heterologous or endogenous biomarker. The present invention is not limited by the biomarker expressed and/or detected in the photoreceptor precursor cell. Indeed, a variety of biomarkers may be utilized including, but not limited to, those described herein (e.g., in FIGS. 5, 11, 12, and 13). In some embodiments, the cell expresses Nrl. In some embodiments, the presence or absence of expression of a biomarker (e.g., Nrl) identifies the cell as a rod photoreceptor precursor cell or a cone photoreceptor precursor cell. In some embodiments, the cell is able to survive and differentiate when placed within a retina. In some embodiments, the retina is an adult retina. In some embodiments, the retina is a degenerating retina. In some embodiments, the cell expresses green fluorescent protein or other detectable molecule. In some embodiments, the cell comprises heterologous nucleic acid sequence encoding a Nrl promoter operatively linked to green fluorescent protein or other detectable molecule. In some embodiments, the promoter comprises 2.5 kB of 5′ untranslated sequence of Nrl (e.g., with or without being operatively linked to a detectable molecule). In some embodiments, the cell is purified from an animal (e.g., a mouse). In some embodiments, the animal is selected from the group comprising an embryonic animal and a post-natal animal. In some embodiments, the embryonic animal is embryonic day 12 or older. In some embodiments, the post-natal animal is a post-natal day 1 through a post-natal day 7 animal. In some embodiments, the cell integrates within the outer nuclear layer of a retina when injected into the subretinal space of the retina. In some embodiments, the integrated cell forms synaptic connections with downstream targets in the retina. In some embodiments, the integrated cell responds to a synapse-dependent stimulus. The present invention is not limited by the type of synaptic-dependent stimulus. Indeed, a variety of stimuli may be utilized including, but not limited to, light.

The present invention also provides a transgenic, non-human animal whose genome comprises a heterologous nucleic acid sequence encoding a Nrl promoter. In some embodiments, the Nrl promote is operatively linked to green fluorescent protein or other detectable molecule. In some embodiments, the promoter comprises 2.5 kB of 5′ untranslated sequence of Nrl. In some embodiments, the genome lacks (e.g., completely) endogenous Nrl expression.

The present invention also provides a method of characterizing a photoreceptor precursor cell comprising: a) providing a photoreceptor precursor cell; and a subject; b) injecting the photoreceptor precursor cells into the subject (e.g., into the subretinal space of a retina); and c) identifying the presence or absence of Nrl expression in the cell. In some embodiments, the presence of Nrl expression in the cell identifies the cell as a rod photoreceptor cell. In some embodiments, the absence of Nrl expression in the cell identifies the cell as a cone photoreceptor cell. The present invention is not limited by the method of detecting biomarker (e.g., Nrl) presence. In some embodiments, detecting biomarker (e.g., Nrl) expression comprises detection of nucleic acid expression or protein expression. In some embodiments, characterizing further comprises detecting the expression of one or more biomarkers selected from the group comprising a gene presented in FIG. 11, a gene presented in FIG. 12, or a gene presented in FIG. 13. In some embodiments, a profile of two or more biomarkers are used to characterize photoreceptor development. In some embodiments, a profile of five or more biomarkers are used to characterize photoreceptor development. In some embodiments, a profile of ten or more biomarkers are used to characterize photoreceptor development.

The present invention further provides a method of purifying (e.g., isolating) a rod photoreceptor precursor cell comprising: providing a transgenic, non-human animal whose genome comprises a heterologous nucleic acid sequence encoding a Nrl promoter operatively linked to a detectable biomolecule (e.g., protein (e.g. green fluorescent protein)); dissecting neural retinas away from surrounding tissues from the animal; dissociating the cells; and sorting detectable protein positive cells away from green fluorescent protein negative cells. In some embodiments, a population of photoreceptor precursor cells are enriched. In some embodiments, cells are sorted using fluorescent activated cell sorting. In some embodiments, the transgenic, non-human animal is an embryonic mouse or a post-natal mouse. In some embodiments, the embryonic mouse is embryonic day 16 or older. In some embodiments, the post-natal mouse is a post-natal day 1 through a post-natal day 28 mouse.

The present invention also provides a method of transplanting a photoreceptor precursor cell into a host subject comprising providing a photoreceptor precursor cell; and a host subject; and injecting the photoreceptor precursor cell into the subject under conditions such that the cell generates rod cell synaptic connections.

The present invention also provides a method of identifying and/or characterizing a test compound comprising: providing a photoreceptor cell (e.g., a photoreceptor precursor cell); transplanting the photoreceptor cell into an animal (e.g., a mouse); exposing the animal to one or more test compounds; and characterizing photoreceptor cell development and/or function in the animal.

The present invention also provides a method of identifying and/or characterizing a test compound comprising: providing a photoreceptor cell comprising a heterologous nucleic acid sequence encoding a Nrl promoter (e.g., operatively linked to a detectable biomolecule (e.g., green fluorescent protein)); exposing the cell to one or more test compounds; and detecting a change in photoreceptor cell development and/or function. In some embodiments, the photoreceptor cell is present within a transgenic, non-human animal whose genome comprises a heterologous nucleic acid sequence encoding a Nrl promoter (e.g., operatively linked to a detectable biomolecule). In some embodiments, detecting a change in photoreceptor cell development and/or function comprises characterizing the expression of Nrl in the cell. In some embodiments, detecting a change in photoreceptor cell development and/or function comprises characterizing the expression of one or more biomarkers selected from the group comprising a gene presented in FIG. 11, a gene presented in FIG. 12, or a gene presented in FIG. 13. In some embodiments, detecting a change in photoreceptor cell development and/or function comprises characterizing the ability of the photoreceptor cell to make synaptic connections (e.g., with downstream targets in a retina). In some embodiments, detecting a change in photoreceptor cell development and/or function comprises characterizing the ability of the photoreceptor cell to integrate within a retina. In some embodiments, detecting a change in photoreceptor cell development and/or function comprises characterizing the ability of the photoreceptor cell to respond to a synapse-dependent stimulus. Thre present invention is not limited by the type of test compound characterized. In some embodiments, the test compound is selected from the group comprising a carbohydrate, a monosaccharide, an oligosaccharide, a polysaccharide, an amino acid, a peptide, an oligopeptide, a polypeptide, a protein, a nucleoside, a nucleotide, an oligonucleotide, a polynucleotide, a lipid, a retinoid, a steroid, a drug, a prodrug, an antibody, an antibody fragment, a glycopeptide, a glycoprotein, a proteoglycan, a small molecule organic compound, or mixtures thereof. In some embodiments, the non-human animal is a rodent. In some embodiments, the rodent is a mouse.

The present invention also provides a method of identifying a photoreceptor cell comprising: providing a cell; and detecting Nrl promoter activity. In some embodiments, the presence of Nrl promoter activity identifies the photoreceptor cell as a rod photoreceptor. In some embodiments, the photoreceptor cell is a photoreceptor precursor cell.

The present invention also provides a method of converting a non-rod cell to a rod photoreceptor cell comprising altering Nrl expression and/or activity in the non-rod cell. In some embodiments, altering Nrl expression and/or activity comprises expressing heterologous Nrl nucleic acid in the cell. In some embodiments, altering Nrl expression and/or activity comprises inducing Nrl expression with a small molecule. The present invention is not limited by the small molecule utilized. Indeed, a variety of small molecules may be utilized to induce Nrl expression and/or activity including, but not limited, test compounds identified using compositions and methods of the present invention. In some embodiments, the small molecule is retinoic acid. In some embodiments, altering Nrl expression and/or activity comprises altering the post-translational modification of Nrl. For example, in some embodiments, phosphorylation of Nrl is altered. In some embodiments, altering Nrl expression and/or activity alters the expression of one or more gene targets of Nrl. In some embodiments, the gene target is Nr2e3.

### DESCRIPTION OF THE DRAWINGS

FIG. 1 shows that the Nrl promoter directs GFP expression to rods and pineal gland in transgenic mice. (a) Nrl-L-EGFP construct. The upstream Nrl segment contains four sequence regions I-IV that are conserved between mouse and human. E1 represents exon 1. (b) Immunoblot of tissue extracts (as indicated) using anti-GFP antibody, showing retina-specific expression of GFP in the Nrl-L-EGFP mouse. c) GFP expression in the pineal gland of Nrl-L-EGFP transgenic mice. (d) GFP expression in outer nuclear layer (ONL) of entire adult retina with (e) some nonfluorescent cells in the outer part of the ONL. (f-h) Immunostaining with rhodopsin antibody showing a complete overlap with GFP expression. (i-k) Cells positive for the cone-specific marker peanut agglutinin do not overlap with GFP-expressing cells. (l-n) Immunostaining with cone arrestin reveals no overlap with GFP. Arrowheads indicate cone photoreceptor cells. As shown, GFP specifically labels the rod population in the retina. RPE, retinal pigment epithelium; OS, photoreceptor outer segments; IS, inner segments; ONL, outer nuclear layer; INL, inner nuclear layer; GCL, ganglion cell layer. (Scale bar, 100 μm (c), 500 μm (d), and 25 μm (e-n)).

FIG. 2 shows the time course of GFP expression corresponds to rod cell birth in developing mouse retina. (a) RT-PCR analysis showing the expression of Nrl and Rho transcripts in developing and adult mouse retina, compared to an Hprt control. E and P indicate embryonic and postnatal day, respectively. W and M represent age in weeks and months, respectively. (b) GFP expression is first observed at E12 in a few cells with longer exposure (b′). (c and c′) Short and long exposures at E14, respectively. (d-g) Progressive increase in the intensity and number of GFP-expressing cells from E16 to P4. (h) Low-magnification view at E16 showing a dorsoventral gradient of GFP expression. (i) Timeline of rod photoreceptor birthdates, major developmental events, and the kinetics of Nrl and rhodopsin (Rho) gene expression. VZ, ventricular zone; NBL, neuroblastic layer. (Scale bars, 25 μm (b-g) and 500 μm (h).)

FIG. 3 shows GFP is expressed shortly after cell cycle exit. (a-c) E16 retinas from the wt-Gfp mice immunostained with antiphosphohistone H3 (pH3) and anti-GFP antibody. There is no colocalization, indicating that GFP+ cells are not in M-phase. (d-l) BrdUrd labeling experiments. (d-f) One hour after BrdUrd injection, no GFP+ cells (arrowheads) were labeled with BrdUrd, demonstrating that GFP+ cells are not in S-phase. (g-i) After 4 h, a small number of colabeled cells (arrows) were observed, indicating that GFP expression starts ˜4 h after the end of S-phase. (j-l) The number of colabeled cells increased 6 h after BrdUrd injection. VZ, ventricular zone; RPE, retinal pigment epithelium. (Scale bars, 10 μm.)

FIG. 4 shows that GFP colocalizes with S-opsin in photoreceptors of the Nrl-ko-Gfp retina. (a) wt-Gfp and Nrl-ko-Gfp retinas (at P6) were immunostained with anti-S-opsin antibody. GFP and S-opsin are colocalized in the Nrl-ko-Gfp but not in the wt-Gfp mouse retina. (b) Dissociated cells from the P10 Nrl-ko-Gfp mouse retina were immunolabeled with S-opsin antibody. Bisbenzimide labels the nuclei. All GFP+ cells express S-opsin. However, ˜40% of S-opsin+ cones do not express GFP. This may reflect the loss of GFP during dissociation and immunostaining; decreased GFP expression in the absence of Nrl, which can activate its own promoter in mature rods; and/or contributions from the cohort of normal cones. Thus, GFP+ cells from the wt-Gfp and Nrl-ko-Gfp retina represent pure populations of rods and cones, respectively. (Scale bars, 50 μm (a) and 10 μm (b).)

FIG. 5 shows gene profiles of FACS-purified GFP+ photoreceptors reveal unique differentially expressed genes and significant advantages over whole retina analysis. (a) Bitmap for gene expressions. The 45,101 probesets were determined as present (black) or absent (white) at each of five developmental stages; all genes were assigned to one of the 2⁵=32 possible expression clusters, which are represented by black/white patterns and correspond to 32 rows in the bitmap. The bitmap of gene expression profiles for wild-type developing rods is shown, with the number of genes in each cluster indicated. The boxed clusters represent molecular signatures for each developmental stage. A similar bitmap was generated for developing cones from the Nrl-ko-Gfp retina. (b) Comparison of gene profiling data from FACS-purified photoreceptors with those from the whole retina (See, e.g., Yoshida et al., (2004) Hum. Mol. Genet 13, 1487-1503). The two data sets were analyzed by using FDR-CI with 2-fold maximum acceptable difference (MAD) constraint. The horizontal axis represents the sorted gene index according to FDR P values, and the vertical axis represents FDR P values. At similar FDR P values, >10 times more differentially expressed genes are extracted in the profiling data identified in the present invention compared to Yoshida et al. (See, e.g., Yoshida et al., (2004) Hum. Mol. Genet 13, 1487-1503), thereby allowing for much stronger discovery power. (c) SOM clustering of selected wt (wt-Gfp) gene expression profiles. Clusters of top 1,000 differentially expressed genes over five developmental stages were projected onto a 2D 2×4 grid. Within each image, expression levels are shown on y axis and the five developmental stages (in a) are shown on x axis from left to right (from earliest to latest). The middle curve is the mean expression profile of genes in that cluster, and the upper/lower curves show the standard deviation (±). The cluster index (c#) and the number of genes in each cluster are indicated. The cluster containing rhodopsin includes genes whose expression increases progressively as photoreceptors mature, from P6 to adult. (d) SOM clustering of selected Nrl^{-/-} (Nrl-ko-Gfp) gene expression profiles. The details are essentially the same as in c.

FIG. 6 shows cluster analysis of differentially expressed genes. (a) Hierarchical clustering of top 1,000 differentially expressed genes across wt, Nrl-ko, and five developmental stages, selected by two-stage filtering. (b) Cluster I includes genes that exhibit increased expression during cone development and show dramatically increased expression in the Nrl^{-/-} photoreceptors, such as Opn1sw (S-cone opsin), Gnb3 (cone transducin), and Elovl2 (long-chain fatty acid synthase). (c) Cluster II includes genes that exhibit increased expression during rod development and show dramatically reduced expression in the cones, such as Rho (rhodopsin), Nr2e3 (nuclear receptor, mutated in rd7 mice), Pde6b (rod GMP phosphodiesterase 6B, mutated in rd1 mice), and Nrl.

FIG. 7 shows expression of Nrl and GFP in the developing retina of the wild-type Gfp (wt-Gfp) mice. RT-PCR analysis shows the expression of Nrl and Gfp transcripts in the wt-Gfp mouse retina at various developmental stages. Hprt serves as control. Embryonic day (E)-b 12-E18 and postnatal day (P)0-P10 indicate embryonic and postnatal day, respectively. A primer set derived from the Nrl promoter and EGFP gene was used as internal control for genomic DNA contamination. Nrl-L-GFP construct (lane G) was used as positive control for this primer set. N is negative control, and L represents a 100-bp ladder.

FIG. 8 shows Expression of cell cycle markers and GFP in P3 wt-Gfp mouse retina. GFP+ cells do not show any labeling with anti-CyclinD1 or anti-Ki67 antibody, providing additional evidence that GFP is expressed in postmitotic cells. VZ, ventricular zone; RPE, retinal pigment epithelium. (Scale bars, 25 μm.)

FIG. 9 shows scatter plots and histograms of flow-sorted dissociated cells from the wt-Gfp mouse retina. In forward (FSC)×side (SSC) scatter plots, yellow dots represent GFP+ cells, pink dots show nonfluorescent cells, and green dots are marginal (noncategorized) cells. The GFP+ cells are significantly smaller (less FSC) than other retinal cells at every stage, consistent with their postmitotic status. In histograms, the gates for GFP+ and GFP− cells were set with a safety margin to avoid crosscontamination. The gate setting was slightly different for each indicated developmental stage (E16 to P28). The number of GFP+ cells increases over time, and this cluster is most distinct in adult retinas. Dissociated retinal cells from an adult Tg(Nrl-L-EGFP):rd1/rd1 mouse [C3H/HeJ (rd1/rd1)], which exhibits extensive photoreceptor degeneration by P28, show no photoreceptor cluster or fluorescence. In a nontransgenic C57BL/6 retina, no photoreceptor fluorescence is detected.

FIG. 10 shows RT-PCR analysis of FACS-purified GFP+ and GFP− retinal cells from indicated stages of development (E16 to P28). β-actin is used as control. Reverse transcriptase (RT) (−) and water lanes serve as control. GFP+ cells from wt-Gfp retina show high expression of rod-specific genes (Nrl, Nr2e3, Rho, and Pdeb), whereas transcripts for genes expressed in cones and other retinal neurons (Arr3, Opn1mw, Grm6, and Thy1) are barely detectable.

FIG. 11 shows a table depicting nonredundant genes in the rhodopsin cluster derived from the top 1,000 genes that were identified by SOM analysis of wt-Gfp developmental gene expression profiles. Average fold change (AFC) in expression in adult GFP+ cells compared to E16 is shown here. Genes associated with human retinopathies are shown in bold.

FIG. 12 shows a table depicting nonredundant genes in the S-opsin cluster derived from the top 1,000 genes that were identified by self-organizing map (SOM) analysis of Nrl-ko-Gfp developmental gene expression profiles.

FIG. 13 shows genes exhibiting altered expression at P6 compared to E16 and P2 rods.

FIG. 14 shows the validation of microarray gene expression profiling using real-time PCR. (a) Thirty-four genes showing differential expression in wt GFP+ rod precursors (E16, early-born rods; P2, late-born rods; and P6, at the time of rhodopsin-expression) were examined by real-time PCR by using GFP+ cells from E16, P2, and P6 retinas. Pearson correlation coefficient was calculated for each gene to quantify the consistency between microarray experiments and real-time PCR. (Left) Distribution of the correlation coefficients. Note that 25/34 genes (including Abca1, Bbs4, Bteb1, Cacna1f, Dkk3, Rdh12, Rpgr, and Tulp4) exhibit high (3/3 time points) to partial (2/3 time points) conformity between the two platforms. To make expression scores measured by microarray and real-time PCR visually comparable (and for presentation), scores were standardized by subtracting mean and dividing standard deviation. Therefore, each gene expression profile over the three developmental stages has a mean of zero and a standard deviation of one. This standardization does not change the correlation of gene expression profiles between two platforms. For selected genes, the standardized expression profiles from the two platforms were then plotted in the same panel for visual comparison. Four different gene comparisons are shown. (b) To validate the results of microarray profiling of GFP+ cells from both wt-Gfp and Nrl-ko-Gfp retinas at five developmental stages, independent samples of GFP+ cells were used at indicated stages for real-time PCR analysis. As stated for a, each gene expression profile over the five developmental stages and for either wild-type or Nrl-ko has a mean of zero and a standard deviation of one. Of the 19 genes examined in both wt-Gfp and Nrl-ko-Gfp samples at all five stages, 10 show complete concordance between the two platforms. Five additional genes exhibit conformity by real-time PCR at three to four of the five developmental stages examined.

FIG. 15 shows morphological integration of P1 retinal cells into immature and adult wildtype recipient retinas. a, GFP-positive P1 donor cells integrated within the ONL of wildtype P1 littermate recipient retinas, three weeks after sub-retinal transplantation. Integrated cells were correctly orientated within the ONL and developed morphological features typical of mature photoreceptors including synaptic boutons (arrow), inner and outer processes (open arrow heads) and inner segments (filled arrow head). b, Low power montage showing the distribution of P1 donor cells integrated within an adult wildtype recipient. Examples of inner (filled arrowheads) and outer segments (open arrowheads) are highlighted. NB. Cytoplasmic localization of GFP is poor in the outer segments of transplanted cells. c, Example of integrated cells in the ONL of adult wildtype retinas. d, example of cells with rod- (open arrow) and cone- (filled arrow) like morphologies. e, Schematic of a mature photoreceptor showing rod morphology and the location of photoreceptor-specific proteins. ONL=outer nuclear layer; INL=inner nuclear layer; IS=inner segments. Scale bar 10 μm.

FIG. 16 shows that transplantation occurs via integration not cell fusion. a, Single confocal sections, taken at the same confocal plane, through the inner segment (arrow) of a GFP-positive cell integrated within a CFP-positive recipient retina. Far right, cross hairs show an absence of CFP fluorescence at the location of the GFP-positive inner segment. b, Integrated cells only have a single nucleus derived from the donor cell. Donor cells were pre-labelled with BrdU 24-48 hrs prior to transplantation into a non-labelled host. Image shows an integrated cell with a single nucleus that was BrdU-positive, demonstrating that it originated from the donor animal. Scale bar 10 μm.

FIG. 17 shows E11.5 cells express markers of progenitor cells. Confocal images of dissociated E11.5 GFP-positive cells stained for the progenitor markers nestin and Pax6 (both 1:20; Developmental Studies Hybridoma Bank) and Sox2 (1:200; AbCam) (red). Scale bars 10 μm.

FIG. 18 shows optimal ontogenetic stage of donor cells is post-mitotic photoreceptor precursor. a, Histogram showing the number of integrated cells as a function of donor age (mean±S.E.M) following sub-retinal injection into adult wildtype recipients. b-c, P1 donor cells transplanted into an adult recipient, which subsequently received repeated BrdU injections. b, donor cells continued to proliferate within the subretinal space, as indicated by BrdU labelling (red; arrowheads). c, integrated cells were not BrdU labeled. d-e, Examples of FACSsorted Nrl.gfp-positive post-mitotic rod precursor cells integrated within the ONL of adult retinas. Scale bars 10 μm.

FIG. 19 shows photoreceptor identity and synaptic connectivity of integrated cells. a-c, confocal projection images of retinal sections from adult wildtype mice 3 weeks post-transplantation with P1 donor cells. Sections were stained with primary antibodies against (a) phosducin, (b) bassoon and (c) Protein Kinase C (PKC). a, phosducin is expressed throughout the cytoplasm including the synapse but is predominantly located in the inner/outer segments. Inserts, high power confocal image through the synaptic bouton and inner segment regions, taken through the region of GFP expression only. b, bassoon, a pre-synaptic anchoring protein associated with ribbon synapses. Note two cells have integrated adjacent to each other (arrow heads) and their synapses are juxtaposed to one another (arrows). Insert, high power confocal image taken through the region of GFP expression of one of the two synapses. c, image shows the synaptic bouton of an Nrl.gfp-positive integrated cell contacting a PKC-positive rod bipolar cell from the recipient retina. Insert, high power confocal image of the synapse. Scale bar 10 μm. d-f, integrated cells respond to the stimulation of the rod-specific glutamate receptor, mGluR8. d, tangential confocal section through the inner level of the ONL of a recipient retina loaded with the calcium indicator FURA-RED, showing the cell bodies of integrated Nrl.gfp^{+/+} cells and host cells selected at random for analysis. Nrl.gfp^{+/+} donors were used to ensure responses were recorded from rod photoreceptors. e, stimulation of mGluR8 causes a decrease in [Ca²⁺]i, which can be blocked by the specific antagonist CPPG. NB, when collected at 660±50 nm, the emission of Fura-Red undergoes an increase in fluorescence as [Ca²⁺]i decreases. f, histogram showing the % of integrated Nrl.gfp-positive cells and recipient photoreceptors that responded to DCPG, DCPG+CPPG, or the agonist NMDA which activates NMDA-receptors, a subtype not usually expressed by photoreceptors.

FIG. 20 shows E11.5 cells survive and are able to differentiate in the subretinal space of adult host retinas. a, Example of unsorted E11.5 cells from an Nrl.gfp^{+/+} donor transplanted into the sub-retinal space of adult wildtype hosts, three weeks post-injection. The cells consistently failed to integrate. However, some form rosette-like structures in the sub-retinal space and start to express Nrl, as indicated by GFP fluorescence. b, differentiated cells express the late photoreceptor marker, rhodopsin when arranged as rosettes. Scale bars 10 μm.

FIG. 21 shows integration and restoration of light sensitivity in degenerating recipient retinas. a, b, integration into the peripherin-2 deficient rds mouse. a, left, Low power image showing co-localization of peripherin-2 staining with GFP-positive cells (arrows) transplanted into an adult rds mice. Peripherin-2 is absent in the mutant retina. Highlighted region shown enlarged, right. b, peripherin-2 expression is maintained at least 10 weeks post-transplantation. Highlighted region shown enlarged, right. c, left, image showing co-localization of rhodopsin staining with GFP-positive cells (arrows) three weeks after transplantation into a 4 wk old rho^{-/-} recipient. Highlighted region shown enlarged, right. NB. cytoplasmic localisation of GFP is poor in the outer segments of GFP cells. Scale bars 10 μm. de, light-evoked extracellular field potentials in the ganglion cell layer of transplanted retinas. d, graph shows the shift in response threshold in treated (Nrl.gfp^{+/+}/rho^{+/+} cells) versus sham-injected (rho^{-/-} cells) eyes. Average light intensity plots were made from all eyes tested and the threshold for a light-evoked response was determined as being the stimulus intensity that evoked a response magnitude that was 10% of the potential evoked by the maximum stimulus. Light intensity plots for uninjected wildtype (circles) and rho^{-/-} (diamonds) eyes are shown for comparison. e, representative recordings from treated and sham-injected eyes of the same animal. Traces show averaged voltage responses to light stimuli of increasing intensity. f-i, light-evoked pupillary responses in transplanted eyes. f, example of light-evoked pupil response, where infra-red images show the pupil area measured in dark (a0; top) and in illumination (ai; bottom). Images correspond to shaded circles in(g). g-h, pupillary response plots [(ai/a0) against log (i)] for an uninjected wildtype mouse (g), and a rho^{-/-} mouse (h) that received Nrl.gfp (rho^{+/+}) cells in one eye and a sham injection (rho^{-/-} cells) in the other. Note the increased sensitivity of the Nrl.gfp (rho^{+/+})-injected eye compared with the sham-injected eye. i, the difference in log irradiance required to elicit a 50% pupil constriction between the transplanted eye and sham-injected control eye (δi) is plotted against the number of integrated rod photoreceptors identified histologically. Increasing values on the y-axis represent an increase in the sensitivity of the treated eye, relative to the sham-injected eye. There is a significant correlation between the number of cells integrated and the sensitivity of the pupil response (Pearson product moment correlation co-efficient R=0.87, P=0.0013).

FIG. 22 shows transplantation into the rd mouse. Confocal projection images of P1 GFP-positive cells transplanted into the rd mouse subretinal space. The transplanted cells persist at 3 weeks post-transplantation but adopt variable morphologies due to the collapse of the surrounding host ONL. Scale bar 10 μm.

FIG. 23 shows (A, B) confocal micrographs of retinas from mice that had received an intraperitoneal injection of MNU 1 week prior or age-matched control mice stained with Cytox blue and anti-VGluT1 antibody. VgluT1 and Cytox blue immunoreactivity was observed in the inner plexiform layer (IPL), outer plexiform layer (OPL) and ganglion cell layer (GCL), inner nuclear layer (INL), outer nuclear layer (ONL), respectively, in the control retina whereas immunoreactivity was localized in the IPL and GCL, INL, respectively in the MNU-treated retina, indicating that the photoreceptor layer had been completely destroyed. SUB, subretina. Scale bars, 20 μm. (C, D) Representative dark-adapted ERG recordings from MNU-treated mice or age-matched control mice at that time point. Note the ERG trace from mice 1 week after MNU injection does not detect a response.

FIG. 24 shows (A, B) representative fluorescence images of retinal sections at the site of injection double-stained with CS-56 and GFAP from MNU-treated mice 2 days after vehicle injection or non-transplanted MNU-treated mice. Note that the expression of CS-56 and GFAP are characteristics of host glial scarring at the margin of host retina around the transplantation site.

FIG. 25 shows (A, B) confocal micrographs of retinal sections stained with Cytox blue from MNU-treated mice 4 weeks after transplantation with or without chondroitinase treatment. The majority of the grafted Nrl-GFP+ photoreceptor cells are distributed at the outer margin of the host retina in both groups. R, retina; RPE, retinal pigment epithelium. Scale bars, 100 μm. (C, D and insets) High magnification of confocal micrographs shown in (A, B). Arrows indicate examples of graft-derived neurites sprouting into the host retina (C and inset), a phenomenon rarely observed when in transplants without chondroitinase treatment (D and inset). Scale bars: C, D, 20 μm. (E) Quantification of cell distribution patterns in transplanted MNU-treated mouse subretina at 4 weeks after transplantation. (F) Comparison of the ratio of GFP-positive cells that were distributed at the outer margin of the host retina where the photoreceptor layer had originally existed to all the GFP+ cells residing within the entire host retina. (G) Comparison of the ratio of GFP-positive cells bearing neurites to the GFP-positive cells integrated in host retina. (H) Comparison of the ratio of GFP-positive cells sprouting neurites toward the host retina to the integrated GFP-positive cells. Statistical significance: *P<0.05. (I,J) Confocal micrographs of retinal sections immunolabeled for CS-56 from MNU-treated mice 4 weeks after transplantation with or without chondroitinase treatment. An arrow indicates an example of graft-derived neurite that extended the CSPG-rich ECM at the outer margin of host retina and entered the host retina in Nrl/ChABC group (I). Note that those graft-derived neurites failed to cross the CSPG-rich ECM without chondroitinase treatment (J). Scale bars, 5 μm.

FIG. 26 shows (A, B) confocal micrographs of retinal sections immunolabeled for VGluT1 obtained from MNU-treated mice 4 weeks after transplantation with or without chondroitinase treatment. Arrows indicate examples of graft-derived neurite colocalizing with VGluT1 in the Nrl/ChABC group (A), a phenomenon that was rarely observed in the Nrl group (B). Scale bars, 5 μm. (C) Three-dimensional analysis of a z-series of confocal images from sections stained for VgluT1 shown in (A, arrows). The two-color colocalization obtained in the x-y plane was also verified by two-dimensional cross-sectional images (x-z scan, y-z scan).

FIG. 27 shows (A,B) dark-adapted, full-field ERGs from a MNU-treated eye that had received a transplant with ChABC 4 weeks before compared with the contralateral eye. Representative case of an ERG trace in a cell transplanted eye with chondroitinase treatment (A) and the non-responsive ERG trace in the contralateral control eye. Note that a-wave-like response increased proportionally to the extent of light intensity (ND0-ND3).

FIG. 28 shows examination of the rd16 mouse retina. (A) Fundus photographs of WT C57BL/6J mouse and the rd16 homozygote mutants (rd16/rd16) demonstrating retinal degeneration at 1 month of age and at 2 months. (B) ERG responses of WT and mutant (rd16/rd16) mice under dark- (SCOTOPIC) and light- (PHOTOPIC) adapted conditions. Arrows indicate the A-wave and arrowheads the B-wave. (C) Histology of retina of WT and rd16 homozygotes mice at indicated ages. RPE, retinal pigment epithelium; OS, outer segments; IS, inner segments; ONL, outer nuclear layer; OPL, outer plexiform layer; INL, inner nuclear layer; GCL, ganglion cell layer.

FIG. 29 shows Cep290 mutation in rd16. (A) Linkage cross-data: 165 back-cross progeny from the (rd16×CAST/EiJ)F1×rd16/rd16 were phenotyped for ERG phenotype and genotyped for the indicated microsatellite markers. Black boxes represent homozygosity for rd16-derived alleles and white boxes represent heterozygosity for rd16- and CAST-derived alleles. The number of animals sharing the corresponding haplotype is indicated below each column of squares. The order of marker loci was determined by minimizing the number of crossovers. The rd16 locus was inferred from the ERG phenotype of mice showing recombinations. (B) Genetic map of mouse chromosome 10 showing the rd16 critical region, which is syntenic to human chromosome 12q21.1. (C) Real-time RT-PCR analysis of BC004690 (Cep290, exons 27-48) in the retina of WT mice. The expression levels at different developmental stages were calculated as relative fold change with respect to embryonic day, E14, after normalization to Hprt levels. P, postnatal day. Each bar represents the mean±SE. (D) Real-time RT-PCR analysis of BC004690 in the retina of Crx^{-/-} and Nrl^{-/-} versus WT mice. The expression levels in the Crx^{-/-} and NH^{-/-} retina were calculated as percentage of the level in the WT mouse retina after normalization to Hprt levels. Each bar represents the mean±SE. (E) RT-PCR analysis (with F2-R2 primer set) of BC004690 using rd16 and WT retinal RNA. A 1.2 kb band is detected in rd16 compared with a 2.1 kb product in WT. DNA size markers are shown on the left (in kb). (F) BC004690 sequence in rd16 showing an in-frame deletion of 897 by encompassing exons 35-39. Three-letter codes for amino acids were used. (G) Southern analysis of WT and rd16 DNA using an exon 34 probe. DNA was digested with EcoRV, which cuts the WT DNA five times between exons 34 and 40, whereas in the rd16 DNA, only three EcoRV sites remain. WT DNA gave the expected band of 10.6 kb, whereas with the rd16 DNA, a heavier band at ˜15 kb (indicated by arrows) is seen. Molecular weight markers are in kilobases. (H) Schematic representation of the Cep290 gene and the CEP290 and ΔCEP290 proteins showing putative domains and motifs. CC, coiled-coil; KID, RepA/Rep+ protein KID; P-loop, ATP-GTP-binding site motif A; spindle association (SA) domain; MYO-Tail, myosin tail homology domain.

FIG. 30 shows evolutionary conservation of CEP290. CLUSTAL analysis of protein sequences from different species was performed using the CLUSTALW alignment program. The CEP290 protein is conserved in evolution, with the region that is deleted in rd16, showing high degree of identity (shaded amino acid sequence) among mammalian species (Alignment scores between 83% and 89%). Major putative domains and motifs are represented with bars. The deletion removes majority of the myosin-tail homology domain and KID domains I and II.

FIG. 31 shows expression and localization of CEP290. (A) COS-1 cells were transfected with empty vector (mock) or a vector encoding full-length human CEP290 protein fused to a myc-tag. Cells were lysed and analyzed by immunoblotting (IB), using anti-myc (upper panel) or anti-CEP290 antiserum (lower panel). Arrows indicate specific bands. The immunoreactive band in the mock transfected lane (lower panel) is endogenous CEP290 protein. Pre-immune serum showed no signal. (B) Immunoblots of protein extracts from WT (20 μg) and rd16 (200 μg) retina were analyzed using CEP290 antibody. Arrows indicate the full length and predicted alternatively spliced products of CEP290. (C) Immunohistochemical analysis of WT mouse retina. The sections were incubated with the CEP290 antibody followed by secondary antibody incubation. (a) and (c) Nomarski image of the retinal sections. (b) and (d) Staining with the CEP290 antibody (green) reveals intense labeling of the connecting cilium (indicated by arrows). Labeling in the IS is also observed. Scale bar: 40 μm for (a), (b); 10 μm for (c), (d). (D) CEP290 co-localizes with γ-tubulin (upper panel) and PCM1 (lower panel) at the centrosomes (arrows; merge) in IMCD-3 cells. Bisbenzimide (BIS) was used to stain the DNA. (E) CEP290 is associated with centrosomes during cell cycle. Synchronized HeLa cells were co-stained with antibodies against γ-tubulin and CEP290 and analyzed by confocal microscopy. Arrows indicate the centrosomal staining of CEP290 (merge) at all indicated stages of cell division. (F) IMCD-3 cells were transfected with p50-dynamitin expression construct. Cells were stained with p50, CEP290 or γ-tubulin antibodies. Arrows denote centrosomal CEP290 and γ-tubulin in untransfected cells, whereas arrowheads denote the localization of CEP290 and γ-tubulin to centrosomes in p50-overexpressing cells. Merge image shows nuclear staining.

FIG. 32 shows immunogold labeling of CEP290 in WT mouse retina. The signal is concentrated in the connecting cilium (CC) (see inset); although some labeling is detected in the inner segments (IS) and outer segments (OS) as well. Quantitative analysis of the label revealed a four times higher concentration of CEP290 in the connecting cilium than that in the IS and OS of mouse retina.

FIG. 33 shows CEP290 and ΔCEP290 associate with RPGR-ORF15 and other centrosomal/microtubule-associated proteins in the retina. (A, B) IP was performed using ORF15^{CP} (A), CEP290 (B) antibodies or normal IgG from WT and rd16 retinal extract (200 μg each). The immunoprecipitated proteins were analyzed by IB using CEP290 (A) or ORF15^{CP} (B) antibodies. Input lane contains 20% of the protein extract used for IP. Longer exposure of the blot in (A) shows an immunoreactive band for ΔCEP290 in rd16 input lane. Molecular weight markers are shown in kilo Daltons (kD). Asterisk indicates the faint full-length CEP290-immunoreactive band (290 kDa) immunoprecipitated from the WT retina using the ORF15^{CP} antibody. Arrow in (A) points to the ACEP290 protein immunoprecipitated from rd16 retina using ORF15^{CP}. Arrows in (B) indicate multiple RPGR-ORF15 isoforms recognized by the ORF15^{CP} antibody (See, e.g., Khanna et al., (2005) J. Biol. Chem., 280, 33580-33587). Less high molecular weight (120-220 kDa) RPGR-ORF15 isoforms are immunoprecipitated by the CEP290 antibody in rd16. (C) Immunocytochemistry using the CEP290 and ORF15^{CP} antibodies shows co-localization of endogenous CEP290 and RPGR-ORF15 in IMCD-3 cells. Arrows indicate co-localization (Merge). (D) WT and rd16 retinal extracts were subjected to IP using the CEP290 antibody and analyzed by immunoblot (IB) using indicated antibodies. Input lane represents 5% of the total protein extract used for immunoprecipitation (IP). Molecular weight markers are shown in kD. Lanes 1 and 2: input from WT and rd16 retinal extracts; 3 and 4: IP using the CEP290 antibody from WT and rd16, respectively; 5: IP with normal IgG from WT retina. (E) Reverse IP was performed by incubating protein extracts of WT retina with indicated antibodies for IP followed by IB using the CEP290 antibody. Molecular weight markers are shown in kD.

FIG. 34 shows localization of RPGR-ORF15, rhodopsin and arrestin in rd16 retinas. (A-D) Immunogold EM of WT or rd16 retinas with indicated antibodies. Labeling with ORF15^{CP} antibody showed a predominant connecting cilium (CC) staining of RPGR-ORF15 (A) as opposed to abnormal extensive labeling throughout the photoreceptor IS in the rd16 retina (B, C). Arrows indicate clusters of immunogold particles. Labeling of rhodopsin in the rd16 retina (D) is evident around the photoreceptor cell bodies (indicated by arrows) with no exclusive OS localization; N, nucleus. (E, F) Immunohistochemical analysis of the WT and rd16 retinas at P12, dissected under normal light/dark cycle, with antibodies against rhodopsin (E) or arrestin (F). As shown, both rhodopsin and arrestin are localized primarily in the OS of WT retina, whereas in rd16, rhodopsin and arrestin are also detected in the ONL and ISs of photoreceptors. OS in the rd16 retina degenerate at P12 and therefore are represented in conjunction with the inner segments (OS/IS). Scale bar: 50 μm.

FIG. 35 shows temporal and spatial expression of NR2E3 in the Crx::Nr2e3/Nrl^{-/-} transgenic mice. (A) Crx::Nr2e3 construct. (B) Southern analysis of genomic DNA from Nrl^{-/-} (lane 1) and Crx::Nr2e3/Nrl^{-/-} (lane 2) mice. The endogenous Nr2e3 gene is represented by a 9 kb and the transgene by a 2.8 kb band. (C) Immunoblot analysis of neural retina extract shows the temporal expression of NR2E3 in the Crx::Nr2e3/Nrl^{-/-} mice during the early developmental stages, compared with Nrl^{-/-} and WT mice. γ-tubulin is used as an internal control. (D) Immunostaining with anti-NR2E3 antibody (indicated as arrowhead) showing spatial expression of NR2E3 in the Crx::Nr2e3/Nrl^{-/-} mice, compared with WT and Nrl^{-/-} mice, at E11, E16, E18 and 4 week. In the WT retina, NR2E3 is expressed only in the rods and not cones. In the Crx::Nr2e3/Nrl^{-/-} retina, NR2E3 is expressed in both rods and cones because of the Crx promoter used. (E) Immunostaining with anti-NR2E3 and BrdU antibodies after 1 h pulse of BrdU injection at E16. No colocalization is observed in the retinal section. ON, optic nerve; NR, neural retina; D, dorsal; L, lens; V, ventral; NBL, neuroblastic layer; ONBL, outer neuroblastic layer; INBL, inner neuroblastic layer; RPE, retinal pigment epithelium; RGC, retinal ganglion cells. Scale bars are indicated.

FIG. 36 shows IHC of photoreceptor markers in the WT, Nrl^{-/-} and Crx::Nr2e3/Nrl^{-/-} mice. (A-C) Immunostaining with anti-S-opsin (A), M-opsin (B), cone arrestin (C) and rhodopsin antibodies. Rhodopsin is detected in the ONL and OS of the WT and Crx::Nr2e3/Nrl^{-/-} retina. S-opsin and cone arrestin are enriched in the Nrl^{-/-} retina but are undetectable in the Crx::Nr2e3/Nrl^{-/-} retina. M-opsin is undetectable in the transgenic mice. RPE, retinal pigment epithelium; RGC, retinal ganglion cells. Scale bars are indicated.

FIG. 37 shows rescue of rod morphology but not function in the Crx::Nr2e3/Nrl^{-/-} mice by NR2E3. (A) Toluidine blue staining of the retina section demonstrates that the nuclei of photoreceptors in the Crx::Nr2e3/Nrl^{-/-} retina exhibit a rod-like morphology, unlike the cones observed in the Nrl^{-/-} retina. Arrows in the WT section refer to staining of cone nuclei. (B) TEM shows closed discs with distorted orientation in the photoreceptor outer segments of the Crx::Nr2e3/Nrl^{-/-} retina, compared with WT and Nrl^{-/-} mice. Arrows indicate OS membrane surrounding the discs, whereas arrowheads indicate the open discs of cones. (C) Light-adapted, spectral ERGs that evoke nearly matched responses from S-cones (360 nm, black traces) or M-cones (510 nm, gray traces) in WT are not detectable in a Crx::Nr2e3/Nrl^{-/-} mouse and are largely mismatched in Nrl^{-/-}. (D) Spectral ERG amplitudes demonstrate the enrichment of S-cone activity (360 nm) in Nrl^{-/-} mice compared with WT. Crx::Nr2e3/Nrl^{-/-} mice (gray symbols) show responses indistinguishable from noise. (E) Dark-adapted ERGs evoked by increasing intensities of blue flashes in Nrl^{-/-} mice show elevated thresholds (by ˜3 log units) compared with WT. The Crx::Nr2e3/Nrl^{-/-} mouse shows no detectable ERGs. (F) Leading edges of dark-adapted ERG photoresponses evoked by a pair of white flashes (3.6 log scot-cd.s.m⁻²) presented 4 s apart and fit with a model of phototransduction activation (smooth grey lines). In WT mice, rods dominate the first flash photoresponse (dark line); the paired-flash has a smaller, cone-mediated response (grey line). In Nrl^{-/-} mice, dark-adapted photoresponses are smaller and slower than WT; the paired-flash response closely tracks the first flash response. ERG photoresponses are not detectable in the Crx::Nr2e3/Nrl^{-/-} mice. RPE, retinal pigment epithelium; IS, photoreceptor inner segment. Scale bars are indicated.

FIG. 38 shows qPCR analysis of the selected phototransduction genes. qPCR analysis using WT, Nrl^{-/-} and Crx::Nr2e3/Nrl^{-/-} retinal RNA shows that the expression of cone-specific genes is suppressed while those of rod genes, except Gnat1, restored to varying degree. Expression levels are normalized to the housekeeping gene Hprt first and then compared with WT. Error bars show the standard deviation. The actual fold change of gene expression levels revealed by microarray assays is shown in the table. NC, no change. Gene symbols are: M-opsin or green cone opsin (Opn1mw), S-opsin or blue cone opsin (Opn1sw), cone arrestin (Arr3), cone transducin (Gnat2), phosphodiesterase 6c (Pde6c), chloride channel calcium-activated 3 (Clca3), rhodopsin (Rho), cyclic nucleotide-gated channel a-1 (Cnga1), phosphodiesterase b subunit (Pde6b) and rod transducin (Gnat1).

FIG. 39 shows Nrl-knockout (Nrl::GFP/Nrl^{-/-}) versus WT (Nrl::GFP/WT) retina; and (ii) NR2E3-expressing (Nrl::GFP/Crx::Nr2e3/Nrl^{-/-}) transgenic versus Nrl-knockout (Nrl::GFP/Nrl^{-/-}) retina. FACS-sorted GFP+cells from 4-week-old mouse retina were used for gene profiling. Only genes with a minimum fold change of 4 and FDRCI P-value of <0.1 from comparison (ii) are selected. AFC, average fold change; NC, no change.

FIG. 40 shows IHC of photoreceptor markers in the Nrl^{-/-}/Crx^{-/-} and Crx::Nr2e3/Nrl^{-/-}/Crx^{-/-} mice. Immunostaining with anti-S-opsin and rhodopsin antibodies, showing that S-opsin is increased and rhodopsin is absent in the Nrl^{-/-}/Crx^{-/-} retina. However, in the Crx::Nr2e3/Nrl^{-/-}/Crx^{-/-} retina, S-opsin is absent and rhodopsin is expressed. RPE, retinal pigment epithelium; RGC, retinal ganglion cells. Scale bars are indicated.

FIG. 41 shows Crx::Nr2e3 transgene in the WT background. (A) Immunostaining with anti S-opsin, M-opsin, cone arrestin and rhodopsin antibodies of WT, and Crx::NR2e3/WT retina shows that cone markers are undetectable in the transgenic mice. (B) Toluidine blue staining of the WT and Crx::Nr2e3/WT retina demonstrates the cone-like nuclear staining (indicated by arrows) in the WT retina but not in the transgenic mice. The image in black rectangle shows higher magnification. (C) Anti-BrdU labeling of 3 week retina after a single injection of BrdU at E14. The amount of strongly BrdU-labeled cells in the ONL is not significantly different between WT and transgenic groups. In WT mice, these cells are located to either outer or inner part of ONL, with cells in the outermost regions co-localizing with S-opsin. However, in the transgenic retina, most of these cells are present in the inner part of ONL. Dashed lines demonstrate the inner and outer half of the ONL. (D) Crx::Nr2e3/WT mice show normal rod function but undetectable cone function. Rod ERGs elicited by a dim (b-wave) and bright flash (a-wave) in the dark show similar responses in Crx::Nr2e3/WT and WT mice. A model (smooth gray lines) fit to the responses show normal phototransduction activation. Light-adapted, cone-mediated spectral ERGs (evoked as in FIG. 36C) are not detectable in the Crx::Nr2e3/WT mouse. RPE, retinal pigment epithelium; IS, photoreceptor inner segment; RGC, retinal ganglion cells. Scale bars as indicated.

FIG. 42 shows dual function of ectopically expressed Nr2e3 in the S-opsin::Nr2e3 transgenic mice. (A) S-opsin::Nr2e3 construct. (B) Southern blotting of genomic DNA from Nrl2/2 (lane 1) and S-opsin::Nr2e3/Nrl^{-/-} (lane 2) mice. The endogenous Nr2e3 gene is represented by a 9 kb and the transgene by a 2.8 kb band. (C) Immunoblot analysis of neural retina extract shows the expression of NR2E3 protein in the S-opsin::Nr2e3/Nrl^{-/-} mice at P6, compared with the Nrl^{-/-} and WT mice. γ-tubulin is used as an internal control. (D) Immunostaining with anti-NR2E3 antibody (indicated as arrows) showing signal of NR2E3 staining in S-opsin::Nr2e3/Nrl^{-/-} mice (c), compared with WT (a) and Nrl^{-/-} mice (b), at P6. (E) Toluidine blue staining of the retina section demonstrates that several nuclei of photoreceptors in S-opsin::Nr2e3/Nrl^{-/-} mouse change from cone-like to rod-like morphology. Photoreceptors in the Nrl^{-/-} retina exhibit cone morphology (see FIG. 36A). Rod-like nuclei are indicated by arrows. (F) TEM shows closed discs with distorted orientation in the photoreceptor outer segment of the S-opsin::Nr2e3/Nrl^{-/-} mouse, compared with WT and Nrl^{-/-} mice (see FIG. 36B). Arrows indicate OS membrane surrounding the discs. (G-J) Immunostaining with anti-S-opsin (G, J), M-opsin (H), cone arrestin (I) and rhodopsin antibodies. Rhodopsin is detected in the ONL and OS of the S-opsin::Nr2e3/Nrl^{-/-} retina. No obvious co-localization of S-opsin and rhodopsin is observed in the retinal flat mount (J). (K) Immunostaining with cone photoreceptor marker (S-opsin) antibody in the WT and S-opsin::Nr2e3/WT flat mount retina. Dorsal-ventral pattern of S-opsin gradient is still preserved in the transgenic mice. Reduced numbers of S-opsin positive cells are observed in the S-opsin::Nr2e3/WT retina. (L) Cell counting of S-opsin positive cells on the WT and S-opsin/WT flat mount retina stained with anti S-opsin antibody. S-opsin positive cells were counted in two regions: in the middle of ventral retina (V), and in the middle of dorsal retina (D). A square of 100 mm×100 mm area, indicated in (K) was used to count the S-opsin positive cells and three mice were tested. ONBL, outer neuroblastic layer; INBL, inner neuroblastic layer; RPE, retinal pigment epithelium; IS, inner segments; RGC, retinal ganglion cells; V, ventral; D, dorsal. Scale bars as indicated.

FIG. 43 shows expression of Nrl in cone precursors. (A-L) Toluidine blue stainings of WT (A), Crxp-Nrl/WT (B), Nrl^{-/-} (C), and Crxp-Nrl/Nrl^{-/-} (D) retinal sections demonstrate unique chromatin pattern in the photoreceptor layer for cones (indicated by arrowhead) and rods. Normal laminar structure is observed in both Crxp-Nrl/WT (B) and Crxp-Nrl/Nrl^{-/-} (D) plastic sections. Immunohistochemical markers for rod photoreceptors (rhodopsin) can be detected in WT(E), Crxp-Nrl/WT (F) and Crxp-Nrl/Nrl^{-/-} (II) retina but not in Nrl^{-/-} (G). The pan cone photoreceptor marker, cone arrestin, is present only in WT (I) and Nrl^{-/-} (K) retina, but is largely absent in the Crxp-Nrl/WT (J) and Crxp-Nrl/Nrl^{-/-} (L). (M-P) ERG intensity series and responses were recorded from 2-mo-old WT, Nrl^{-/-}, Crxp-Nrl/WT, and Crxp-Nrl/Nrl^{-/-} mice under dark- (scotopic ERG; M and N) and light-adapted (photopic ERG; O and P) conditions. The x axes for M and O indicate time lapsed after flash. Stimulus energy is indicated (log cd-s/m²). OS, outer segments; IS, inner segments, ONL, outer nuclear layer; INL, inner nuclear layer. (Scale bars=25 μm and 50 μm).

FIG. 44 shows nuclear morphology in the outer nuclear layer of WT (A) and Crxp-Nrl/WT (B) retina. Flat-mounts of retina were stained with the nuclear dye YOYO 1. The focal plane is set at the height of cone nuclei illustrating their larger size and nonhomogeneous chromatin in the wild type retina but not in the Crxp-Nrl/WT retina. (C). Gene expression analysis. Quantitative RT-PCR profiles show loss of conespecific gene expression in both Crxp-Nrl/WT and Crxp-Nrl/Nrl−/− retinas, while rod specific expression is largely unchanged. WT and Nrl−/− retinas show changes in gene expression. Expression levels are normalized to Hprt.

FIG. 45 shows the synaptic organization of the inner retina in the absence of cones. (A) The glutamatergic receptor mGluR6 is clustered selectively at puncta in the OPL, on the dendritic tips of ON bipolar cells, labeled by G0α antibodies. (B) G0α antibody labels the whole population of ON bipolar cells, whereas PKCα labels rod bipolar cells only (RBC). Rod bipolar neurons are therefore double-labeled by both antibodies. ON cone bipolar cells are indicated as CBC). (C) mGluR6 receptors are labeled as puncta located at the dendritic tips of rod bipolar cells. In addition, clusters of mGluR6 are visible in the OPL, but not in association with rod biolar cell dendrites. These clusters are likely to be associated to the dendrites of ON cone bipolar cells. (D) Rod bipolar cells (RBC) are postsynaptic to photoreceptors in the OPL at ribbon synapses (indicated by R). (E) High magnification of one type of cone bipolar cell (CBC). Rod spherules (RS) are indicated. Few dendrites of cone bipolar cells reach the basal aspect of some spherules (arrows); however, many spherules do not appear apposed to CBC dendrites, although these belong to one of the most abundant types of retinal cone bipolar cell. (F). Calbindin staining of the Crxp-Nrl/WT retina shows a normal distribution of intensely labeled horizontal cells and weakly fluorescent amacrine cells with their processes in the IPL. Occasionally, horizontal cell sprouts are observed (arrow). (G). All amacrine cells (the most abundant population of mammalian amacrines) are shown. They exhibit a typical, bistratified morphology. The innermost dendrites terminate in apposition to the axonal endings of rod bipolar cells, stained green by PKCα antibodies. (H) Cholinergic amacrine cells are stained in the transgenic retina by ChAT antibodies. The cells form two mirror symmetric populations of neurons. Axonal complexes of horizontal cells are labeled with neurofilament antibodies. Axonal fascicles of ganglion cells are also intensely stained in the optic fiber layer. (H) Ethidium bromide nuclear staining and ChAT immunostaining demonstrate the normal layering and lamination of the transgenic retina. OS, outer segments; ONL, outer nuclear layer; INL, inner nuclear layer; OPL, outer plexiform layer; IPL, inner plexiform layer.

FIG. 46 shows NK3-R immunostaining of OFF cone bipolar cells in the WT retina. Using NK3-R antibody, the morphology and flat dendritic arbors of OFF cone bipolars are illustrated in WT P20 (A) and 7 month (B) retinas. PNA lectin and NK3-R staining (C) show the proximity of OFF cone bipolars to cone pedicles (inset).

FIG. 47 shows ectopic expression of Nrl in S-opsin-expressing cone photoreceptors. (A and B) Quantification of S-cones in the inferior domain of flat-mounted retinas from WT and BPp-Nrl/WT mice with anti-S-opsin antibody (A) revealed a 40% decrease in S-cones. Light-adapted ERG photoresponses from WT and BPp-Nrl/WT mice are shown in B (photopic b-wave (Left) and photopic b-wave at maximum intensity (Right)). In BPp-Nrl/WT mice, ˜50% reduction in the photopic b-wave amplitude is observed compared with the WT mice. (C-N) Immunostaining of cryosections from Nrl^{-/-} retina show the lack of rhodopsin expression and higher S-opsin expression in the ONL (C-F). In the BPp-Nrl/Nrl−/− retina rhodopsin expression can be detected in the ONL and the OS (G and K). Hybrid photoreceptors expressing both S-opsin (H and L) and rhodopsin can be observed in the ONL, INL, and the GCL (G-N). OS, outer segments; ONL, outer nuclear layer; INL, inner nuclear layer; GCL, ganglion cell layer; BBZ, bisbenzamide. (Scale bar=25 μm and 50 μm).

FIG. 48 shows quantification of photoreceptors and fate mapping experiments. Adult retinas were dissociated, and assayed for rhodopsin and s-opsin expression (A). A schematic illustration of transgenic constructs and breeding for the fate mapping is shown in (B). Presumptive cone precursors showing β-galactosidase immunoreactivity exhibit high degree of coexpression with Cre in the superior domain of the retina (C-E). However, central and inferior domains reveal an increase in β-galactosidase labeled cells that do not overlay with Cre and are presumably rods based on their position in the ONL (F-K).

FIG. 49 shows association of Nrl to cone-specific promoters. (A and B) EMSA. Radiolabeled double-stranded oligonucleotides from Thrb and S-opsin promoters were incubated with RNE, followed by nondenaturing PAGE. Lanes are as indicated. Arrows represent specific shifted bands. Competition experiments were performed with increasing concentration (1-, 5-, or 50-fold molar excess, respectively) of unlabeled specific oligonucleotide or 50-fold higher concentration of nonspecific (ns) oligonucleotide, to validate the specificity of band shift. Anti-NRL or normal rabbit IgG was added in some of the reactions, as indicated. Disappearance (see A) or increased mobility of the shifted band (B; shown by asterisk) was detected with anti-NRL antibody but not IgG. (C) ChIP assay. WT or Nrl−/− mouse retina was used for ChIP with anti-NRL or rabbit IgG antibody. The positive and negative controls for ChIP assays are Pde6a and albumin, respectively. Lanes are as indicated. Input DNA served as positive control for PCR.

FIG. 50 shows immunoblot analysis to examine NRL expression in Crxp-Nrl/Nrl−/− and BPp- Nrl/Nrl−/− retinas. Expression levels of the NRL protein were compared in retinas of transgenic mice. In contrast to Crxp-Nrl/Nrl−/−, BPp-Nrl/Nrl−/− retinas contain approximately 5% of the NRL protein.

FIG. 51 shows a schematic of the human NRL protein, and amino acid sequence alignment of NRL orthologs. (A) Arrows indicate altered NRL amino acid residues identified in individuals with retinopathies. MTD, minimal transactivation domain; Hinge, hinge domain; EHD, extended homology domain; BD, basic domain; Leu. Zipper, leucine zipper (Genbank accession #NM_{—}006177). (B) The amino acid sequence of human NRL is aligned with those of chimp, rhesus, cow, dog, mouse, rat, frog, zebrafish and fugu using ClustalW. Amino acid residues conserved in all orthologs are indicated by an asterisk and reduced identity is shown using either a colon or a dot. Residues with human changes described in the text are shown by arrows.

FIG. 52 shows isoform and phosphorylation analysis of WT and mutant NRL proteins. (A) Immunoblot analysis of COS-1 whole cell extracts expressing WT or mutant NRL constructs. NRL protein isoforms were detected using an ANTI-XPRESS antibody. FIG. 52A is a composite image from multiple immunoblots. (B) Metabolic labeling of NRL with ³²P. WT, p.S50T and p.P51S NRL transfected COS-1 cells were radiolabeled with ³²P. After solubilization, the NRL proteins were immunoprecipitated using ANTI-XPRESS antibody. (C) Alkaline phosphatase treatment of NRL. COS-1 whole cell extracts expressing WT, p.S50T or p.P51S NRL were treated with or without phosphatase (PPase) and detected with the ANTI-XPRESS antibody.

FIG. 53 shows subcellular localization of WT and mutant NRL proteins in COS-1 cells. COS-1 cells transiently transfected with the cDNA encoding WT or mutant NRL constructs, were stained, incubated with ANTI-XPRESS antibody and visualized using anti-mouse IgG-Alexa488 antibody (top panels). Bisbenzimide-labeled nuclei are shown in the central panels, and the bottom panel displays the merged images. Scale bar, 50 μm.

FIG. 54 shows effect of NRL mutations on binding to rhodopsin-NRE. (A) EMSA using the ³²P -labeled NRE was incubated with WT NRL containing COS-1 nuclear extracts. DNA-NRL complex formation is sequence specific for double-stranded DNA, as demonstrated by the competition with unlabeled rhodopsin-NRE oligonucleotide (1-50×) and using the non-specific (NS) oligonucleotide (50×). The thick arrow shows the position of a specific DNA-protein binding complex between NRL and rhodopsin-NRE. Thin arrows indicate non-specific oligo-shifts. (B) Binding of mutant NRL proteins to rhodopsin-NRE. The extracts were first equalized to WT NRL by immunoblot analysis, and pre-cleared with NS oligonucleotide (50×), prior to EMSA.

FIG. 55 shows transactivation of the bovine rhodopsin promoter with WT or mutant NRL cDNA together with CRX. (A-D) Different concentrations of WT or mutant NRL expression constructs (0.01-0.3 μg) were co-transfected into HEK293 cells with bovine rhodopsin −130 to +72-luciferase fusion construct (pGL2-pBR130) and CRX expression construct (pcDNA4-CRX). Fold change is relative to the empty expression vector control. Error bars indicate the SE. WT is indicated by a dark dotted line. Mutations were grouped based on, A higher, B similar, C somewhat lower, and D substantially lower, activity relative to WT NRL. Groups were assigned in part by the number of times the alterations were statistically different from WT NRL.

FIG. 56 shows transactivation of the bovine rhodopsin promoter with WT or mutant NRL cDNA, together with NR2E3 and/or CRX. (A) Different concentrations of WT or mutant NRL expression constructs (0.01-0.3 μg) were co-transfected into HEK293 cells with pGL2-pBR130 and NR2E3 expression construct (pcDNA4-NR2E3). (B) Includes both CRX and NR2E3 expression constructs. Fold change is relative to the empty expression vector control. Error bars indicate the SE. WT is indicated by a dark dotted line.

FIG. 57 shows serum induces NRL expression in Y79 cells. Y79 cells were grown in RPMI media without (A) or with (B) FBS (15%) for indicated time intervals, and protein extracts were analyzed by immunoblotting using anti-NRL antibody. Multiple isoforms of NRL are indicated by a bracket. Lanes are as indicated. Lower panel in A shows that the same blot was probed with anti-β-tubulin antibody, which served as a loading control. Molecular masses of markers are shown in kDa. The positive control(+ve) represents Y79 cells grown in 15% FBS.

FIG. 58 shows that RA stimulates expression of NRL protein in Y79 cells. Serum-starved Y79 cells were incubated with indicated concentrations of 9-cis at RA, 15% FBS(A) or TTNPB(B) for 24 h. Cell extracts were analyzed by SDS-PAGE and immunoblotting using anti-NRL antibody. Negative controls included 1% ethanol or Me2SO in lieu of the soluble factors. A bracket indicates multiple phosphorylated NRL isoforms. Lanes are as indicated. Molecular mass markers are indicated on the left. Additional bands in the higher molecular mass range may represent cross-reacting proteins. C, time-dependent effect of RA: serum-deprived Y79 cells were incubated with medium containing 10 μM RA for indicated time intervals. At the end of incubation, cells extract was analyzed by SDS-PAGE and immunoblotting using anti-NRL antibody. Lanes are as indicated. D, effect of protein synthesis inhibitor CHX on RA-mediated NRL induction was studied by incubating serum-starved Y79 cells with media containing at RA (10 μ) and CHX (20 μg/ml)(left panel; RA-treated simultaneously). In a similar experiment, cells were pretreated with RA for 24 h followed by addition of CHX(right panel). Cell extracts were analyzed by SDS PAGE and immunoblotting using anti-NRL antibody.

FIG. 59 shows RA increases NRL protein levels in cultured rat and porcine photoreceptors. Analyses of rat(A) and porcine(B) retinal cultures after incubation with indicated concentrations of RA or FBS. Newborn rat retinal cells and adult pig photoreceptors were cultured in vitro, as described under “Experimental Procedures.” Cell extracts were analyzed by SDS-PAGE and immunoblotting using anti-NRL antibody. In both panels, the intensity of the NRL immunoreactive band was reduced in serum-free culture compared with +FBS, and was partially restored by increasing doses of RA. This reduction was significantly different (p<0.05) compared with serum-supplemented controls (*). For rat cultures, this reduction was also significantly different from 20 μMRA, but not for other values. 40 μMRA was toxic for cell survival in newborn rat retina. For pig cultures, the decrease was significantly different compared with all RA concentrations, except 20 μM. The arrow in B indicates the major NRL immunoreactive band used for scanning. Histograms show densitometric scan of representative blots for each culture model. C, adult pig photoreceptor cultures were prepared and immuno stained as described under “Experimental Procedures.” Nomarski differential contrast images of cells are depicted in panels a, e, and i; DAPI staining of the nuclei in the same fields is shown in panels b, f, and j; NRL immunolabeling of the same fields is shown in panels c, g, and k; and anti-rhodopsin immunolabeling of the same fields is shown in panels d, h, and l. Positive control cultures, maintained in chemically defined medium to which serum-supplemented medium was added for 24 h, revealed strong nuclear NRL immunoreactivity (panel c), as did cells treated with RA (10 μM) for 24 h (panel k); however cells maintained in chemically defined medium demonstrated less intense nuclear staining (panel g). In all cases, rhodopsin staining was not detectably different. Scale bar in panel l is 4 μm for all panels.

FIG. 60 shows putative RAREs within the Nrl promoter are protected by retinal nuclear proteins. A, schematic representation of the Nrl promoter showing regions of homology (I, II, III, and IV) between human (h) and mouse (m) Nrl. E1 denotes exon 1 of the Nrl gene. B, DNaseI footprinting using bovine RNE was performed as described under “Experimental Procedures.” Footprints corresponding to regions II and III are shown. Vertical lines indicate footprinted regions. (−) denotes footprint in the absence of RNE whereas(+)indicates the experiment in the presence of RNE. Footprints containing the putative RAREs are indicated by III-1, III-2, and II-1. C, sequence of the putative RAREs in the footprints (II and III) of both mouse and human Nrl promoter region. Regions III-1 and III-2 contain putative ROR (orphan receptor) and RAR response elements whereas region II-1 contains a putative RXR binding element. D, EMSA, oligonucleotides corresponding to the regions III-2 (Oligo III-2) and II-1 (Oligo II-1) were radiolabeled using [γ-³²P]dATP and incubated with bovine retinal nuclear extract followed by analysis using non-denaturing PAGE, as described under “Experimental Procedures.” Competition experiments were performed with unlabeled oligonucleotides to validate the specificity of the band shift. Experiments in the presence of antibody against various receptor ligands showed the presence or absence of the specific proteins. Arrow indicates a nonspecific band shift. * indicates radiolabeled oligo used in the experiment; mt-Oligo represents mutant oligonucleotide from which the putative RAREs have been deleted. Lanes are as indicated. Brackets indicate specific gel-shifted bands.

FIG. 61 shows RA receptors bind to and activate Nrl promoter. A, schematic representation of the mouse Nrl promoter-luciferase constructs used to study the response to RA. The deletion fragments were cloned into pGL3-basic plasmid in-frame with the luciferase reporter gene. RAR and RXR response elements in regions III and II, respectively are depicted. These constructs were used in a separate assay to check for intrinsic promoter activity. B, Nrl promoter-luciferase constructs were transfected into Y79 cells as described under “Experimental Procedures.” Promoterless vector, pGL3 vector was used as negative control and the value of luciferase activity was set to 1. Results are expressed as a ratio of luciferase values obtained in the presence or absence of RA. C, site-directed mutants of the pGL3-N1 construct (pGL3-N1-mut III-1, III-2, or II-1), containing deletions of the putative RAREs, were used to transfect HEK293 cells in the presence of indicated concentrations of at RA. The value of the control (transfected with the wild-type pGL3-N1 with no at RA) was set at 100% luciferase activity. Results are expressed as percent luciferase activity as compared with the controls.

FIG. 62 shows a model of photoreceptor specification/differentiation of one of the embodiments of the present invention. Otx2 and Rb influence multipotent retinal neuroepithelial cells to exit cell cycle. In some embodiments, the present invention provides that Crx is the competence factor in postmitotic photoreceptor precursors. The cells that express Nrl are committed to rod photoreceptor fate, with subsequent expression of Nr2e3. The cells expressing only Crx are cone precursors. In some embodiments, the present invention provides a degree of plasticity exists in early retinal development, such that changes in Nrl and/or Nr2e3 expression can lead to alterations in final ratio of rod and cone photoreceptors, and that the expression of other transcription factors (e.g., regulated (e.g., directly or indirectly) by the expression of Nrl) guide the development to mature photoreceptors.

FIG. 63 shows NRL directly binds to and activates the Nr2e3 promoter. (A) Schematic of approximately 4.5 kb genomic DNA upstream of the Nr2e3 transcription start site (denoted as +1). The four boxes indicate sequence regions conserved in mammals. A comparison of sequences in the second conserved region including a putative NRE (highlighted in grey) is shown. (B) EMSA. NRL containing COS-1 nuclear extract and ³²P-labeled NRE probe (−2820 nt to −2786 nt) were used in EMSA. Lanes 1 to 8, 40 000 cpm ³²P-labeled probe; lane 2, 10 μg nuclear extract (NE) from untransfected COS-1 cells; lanes 3 to 8, 10 μg nuclear extract from COS-1 cells transfected with Nrl cDNA expression plasmid (NRL NE); lane 4, 50-fold excess wild-type unlabeled NRE probe; lane 5, 100-fold excess wild-type unlabeled NRE probe; lane 6, 100-fold unlabeled mutant NRE probe; lane 7, 2.0 μg anti-NRL antibody; and lane 8, 2.0 μg normal rabbit IgG. (C) ChIP assays with chromatin from adult C57BL/6J retinas. Lane 1, NRL antibody used for IP; lane 2, normal rabbit IgG used for IP, a negative control; and lane 3, input DNA used as template for PCR. Top panel: primers amplifying the NRE containing region (−2989 nt to −2742 nt) in the Nr2e3 promoter region were used for PCR. Bottom panel: primers amplifying an irrelevant region (1230 nt to 1438 nt) in the Nr2e3 gene were used for PCR. (D) Luciferase transactivation assays showing the activation of Nr2e3 promoter by NRL and CRX.

FIG. 64 shows NRL does not completely suppress S-opsin expression in the absence of NR2E3. WT adult retina whole mounts were analyzed for S-opsin expression (A). The inferior to superior gradient of S-opsin expression can be readily observed (B-C). In the absence of NRL, whorls (arrows) and S-opsin can be detected throughout the retina (D-F); while the expression of NRL in early cone precursors (Crxp-Nrl/WT) results in the complete absence of S-opsin (G-I). In rd7 mice, enhanced S-opsin expression and whorls (arrows) are observed in both the superior and inferior domain (J-L). When Crxp-Nrl/WT mice were crossed with rd7 mice, the resultant transgenic line revealed whorls (arrows) throughout the retina and significantly less S-opsin expression in the superior domain (M-O). Scale bar: 200 μm (A, D, G, J, M) and 50 μm (B, C, E, F, H, I, K, L, N, O).

FIG. 65 shows expression of cone-specific markers and targeting of some photoreceptors to the ONL is perturbed in the absence of NRL and NR2E3. Immunostaining with mCAR, S-opsin, and M-opsin from WT (A: a-c), Nrl−/− (A: d-f), Crxp-Nrl/WT (A: g-i), rd7 (A: j-l) and Crxp-Nrl/rd7 (A: m-o) retinal cryosections. Compared to WT (B: a-b) and Crxp-Nrl/WT (B: e-f), targeting of S-cones (arrows) to the ONL is perturbed in Nrl−/− (B: c-d) and rd7 retinas (B: g-h), and S-opsin positive nuclei are present in the INL. S-cone staining (arrowheads) in the Crxp-Nrl/rd7 retinas (B: i-j) is observed in cells closest to the outer plexiform layer. OS, outer segments; ONL, outer nuclear layer; INL, inner nuclear layer; BBZ, 25 bisbenzamide. Scale bar: 25 μm.

FIG. 66 shows absence of normal cone function in cone photoreceptors expressing NRL but not NR2E3. Dark-adapted (A) or light-adapted (C) ERG waveform series are from 2-3-month-old WT, Nrl−/−, Crxp-Nrl/WT, rd7 and Crxp-Nrl/rd7 mice. (B) and (D) show ERG amplitude versus stimulus intensity series for dark- or light-adapted conditions, respectively. Bars indicate±SE. 26

FIG. 67 shows non-redundant differentially expressed genes in Crxp-Nrl/WT or Crxp- Nr2e3/WT samples compared to WT retinas. Gene profiles of P28 retinal samples from Crxp-Nrl/WT or Crxp-Nr2e3/WT mice were compared to those from the WT retina. Common genes in Crxp-Nrl/WT and Crxp-Nr2e3/WT, or unique genes from Crxp-Nrl/WT or Crxp-Nr2e3/WT with a minimum fold change of 4 and FDRCI P-value of <0.1 are shown.

FIG. 68 shows non-redundant differentially expressed genes in Crxp-Nrl/WT or Crxp-Nr2e3/WT samples compared to Nrl−/− retinas. Gene profiles of P28 retinal samples from Crxp-Nrl/WT or Crxp-Nr2e3/WT were compared to the profiles from the Nrl−/− retina. Common differentially expressed genes in Crxp-Nrl/WT and Crxp-Nr2e3/WT retina, or unique genes from Crxp-Nrl/WT or Crxp-Nr2e3/WT, with a minimum fold change of 10 and FDRCI P-value of <0.1, are shown.

FIG. 69 shows non-redundant differentially expressed genes in Crxp-Nrl/WT or Crxp-Nr2e3/WT samples compared to rd7 retinas. Gene profiles of P28 retinal samples from Crxp-Nrl/WT or Crxp-Nr2e3/WT were compared to those of rd7 retina. Common genes in Crxp-Nrl/WT and Crxp-Nr2e3/WT, or unique genes from Crxp-Nrl/WT or Crxp-Nr2e3/WT with a minimum fold change of 10 and FDRCI P-value of <0.1 are shown.

### DEFINITIONS

As used herein, the term “animal” refers to any animal (e.g., a mammal), including, but not limited to, humans, non-human primates, rodents (e.g., mice, rats, etc.), flies, and the like.

As used herein, the term “non-human animals” refers to all non-human animals including, but not limited to, vertebrates such as rodents, non-human primates, ovines, bovines, ruminants, lagomorphs, porcines, caprines, equines, canines, felines, ayes, etc.

As used herein, the term “immunoglobulin” or “antibody” refer to proteins that bind a specific antigen. Immunoglobulins include, but are not limited to, polyclonal, monoclonal, chimeric, and humanized antibodies, Fab fragments, F(ab′)₂ fragments, and includes immunoglobulins of the following classes: IgG, IgA, IgM, IgD, IbE, and secreted immunoglobulins (sIg). Immunoglobulins generally comprise two identical heavy chains and two light chains. However, the terms “antibody” and “immunoglobulin” also encompass single chain antibodies and two chain antibodies.

As used herein, the term “antigen binding protein” refers to proteins that bind to a specific antigen. “Antigen binding proteins” include, but are not limited to, immunoglobulins, including polyclonal, monoclonal, chimeric, and humanized antibodies; Fab fragments, F(ab′)₂ fragments, and Fab expression libraries; and single chain antibodies.

The term “epitope” as used herein refers to that portion of an antigen that makes contact with a particular immunoglobulin.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “specifically binding to Nrl with low background binding” refers to an antibody that binds specifically to Nrl protein (e.g., in an immunohistochemistry assay) but not to other proteins (e.g., lack of non-specific binding).

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject is suspected of having photoreceptor loss” refers to a subject that presents one or more symptoms indicative of a medically relevant photoreceptor loss (e.g., caused by a disorder, disease, aging, genetic predisposition, or injury). A subject suspected of having photoreceptor loss has generally not been tested for photoreceptor loss. However, a “subject suspected of having photoreceptor loss” encompasses an individual who has received a preliminary diagnosis but for whom a confirmatory test has not been done or for whom the degree of photoreceptor loss is not known. A “subject suspected of having photoreceptor loss” is sometimes diagnosed with photoreceptor loss and is sometimes found to not have photoreceptor loss.

As used herein, the term “subject diagnosed with a photoreceptor loss” refers to a subject who has been tested and found to have photoreceptor (e.g., rod cell or cone cell) loss. Examples of such subjects include, but are not limited to, subjects with retinal or macular degeneration.

As used herein, the term “subject at risk for photoreceptor loss” refers to a subject with one or more risk factors for developing photoreceptor loss. Risk factors include, but are not limited to, gender, age, genetic predisposition (e.g., genetic disorder), environmental exposure, and previous incidents of diseases, and lifestyle.

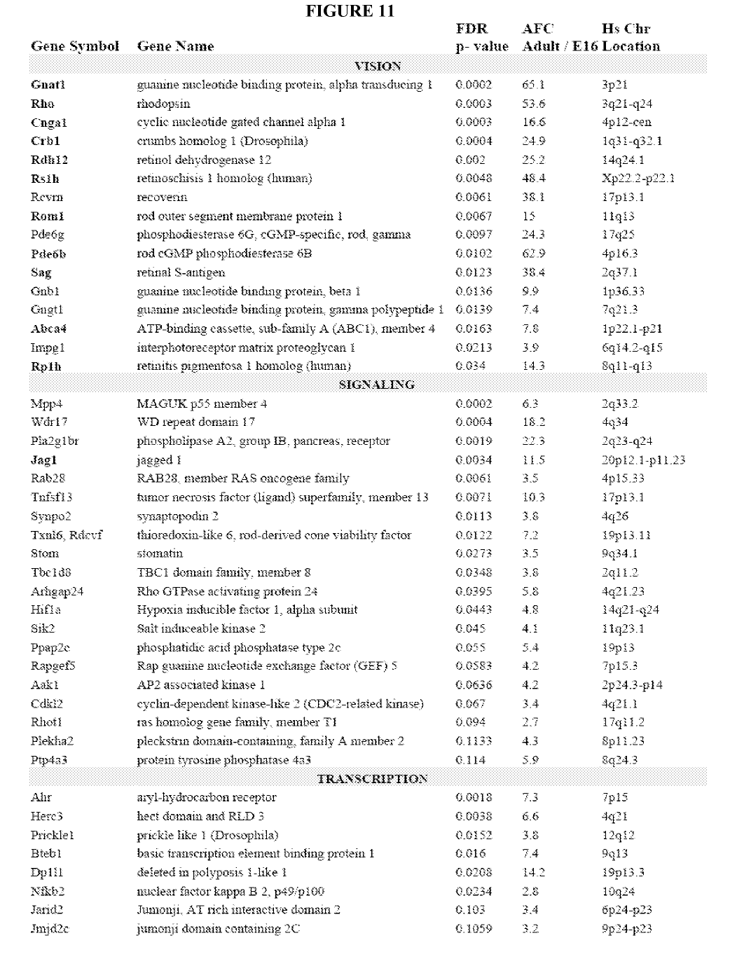

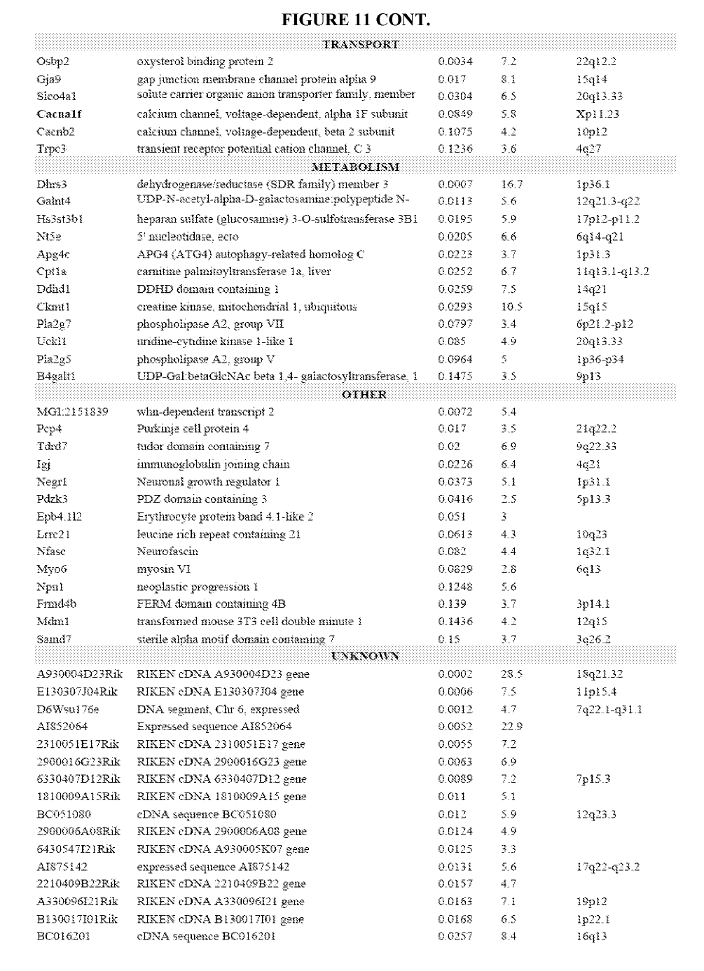

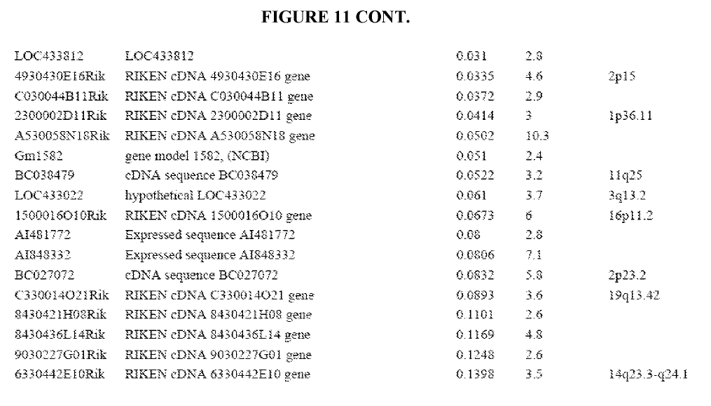

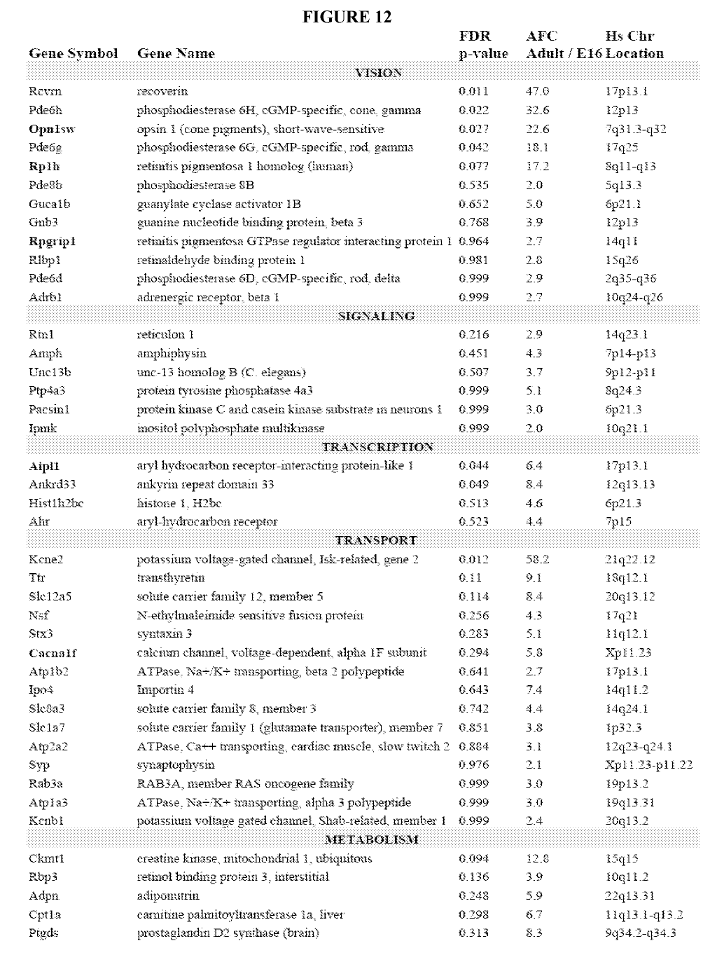

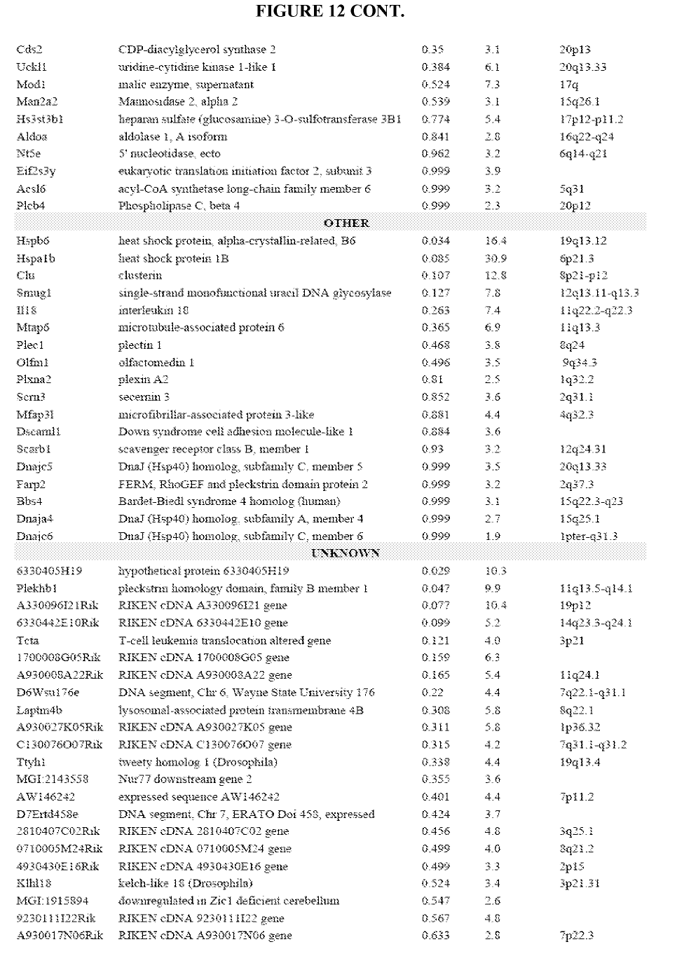

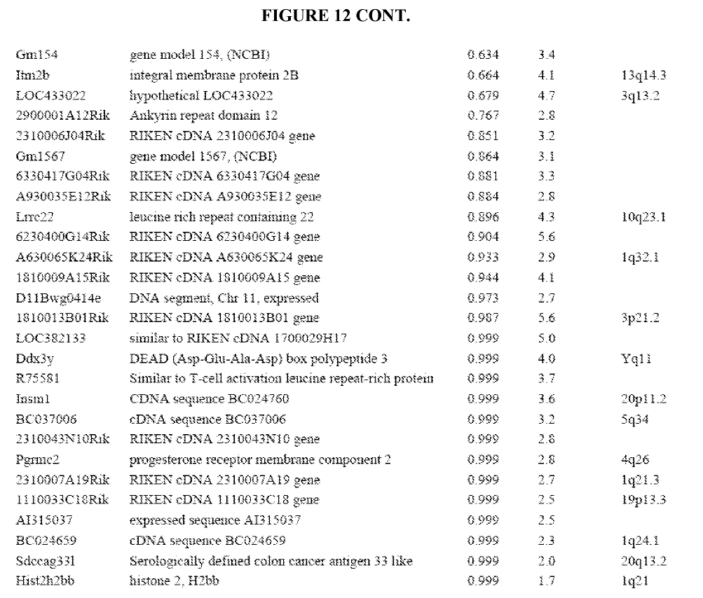

As used herein, the term “characterizing photoreceptor cells in subject” refers to the identification of one or more properties of a photoreceptor cell (e.g., in a subject), including but not limited to, the ability of the cells to form synaptic connections (e.g., with the brain) and the ability of the cells to integrate into the retina (e.g., the outer nuclear layer of the retina). Photoreceptor cells may be characterized by the identification of the expression level of one or more biomarkers (e.g., Nrl or biomarker described in FIGS. 11, 12 and/or 13) in the photoreceptor cells.

As used herein, the term “characterizing tissue in a subject” refers to the identification of one or more properties of a tissue sample (e.g., including but not limited to, morphology and cellular localization (e.g., within the retina)). In some embodiments, tissues are characterized by the identification of the expression level of one or more biomarkers (e.g., Nrl or biomarker described in FIGS. 11, 12 and/or 13) in the tissue.

As used herein, the term “reagent(s) capable of specifically detecting biomarker expression” refers to reagents used to detect (e.g., sufficient to detect) the expression of biomarkers of the present invention (e.g., Nrl or biomarker described in FIGS. 11, 12 and/or 13). Examples of suitable reagents include, but are not limited to, nucleic acid probes capable of specifically hybridizing to biomarker mRNA or cDNA, and antibodies.

As used herein, the term “instructions for using said kit for detecting photoreceptor cell status” includes instructions for using the reagents contained in the kit for the detection and characterization of photoreceptor cells in a sample (e.g., derived from a subject or from stem cells).

As used herein, the term “effective amount” refers to the amount of a composition (e.g., inducer of Nrl expression and/or activity) sufficient to effect beneficial or desired results. An effective amount can be administered in one or more administrations, applications or dosages and is not intended to be limited to a particular formulation or administration route.

As used herein, the terms “photoreceptor precursor cell” and “photoreceptor precursors” refer to post-mitotic, not fully differentiated, non-dividing cells (e.g., identified and purified utilizing the compositions and methods of the present invention (e.g., biomarkers described herein)) committed to become photoreceptor cells. Photoreceptor precursor can be characterized in that the cells are not only able to survive when transplanted into the subretinal space of a host subject, but are also able to integrate into the outer nuclear layer of the retina. They may also form synaptic connections. A photoreceptor precursor cell may be a rod photoreceptor precursor cell or cone photoreceptor precursor cell. The present invention is not limited by the ontogenic stage of the photoreceptor precursor cell. As described herein, the expression of one or more biomarkers within (e.g., Nrl, Nr2e3 or other protein that is a target of Nrl expression) or on the surface of (e.g., CD24a, CD1d1, Chrnb4, Clic4, Ddr1, F2r, Gpr137b, Igsf4b, LRP4, Nope, Nrp1, Pdpn, Ptpro, St8sia4, Tmem46) the photoreceptor precursor cell can be utilized for identifying photoreceptor precursor cells (e.g., in embryonic day 11 (E11) through post natal day 7 (P7) subjects (e.g., mice)).

As used herein, the term “administration” refers to the act of giving a drug, prodrug, or other agent (e.g., a test compound or photoreceptor precursor cell), or therapeutic treatment to a subject (e.g., a subject or in vivo, in vitro, or ex vivo cells, tissues, and organs). Exemplary routes of administration to the human body can be through the eyes (ophthalmic (e.g., via sub-retinal injection)), mouth (oral), skin (transdermal), nose (nasal), lungs (inhalant), oral mucosa (buccal), ear, by injection (e.g., intravenously, subcutaneously, intratumorally, intraperitoneally, etc.) and the like.

As used herein, the term “co-administration” refers to the administration of at least two agent(s) (e.g., photoreceptor precursor cells and one or more other agents—e.g., a test compound) or therapies to a subject (e.g., a human or mouse). In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

As used herein, the term “toxic” refers to any detrimental or harmful effects on a subject, a cell, or a tissue as compared to the same cell or tissue prior to the administration of the toxicant.

As used herein, the term “pharmaceutical composition” refers to the combination of an active agent (e.g., photoreceptor cell or test compound) with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “topically” refers to application of the compositions of the present invention to the surface of the skin and mucosal cells and tissues (e.g., alveolar, buccal, lingual, masticatory, or nasal mucosa, and other tissues and cells that line hollow organs or body cavities).

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintrigrants (e.g., potato starch or sodium starch glycolate), and the like. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. (1975), incorporated herein by reference).

As used herein, the term “pharmaceutically acceptable salt” refers to any salt (e.g., obtained by reaction with an acid or a base) of a compound of the present invention that is physiologically tolerated in the target subject (e.g., a mammalian subject, and/or in vivo or ex vivo, cells, tissues, or organs). “Salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the term “gene transfer system” refers to any means of delivering a composition comprising a nucleic acid sequence (e.g., encoding Nrl) to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1-3 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “transgene” refers to a heterologous gene that is integrated into the genome of an organism (e.g., a non-human animal) and that is transmitted to progeny of the organism during sexual reproduction.

As used herein, the term “transgenic organism” refers to an organism (e.g., a non-human animal) that has a transgene integrated into its genome and that transmits the transgene to its progeny during sexual reproduction.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (e.g., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (e.g., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (e.g., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization (1985)). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5× Denhardt's reagent (50× Denhardt's contains per 500 ml: 5 g Ficoll (Type 400, Pharamcia), 5 g BSA (Fraction V; Sigma)) and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the terms “restriction endonucleases” and “restriction enzymes” refer to bacterial enzymes, each of which cut double-stranded DNA at or near a specific nucleotide sequence.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

When used in reference to a cell, isolated refers to a cell (e.g., photoreceptor cell (e.g., photoreceptor precursor cell)) that is identified and separated from at least one other component (e.g., non-photoreceptor precursor cells). The term “isolated” when used in reference to a photoreceptor precursor cell refers to a photoreceptor precursor cell that is removed from its natural environment (e.g., a developing retina) and that is separated (e.g., is at least about 50-70% free, and most preferably about 90% free), from other cells with which it is naturally present, but that lack the marker (e.g., Nrl) based on which the photoreceptor precursor cells were isolated.

The term “enriched”, as in an enriched population of cells, can be defined based upon the increased number of cells having a particular marker in a fractionated set of cells as compared with the number of cells having the marker in the unfractionated set of cells.

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample. In another example, a cell (e.g., a photoreceptor cell (e.g., a photoreceptor precursor cell)) may be purified (e.g., other non-photoreceptor cells may be removed from the cells). Thus, “purified” photoreceptor precursor cells may be isolated or enriched cells.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression approximately 3-fold higher (or greater) than that observed in a given tissue in a control or non-transgenic animal.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

As used herein, the term “selectable marker” refers to the use of a gene that encodes an enzymatic activity that confers the ability to grow in medium lacking what would otherwise be an essential nutrient (e.g. the HIS3 gene in yeast cells); in addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line. Examples of dominant selectable markers include the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. Other selectable markers are not dominant in that their use must be in conjunction with a cell line that lacks the relevant enzyme activity. Examples of non-dominant selectable markers include the thymidine kinase (tk) gene that is used in conjunction with tk⁻ cell lines, the CAD gene that is used in conjunction with CAD-deficient cells and the mammalian hypoxanthine-guanine phosphoribosyl transferase (hprt) gene that is used in conjunction with hprt⁻ cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp. 16.9-16.15.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g.,photoreceptor loss). Test compounds comprise both known and potential therapeutic compounds. A test compound can be determined to be therapeutic by screening using the screening methods of the present invention. Examples of test compounds include, but are not limited to, carbohydrates, monosaccharides, oligosaccharides, polysaccharides, amino acids, peptides, oligopeptides, polypeptides, proteins, nucleosides, nucleotides, oligonucleotides, polynucleotides, including DNA and DNA fragments, RNA and RNA fragments and the like, lipids, retinoids, steroids, drug, antibody, prodrug, glycopeptides, glycoproteins, proteoglycans and the like, and synthetic analogues or derivatives thereof, including peptidomimetics, small molecule organic compounds and the like, and mixtures thereof (e.g., that is a candidate for use to treat or prevent a disease, illness, sickness, or disorder of bodily function (e.g., photoreceptor loss (e.g.,due to macular degeneration)). Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

The term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “target RNA molecule” refers to an RNA molecule to which at least one strand of the short double-stranded region of an siRNA is homologous or complementary. Typically, when such homology or complementary is about 100%, the siRNA is able to silence or inhibit expression of the target RNA molecule. Although it is believed that processed mRNA is a target of siRNA, the present invention is not limited to any particular hypothesis, and such hypotheses are not necessary to practice the present invention. Thus, it is contemplated that other RNA molecules may also be targets of siRNA. Such targets include unprocessed mRNA, ribosomal RNA, and viral RNA genomes.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the term “entering” as in “entering said growth rate information into said computer” refers to transferring information to a “computer readable medium.” Information may be transferred by any suitable method, including but not limited to, manually (e.g., by typing into a computer) or automated (e.g., transferred from another “computer readable medium” via a “processor”).

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “computer implemented method” refers to a method utilizing a “CPU” and “computer readable medium.”

### DETAILED DESCRIPTION OF THE INVENTION

The present invention relates to photoreceptor cells. In particular, the present invention provides photoreceptor cells comprising heterologous nucleic acid sequences and transgenic animals comprising the same. The present invention also provides photoreceptor precursor cells (e.g., rod photoreceptor precursor cells), and methods of identifying, characterizing, isolating and utilizing the same. Compositions and methods of the present invention find use in, among other things, research, clinical, diagnostic, drug discovery, and therapeutic applications.

Evolution of higher-order sensory and behavioral functions in mammals is accompanied by increasingly complex regulation of gene expression (See, e.g., Levine and Tjian, (2003) Nature 424, 147-151). As much as 10% of the human genome is presumably dedicated to the control of transcription. Exquisitely timed expression of cell-type-specific genes, together with spatial and quantitative precision, depends on the interaction between transcriptional control machinery and extracellular signals (See, e.g., Brivanlou and Darnell, (2002) Science 295, 813-818; Ptashne, Gann, A. (2001) Essays Biochem 37, 1-15). Neuronal heterogeneity and functional diversity result from combinatorial and cooperative actions of regulatory proteins that form complicated yet precise transcriptional networks to generate unique gene expression profiles. A key transcription factor, combined with its cognate regulatory cis-sequence codes, specifies a particular node in the gene regulatory networks that guide differentiation and development (See, e.g., Davidson et al., (2003) Proc. Natl. Acad. Sci. USA 100, 1475-1480).

The retina offers an ideal paradigm for investigating regulatory networks underlying neuronal differentiation. The genesis of six types of neurons and Müller glia in the vertebrate retina proceeds in a characterized sequence during development (See, e.g., Livesey and Cepko, (2001) Nat. Rev. Neurosci 2, 109-118). Subsets of multipotent retinal neuroepithelial progenitors exit the cell cycle at specific time points and acquire a particular cell fate under the influence of intrinsic genetic program and extrinsic factors (See, e.g., Livesey and Cepko, (2001) Nat. Rev. Neurosci 2, 109-118; Cayouette et al., (2003) Neuron 40, 897-904; Levine et al., (2000) Cell Mol. Life Sci 57, 224-234). Pioneering studies using thymidine labeling and retroviral vectors established the order and birthdates of neurons in developing retina (See, e.g., Livesey and Cepko, (2001) Nat. Rev. Neurosci 2, 109-118; Carter-Dawson and LaVail, (1979) J. Comp. Neurol 188, 263-272; Young, (1985) Anat. Rec 212, 199-205; Young, (1985) Brain Res 353, 229-239). One hypothesized model of retinal differentiation proposes that a heterogeneous pool of progenitors passes through states of competence, where it can generate a distinct subset of neurons (See, e.g., Livesey and Cepko, (2001) Nat. Rev. Neurosci 2, 109-118). Thus, at the molecular level, this competence may be acquired by combinatorial action of specific transcriptional regulatory proteins. Genetic ablation studies of transcription factors involved in early murine eye specification are consistent with a combinatorial regulation model (See, e.g., Brown et al., (2001) Development (Cambridge, U.K.) 128, 2497-2508; Hatakeyama et al., (2001) Development (Cambridge, U.K.) 128, 1313-1322; Wang et al., (2001) Genes Dev 15, 24-29).

Rod and cone photoreceptors account for 70-80% of all cells in the adult neural retina. In most mammals, rods greatly outnumber cones (95-97% of photoreceptors in mouse and human). Rods are born over a broad developmental window and, in mice, the majority are generated postnatally (See, e.g., Livesey and Cepko, (2001) Nat. Rev. Neurosci 2, 109-118; Young, (1985) Anat. Rec 212, 199-205; Cepko et al., (1996) Proc. Natl. Acad. Sci. USA 93, 589-595). Depending upon the time of their birth (“early” or “late”), postmitotic rod precursors exhibit variable delays before expressing the photopigment rhodopsin, a definitive marker of mature rods (See, e.g., Cayouette et al., (2003) Neuron 40, 897-904; Molday and MacKenzie, (1983) Biochemistry 22, 653-660; Cepko, C. (2000) Nat. Genet 24, 99-100; Morrow et al., (1998) J. Neurosci 18, 3738-3748). Prior to experiments conducted during development of the present invention, the molecular differences between early- and late-born rods and the mechanism(s) underlying the “delay” had remained uncharacterized.

Photoreceptor loss (e.g., caused by a disorder, disease, aging or injury) causes irreversible blindness. Cell transplantation was initially thought to be a feasible type of central nervous system repair. For example, photoreceptor degeneration initially leaves the inner retinal circuitry intact and new photoreceptors only need to make a single, short synaptic connection to contribute to the retinotopic map. However, prior to the development of the present invention, there had been no success transplanting cells (e.g., brain or retina derived stem cells) into a mature, adult retina resulting in the integration of the cells and formation of synaptic connections, nor the restoration of visual function. (See, e.g., Chacko et al., Biochem. Biophys. Res. Commun. 268, 842-846 (2000); Sakaguchi et al., Dev. Neurosci. 26, 336-345 (2004); Van Hoffelen et al., Invest Ophthalmol. Vis. Sci. 44, 426-434 (2003); Young et al., Mol. Cell Neurosci. 16, 197-205 (2000).

Nrl is a basic motif-leucine zipper transcription factor (See, e.g., Swaroop et al., (1992) Proc. Natl. Acad. Sci. USA 89, 266-270), specifically expressed in rod photoreceptors (See, e.g., Swain et al., (2001) J. Biol. Chem 276, 36824-36830; Coolen et al., (2005) Dev. Genes Evol 215, 327-339) and pinealocytes. Nrl interacts with cone rod homeobox (Crx), photoreceptor-specific orphan nuclear receptor (Nr2e3), and other proteins to regulate the expression of rod-specific genes (See, e.g., Rehemtulla et al., (1996) Proc. Natl. Acad. Sci. USA 93, 191-195; Chen et al., (1997) Neuron 19, 1017-1030; Mitton et al., (2000) J. Biol. Chem 275, 29794-29799; Lerner et al., (2001) J. Biol. Chem 276, 34999-35007; Cheng et al., (2004) Hum. Mol. Genet 13, 1563-1575; Yoshida et al., (2004) Hum. Mol. Genet 13, 1487-1503)). Missense mutations in the human NRL gene are associated with retinopathies (See, e.g., Bessant et al., (1999) Nat. Genet 21, 355-356; Nishiguchi et al., (2004) Proc. Natl. Acad. Sci. USA 101, 17819-17824). Deletion of Nrl in mice results in a cone-only outer nuclear layer in the retina (See, e.g., Mears et al., (2001) Nat. Genet 29, 447-452; Daniele et al., (2005) Invest. Ophthalmol. Visual Sci 46, 2156-2167).

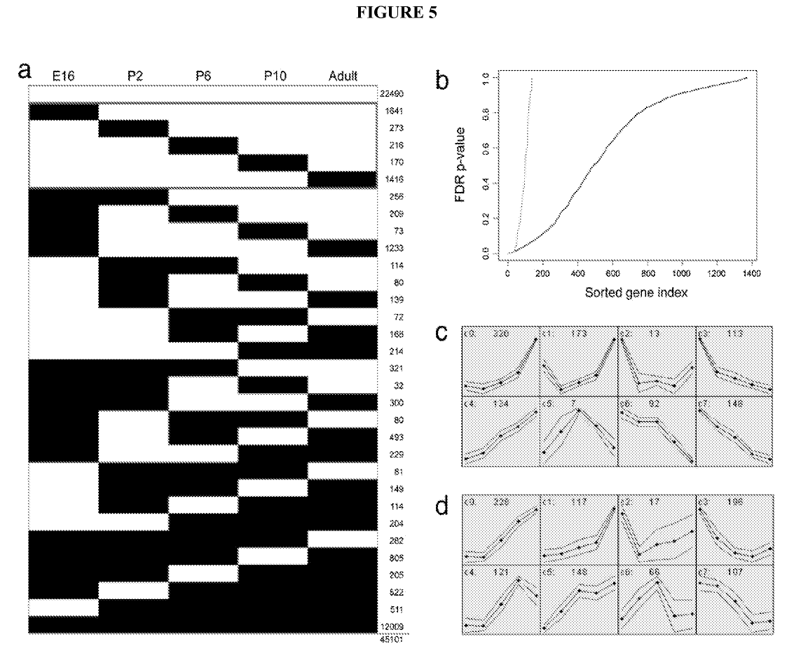

Experiments were conducted during development of the present invention in order to determine if Nrl could provide insight into photoreceptor development (e.g., into gene expression changes and regulatory networks underlying photoreceptor development). Accordingly, experiments were conducted using the Nrl-promoter to express GFP in transgenic mice. The present invention provides that Nrl is indeed the earliest rod lineage-specific marker (See Example 1). The present invention provides that Nrl can be detected as early as embryonic day 12 (E12) in mice. Furthermore, the present invention provides that cells fated to become rods acquire a cone phenotype in the absence of Nrl, thereby establishing Nrl as a major cell-autonomous regulatory gene for rod differentiation (See, e.g., Example 1). In some embodiments, the present invention provides isolated photoreceptor precursor cells (e.g., rod photoreceptor precursor cells (e.g., GFP+ photoreceptor cells isolated by fluorescent activated cell sorting (FACS), See, e.g., Example 1)). The present invention also provides additional markers of photoreceptor development. For example, the present invention provides gene profiles of GFP+ photoreceptors, isolated by FACS, from wild-type and Nrl^{-/-} retinas at five distinct stages of differentiation (See, e.g., Example 1, and FIGS. 5, 11, 12, and 13). Thus, in some embodiments, the present invention provides tools (e.g., photoreceptor precursor cells) for characterizing photoreceptors (e.g., photoreceptor development (e.g., from photoreceptor precursor cells (e.g., postmitotic precursor cells))). In some embodiments, the present invention provides compositions and methods for generating, monitoring and/or characterizing differentiated cells (e.g., neuronal stem cells) comprising introducing a heterologous nucleic acid comprising Nrl (e.g., Nrl promoter and/or coding sequences) (e.g., via transfection or infection of a virus comprising a heterologous nucleic acid sequence) into the cells (e.g., stem cells) and monitoring differentiation of the cells. In some embodiments, Nrl promoter sequence introduced into a cell can be regulated by factors added to the cell. In some embodiments, the activity of the Nrl promoter sequence is utilized to identify the birth of and/or differentiation of photoreceptor precursor cells (e.g., rod photoreceptor precursor cells) and/or mature photoreceptor cells (e.g., rod cells).

Additionally, in some embodiments, the present invention provides biological markers (biomarkers (e.g., Nrl, Nr2e3, as well as genes described in FIGS. 11, 12, and 13)) that can be utilized to characterize photoreceptor cells (e.g., photoreceptor precursor cells (e.g., rod or cone photoreceptor precursor cells)). For example, the present invention provides distinct patterns of biomarker expression (e.g., described in FIGS. 11, 12, and 13) that can be utilized to identify photoreceptor precursor cells and/or characterize photoreceptor cells (e.g., photoreceptor precursor cells (e.g., rod or cone photoreceptor precursor cells)) that have been administered a test compound or agent or that are derived from stem cells (in culture or in vivo).

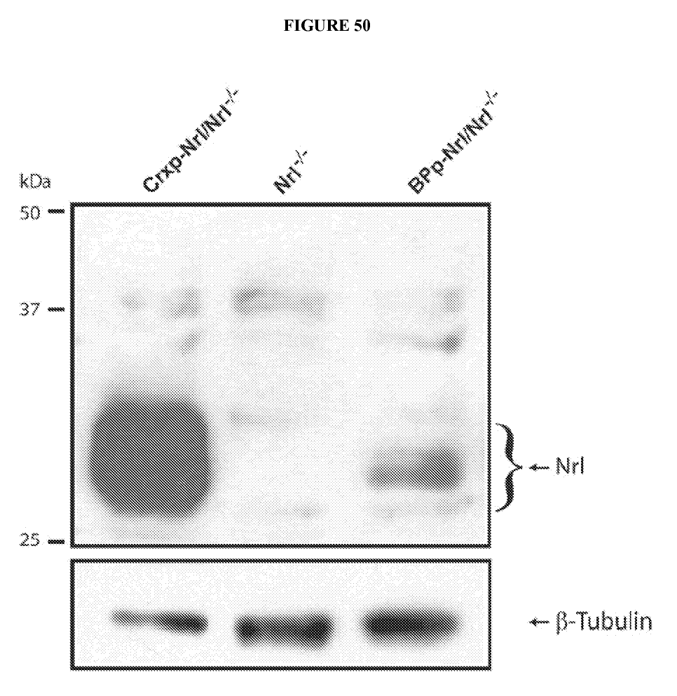

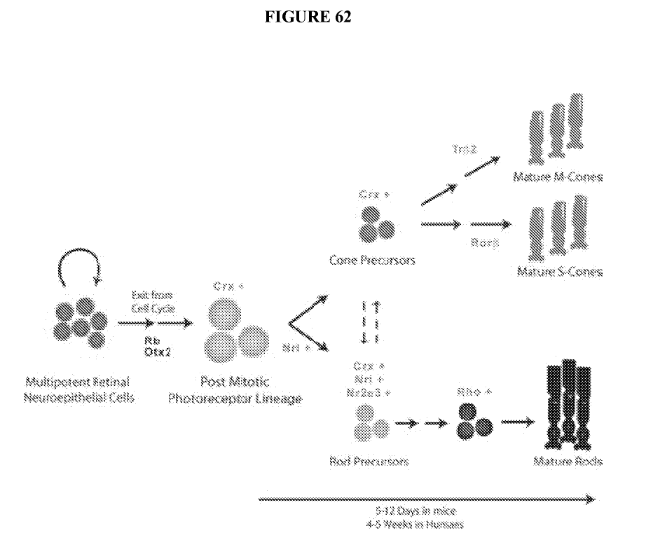

The present invention provides that the functionality of the Nrl promoter in a developing Nrl^{-/-} retina indicates the availability of factors (e.g., signaling factors) important for rod determination, but in the absence of Nrl, rod precursors (e.g., GFP-tagged precursors) acquire the identity of S-cones. Although an understanding of the mechanism is not necessary to practice the present invention and the present invention is not limited to any particular mechanism of action, in some embodiments, the present invention identifies the existence of pool(s) of progenitor cells with competence to become either a rod or a cone (e.g., binary cell fate choice) at an early step in retinal development. Although an understanding of the mechanism is not necessary to practice the present invention and the present invention is not limited to any particular mechanism of action, in some embodiments, during early stages of development, postmitotic precursor cells are not completely committed to a specific photoreceptor fate (e.g., they display plasticity) and transcriptional regulators, such as Nrl and/or Trb2 (See, e.g., Ng et al., (2001) Nat. Genet 27, 94-98), instruct the cells to produce rods or M-cones, respectively. In some embodiments, S-cones represent the “default” state (e.g., without the expression of Nrl, photoreceptor precursor cells develop into cone cells) or require another activator for differentiation (e.g., an activator selected from the group comprising the biomarkers identified in FIGS. 11, 12 and 13). Thus, in some embodiments, the present invention provides that photoreceptor precursor cells display postmitotic plasticity (e.g., expression of NRL even in CRX-expressing cone precursors produces functional rods (See Example 6). Thus, the present invention provides that the timing of expression, availability, amount and/or activity of NRL determines whether a postmitotic precursor cell will acquire a rod or a cone fate (e.g., that expression of NRL is essential and sufficient for rod genesis; See, e.g., Example 6, FIGS. 50 and 62). Furthermore, in some embodiments, the present invention provides that expression of NRL or other protein downstream of NRL in regulatory hierarchy of photoreceptor differentiation (e.g., NR2E3) can be used to suppress the expression of cone differentiation in vivo (e.g., can be used to bind to and suppress cone gene expression (e.g., Thrb and S-opsin gene expression)) (See Example 6).

In some embodiments, the present invention provides compositions and methods for genome-wide profiling (e.g., of biomarkers identified herein) to characterize expression dynamics of specific neurons developing within a single lineage over time, from commitment to maturation, using purified cell populations. The present invention also provides a comprehensive view of genetic determinants (e.g., biomarkers) that specify rod and cone morphology and function (See, e.g., biomarkers described in FIGS. 11, 12, and 13). The present invention also provides the ability to profile gene expression in wild-type photoreceptor cells versus expression of the same genes in diseased (e.g., degenerative) photoreceptor cells (for example, after tagging the diseased photoreceptors with GFP or using specific biomarkers described herein).

In addition, the present invention provides transgenic animals (e.g., comprising heterologous nucleic acid sequence encoding Nrl) that can be used, among other things, to characterize progenitor cell plasticity, determine the role of individual genetic mutations on rod and cone differentiation or function, evaluate cellular treatment paradigms for retinal and macular degeneration, and test compounds, agents or other interventions that alter photoreceptor cell differentiation and/or function. In some embodiments, the animals are transgenic mice (e.g., wt-Gfp transgenic mice described in Examples 1 and 2). In some embodiments, animals comprising transplanted photoreceptor cells are utilized (See, e.g., Example 2).

In some embodiments, the present invention provides a method of identifying a photoreceptor cell that, when transplanted into a host subject, is capable of integrating into the retina (e.g., in the outer nuclear layer (ONL)) and/or that is capable of forming functional synapses within the host.

For example, experiments were conducted during the development of the present invention in order to determine if committed progenitor or precursor cells at later ontogenetic stages of retinal development might have a higher probability of success upon transplantation. Several surprising and unexpected observations were made. The present invention identified that photoreceptor precursor cells can integrate into a retina (e.g., an adult and/or degenerating retina) if the cells are taken from the developing retina at a time that coincides with the peak of rod genesis (See, e.g., Example 2; and Young, Anat. Rec. 212, 199-205 (1985)). The present invention also identified that the transplanted cells integrate, differentiate into rod photoreceptors, form synaptic connections and improve visual function (See Example 2). Furthermore, the present invention identified that successfully integrated rod photoreceptors are derived from immature post-mitotic rod precursors and not from proliferating progenitor or stem cells (e.g., as shown in Example 2 using genetically-tagged post-mitotic rod photoreceptor precursor cells expressing the transcription factor Nrl described in Example 1). Thus, the present invention provides the identification, characterization (e.g., of ontogenetic stage (e.g., characterized by biomarkers (e.g., Nrl, Nr2e3 and other biomarkers described in FIGS. 11, 12, and 13))), and isolation of photoreceptor precursor cells (e.g., that can be used for research and clinical (e.g., therapeutic (e.g., rod photoreceptor transplantation)) applications).

Thus, the present invention provides that adult wild-type and degenerating mammalian retinas are capable of effectively incorporating rod and/or cone photoreceptor precursor cells (e.g., into the outer nuclear layer (ONL); See Examples 1 and 2). These cells can differentiate and form functional synaptic connections with downstream targets in the recipient retina and contribute to visual function (See Example 2). Furthermore, the present invention provides that transplantation of photoreceptor precursor cells (e.g., with and without co-administration with chondroitinase ABC) can provide a morphological and functional recovery in chemically induced photoreceptor degraded eyes (See Example 3).

The present invention also provides NRL post-translational modification(s) that function to alter NRL activity. For example, the present invention provides that NRL activity can be altered by phosphorylation status (See, e.g., Example 7). In some embodiments, phosphorylation of specific residues (e.g., S50 and P51 located in NRL's minimal transactivation domain) is important for interaction of NRL with TATA-binding protein (TBP). Thus, in some embodiments, the present invention provides that phosphorylation of NRL alters NRL's ability to bind TBP and other components of the general transcriptional machinery, thereby altering NRL's ability to regulate downstream gene expression (e.g., and photoreceptor cell fate). In some embodiments, the higher molecular mass isoforms of NRL have additional phosphorylated residues (e.g., in addition to S50 and P51) and exhibit less transcriptional activation capacity (e.g., of the rhodopsin promoter) (See, e.g., Example 7). In some embodiments, phosphorylation of residue S50 of NRL plays a role in triggering additional modification (e.g., phosphorylation, acetylation, glycosylation, etc.) of NRL. Accordingly, in some embodiments, the present invention provides that compositions (e.g., kinases, phosphatases and/or nucleic acid sequences encoding the same) can be utilized to alter (e.g., increase and/or decrease) NRL activity (e.g., in vivo, in vitro, or ex vivo; e.g., by post-translationally modifying NRL (e.g., at any of the amino acid residues identified in Example 7)). Thus, in some embodiments, controlling NRL activity (e.g., with a kinase, phosphatase, etc.) can be utilized to modulate NRL function (e.g., its interaction with transcription regulatory proteins) and in turn alter photoreceptor development (e.g., differentiation of photoreceptor precursor cells).

Rather than the environment of the mature retina inhibiting photoreceptor maturation, the present invention provides that transplantation of cells at a specific ontogenetic stage (e.g., defined by expression of one or more biomarkers described herein (e.g., Nrl, Nr2e3, or other biomarker described in FIGS. 11, 12, and 13)) results in their integration and subsequent differentiation into rod photoreceptors, even in mice with degenerating retina. Conversely, progenitor or stem cells that do not exhibit biomarker expression patterns that identify photoreceptor precursor cells described herein (e.g., Nrl, Nr2e3, and/or other biomarker expression) do not exhibit this property and fail to integrate. Thus, the present invention provides biomarkers (e.g., Nrl, Nr2e3, and/or other biomarkers described in FIGS. 11, 12, and 13) that can be used to identify, isolate, characterize and/or otherwise define photoreceptor precursor cells (e.g., the optimal ontogenetic stage for photoreceptor donor cells (e.g., for transplantation (e.g., that may facilitate the identification and/or generation of appropriate cells for transplantation (e.g., from stem cells (e.g., adult- or embryonic-derived stem cells)))).

### I. Biomarkers for Photoreceptor Cells

The present invention provides biomarkers whose presence and/or expression is specifically detectable and/or altered during photoreceptor cell development. Such biomarkers find use in the identification, isolation and characterization of photoreceptor cells (e.g., for use in clinical and/or basic research applications).

A. Identification of Markers

The present invention provides a comprehensive view of genetic determinants (e.g., biomarkers) that specify rod and cone morphology and function (See, e.g., biomarkers described in FIGS. 11, 12, and 13). In particular, the present invention provides that Nrl exists as the earliest detectable rod lineage-specific biomarker (See Example 1). Furthermore, the present invention provides that cells fated to become rods acquire a cone phenotype in the absence of Nrl, thereby establishing Nrl as a major cell-autonomous regulatory gene for rod differentiation (See, e.g., Example 1). The present invention also provides additional markers of photoreceptor development.

Thus, the present invention provides that the expression levels of Nrl, Nr2e3 and other biomarkers can be altered (increased or decreased) in order to regulate and/or alter photoreceptor development (e.g., post mitotic development) and photoreceptor loss (e.g., in a subject with a disease and/or disorder). The present invention therefore provides a method for altering photoreceptor (e.g., photoreceptor precursor) cell development comprising altering Nrl, Nr2e3 or other biomarker identified herein (e.g., in FIGS. 11, 12, and 13). Such a method can be used to induce photoreceptor development (e.g., photoreceptor integration and/or synaptic connectivity) and/or used to treat photoreceptor loss by promoting the responsiveness of photoreceptors to therapeutic treatment (e.g., with a test compound identified herein). For example, in some embodiments, the present invention provides a method of enhancing photoreceptor development comprising expressing Nrl and/or inducing Nrl activity in cells.

Furthermore, from gene profiling comparisons of purified photoreceptors from wild type and mutant mice and from various developmental stages, the present invention provides a number of biomarkers that can be utilized for identifying photoreceptor precursors as well as to assess photoreceptor differentiation. These biomarkers exhibit higher expression in immature yet committed cells compared to fully differentiated or functional photoreceptors. The present invention provides several categories of biomarkers including, but not limited to, cell surface protein biomarkers, nuclear protein biomarkers and other types of biomarkers. The present invention provides the cell surface proteins CD24a, CD1d1, Chrnb4, Clic4, Ddr1, F2r, Gpr137b, Igsf4b, LRP4, Nope, Nrp1, Pdpn,

Ptpro, St8sia4, and Tmem46 as biomarkers useful in the compositions and methods of the present invention. The present invention also provides the nuclear proteins Pax7, Sox4, Sox11, Nr1, Crx and Nr2e3 as biomarkers useful in the compositions and methods of the present invention. In some embodiments, Prss11 or Htra1, Marcks11, Prr15, and Tmeff1 are also useful as biomarkers in the compositions and methods of the present invention. In some embodiments, transcription factors or other proteins, the expression and/or activity of which is dependent upon Nrl expression (e.g., proteins downstream of Nrl such as Nr2e3) serve as biomarkers.

One example of a protein that is a downstream target of Nrl is Nr2e3. Nr2e3 has recently been identified as a rod-specific, orphan nuclear receptor that is involved with controlling photoreceptor differentiation (See Example 5). Nr2e3 suppresses the expression of cone genes, and activates a subset of rod genes including rhodopsin in vivo. In some embodiments, compositions and methods of the present invention can be utilized to identify a ligand(s) for Nr2e3. For example, in some embodiments, test compounds that are able to activate Nr2e3 expression and/or activity can be identified by monitoring photoreceptor cell development (e.g., differentiation into rod cells). The present invention is not limited by the type of test compound analyzed. In some embodiments, the test compound is a retinoid, a fatty acid (e.g., long chain fatty acid), a small molecule (e.g., small lipid), a vitamin or other type of test compound described herein.

Biomarker proteins may also be associated with certain diseases. For example, the biomarker Prss11 or Htra1 identified herein is also associated with wet age-related macular degeneration. It is contemplated that the expression of Htra1, a serine protease, allows neurons to grow properly (e.g., to make synaptic connections). Thus, the ability to alter the expression levels of Htra1 and other biomarkers of the present invention permits the regulation of photoreceptor development (e.g., post mitotic development and connectivity) and photoreceptor death (e.g., in a subject with a disease and/or disorder).

Additionally, experiments conducted during development of the present invention identified mutations with the rd3 gene that are associated with various retinopathies. For example, a homozygous alteration in the invariant G nucleotide of the rd3 exon 2 donor splice site in two siblings with Leber congenital amaurosis (LCA) was identified. This mutation results in premature truncation of the RD3 protein, segregates with the disease, and was not detected in 100 ethnically-matched control individuals. Although an understanding of the mechanism is not necessary to practice the present invention and the present invention is not limited to any particular mechanism of action, in some embodiments, the retinopathy-associated RD3 protein is part of sub-nuclear protein complexes involved in diverse processes, such as transcription and splicing.

B. Biomarker Detection and Treatment Options

In some embodiments, the present invention provides methods for detection of expression of a photoreceptor precursor cell biomarker (e.g., Nrl, Nr2e3 or other biomarker described in FIGS. 11, 12, and 13). In some embodiments, expression is measured directly (e.g., at the nucleic acid or protein level). In some embodiments, expression is detected in tissue samples (e.g., biopsy tissue). In other embodiments, expression is detected in bodily fluids. The present invention further provides panels and kits for the detection of biomarkers. In preferred embodiments, the presence of a biomarker is used to provide information related to retinal organization and status to a subject. For example, the detection of Nrl may be indicative of photoreceptor cells that have a greater likelihood to transplant successfully (e.g., integrate and form synaptic connections (e.g., to become rod cells)) in a host compared to photoreceptor cells lacking Nrl expression and/or activity (e.g., to become cone cells). In addition, the expression level of one or more biomarkers identified herein (e.g., loss of Nrl expression and/or CEP290 (See Example 4)) may be indicative of a retinopathy, disease or disorder in a subject.

The information provided can also be used to direct a course of treatment. For example, if a subject is found to possess or lacks a biomarker (e.g., Nrl), therapies can be chosen to optimize the response to treatment.

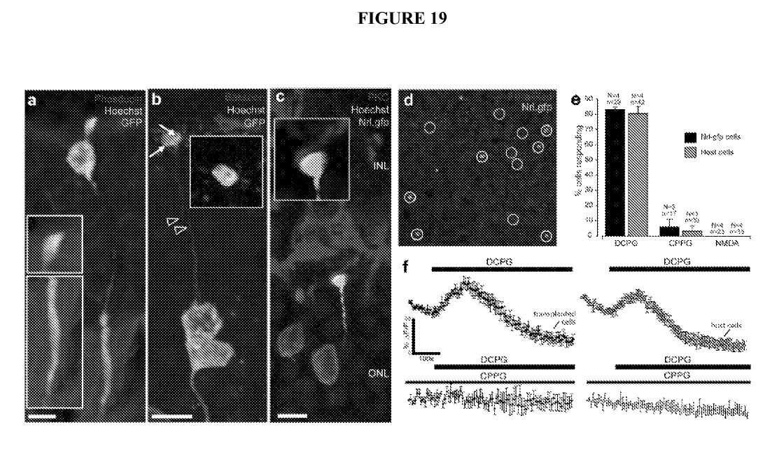

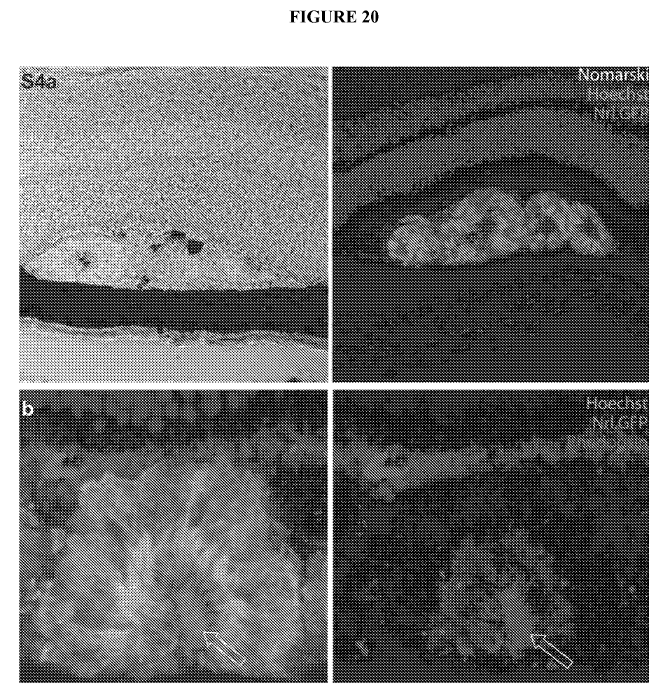

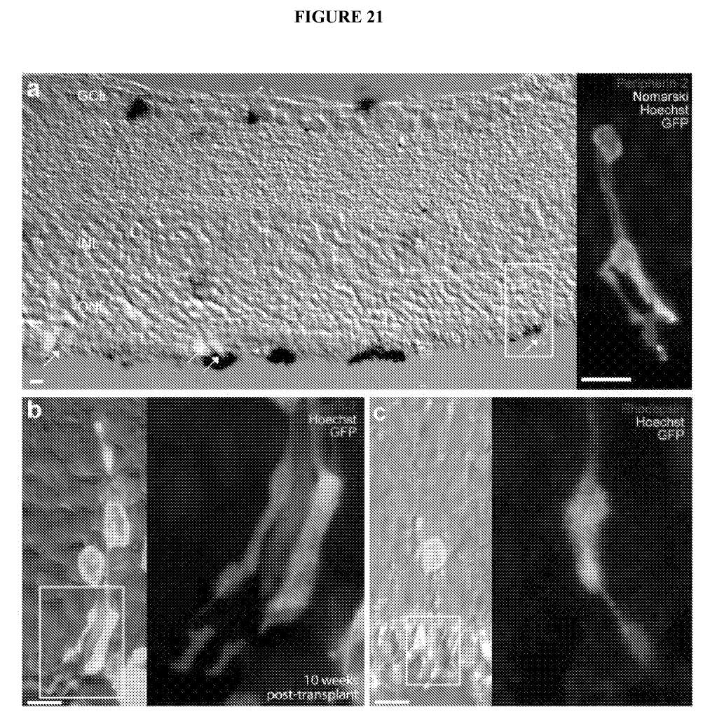

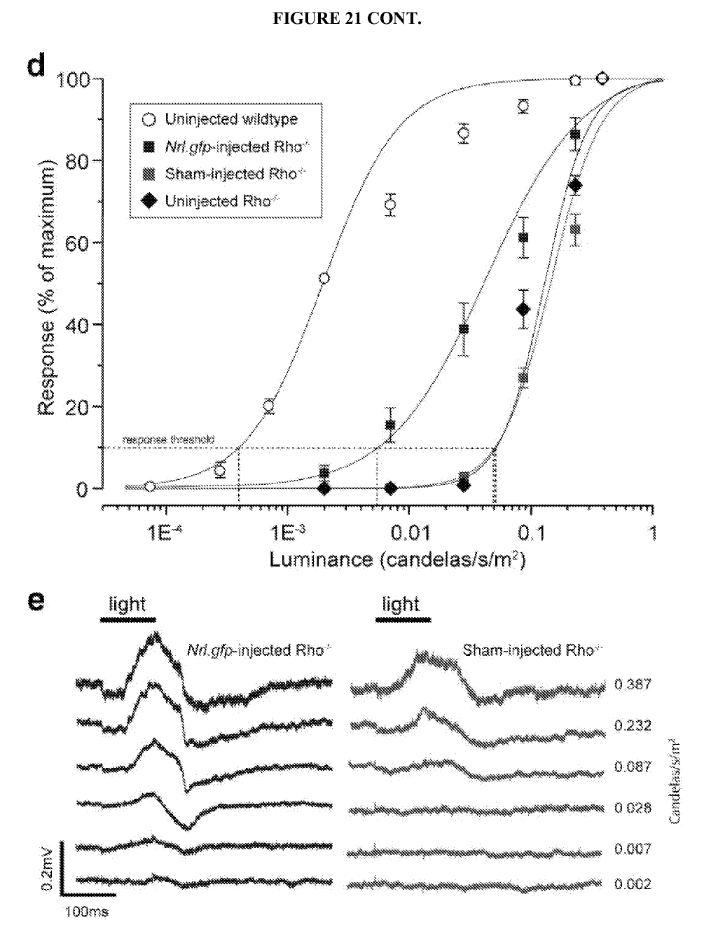

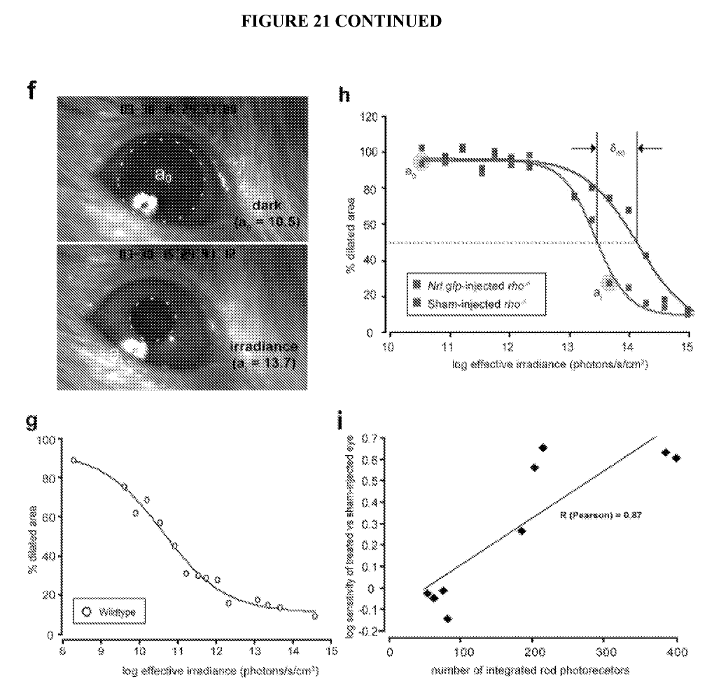

The present invention is not limited to any particular biomarker. Indeed, any biomarker identified herein that correlates with photoreceptor development and/or activity may be utilized, alone or in combination, including, but not limited to, Nrl (See Examples 1 and 2), rhodopsin (See FIG. 21c), CEP290 (See Example 4), bassoon (See FIG. 19b), phosducin (See FIG. 19a), protein kinase C, mGluR8, or a biomarker described in FIG. 11, 12, or 13. Additional biomarkers are also contemplated to be within the scope of the present invention. Any suitable method may be utilized to identify and characterize biomarkers suitable for use in the methods of the present invention, including but not limited to, those described in illustrative Examples 1-4 below. For example, in some embodiments, biomarkers identified as being up or down-regulated using the methods of the present invention are further characterized using microarray (e.g., nucleic acid or tissue microarray), immunohistochemistry, Northern blot analysis, siRNA or antisense RNA inhibition, mutation analysis, investigation of expression with clinical outcome, as well as other methods disclosed herein.

In some embodiments, the present invention provides a panel for the analysis of a plurality of biomarkers. The panel allows for the simultaneous analysis of multiple biomarkers correlating with photoreceptor development and/or activity. For example, a panel may include biomarkers identified as correlating with the likelihood of a photoreceptor cell to integrate post transplantation and/or the likelihood that the integrated cell will form synaptic connections with a host subject. Depending on the subject, panels may be analyzed alone or in combination in order to provide the best possible diagnosis and prognosis. Markers for inclusion on a panel are selected by screening for their predictive value using any suitable method including, but not limited to, those described in the illustrative examples below.

In other embodiments, the present invention provides an expression profile map comprising expression profiles of photoreceptor cells of various stages of development and/or activity. Such maps can be used for comparison with patient samples. Any suitable method may be utilized including, but not limited to, computer comparison of digitized data. The comparison data may be used for research purposes or to provide diagnoses and/or prognoses to patients.

1. Detection of Nucleic Acids (e.g., DNA and RNA)

In some preferred embodiments, detection of biomarkers (e.g., including, but not limited to, those disclosed herein) is detected by measuring the levels of the biomarker (e.g., Nrl, Nr2e3 or other biomarker) in cells and tissue (e.g., photoreceptor cells and tissues). For example, in some embodiments, Nrl can be monitored using antibodies (e.g., antibodies generated according to methods described below) or by detecting Nrl protein. In some embodiments, detection is performed on cells or tissue after the cells or tissues are removed from the subject. In other embodiments, detection is performed by visualizing the biomarker (e.g., Nrl) in cells and tissues residing within the subject.

In some preferred embodiments, detection of biomarkers (e.g., Nrl, Nr2e3) is detected by measuring the expression of corresponding mRNA in a tissue sample (e.g., retina). mRNA expression may be measured by any suitable method, including but not limited to, those disclosed herein.

In some embodiments, RNA is detected by Northern blot analysis. Northern blot analysis involves the separation of RNA and hybridization of a complementary labeled probe.

In still further embodiments, RNA (or corresponding cDNA) is detected by hybridization to a oligonucleotide probe). A variety of hybridization assays using a variety of technologies for hybridization and detection are available. For example, in some embodiments, TAQMAN assay (PE Biosystems, Foster City, Calif.; See e.g., U.S. Pat. Nos. 5,962,233 and 5,538,848, each of which is herein incorporated by reference) is utilized. The assay is performed during a PCR reaction. The TAQMAN assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR (RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

In some embodiments, profiles from healthy photoreceptor cells can be compared with profiles from diseased photoreceptor cells. For example, in some embodiments, a profile from a single cell is generated (e.g., isolated from a cell biopsy). Such a profile may characterize the expression of all genes in the cell. In some embodiments, a profile characterizes the expression of a subset of the genes expressed in the cell (e.g., characterizes the expression of biomarkers identified herein). Thus, a gene chip or RT-PCR or other quantitative assay described herein or well known in the art could be used to generate a profile (e.g., for use in diagnostic or treatment settings).

2. Detection of Protein

In other embodiments, gene expression of biomarkers is detected by measuring the expression of the corresponding protein or polypeptide. Protein expression may be detected by any suitable method. In some embodiments, proteins are detected by immunohistochemistry. In other embodiments, proteins are detected by their binding to an antibody raised against the protein (e.g., against Nrl or other downstream target biomarkers (e.g., Nr2e3). The generation of antibodies is described below.

Antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to biomarkers is utilized.

In other embodiments, an immunoassay described in U.S. Pat. Nos. 5,599,677 and 5,672,480; each of which is herein incorporated by reference, is utilized.

3. Data Analysis

The present invention also provides methods of analyzing, processing and presenting data regarding detection using a biomarker of the present invention (e.g., correlating gene profile of a diseased photoreceptor to that of a healthy photoreceptor using the specific biomarkers described herein (e.g., to provide diagnostic information and/or treatment options).

In some embodiments, a computer-based analysis program is used to translate the raw data generated by the detection assay (e.g., the presence, absence, or amount of a given biomarker or biomarkers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information providers, medical personal, and subjects. For example, in some embodiments of the present invention, a sample (e.g., a biopsy or other sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (e.g., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., degree of photoreceptor loss or the likelihood of responding to a particular treatment) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of biomarkers as useful indicators of a particular condition or stage of disease.

4. Kits

In yet other embodiments, the present invention provides kits for the detection and characterization of biomarkers. In some embodiments, the kits contain antibodies specific for a biomarker (e.g., Nrl), in addition to detection reagents and buffers. In other embodiments, the kits contain reagents specific for the detection of mRNA or cDNA (e.g., oligonucleotide probes or primers). In preferred embodiments, the kits contain all of the components necessary and/or sufficient to perform a detection assay, including all controls, directions for performing assays, and any necessary software for analysis and presentation of results.

5. In Vivo Imaging

In some embodiments, in vivo imaging techniques are used to visualize the expression of biomarkers in an animal (e.g., a human or non-human mammal). For example, in some embodiments, biomarker mRNA or protein is labeled using a labeled antibody specific for the biomarker. A specifically bound and labeled antibody can be detected in an individual using an in vivo imaging method, including, but not limited to, radionuclide imaging, positron emission tomography, computerized axial tomography, X-ray or magnetic resonance imaging method, fluorescence detection, and chemiluminescent detection. Methods for generating antibodies to the biomarkers of the present invention are described herein.

The in vivo imaging methods of the present invention are useful in identifying cells that express the biomarkers of the present invention (e.g., photoreceptor precursor cells). In vivo imaging is used to visualize the presence of a biomarker indicative of photoreceptor cell status. Such techniques allow for identification and characterization without the use of a biopsy. The in vivo imaging methods of the present invention are also useful for providing prognoses to patients (e.g., likelihood of photoreceptor cell loss).

In some embodiments, reagents (e.g., antibodies) specific for the biomarkers of the present invention are fluorescently labeled. The labeled antibodies can be introduced into a subject (e.g., parenterally). Fluorescently labeled antibodies are detected using any suitable method (e.g., using the apparatus described in U.S. Pat. No. 6,198,107, herein incorporated by reference).

In other embodiments, antibodies are radioactively labeled. The use of antibodies for in vivo diagnosis is well known in the art. Sumerdon et al., (Nucl. Med. Biol 17:247-254 (1990) have described an optimized antibody-chelator for the radioimmunoscintographic imaging of tumors using Indium-111 as the label. Griffin et al., (J Clin Onc 9:631-640 (1991)) have described the use of this agent in detecting tumors in patients suspected of having recurrent colorectal cancer. The use of similar agents with paramagnetic ions as labels for magnetic resonance imaging is known in the art (Lauffer, Magnetic Resonance in Medicine 22:339-342 (1991)). The label used will depend on the imaging modality chosen. Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium (III) or Manganese (II) can be used.

Radioactive metals with half-lives ranging from 1 hour to 3.5 days are available for conjugation to antibodies, such as scandium-47 (3.5 days) gallium-67 (2.8 days), gallium-68 (68 minutes), technetiium-99m (6 hours), and indium-111 (3.2 days), of which gallium-67, technetium-99m, and indium-111 are preferable for gamma camera imaging, gallium-68 is preferable for positron emission tomography.

A useful method of labeling antibodies with such radiometals is by means of a bifunctional chelating agent, such as diethylenetriaminepentaacetic acid (DTPA), as described, for example, by Khaw et al. (Science 209:295 (1980)) for In-111 and Tc-99m, and by Scheinberg et al. (Science 215:1511 (1982)). Other chelating agents may also be used, but the 1-(p-carboxymethoxybenzyl)EDTA and the carboxycarbonic anhydride of DTPA are advantageous because their use permits conjugation without affecting the antibody's immunoreactivity substantially.

Another method for coupling DPTA to proteins is by use of the cyclic anhydride of DTPA, as described by Hnatowich et al. (Int. J. Appl. Radiat. Isot. 33:327 (1982)) for labeling of albumin with In-111, but which can be adapted for labeling of antibodies. A suitable method of labeling antibodies with Tc-99m which does not use chelation with DPTA is the pretinning method of Crockford et al., (U.S. Pat. No. 4,323,546, herein incorporated by reference).

A preferred method of labeling immunoglobulins with Tc-99m is that described by Wong et al. (Int. J. Appl. Radiat. Isot., 29:251 (1978)) for plasma protein, and recently applied successfully by Wong et al. (J. Nucl. Med., 23:229 (1981)) for labeling antibodies. In the case of the radiometals conjugated to the specific antibody, it is likewise desirable to introduce as high a proportion of the radiolabel as possible into the antibody molecule without destroying its immunospecificity. A further improvement may be achieved by effecting radiolabeling in the presence of the specific biomarker of the present invention, to insure that the antigen binding site on the antibody will be protected. The antigen is separated after labeling.

In still further embodiments, in vivo biophotonic imaging (Xenogen, Almeda, Calif.) is utilized for in vivo imaging. This real-time in vivo imaging utilizes luciferase. The luciferase gene is incorporated into cells, microorganisms, and animals (e.g., as a fusion protein with a biomarker of the present invention). When active, it leads to a reaction that emits light. A CCD camera and software is used to capture the image and analyze it.

### II. Antibodies

The present invention provides isolated antibodies. In preferred embodiments, the present invention provides monoclonal or polyclonal antibodies that specifically bind to either an isolated polypeptide comprised of at least five amino acid residues of the biomarkers described herein (e.g., Nrl). These antibodies find use in the diagnostic methods described herein.

An antibody against a biomarker of the present invention may be any monoclonal or polyclonal antibody, as long as it can recognize the biomarker. Antibodies can be produced by using a biomarker of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, biomarkers, as such, or together with a suitable carrier or diluent is administered to an animal (e.g., a mammal) under conditions that permit the production of antibodies. For enhancing the antibody production capability, complete or incomplete Freund's adjuvant may be administered. Normally, the biomarker is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 (1975)). As a fusion promoter, for example, polyethylene glycol (PEG) or Sendai virus (HVJ), preferably PEG is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a biomarker of the present invention). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody (e.g., against a biomarker of the present invention) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to a hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, a biomarker of the present invention (further including a gene having a nucleotide sequence partly altered) can be used as the immunogen. Further, fragments of the protein may be used. Fragments may be obtained by any method including, but not limited to expressing a fragment of the gene, enzymatic processing of the protein, chemical synthesis, and the like.

### III. Drug Screening

In some embodiments, the present invention provides drug screening assays (e.g., to screen for photoreceptor development and/or activity altering compounds). The screening methods of the present invention utilize biomarkers identified using the methods of the present invention (e.g., including but not limited to Nrl, Nr2e3 and those described in FIGS. 11, 12, and 13).

For example, in some embodiments, the present invention provides a method of screening for a compound that alters (e.g., increases or decreases) the presence of biomarkers (e.g., Nrl or downstream target molecules). In some embodiments, candidate compounds are antisense agents (e.g., oligonucleotides) directed against biomarkers (e.g., Nrl or downstream target molecules) or proteins that interact with a biomarker (e.g., that inhibit or augment biomarker activity). In other embodiments, candidate compounds are antibodies that specifically bind to a biomarker of the present invention (e.g., Nrl) or proteins that interact with a biomarker (e.g., that inhibit biomarker activity). The present invention is not limited by the type of candidate compound utilized. Indeed, a variety of candidate compounds may be tested including, but are not limited to, carbohydrates, monosaccharides, oligosaccharides, polysaccharides, amino acids, peptides, oligopeptides, polypeptides, proteins, nucleosides, nucleotides, oligonucleotides, polynucleotides, including DNA and DNA fragments, RNA and RNA fragments and the like, lipids, retinoids, steroids, drug, antibody, prodrug, glycopeptides, glycoproteins, proteoglycans and the like, and synthetic analogues or derivatives thereof, including peptidomimetics, small molecule organic compounds and the like, and mixtures thereof.

In some embodiments, test compounds are screened (e.g., characterized) for their ability to alter (e.g., enhance or inhibit) differentiation of a transplanted photoreceptor cell (e.g., a photoreceptor precursor cell). In some embodiments, a test compound is administered (e.g., to a subject receiving transplanted cells, or, to transplanted cells) prior to transplantation. In some embodiments, a test compound is administered (e.g., to a subject receiving transplanted cells, or, to transplanted cells) subsequent to transplantation. In some embodiments, a test compound is administered (e.g., to a subject receiving transplanted cells, or, to transplanted cells) both prior to as well as after transplantation. In some embodiments, one or more types of test compounds are administered to a subject, and/or one or more test compounds are administered to transplanted cells (e.g., before, during and/or after transplantation). In some embodiments, compositions and methods of the present invention are used to characterize the affect of other conditions (e.g., age, diet, environmental exposure, etc.) on photoreceptor cell (e.g., differentiation, response to test compounds, efficacy of transplantation, ability to integrate within the retina, etc.).

In one screening method, test compounds are evaluated for their ability to alter biomarker presence, activity or expression by contacting a test compound with a cell (e.g., a cell expressing or capable of expressing biomarker nucleic acid and/or protein (e.g., a photoreceptor cell (e.g., a photoreceptor precursor cell)) and then assaying for the effect of the test compounds on the presence or expression of a biomarker. In some embodiments, the effect of candidate compounds on expression or presence of a biomarker is assayed for by detecting the level of biomarker mRNA expressed by the cell. mRNA expression can be detected by any suitable method.

In other embodiments, the effect of test/candidate compounds on expression or presence of biomarkers is assayed by measuring the level of polypeptide encoded by the biomarkers. The level of polypeptide expressed can be measured using any suitable method including, but not limited to, those disclosed herein.

Specifically, the present invention provides screening methods for identifying modulators, i.e., candidate or test compounds or agents (e.g., proteins, peptides, peptidomimetics, peptoids, small molecules or other drugs) that bind to or otherwise directly or indirectly affect biomarkers of the present invention, have an inhibitory (or stimulatory) effect on, for example, biomarker (e.g., Nrl, Nr2e3, etc.) expression, biomarker activity or biomarker presence, or have a stimulatory or inhibitory effect on, for example, the expression or activity of a biomarker substrate. Compounds thus identified can be used to modulate the activity of target gene products (e.g., biomarker genes) either directly or indirectly in a therapeutic protocol, to elaborate the biological function of the target gene product, or to identify compounds that disrupt normal target gene interactions. Compounds that inhibit or enhance the activity, expression or presence of biomarkers are useful in the treatment of disorders, diseases or the like characterized by photoreceptor loss or loss of photoreceptor activity.

In some embodiments, the present invention provides assays for screening test compounds that can change cell fate (e.g., from a neural progenitor cell into a photoreceptor precursor cell). For example, any one of the biomarkers idenfied herein can be used to determine if a cell has acquired characteristics that identify it as a photoreceptor precursor (e.g., post exposure to a test compound).

In one embodiment, the invention provides assays for screening candidate or test compounds that are substrates of a biomarker protein or polypeptide or a biologically active portion thereof. In another embodiment, the invention provides assays for screening candidate or test compounds that bind to or modulate the activity of a biomarker protein or polypeptide or a biologically active portion thereof.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 (1994)); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (See, e.g., Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 (1993); Erb et al., Proc. Nad. Acad. Sci. USA 91:11422 (1994); Zuckermann et al., J. Med. Chem. 37:2678 (1994); Cho et al., Science 261:1303 (1993); Carrell et al., Angew. Chem. Int. Ed. Engl. 33.2059 (1994); Carell et al., Angew. Chem. Int. Ed. Engl. 33:2061 (1994); and Gallop et al., J. Med. Chem. 37:1233 (1994).

Libraries of compounds may be presented in solution (e.g., Houghten, Biotechniques 13:412-421 (1992)), or on beads (Lam, Nature 354:82-84 (1991)), chips (Fodor, Nature 364:555-556 (1993)), bacteria or spores (U.S. Pat. No. 5,223,409; herein incorporated by reference), plasmids (Cull et al., Proc. Nad. Acad. Sci. USA 89:18651869 (1992)) or on phage (Scott and Smith, Science 249:386-390 (1990); Devlin Science 249:404-406 (1990); Cwirla et al., Proc. Natl. Acad. Sci. 87:6378-6382 (1990); Felici, J. Mol. Biol. 222:301 (1991)).

In one embodiment, an assay is a cell-based assay in which a cell that expresses or is capable of generating a biomarker is contacted with a test compound, and the ability of the test compound to modulate biomarker presence, expression or activity is determined. Determining the ability of the test compound to modulate biomarker presence, expression or activity can be accomplished by monitoring, for example, changes in enzymatic activity or downstream products of expression (e.g., cellular integration and/or synaptic connectivity).

The ability of the test compound to modulate biomarker binding to a compound (e.g., a biomarker substrate or binding partner) can also be evaluated (e.g. the capacity of Nrl binding to a substrate). This can be accomplished, for example, by coupling the compound (e.g., the substrate or binding partner) with a radioisotope or enzymatic label such that binding of the compound (e.g., the substrate) to a biomarker can be determined by detecting the labeled compound (e.g., substrate) in a complex.

Alternatively, the biomarker can be coupled with a radioisotope or enzymatic label to monitor the ability of a test compound to modulate biomarker binding to a biomarker substrate in a complex. For example, compounds (e.g., substrates) can be labeled with ¹²⁵I, ³⁵_{S} ¹⁴C or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a biomarker substrate) to interact with a biomarker with or without the labeling of any of the interactants can be evaluated. For example, a microphysiometer can be used to detect the interaction of a compound with a biomarker without the labeling of either the compound or the biomarker (McConnell et al. Science 257:1906-1912 (1992)). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and a biomarker.

In yet another embodiment, a cell-free assay is provided in which a biomarker protein, or biologically active portion thereof, or nucleic acid is contacted with a test compound and the ability of the test compound to bind to the biomarker protein, or biologically active portion thereof, or nucleic acid is evaluated. Preferred biologically active portions of the biomarker proteins to be used in assays of the present invention include fragments that participate in interactions with substrates or other proteins (e.g., fragments with high surface probability scores).

Cell-free assays involve preparing a reaction mixture of the target gene protein and the test compound under conditions and for a time sufficient to allow the two components to interact and bind, thus forming a complex that can be removed and/or detected.

The interaction between two molecules (e.g., a biomarker protein and a test compound) can also be detected (e.g., using fluorescence energy transfer (FRET) (See, e.g., Lakowicz et al., U.S. Pat. No. 5,631,169; Stavrianopoulos et al., U.S. Pat. No. 4,968,103; each of which is herein incorporated by reference). A fluorophore label is selected such that a first donor molecule's emitted fluorescent energy will be absorbed by a fluorescent label on a second, ‘acceptor’ molecule, which in turn is able to fluoresce due to the absorbed energy.

Alternately, the ‘donor’ molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A FRET binding event can be conveniently measured through standard fluorometric detection means well known in the art (e.g., using a fluorimeter).

In another embodiment, determining the ability of a biomarker to bind to a target molecule can be accomplished using real-time Biomolecular Interaction Analysis (BIA) (see, e.g., Sjolander and Urbaniczky, Anal. Chem. 63:2338-2345 (1991) and Szabo et al. Curr. Opin. Struct. Biol. 5:699-705 (1995)). “Surface plasmon resonance” or “BIA” detects biospecific interactions in real time, without labeling any of the interactants (e.g., BIACORE). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize biomarkers, an anti-biomarker antibody or its target molecule to facilitate separation of complexed from non-complexed forms of one or both of the molecules, as well as to accommodate automation of the assay. Binding of a test compound to a biomarker (e.g., protein or nucleic acid), or interaction of a biomarker with a target molecule in the presence and absence of a candidate compound, can be accomplished in any vessel suitable for containing the reactants. Examples of such vessels include microtiter plates, test tubes, and micro-centrifuge tubes.

For example, in one embodiment, a fusion protein can be provided which adds a domain that allows one or both of the molecules to be bound to a matrix. For example, glutathione-S-transferase- biomarker fusion proteins or glutathione-S-transferase/target fusion proteins can be adsorbed onto glutathione Sepharose beads (Sigma Chemical, St. Louis, Mo.) or glutathione-derivatized microtiter plates, which are then combined with the test compound or the test compound and either the non-adsorbed target protein or biomarker protein, and the mixture incubated under conditions conducive for complex formation (e.g., at physiological conditions for salt and pH). Following incubation, the beads or microtiter plate wells are washed to remove any unbound components, the matrix immobilized in the case of beads, complex determined either directly or indirectly, for example, as described above.

Alternatively, the complexes can be dissociated from the matrix, and the level of biomarkers binding or activity determined using standard techniques. Other techniques for immobilizing either biomarker molecule (e.g., nucleic acid or protein) or a target molecule on matrices include using conjugation of biotin and streptavidin. Biotinylated biomarker or target molecules can be prepared from biotin-NHS (N-hydroxy-succinimide) using techniques known in the art (e.g., biotinylation kit, Pierce Chemicals, Rockford, EL), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-IgG antibody).

This assay is performed utilizing antibodies reactive with biomarker or target molecules but which do not interfere with binding of the biomarker to its target molecule. Such antibodies can be derivatized to the wells of the plate, and unbound target or biomarkers trapped in the wells by antibody conjugation. Methods for detecting such complexes, in addition to those described above for the GST-immobilized complexes, include immunodetection of complexes using antibodies reactive with the biomarker or target molecule, as well as enzyme-linked assays which rely on detecting an enzymatic activity associated with the biomarker or target molecule.

Alternatively, cell free assays can be conducted in a liquid phase. In such an assay, the reaction products are separated from unreacted components, by any of a number of standard techniques, including, but not limited to: differential centrifugation (See, e.g., Rivas and Minton, Trends Biochem Sci 18:284-7 (1993)); chromatography (gel filtration chromatography, ion-exchange chromatography); electrophoresis (See, e.g., Ausubel et al., eds. Current Protocols in Molecular Biology 1999, J. Wiley: New York.); and immunoprecipitation (See, e.g., Ausubel et al., eds. Current Protocols in Molecular Biology 1999, J. Wiley: New York). Such resins and chromatographic techniques are known to one skilled in the art (See, e.g., Heegaard J. Mol. Recognit 11:141-8 (1998); Hageand Tweed J. Chromatogr. Biomed. Sci. Appl 699:499-525 (1997)). Further, fluorescence energy transfer may also be conveniently utilized, as described herein, to detect binding without further purification of the complex from solution.

The assay can include contacting the biomarker protein, or biologically active portion thereof, or nucleic acid with a known compound that binds the biomarker to form an assay mixture, contacting the assay mixture with a test compound, and determining the ability of the test compound to interact with a biomarker, wherein determining the ability of the test compound to interact with a biomarker includes determining the ability of the test compound to preferentially bind to biomarker protein, or biologically active portion thereof, or nucleic acid, or to modulate the activity of a target molecule, as compared to the known compound.

To the extent that biomarkers can, in vivo, interact with one or more cellular or extracellular macromolecules, such as proteins, inhibitors or inducers of such an interaction are useful. A homogeneous assay can be used to identify inhibitors.

For example, a preformed complex of the target gene product and the interactive cellular or extracellular binding partner product is prepared such that either the target gene products or their binding partners are labeled, but the signal generated by the label is quenched due to complex formation (See, e.g., U.S. Pat. No. 4,109,496, herein incorporated by reference, that utilizes this approach for immunoassays). The addition of a test substance that competes with and displaces one of the species from the preformed complex will result in the generation of a signal above background. In this way, test substances that disrupt target gene product-binding partner interaction can be identified. Alternatively, biomarkers can be used as a “bait” in a two-hybrid assay or three-hybrid assay (See, e.g., U.S. Pat. No. 5,283,317; Zervos et al., Cell 72:223-232 (1993); Madura et al., J. Biol. Chem. 268.12046-12054 (1993); Bartel et al., Biotechniques 14:920-924 (1993); Iwabuchi et al., Oncogene 8:1693-1696 (1993); and Brent WO 94/10300; each of which is herein incorporated by reference), to identify proteins that bind to or interact with biomarkers (“biomarker-binding proteins” or “biomarker-bp”) and are involved in biomarker activity. Such biomarker-bps can be activators or inhibitors of signals by the biomarkers or targets as, for example, downstream elements of a biomarkers-mediated signaling pathway (e.g. synaptic activity (e.g., PKC)).

Modulators of biomarker expression can also be identified. For example, a cell or cell free mixture can be contacted with a candidate compound and the expression of biomarker nucleic acid (e.g., Nrl DNA or mRNA) or protein evaluated relative to the level of expression of biomarker nucleic acid (e.g., DNA or mRNA) or protein in the absence of the candidate compound. When expression of biomarker nucleic acid (e.g., DNA or mRNA) or protein is greater in the presence of the candidate compound than in its absence, the candidate compound is identified as a stimulator of biomarker nucleic acid (e.g., DNA or mRNA) or protein expression. Alternatively, when expression of biomarker nucleic acid (e.g., DNA or mRNA) or protein is less (e.g., statistically significantly less) in the presence of the candidate compound than in its absence, the candidate compound is identified as an inhibitor of biomarker nucleic acid (e.g., DNA or mRNA) or protein expression. The level of biomarker nucleic acid (e.g., DNA or mRNA) or protein expression can be determined by methods described herein for detecting biomarker nucleic acid (e.g., DNA or mRNA) or protein.

A modulating agent can be identified using a cell-based or a cell free assay, and the ability of the agent to modulate the activity of a biomarker nucleic acid (e.g., DNA or mRNA) or protein can be confirmed in vivo, for example, in an animal such as an animal model for a disease (e.g., an animal with a retinopathy caused by disease or disorder); or an animal harboring transplanted photoreceptor cells (e.g., from an animal (e.g., a mouse or human)).

This invention further pertains to novel agents identified by the above-described screening assays. Accordingly, it is within the scope of this invention to further use an agent (e.g., test compound) identified as described herein (e.g., a biomarker modulating agent, an antisense biomarker nucleic acid molecule, a siRNA molecule, a biomarker specific antibody, or a biomarker-binding partner) in an appropriate animal model (such as those described herein) to determine the efficacy, toxicity, side effects, or mechanism of action, of treatment with such an agent. Furthermore, novel agents identified by the above-described screening assays can be, for example, used for treatments as described herein.

### IV. Photoreceptor Cell Therapies

In some embodiments, the present invention provides therapies for photoreceptor cells (e.g., photoreceptor cell loss). In some embodiments, therapies provide biomarkers (e.g., including but not limited to, Nrl) for the treatment of photoreceptor cells (e.g., inducing integration of photoreceptor cells and/or synaptic connectivity of photoreceptor cells). In some embodiments, therapies provide photoreceptor precursor cells for the treatment of photoreceptor cell loss.

A. Administering Therapeutics Comprising Biomarker Protein or Peptides

It is contemplated that a biomarker (e.g., Nrl, Nr2e3, etc.), biomarker-derived peptides and biomarker-derived peptide analogues or mimetics, can be administered (e.g., locally) to induce photoreceptor cell (e.g., photoreceptor precursor cell) development (e.g., in vitro, in vivo or ex vivo). Moreover, they can be administered alone or in combination with test compounds described and identified herein.

Where combinations are contemplated, it is not intended that the present invention be limited by the particular nature of the combination. The present invention contemplates combinations as simple mixtures as well as chemical hybrids. An example of the latter is where the peptide or drug is covalently linked to a targeting carrier or to an active pharmaceutical. Covalent binding can be accomplished by any one of many commercially available crosslinking compounds.

It is not intended that the present invention be limited by the particular nature of the therapeutic preparation. For example, such compositions can be provided together with physiologically tolerable liquid, gel or solid carriers, diluents, adjuvants and excipients.

These therapeutic preparations can be administered to mammals for veterinary use, such as with domestic animals, and clinical use in humans in a manner similar to other therapeutic agents. In general, the dosage required for therapeutic efficacy will vary according to the type of use and mode of administration, as well as the particularized requirements of individual hosts.

Such compositions are typically prepared as liquid solutions or suspensions, or in solid forms. Oral formulations usually will include such normally employed additives such as binders, fillers, carriers, preservatives, stabilizing agents, emulsifiers, buffers and excipients as, for example, pharmaceutical grades of mannitol, lactose, starch, magnesium stearate, sodium saccharin, cellulose, magnesium carbonate, and the like. These compositions take the form of solutions, suspensions, tablets, pills, capsules, sustained release formulations, or powders, and typically contain 1%-95% of active ingredient, preferably 2%-70%.

The compositions are also prepared as injectables, either as liquid solutions or suspensions; solid forms suitable for solution in, or suspension in, liquid prior to injection may also be prepared.

The compositions of the present invention are often mixed with diluents or excipients which are physiological tolerable and compatible. Suitable diluents and excipients are, for example, water, saline, dextrose, glycerol, or the like, and combinations thereof. In addition, if desired the compositions may contain minor amounts of auxiliary substances such as wetting or emulsifying agents, stabilizing or pH buffering agents.

Additional formulations which are suitable for other modes of administration, such as topical administration, include salves, tinctures, creams, and lotions, and, in some cases, suppositories. For salves and creams, traditional binders, carriers and excipients may include, for example, polyalkylene glycols or triglycerides.

B. Designing Mimetics

It may be desirable to administer an analogue of a biomarker (e.g., Nrl or downstream regulatory protein (e.g., Nr2e3)))-derived peptide. In some embodiments, it may be desirable to administer an analogue of a specific biomarker (e.g., Nrl downstream protein (e.g., N2e3)) in order to manipulate expression of only selected genes associated with a specific disease (e.g., CEP290 for Leber congenital amaurosis, or any one or more like rd1, rd 2, rd 3, rd 6, rd 7, rd 9, or rd 11, or for retinal degeneration associated with aging). A variety of designs for such mimetics are possible. For example, cyclic peptide mimetics, in which the necessary conformation for binding is stabilized by nonpeptides, are specifically contemplated. (See, e.g., U.S. Pat. No. 5,192,746 to Lobl et al., U.S. Pat. No. 5,169,862 to Burke, Jr. et al., U.S. Pat. No. 5,539,085 to Bischoff et al., U.S. Pat. No. 5,576,423 to Aversa et al., U.S. Pat. No. 5,051,448 to Shashoua, and U.S. Pat. No. 5,559,103 to Gaeta et al., each of which is hereby incorporated by reference, describe multiple methods for creating such compounds.

Synthesis of nonpeptide compounds that mimic peptide sequences is also known in the art. For example, Eldred et al., J. Med. Chem. 37:3882 (1994), describe nonpeptide antagonists that mimic the Arg-Gly-Asp sequence. Likewise, Ku et al., J. Med. Chem. 38:9 (1995) give further elucidation of the synthesis of a series of such compounds. Such nonpeptide compounds are specifically contemplated by the present invention.

The present invention also contemplates synthetic mimicking compounds that are multimeric compounds that repeat the relevant peptide sequence. As is known in the art, peptides can be synthesized by linking an amino group to a carboxyl group that has been activated by reaction with a coupling agent, such as dicyclohexyl-carbodiimide (DCC). The attack of a free amino group on the activated carboxyl leads to the formation of a peptide bond and the release of dicyclohexylurea. It may be important to protect potentially reactive groups other than the amino and carboxyl groups intended to react. For example, the x-amino group of the component containing the activated carboxyl group can be blocked with a tertbutyloxycarbonyl group. This protecting group can be subsequently removed by exposing the peptide to dilute acid, which leaves peptide bonds intact.

With this method, peptides can be readily synthesized by a solid phase method by adding amino acids stepwise to a growing peptide chain that is linked to an insoluble matrix, such as polystyrene beads. The carboxyl-terminal amino acid (with an amino protecting group) of the desired peptide sequence is first anchored to the polystyrene beads. The protecting group of the amino acid is then removed. The next amino acid (with the protecting group) is added with the coupling agent. This is followed by a washing cycle. The cycle is repeated as necessary.

The methods of the present invention can be practiced in vitro or in vivo.

For example, the method of the present invention can be used in vitro to screen for compounds that are potentially useful for combinatorial use with Nrl or other biomarker peptides for treating photoreceptor cells (e.g., photoreceptor precursor cells); to evaluate a compound's efficacy in treating photoreceptor cells; or to investigate the mechanism by which a compound alters photoreceptor cell development and/or activity (e.g., photoreceptor cell integration and/or synaptic connectivity). For example, once a compound has been identified as a compound that works in combination with biomarker (e.g., Nrl, Nr2e3 or downstream genes) peptides, one skilled in the art can apply the method of the present invention in vitro to evaluate the degree to which the compound induces photoreceptor cell activity and/or development; or one skilled in the art can apply the method of the present invention to determine a mechanism by which the compound operates, or by a combination of these methods.

Alternatively, a method of the present invention can be used in vivo (e.g., to treat retinopathies (e.g., comprising photoreceptor cell loss and/or loss of activity). In the case where the method of the present invention is carried out in vivo, for example, where the photoreceptor cells are present in a subject (e.g., a mouse or a human subject), contacting can be carried out by administering a therapeutically effective amount of the compound to the human subject (e.g., by directly injecting the compound or through systemic administration).

Suitable subjects include, for example mammals, such as rats, mice, cats, dogs, monkeys, and humans. Suitable human subjects include, for example, those that have previously been determined to be at risk of retinal disease or disorder and those who have been diagnosed as having retinal disease or disorder or injury.

In subjects who are determined to be at risk of having retinal disease or disorder, a composition of the present invention can be administered to the subject preferably under conditions effective to decrease symptoms associated with retinopathy (e.g., photoreceptor cell loss) in the event that they develop.

In addition to a biomarker of the present invention or test compound identified herein, these compositions can include other active materials, particularly, actives that have been identified as useful in the treatment retinopathies. Various types of retinopathies exist.

Many types of retinopathy are progressive and may result in blindness or severe vision loss or impairment, particularly if the macula becomes affected. Retinopathy can be diagnosed by an optometrist or an ophthalmologist (e.g., using ophthalmoscopy). Thus, one of skill in the art knows well the types of actives that may find use in treatment (e.g., that may depend upon the cause of the disease).

Thus, one of skill in the art immediately appreciates that the actual preferred amount of composition comprising a biomarker to be administered according to the present invention may vary according to the particular composition formulated, and the mode of administration. Many factors that may modify the action of the compositions (e.g., body weight, sex, diet, time of administration, route of administration, rate of excretion, condition of the subject, drug combinations, and reaction sensitivities and severities) can be taken into account by those skilled in the art. Administration can be carried out continuously or periodically within the maximum tolerated dose. Optimal administration rates for a given set of conditions can be ascertained by those skilled in the art using conventional dosage administration tests.

C. Therapeutic Agents Combined or Co-Administered with Biomarker (e.g., Nrl, Nr2e3 or a Downstream Regulatory Gene) Peptides

A wide range of therapeutic agents find use with the present invention. For example, any therapeutic agent that can be co-administered with biomarker (e.g., Nrl, Nr2e3 or a downstream regulatory gene) peptides, or associated with biomarker (e.g., Nrl) is suitable for use in the present invention.

Some embodiments of the present invention provide administering to a subject an effective amount of biomarker (e.g., Nrl, Nr2e3 or a downstream regulatory gene) peptides (and enantiomers, derivatives, and pharmaceutically acceptable salts thereof) and at least one agent.

Any pharmaceutical that is routinely used in a retinopathy therapy context finds use in the present invention (e.g., neovascularization inhibitors (e.g., AVASTIN or LUCENTIS from Genentech, San Francisco, Calif.), cell therapy, steroids, etc.). These agents may be prepared and used as a combined therapeutic composition, or kit, by combining it with an immunotherapeutic agent, as described herein.

In some embodiments, the agents are attached to Nrl or other biomarker with photocleavable linkers. For example, several heterobifunctional, photocleavable linkers that find use with the present invention are described (See, e.g., Ottl et al., Bioconjugate Chem., 9:143 (1998)). These linkers can be either water or organic soluble. They contain an activated ester that can react with amines or alcohols and an epoxide that can react with a thiol group. In between the two groups is a 3,4-dimethoxy6-nitrophenyl photoisomerization group, which, when exposed to near-ultraviolet light (365 nm), releases the amine or alcohol in intact form. Thus, the therapeutic agent, when linked to the compositions of the present invention using such linkers, may be released in biologically active or activatable form through exposure of the target area to near-ultraviolet light.

An alternative to photocleavable linkers are enzyme cleavable linkers. The linkers are stable outside of the cell, but are cleaved by thiolproteases once within the cell. In a preferred embodiment, the conjugate PK1 is used. As an alternative to the photocleavable linker strategy, enzyme-degradable linkers, such as Gly-Phe-Leu-Gly may be used.

Antimicrobial therapeutic agents may also be used in combination with Nrl or other biomarkers as therapeutic agents in the present invention. Any agent that can kill, inhibit, or otherwise attenuate the function of microbial organisms may be used, as well as any agent contemplated to have such activities. Antimicrobial agents include, but are not limited to, natural and synthetic antibiotics, antibodies, inhibitory proteins, antisense nucleic acids, membrane disruptive agents and the like, used alone or in combination. Indeed, any type of antibiotic may be used including, but not limited to, anti-bacterial agents, anti-viral agents, anti-fungal agents, and the like.

In still further embodiments, another component of the present invention is that the biomarker be associated with targeting agents (Nrl or other biomarker-targeting agent complex) that are able to specifically target a particular cell type (e.g., photoreceptor precursor cell or differentiating photoreceptor). Cell surface biomarker proteins of the present invention serve as ideal candidates for assessing the effects of the therapy and to identify appropriate intermediate cell stages for therapy. These biomarkers include CD24a, CD1d1, Chrnb4, Clic4, Ddr1, F2r, Gpr137b, Igsf4b, LRP4, Nope, Nrp1, Pdpn, Ptpro, St8sia4, and Tmem46.

Any moiety known to be located on the surface of target cells (e.g., photoreceptor cells) finds use with the present invention. For example, an antibody directed against such a moiety targets the compositions of the present invention to cell surfaces containing the moiety. Alternatively, the targeting moiety may be a ligand directed to a receptor present on the cell surface or vice versa.

In some embodiments of the present invention, a number of photoreceptor cell targeting groups are associated with a cell surface or other biomarker described herein. Thus, cell surface or other biomarker associated with targeting groups are specific for targeting photoreceptor cells (i.e., much more likely to attach to photoreceptor cells and not to other types of cells).

In preferred embodiments of the present invention, targeting groups are associated (e.g., covalently or noncovalently bound) to a cell surface or other biomarker with either short (e.g., direct coupling), medium (e.g., using small-molecule bifunctional linkers such as SPDP, sold by Pierce Chemical Company), or long (e.g., PEG bifunctional linkers) linkages.

In preferred embodiments of the present invention, the targeting agent is an antibody or antigen binding fragment of an antibody (e.g., Fab units). Antibodies can be generated to allow for the targeting of antigens or immunogens. Such antibodies include, but are not limited to polyclonal, monoclonal, chimeric, single chain, Fab fragments, and a Fab expression library.

Various procedures known in the art are used for the production of polyclonal antibodies. For the production of antibody, various host animals can be immunized by injection with the peptide corresponding to the desired epitope including but not limited to rabbits, mice, rats, sheep, goats, etc. In a preferred embodiment, the peptide is conjugated to an immunogenic carrier (e.g., diphtheria toxoid, bovine serum albumin (BSA), or keyhole limpet hemocyanin (KLH)). Various adjuvants are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies, any technique that provides for the production of antibody molecules by continuous cell lines in culture may be used (See e.g., Harlow and Lane, Antibodies: A Laboratory Manual, Cold Spring Harbor Laboratory Press, Cold Spring Harbor, N.Y.). These include, but are not limited to, the hybridoma technique originally developed by Kohler and Milstein (Kohler and Milstein, Nature 256:495-497 (1975)), as well as the trioma technique, the human B-cell hybridoma technique (See e.g., Kozbor et al., Immunol. Today 4:72 (1983)), and the EBV-hybridoma technique to produce human monoclonal antibodies (Cole et al., in Monoclonal Antibodies and Cancer Therapy, Alan R. Liss, Inc., pp. 77-96 (1985)).

In an additional embodiment of the invention, monoclonal antibodies can be produced in germ-free animals utilizing recent technology (See e.g., PCT/US90/02545). According to the invention, human antibodies may be used and can be obtained by using human hybridomas (Cote et al., Proc. Natl. Acad. Sci. U.S.A. 80:2026-2030 (1983)) or by transforming human B cells with EBV virus in vitro (Cole et al., in Monoclonal Antibodies and Cancer Therapy, Alan R. Liss, pp. 77-96 (1985)).

According to the invention, techniques described for the production of single chain antibodies (U.S. Pat. No. 4,946,778; herein incorporated by reference) can be adapted to produce specific single chain antibodies. An additional embodiment of the invention utilizes the techniques described for the construction of Fab expression libraries (Huse et al., Science 246:1275-1281 (1989)) to allow rapid and easy identification of monoclonal Fab fragments with the desired specificity.

Antibody fragments that contain the idiotype (antigen binding region) of the antibody molecule can be generated by known techniques. For example, such fragments include but are not limited to: the F(ab′)2 fragment that can be produced by pepsin digestion of the antibody molecule; the Fab′ fragments that can be generated by reducing the disulfide bridges of the F(ab′)2 fragment, and the Fab fragments that can be generated by treating the antibody molecule with papain and a reducing agent.

In the production of antibodies, screening for the desired antibody can be accomplished by techniques known in the art (e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western Blots, precipitation reactions, agglutination assays (e.g., gel agglutination assays, hemagglutination assays, etc.), complement fixation assays, immunofluorescence assays, protein A assays, and immunoelectrophoresis assays, etc.).

A very flexible method to identify and select appropriate peptide targeting groups is the phage display technique (See e.g., Cortese et al., Curr. Opin. Biotechol., 6:73 (1995)), which can be conveniently carried out using commercially available kits. The phage display procedure produces a large and diverse combinatorial library of peptides attached to the surface of phage, which are screened against immobilized surface receptors for tight binding. After the tight-binding, viral constructs are isolated and sequenced to identify the peptide sequences. The cycle is repeated using the best peptides as starting points for the next peptide library. Eventually, suitably high-affinity peptides are identified and then screened for biocompatibility and target specificity. In this way, it is possible to produce peptides that can be conjugated to Nrl or other biomarker describe herein, producing multivalent conjugates with high specificity and affinity for the target cell receptors (e.g., photoreceptor cell receptors) or other desired targets.

In some embodiments of the present invention, the targeting agents (moities) are preferably nucleic acids (e.g., RNA or DNA). In some embodiments, the nucleic acid targeting moities are designed to hybridize by base pairing to a particular nucleic acid (e.g., chromosomal DNA, mRNA, or ribosomal RNA). In other embodiments, the nucleic acids bind a ligand or biological target. Nucleic acids that bind ligands are preferably identified by the SELEX procedure (See e.g., U.S. Pat. Nos. 5,475,096; 5,270,163; and 5,475,096; and in PCT publications WO 97/38134, WO 98/33941, and WO 99/07724, each of which is herein incorporated by reference), although many methods are known in the art.

D. Pharmaceutical Compositions

The present invention further provides pharmaceutical compositions (e.g., comprising Nrl, Nr2e3, their agonists or ligands, or other biomarker compositions described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; intracranial; sub-retinal; intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188, hereby incorporated by reference), cationic glycerol derivatives, and polycationic molecules, such as polylysine (WO 97/30731, hereby incorporated by reference), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Certain embodiments of the invention provide pharmaceutical compositions containing (a) one or more Nrl or other biomarker compounds (e.g., mimetic or portion thereof) and (b) one or more other agents. Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Other non-antisense agents are also within the scope of this invention. Two or more combined compounds may be used together or sequentially.

Dosing is dependent on severity and responsiveness of the disease state to be treated (e.g., determined using compositions and methods of the present invention), with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual agents, and can generally be estimated based on EC₅₀s found to be effective in in vitro and in vivo animal models. In general, dosage is from 0.01 μg to 100 kg per kg of body weight, and may be given once or more daily, weekly, monthly or yearly. The treating physician can estimate repetition rates for dosing based on measured residence times and concentrations of the agent in bodily fluids or tissues. Following successful treatment, it may be desirable to have the subject undergo maintenance therapy to prevent the recurrence of the disease state, wherein the agent is administered in maintenance doses, ranging from 0.01 μg to 100 kg per kg of body weight, once or more daily, to once every 20 years.

E. Introduction of Biomarkers to Photoreceptor Cells and Tissue

In some embodiments, the present invention provides methods for determining how to treat retinopathy comprising determining the level of biomarker expression and/or activity in photoreceptor cells and providing a treatment selected based upon biomarker status. The present invention further provides a method for altering photoreceptor activity and/or development comprising altering the levels of biomarker in the photoreceptor cell. The art knows well multiple methods of altering the level of expression of a biomarker gene or protein in a cell (e.g., ectopic or heterologous expression of a gene). The following are provided as exemplary methods, and the invention is not limited to any particular method.

In some embodiments, the present invention provides a method of treating photoreceptor cells comprising altering responsiveness of the photoreceptor cell to treatment comprising making the photoreceptor cell either more or less responsive (e.g., sensitive) to the treatment. In some embodiments, making the photoreceptor cell more or less responsive (e.g., sensitive) to treatment comprises altering the level of Nrl, Nr2e3 or other biomarker in the photoreceptor cell. In some embodiments, altering the level of Nrl, Nr2e3, or other biomarker in the photoreceptor cell comprises altering the level of or activity of Nrl, Nr2e3 or other biomarker protein in a photoreceptor cell (e.g., using the compositions and methods described herein). In some embodiments, the altering increases the level of activity of Nrl, Nr2e3 or other biomarker. The present invention further provides a method of customizing a photoreceptor cell for treatment by altering Nrl, Nr2e3 or other biomarker levels in the photoreceptor cell.

In some embodiments, the activity (e.g., the presence or absence of activity) of Nrl promoter identifies a photoreceptor precursor (e.g., a rod precursor or a cone precursor (See, e.g., Example 1)). In some embodiments, the expression of Nrl in a non-rod cell (e.g., a cone cell) can convert the non-rod cell to a rod photoreceptor (See, e.g., Example 6). In some embodiments, suppressing the expression and/or activity of Nrl or one or more of its downstream targets (e.g., Nr2e3) can be used to generate and/or identify a photoreceptor cell (e.g., a rod or cone cell) (See, e.g., Example 9). In some embodiments, the expression of Nrl can be induced by small molecules (e.g., retinoic acid) to generate a rod photoreceptor (See, e.g., Example 8). In some embodiments, the activity of Nrl can be altered (e.g., enhanced or suppressed) by altering post-translational modification (e.g., phosphorylation, acetylation, glycosylation, etc.) of Nrl (e.g., in order to activate or suppress specific genes or their products (See, e.g., Example 7)). In some embodiments, full-length or a portion of Nrl can be used to activate or suppress a gene or protein and/or to manage or treat an eye disease. In some embodiments, the targets of Nrl (e.g., Nr2e3) or other biomarkers described herein can be used to activate or suppress a gene or protein and/or to manage or treat an eye disease. The present invention is not limited to any particular target of Nrl. In some embodiments the target of Nrl is Nr2e3.

In some embodiments, the present invention provides that Nrl binds to a sequence element in the Nr2e3 promoter and enhances its activity (e.g., alone, or together with the homeodomain protein CRX (See, e.g., Example 9)). CRX is a photoreceptor-specific homeodomain protein that plays a critical role in the maturation of photoreceptors (See, e.g., Chen et al., Neuron 19 (1997) 1017-1030; Furukawa et al., Cell 91 (1997) 531-541). Although an understanding of a mechanism is not necessary to practice the present invention, and the present invention is not limited to any particular mechanism, in some embodiments, CRX acts as a photoreceptor competence factor before NRL defines rod identity.

In some embodiments, the present invention provides expression profiles of retinas from transgenic mice that ectopically express either NRL and NR2E3 or NR2E3 alone in cone precursors (See, e.g., Example 9). In some embodiments, the present invention provides cone enriched genes (See, e.g., Example 9). In some embodiments, the present invention provides that regulatory networks that define rod versus cone identity are under the direct control of NRL. In some embodiments, the present invention provides that NR2E3 is a direct transcriptional target of NRL and that specification of rod cell fate over cone differentiation is dictated by the activation of NR2E3 in response to NRL (See, e.g., Example 9). In some embodiments, ectopic NR2E3 function is sufficient to inhibit the development of S and M-cones and necessary to repress M and some S-cones; however, expression of NRL is only sufficient to repress a subset of S-cones. The present invention also identifies the presence of ectopic S-opsin positive cells that persist and survive in the adult retinas from Nrl−/− and rd7 mice. Although an understanding of the mechanism is not necessary to practice the present invention and the present invention is not limited to any particular mechanism of action, in some embodiments, NRL and NR2E3 dictate the expression of specific guidance cues that facilitate photoreceptor path finding to the vicinity of their appropriate target regions in a highly stereotyped and directed manner. For example, in some embodiments, the present invention provides several target proteins that show an altered expression profile in the Nrl−/− retina that correlate with the role of an axonal guidance cue (See, e.g., Example 9 and Yoshida et al., Hum Mol Genet 13 (2004) 1487-1503; Yu et al., J Biol Chem 279 (2004) 42211-42220). Targets include, but are not limited to, members of families of secreted signaling molecules, such as Wingless/Wnt and Decapentaplegic/Bone Morphogenic Protein/Transforming Growth Factor B (Dpp/BMP/TGFb) (See, e.g., Example 9). Although an understanding of a mechanism is not necessary to practice the present invention, and the present invention is not limited to any particular mechanism, in some embodiments, the absence of NRL, and consequently NR2E3, lead to changes in Wnt and BMP pathway that create noise in a homing signal that is required to (i) bring all photoreceptors to the ONL, and/or (ii) promote the appropriate wiring of rods and cones to bipolar and horizontal neurons. In some embodiments, the present invention provides that NRL and/or NR2E3 can be used to shut off pathways (e.g., receptor mediated pathways, signaling pathways, developmental pathways, etc.) involved in photoreceptor progenitor cell development (e.g., development and/or differentiation of progenitor cells (e.g., into cones)). For example, in some embodiments, the present invention provides that alteration of NRL and/or NR2E3 expression and/or activity can be used to activate or shut off pathways (e.g., receptor mediated pathways, signaling pathways, developmental pathways, etc.) involved in photoreceptor progenitor cell development (e.g., development and/or differentiation of progenitor cells (e.g., into cones)).

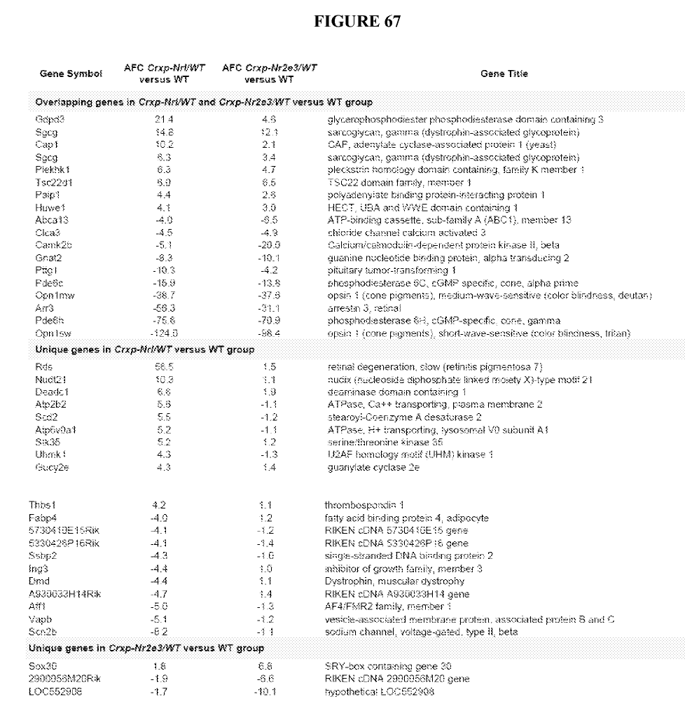

The present invention provides many targets of Nrl, Nrl and Nr2e3, and/or Nr2e3 alone. For example, targets include, but are not limited to, genes identified herein (e.g., in Example 9) and listed in FIGS. 67, 68 and 69. Although an understanding of the mechanism is not necessary to practice the present invention and the present invention is not limited to any particular mechanism of action, in some embodiments, a target of Nrl, Nrl and Nr2e3, and/or Nr2e3 alone is under direct transcriptional control of Nrl and/or Nr2e3 (e.g., See Example 9, Nr2e3). In some embodiments, the target of Nrl is under indirect transcriptional control of Nrl and/or Nr2e3 (e.g., in some embodiments, Nrl activates transcription and expression a gene, and the expression of the gene then acts to activate transcription and expression of the target).

While it is contemplated that Nrl, Nr2e3 or other biomarker protein may be delivered directly, a preferred embodiment involves providing a nucleic acid encoding Nrl or other biomarker protein of the present invention to a cell. Following this provision, the Nrl or other biomarker protein is synthesized by the transcriptional and translational machinery of the cell. In some embodiments, additional components useful for transcription or translation may be provided by the expression construct comprising Nrl or other biomarker nucleic acid sequence (e.g., wild-type or mutant Nrl or other biomarker, or portions thereof).

In some embodiments, the nucleic acid encoding Nrl, Nr2e3 or other biomarker protein may be stably integrated into the genome of the cell. In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on, among other things, the type of expression construct employed.

The ability of certain viruses to infect cells or enter cells via receptor-mediated endocytosis, and to integrate into host cell genome and express viral genes stably and efficiently have made them attractive candidates for the transfer of foreign genes into mammalian cells. In some embodiments, vectors of the present invention are viral vectors (e.g., phage or adenovirus vectors).

Although some viruses that can accept foreign genetic material are limited in the number of nucleotides they can accommodate and in the range of cells they infect, these viruses have been demonstrated to successfully effect gene expression. However, adenoviruses do not integrate their genetic material into the host genome and therefore do not require host replication for gene expression, making them ideally suited for rapid, efficient, heterologous gene expression. Techniques for preparing replication-defective infective viruses are well known in the art.

Of course, in using viral delivery systems, one will desire to purify the virion sufficiently to render it essentially free of undesirable contaminants, such as defective interfering viral particles or endotoxins and other pyrogens such that it will not cause any untoward reactions in the cell, animal or individual receiving the vector construct. A preferred means of purifying the vector involves the use of buoyant density gradients, such as cesium chloride gradient centrifugation.

A particular method for delivery of the expression constructs involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein.

The expression vector may comprise a genetically engineered form of adenovirus. Knowledge of the genetic organization or adenovirus, a 36 kb, linear, double-stranded DNA virus, allows substitution of large pieces of adenoviral DNA with foreign sequences up to 7 kb (See Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 map units (m.u.)) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (E1A and E1B; See, e.g., Graham et al., 1977). Since the E3 region is dispensable from the adenovirus genome (See, e.g., Jones and Shenk, 1978), the current adenovirus vectors, with the help of 293 cells, carry foreign DNA in either the E1, the D3 or both regions (See, e.g., Graham and Prevec, 1991). Recently, adenoviral vectors comprising deletions in the E4 region have been described (See, e.g., U.S. Pat. No. 5,670,488, incorporated herein by reference).

In nature, adenovirus can package approximately 105% of the wild-type genome (See, e.g., Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical adenovirus vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells.

Adenovirus vectors have been used in eukaryotic gene expression (See, e.g., Levrero et al., 1991; Gomez-Foix et al., 1992) and vaccine development (See, e.g., Grunhaus and Horwitz, 1992; Graham and Prevec, 1992). Recombinant adenovirus and adeno-associated virus (see below) can both infect and transduce non-dividing human primary cells.

Adeno-associated virus (AAV) is an attractive vector system for use in the cell transduction of the present invention as it has a high frequency of integration and it can infect nondividing cells, thus making it useful for delivery of genes into mammalian cells, for example, in tissue culture (See, e.g., Muzyczka, 1992) or in vivo. AAV has a broad host range for infectivity (See, e.g., Tratschin et al., 1984; Laughlin et al., 1986; Lebkowski et al., 1988; McLaughlin et al., 1988). Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Yoder et al., 1994; Zhou et al., 1994; Hermonat and Muzyczka, 1984; Tratschin et al., 1985; McLaughlin et al., 1988) and genes involved in human diseases (See, e.g., Flotte et al., 1992; Luo et al., 1994; Ohi et al., 1990; Walsh et al., 1994; Wei et al., 1994).

AAV is a dependent parvovirus in that it requires coinfection with another virus (either adenovirus or a member of the herpes virus family) to undergo a productive infection in cultured cells (See, e.g., Muzyczka, 1992). In the absence of coinfection with helper virus, the wild type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (See, e.g., Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (See, e.g., McLaughlin et al., 1988; Samulski et al., 1989; each incorporated herein by reference) and an expression plasmid containing the wild type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (See, e.g., Yang et al., 1994; Clark et al., 1995). Cell lines carrying the rAAV DNA as an integrated provirus can also be used (Flotte et al., 1995).

Retroviruses have promise as gene delivery vectors due to their ability to integrate their genes into the host genome, transferring a large amount of foreign genetic material, infecting a broad spectrum of species and cell types and of being packaged in special cell-lines (See, e.g., Miller, 1992).

The retroviruses are a group of single-stranded RNA viruses characterized by an ability to convert their RNA to double-stranded DNA in infected cells by a process of reverse-transcription (See, e.g., Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (See, e.g., Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (See, e.g., Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (See, e.g., Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (See, e.g., Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (See, e.g., Markowitz et al., 1988; Hersdorffer et al., 1990).

Gene delivery using second generation retroviral vectors has been reported. Kasahara et al. (1994) prepared an engineered variant of the Moloney murine leukemia virus, that normally infects only mouse cells, and modified an envelope protein so that the virus specifically bound to, and infected, human cells bearing the erythropoietin (EPO) receptor. This was achieved by inserting a portion of the EPO sequence into an envelope protein to create a chimeric protein with a new binding specificity.

Other viral vectors may be employed as expression constructs in the present invention. Vectors derived from viruses such as vaccinia virus (See, e.g., Ridgeway, 1988; Baichwal and Sugden, 1986; Coupar et al., 1988), sindbis virus, cytomegalovirus and herpes simplex virus may be employed. They offer several attractive features for various mammalian cells (See, e.g., Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (See, e.g., Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (See, e.g., Chang et al., 1991).

In certain further embodiments, the vector will be HSV. A factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations. HSV also is relatively easy to manipulate and can be grown to high titers. Thus, delivery is less of a problem, both in terms of volumes needed to attain sufficient MOI and in a lessened need for repeat dosings.

In still further embodiments of the present invention, the nucleic acids to be delivered (e.g., nucleic acids encoding Nrl, Nr2e3 or other biomarker or portions thereof) are housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (See, e.g., Roux et al., 1989). Using antibodies against major histocompatibility complex class I and class II antigens, they demonstrated the infection of a variety of human cells that bore those surface antigens with an ecotropic virus in vitro (See, e.g., Roux et al., 1989).

In various embodiments of the invention, nucleic acid sequence encoding a fusion protein is delivered to a cell as an expression construct. In order to effect expression of a gene construct, the expression construct must be delivered into a cell. As described herein, one mechanism for delivery is via viral infection, where the expression construct is encapsidated in an infectious viral particle. However, several non-viral methods for the transfer of expression constructs into cells also are contemplated by the present invention. In one embodiment of the present invention, the expression construct may consist only of naked recombinant DNA or plasmids (e.g., vectors comprising nucleic acid sequences of the present invention). Transfer of the construct may be performed by any of the methods mentioned which physically or chemically permeabilize the cell membrane. Some of these techniques may be successfully adapted for in vivo or ex vivo use, as discussed below. In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (See, e.g., Ghosh and Bachhawat, 1991). Also contemplated is an expression construct complexed with Lipofectamine (Gibco BRL).

Liposome-mediated nucleic acid delivery and expression of foreign DNA in vitro has been very successful (See, e.g., Nicolau and Sene, 1982; Fraley et al., 1979; Nicolau et al., 1987). Wong et al. (1980) demonstrated the feasibility of liposome-mediated delivery and expression of foreign DNA in cultured chick embryo, HeLa and hepatoma cells.

In certain embodiments of the invention, the liposome may be complexed with a hemagglutinating virus (HVJ). This has been shown to facilitate fusion with the cell membrane and promote cell entry of liposome-encapsulated DNA (See, e.g., Kaneda et al., 1989). In other embodiments, the liposome may be complexed or employed in conjunction with nuclear non-histone chromosomal proteins (HMG-1) (See, e.g., Kato et al., 1991). In yet further embodiments, the liposome may be complexed or employed in conjunction with both HVJ and HMG-1. In other embodiments, the delivery vehicle may comprise a ligand and a liposome. Where a bacterial promoter is employed in the DNA construct, it also will be desirable to include within the liposome an appropriate bacterial polymerase.

In certain embodiments of the present invention, the expression construct is introduced into the cell via electroporation. Electroporation involves the exposure of a suspension of cells (e.g., bacterial cells such as E. coli) and DNA to a high-voltage electric discharge.

Transfection of eukaryotic cells using electroporation has been quite successful. Mouse pre-B lymphocytes have been transfected with human kappa-immunoglobulin genes (See, e.g., Potter et al., 1984), and rat hepatocytes have been transfected with the chloramphenicol acetyltransferase gene (See, e.g., Tur-Kaspa et al., 1986) in this manner.

In other embodiments of the present invention, the expression construct is introduced to the cells using calcium phosphate precipitation. Human KB cells have been transfected with adenovirus 5 DNA (See, e.g., Graham and Van Der Eb, 1973) using this technique. Also in this manner, mouse L(A9), mouse C127, CHO, CV-1, BHK, NIH3T3 and HeLa cells have been transfected with a neomycin marker gene (See, e.g., Chen and Okayama, 1987), and rat hepatocytes were transfected with a variety of marker genes (See, e.g., Rippe et al., 1990).

In another embodiment, the expression construct is delivered into the cell using DEAE-dextran followed by polyethylene glycol. In this manner, reporter plasmids were introduced into mouse myeloma and erythroleukemia cells (See, e.g., Gopal, 1985).

Another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (See, e.g., Klein et al., 1987). Several devices for accelerating small particles have been developed. One such device relies on a high voltage discharge to generate an electrical current, which in turn provides the motive force (See, e.g., Yang et al., 1990). The microprojectiles used have consisted of biologically inert substances such as tungsten or gold beads.

Further embodiments of the present invention include the introduction of the expression construct by direct microinjection or sonication loading. Direct microinjection has been used to introduce nucleic acid constructs into Xenopus oocytes (See, e.g., Harland and Weintraub, 1985), and LTK⁻ fibroblasts have been transfected with the thymidine kinase gene by sonication loading (See, e.g., Fechheimer et al., 1987).

In certain embodiments of the present invention, the expression construct is introduced into the cell using adenovirus assisted transfection. Increased transfection efficiencies have been reported in cell systems using adenovirus coupled systems (See, e.g., Kelleher and Vos, 1994; Cotten et al., 1992; Curiel, 1994).

Still further expression constructs that may be employed to deliver nucleic acid construct to target cells are receptor-mediated delivery vehicles. These take advantage of the selective uptake of macromolecules by receptor-mediated endocytosis that will be occurring in the target cells. In view of the cell type-specific distribution of various receptors, this delivery method adds another degree of specificity to the present invention.

Certain receptor-mediated gene targeting vehicles comprise a cell receptor-specific ligand and a DNA-binding agent. Others comprise a cell receptor-specific ligand to which the DNA construct to be delivered has been operatively attached. Several ligands have been used for receptor-mediated gene transfer (See, e.g., Wu and Wu, 1987; Wagner et al., 1990; Perales et al., 1994; Myers, EPO 0273085), which establishes the operability of the technique. In certain aspects of the present invention, the ligand will be chosen to correspond to a receptor specifically expressed on the EOE target cell population.

In other embodiments, the DNA delivery vehicle component of a cell-specific gene targeting vehicle may comprise a specific binding ligand in combination with a liposome. The nucleic acids to be delivered are housed within the liposome and the specific binding ligand is functionally incorporated into the liposome membrane. The liposome will thus specifically bind to the receptors of the target cell and deliver the contents to the cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the DNA delivery vehicle component of the targeted delivery vehicles may be a liposome itself, which will preferably comprise one or more lipids or glycoproteins that direct cell-specific binding. For example, Nicolau et al. (1987) employed lactosyl-ceramide, a galactose-terminal asialganglioside, incorporated into liposomes and observed an increase in the uptake of the insulin gene by hepatocytes. It is contemplated that the tissue-specific transforming constructs of the present invention can be specifically delivered into the target cells in a similar manner.

### II. Cell Therapy

The present invention also provides therapies for photoreceptor loss (e.g., due to retinal or macular degeneration). For example, photoreceptor cells (e.g., photoreceptor precursor cells (e.g., identified and/or isolated utilizing the compositions and methods of the present invention)) can be administered (e.g., transplanted into) to a subject (e.g., animal or human subject) in need thereof such that functional cells (e.g., functional photoreceptor cells (e.g., functional rod cells)) develop in the subject. In some embodiments, cell development in the subject comprises integration within the retina (e.g., within the outer nuclear layer)). In some embodiments, cell development comprises generation of functional synapses between the cell and the subject. Such therapies find use in research or clinical (e.g., therapeutic) settings. In some embodiments, transplantation of photoreceptor cells into a subject provides trophic support to cells (e.g., photoreceptor cells) of the recipient. Thus, although an understanding of the mechanism is not necessary to practice the present invention and the present invention is not limited to any particular mechanism of action, in some embodiments, transplantation of photoreceptor cells are able to slow down the degeneration of neurons (e.g., retinal degeneration) due to trophic factors (e.g., rod derived cone viability factor (RDCVF) and TAFA-3) released by the transplanted photoreceptor cells.

### III. Transgenic Animals

In experiments conducted during the course of development of the present invention, a transgenic mouse comprising a Nrl-L-EGFP construct (e.g., termed wt-Gfp) was generated (See Example 1).

Accordingly, in some embodiments, the present invention provides animal models of Nrl expression. In other embodiments, the present invention provides animal models comprising Nrl knockouts or loss of function variants (See e.g., Examples 1 and 2). Such knockout animals may be generated using any suitable method. The animal may be heterozygous or, more preferably, homozygous for the Nrl gene disruption. In some embodiments, the gene disruption comprises a deletion of all or a portion of the Nrl gene. In other embodiments, the gene disruption comprises an insertion or other mutation of the Nrl gene. In still other embodiments, the gene disruption is a genetic alteration that prevents expression, processing, or translation of the Nrl gene. In one embodiment, both Nrl gene alleles are functionally disrupted such that expression of the Nrl gene product is substantially reduced or absent in cells of the animal. The term “substantially reduced or absent” is intended to mean that essentially undetectable amounts of normal Nrl gene product are produced in cells of the animal. This type of mutation is also referred to as a “null mutation” and an animal carrying such a null mutation is also referred to as a “knockout animal.” In preferred embodiments, the transgenic animals display a disease phenotype (e.g., vision impairment) similar to that observed in humans.

In some embodiments, the present invention provides transgenic mice, wherein the mice are Nrl knockouts or loss of function variants that have been crossed with wt-Gfp mice to generate Nrl-L-EGFP:Nrl^{-/-} mice.

In view of the observed phenotypes, the transgenic animals of the present invention find use for understanding and characterizing a number of diseases, conditions, and biological processes, including, but not limited to, diabetic retinopathy or other types of retinopathies (e.g., caused by disease or disorder). A number of general screening utilities are provided below.

The present invention is not limited to a particular animal. A variety of human and non-human animals are contemplated. For example, in some embodiments, rodents (e.g., mice or rats) or primates are provided as animal models for alterations in photoreceptor development and function and screening of test compounds.

In other embodiments, the present invention provides commercially useful transgenic animals (e.g., livestock animals such as pigs, cows, or sheep) overexpressing Nrl. Any suitable technique for generating transgenic livestock may be utilized. In some preferred embodiments, retroviral vector infection is utilized (See e.g., U.S. Pat. No. 6,080,912 and WO/0030437; each of which is herein incorporated by reference in its entirety).

In still further embodiments, the present invention provides photoreceptor precursor cells derived from Nrl transgenic animals. Experiments conducted during the course of development of the present invention demonstrated that photoreceptor precursor cells derived from transgenic mice overexpressing Nrl can be used successfully in transplantation settings (e.g., integrate and form synaptic connections within a host subject). While not being limited to a particular mechanism, it is contemplated that photoreceptor cells s comprising such properties find use in clinical and therapeutic research settings.

In some embodiments, the present invention provides a transgenic mouse (e.g., decribed herein) harboring transplanted photoreceptor precursor cells (e.g., a transgenic mouse that has received a subretinal injection of photoreceptor precursor cells (e.g., identified using a biomarker described herein (e.g., Nrl))). In some embodiments, such a transgenic mouse is administered one or more test compounds and the development and/or activity of the transplanted cells monitored.

### III. Applications

The transgenic animals of the present invention find use in a variety of applications, including, but not limited to, those described herein.

### Utilizing Transgenic Animals for Genetic Screens

In some embodiments, the Nrl transgenic animals of the present invention are crossed with other transgenic models or other strains of animals to generate F1 and subsequently F2 animals for disease models that carry GFP tagged photoreceptors. In another embodiment, a disease condition is induced by breeding an animal of the invention with another animal genetically prone to a particular disease. For example, in some embodiments, Nrl transgenic animals are crossed with animal models of other genes associated with retinopathies (e.g., rd1, rd3, or rho^{-/-} mice) or related conditions.

In some embodiments, the Nrl animals are used to generate animals with an active Nrl gene from another species (a “heterologous” Nrl gene). In preferred embodiments, the gene from another species is a human gene. In some embodiments, the human gene is transiently expressed. In other embodiments, the human gene is stably expressed. Such animals find use to identify agents that inhibit or enhance human Nrl activity in vivo. For example, a stimulus that induces production of Nrl or enhances Nrl signaling is administered to the animal in the presence and absence of an agent to be tested and the response in the animal is measured. An agent that inhibits human Nrl in vivo is identified based upon a decreased response in the presence of the agent compared to the response in the absence of the agent.

### Drug Screening

The present invention provides methods and compositions for using transgenic animals as a target for screening drugs that can alter, for example, interaction between a biomarker (e.g., Nrl) and binding partners (e.g., those identified using the above methods) or enhance or inhibit the activity of a biomarker (e.g., Nrl) or its signaling pathway. Drugs or other agents (e.g., test compounds (e.g., from a test compound library)) are exposed to the transgenic animal model and changes in phenotypes or biological markers are observed or identified. For example, in some embodiments, drug candidates are tested for the ability to alter photoreceptor cell development or function in Nrl knockout or overexpressing animals. In some embodiments, test compounds are utilized to determine their ability to alter development (e.g., integration and synaptic connectivity) of photoreceptor precursor cells transplanted into a transgenic animal.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 (1994))); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (See, Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 (1993); Erb et al., Proc. Nad. Acad. Sci. USA 91:11422 (1994); Zuckermann et al., J. Med. Chem. 37:2678 (1994); Cho et al., Science 261:1303 (1993); Carrell et al., Angew. Chem. Int. Ed. Engl. 33.2059 (1994); Carell et al., Angew. Chem. Int. Ed. Engl. 33:2061 (1994); and Gallop et al., J. Med. Chem. 37:1233 (1994).

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assay. These include reagents like salts, neutral proteins (e.g. albumin), detergents, etc. that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening.

In some embodiments, the present invention provides transgenic mice useful for identifying genes, proteins and/or pathways associated with retinal degeneration. For example, in some embodiments, transgenic mice are generated by crossing Nrl-GFP wild type mice with any one of several retinal degenerative diseased mice (e.g., including, but not limited to, mice lacking wild-type rd1, rd2, rd3, rd7, rd9, rd11, rd13, rd14, CEP290, or Nr2e3). In general, it is preferable to generate F2 mice comprising a homozygous null mutation for the gene associated with retinal disease. GFP permits facile isolation and/or purification of photoreceptor cells from these mice. Gene expression profiles can be obtained from photoreceptor cells from each transgenic mouse and compared (e.g., using meta analysis) to identify common proteins and/or pathways associated with disease. Furthermore, these animals and/or photoreceptor cells can be utilized as a target for drug discovery (e.g., via administration of a test compound to the transgenic animal). It is contemplated that such methods will permit identification of early changes within photoreceptor cells that are important in degenerative processes.

### Therapeutic Agents

The present invention further provides agents identified by the above-described screening assays. Accordingly, it is within the scope of this invention to further use an agent identified as described herein (e.g., neuronal modulating agent or biomarker mimetic, a biomarker inhibitor, a biomarker specific antibody, or a biomarker-binding partner) in an appropriate animal model (e.g., Nrl overexpressing transgenic animal, Nrl transgenic knockout animal, hybrid of a Nrl transgenic knockout animal, progeny of Nrl transgenic knockout animal, a transgenic animal into which photoreceptor precursor cells have been transplanted, etc.) to determine efficacy, toxicity, side effects, and/or mechanism of action, of treatment with such an agent. Furthermore, agents identified by the above-described screening assays can be used for treatments of photoreceptor cell related disease (e.g., including, but not limited to, retinopathies caused by disease or disorder).

In some embodiments, biomarkers of the present invention are utilized to identify and/or isolate human photoreceptor precursor cells. For example, one or more cell surface biomarkers (e.g., CD24a, CD1d1, Chrnb4, Clic4, Ddr1, F2r, Gpr137b, Igsf4b, LRP4, Nope, Nrp1, Pdpn, Ptpro, St8sia4, and Tmem46) can be utilized to identify and isolate photoreceptor precursor cells. In some embodiments, one or more of the surface markers are utilized to identify a cell from which a photoreceptor precursor cell can be derived (e.g., a stem cell (e.g., a retinal stem cell)). As used herein, the term “retinal stem cell” refers to distinct, limited (or possibly rare) subset of cells that share many properties of normal “stem cells.” For example, retinal stem cells may be characterized as cells that proliferate extensively or indefinitely and/or that give rise to various lineages of retinal cells (e.g., rod cells and/or cone cells).

In some embodiments, biomarkers can be utilized to identify newly generated photoreceptor precursor cells (e.g., from neuronal or embryonic stem cells that have been administered a test compound in order to alter stem cell fate). Cells identified and/or isolated using cell surface biomarkers may find use in research and/or therapeutic (e.g., transplant) settings. Furthermore, cell surface biomarkers can be used to identify test compounds capable of altering stem cell fate. For example, test compounds that induce expression of cell surface biomarkers (e.g., on stem cells in vitro) can then be utilized in vivo to monitor the ability to alter photoreceptor cell commitment and development.

### Experimental

The following examples are provided in order to demonstrate and further illustrate certain preferred embodiments and aspects of the present invention and are not to be construed as limiting the scope thereof.

### Example 1

### Targeting of Green Fluorescent Protein to New-Born Rods by Nrl Promoter and Temporal Expression Profiling of Flow-Sorted Photoreceptors

### Materials and Methods.

Comparison of 5′-Upstream Sequences of the Human and Mouse Nrl Genes. A mouse Nrl genomic clone was isolated and sequenced from a 129×1/SvJ-derived Lambda Fix II genomic library (Stratagene). Genomic sequences 3 kb upstream of the human NRL (Genbank accession number AL136295) and mouse Nrl transcription start sites (Genbank accession number AY526079) were compared using BLAST2 (See, www.ebi.ac.uk/blastall/vectors.html).

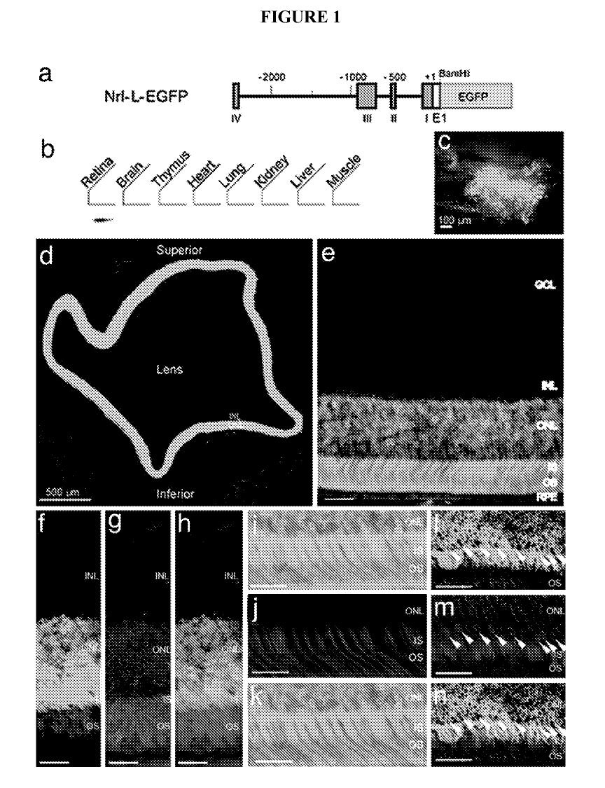

Plasmid Constructs and Generation of Transgenic Mice. A 2.5-kb upstream segment of the mouse Nrl gene (from −2408 to +115) was cloned into the pEGFP1 vector (Clontech) (Nrl-L-EGFP construct; See FIG. 1a). The 3.5-kb insert from Nrl-L-EGFP, excluding the vector backbone, was injected into fertilized (C57BL/6×SJL) F₂ mouse oocytes that were implanted into pseudopregnant females (University of Michigan transgenic core facility). Transgenic founder mice and their progeny were identified by PCR, and transgene copy number was estimated by Southern blot analysis of tail DNA using an EGFP gene probe. The founders were bred to C57BL/6 mice to generate F₁ progeny. A mouse line with three copies of the transgene was used for subsequent studies.

Immunoblotting and Immunostaining. Methods utilized for immunoblotting and immunostaining were as described (See, Swain et al., (2001) J. Biol. Chem 276, 36824-36830; Mears et al., (2001) Nat. Genet 29, 447-452). For immunoblot analysis, the primary antibodies were rabbit anti-GFP pAb (Santa Cruz Biotechnology) or mouse anti-GFP mAb (Covance Research Products, Cumberland, Va.). For immunofluorescence, 10-μm retinal cryosections or retinal cells isolated with papain dissociation system (Worthington) were used. Primary antibodies were: GFP, rabbit pAb (Upstate Biotechnology, Lake Placid, N.Y.) or rabbit pAb conjugated to Alexa Fluor-488 (Molecular Probes); rhodopsin, mouse mAb (Rho4D2, obtained from R. Molday, University of British Columbia, Vancouver); cone arrestin, rabbit pAb (obtained from C. Craft, University of Southern California, Los Angeles); phosphohistone H3, rabbit pAb (Upstate Biotechnology); Cyclin D1, mouse mAb (Zymed); Ki67, mouse mAb (DAKO); BrdUrd, rat mAb (Harlan Sera-Lab, Loughborough, U.K.). Texas red-conjugated peanut agglutinin lectin (PNA) was obtained from Vector Laboratories. Fluorescent detection was performed by using Alexa Fluor-488 or -546 (Molecular Probes) and FITC or Texas red (Jackson ImmunoResearch)-conjugated secondary antibodies. Sections were visualized under a conventional fluorescent microscope and digitized.

BrdUrd Staining. Pregnant females were given single i.p. injection of BrdUrd (Sigma, 0.1 mg/g body weight) on embryonic day 16 (E16). Embryos were dissected 1, 4, 6, or 12 h after injection, fixed in 4% paraformaldehyde, and cryosectioned. Immunostaining was performed sequentially to detect GFP and then BrdUrd. After GFP immunostaining with primary and secondary reagents, sections were washed in PBTx (PBS+0.1% Triton X-100) and incubated in 2.4 M HCl/PBTx for 75 min. Sections were then washed and immunostained for BrdUrd.

RNA Preparation and Real-Time PCR. Total RNA was extracted using TRIZOL (Invitrogen) and treated with RNase-free DNase I before reverse transcription. Quantitative real-time PCR was performed with ICYCLER IQ SYSTEM (Bio-Rad).

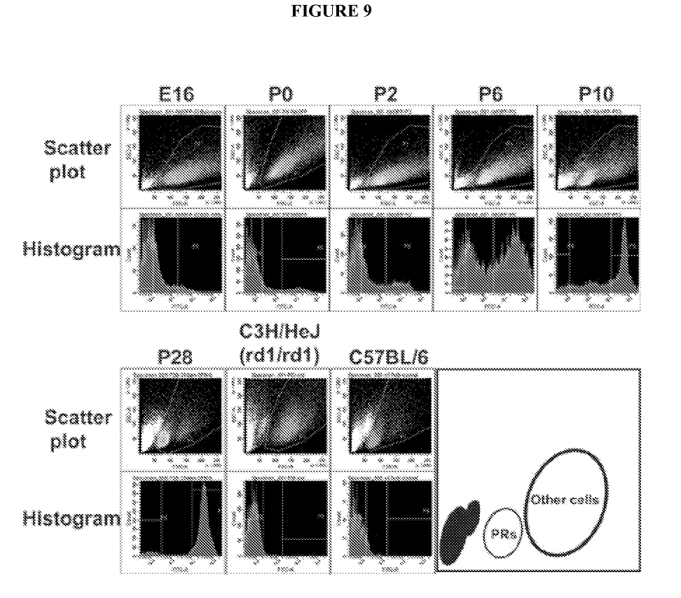

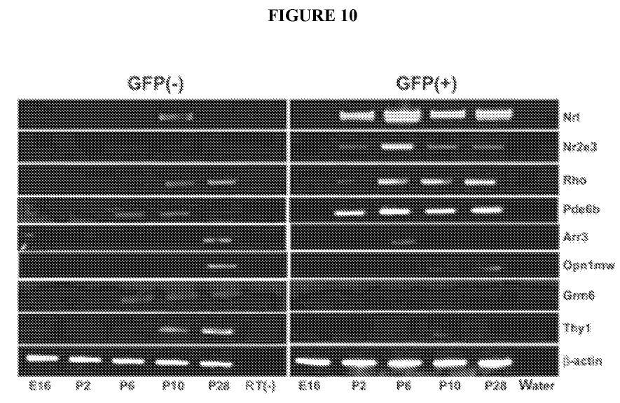

FACS Enrichment and Microarray Hybridization. Mouse retinas were dissected at five time points: E16, post natal day (P) 2 (P2), P6, P10, and P28. GFP+ photoreceptors were enriched by FACS (FACSARIA; BD Biosciences) (See FIG. 9). RNA was extracted from 1−5×10⁵ flow-sorted cells and evaluated by RT-PCR using selected marker genes (See FIG. 10). Total RNA (40-60 ng) was used for linear amplification with OVATION BIOTIN labeling system (Nugen, San Carlos, Calif.), and 2.75 μg of biotin-labeled fragmented cDNA was hybridized to mouse GENECHIPS MOE430.2.0 (Affymetrix) having 45,101 probe sets (corresponding to >39,000 transcripts and 34,000 annotated mouse genes). Four to six independent samples were used for each time point.

Gene Filtering and Analysis. The “AFFY” package (See, e.g., Gautier et al., (2004) Bioinformatics 20, 307-315) was used to generate “present” and “absent” calls, for every gene at each developmental stage, based on a majority rule over the replicates. Each of the 45,101 probe sets was assigned to one of the 32 possible clusters based on its presence/absence pattern across five time points. The 22,611 “present” probe sets are also referred to as genes herein. The Robust Multichip Average method (See, e.g., Irizarry et al., (2003) Biostatistics 4, 249-264) was used for background correction, quantile normalization, and summarization of expression scores. These genes were further subjected to two-stage filtering procedure based on the theory of FDR-CIs (See, e.g., Benjamini, Y. & Yekutieli, D. (2005) J. Am. Stat. Assoc 100, 71-80), as described (See, e.g., Irizarry et al., (2003) Biostatistics 4, 249-264). The FDR-CI P value for a given gene is defined as the minimum significance level q for which the gene's FDR-CI does not intersect the (−fcmin, fcmin) interval (e.g., fcmin=1 corresponds to a 2-fold change in log 2 scale). Microarray data in MIAME format (See, e.g., Brazma et al., (2001) Nat. Genet 29, 365-371) was deposited in the Gene Expression Omnibus database GEO (See www.ncbi.nlm.nih.gov/geo).

SOM and Hierarchical Gene Clusterings. The top 1,000 FDR-CI constrained gene profiles were standardized to have mean of 0 and SD of 1 across five time points and clustered by using SOM implemented in Gene Cluster II (See, e.g., Reich et al., (2004) Bioinformatics 20, 1797-1798) and hierarchical clustering implemented in CLUSTER and TREEVIEW (See, e.g., Eisen et al., (1998) Proc. Natl. Acad. Sci. USA 95, 14863-14868). Euclidean distance was chosen for clustering as the measure of expression profile similarity. For SOM, clusters of similarly expressed genes were projected onto a 2D 2×4 grid, that was selected empirically to capture biologically nonredundant patterns of interest. For hierarchical analysis, clusters were defined by selecting a certain branch length (height) of the dendrogram. Gene Ontology analysis of SOM and hierarchical clusters was performed as described (See, e.g., www.affymetrix.com/analysis/index.affx).

### Nrl Promoter Directs EGFP Expression to Rods.

A comparison of the human and mouse Nrl promoter sequences identified four conserved regions (designated I-IV) (See FIG. 1a). The Nrl-L-EGFP construct, which included all four conserved regions (See FIG. 1a), was used to generate transgenic mice as described above. Six of the seven transgenic lines that were analyzed demonstrated GFP expression only in the retina (See FIG. 1b) and pineal gland (See FIG. 1c). In the adult retina, GFP was detected only in the outer nuclear layer, which contains rod and cone photoreceptor nuclei, and in the corresponding inner and outer segments (See FIGS. 1d and 1e). Immunostaining with anti-rhodopsin antibody (See, e.g., Molday, R. S. & MacKenzie, D. (1983) Biochemistry 22, 653-660) showed complete colocalization with GFP (See FIGS. 1f-1h), whereas no overlap was observed between GFP and the cone-specific markers, peanut agglutinin (See, e.g., Blanks, J. C. & Johnson, L. V. (1983) J. Comp. Neurol 221, 31-41) and cone arrestin (See, e.g., Akimoto et al., (2004) Invest. Ophthalmol. Visual Sci 45, 42-47) (See FIGS. 1i-1n). Thus, all GFP-expressing cells were rod photoreceptors.

### GFP Expression Corresponds to Rod Genesis in Developing Retina.

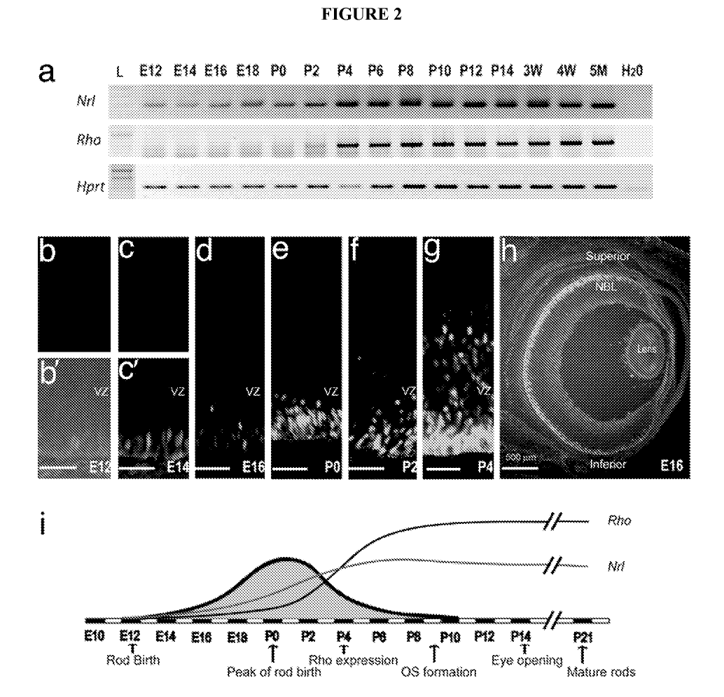

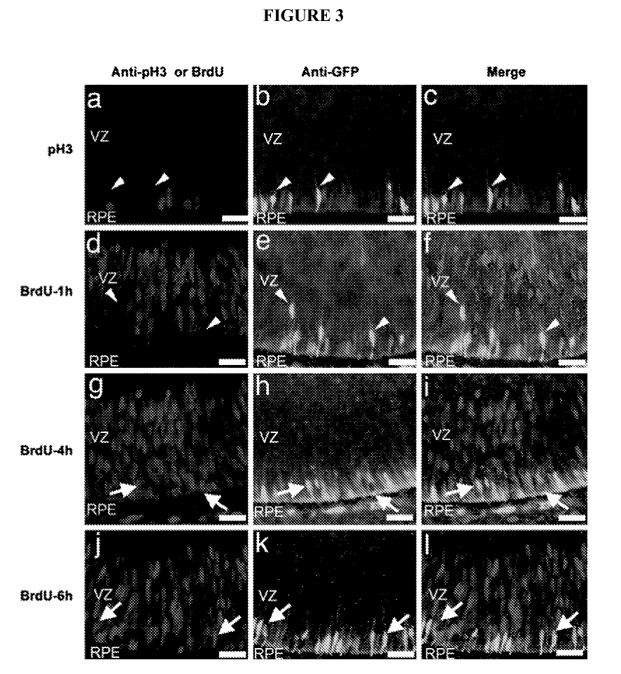

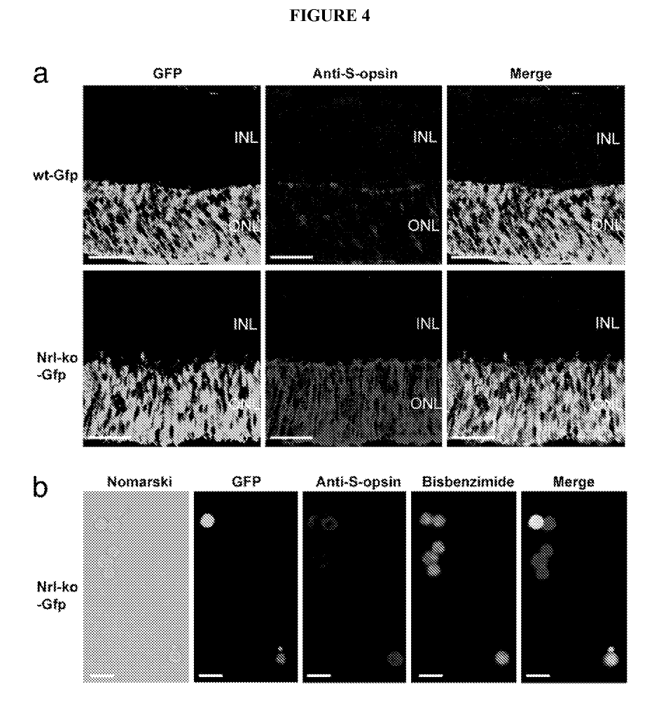

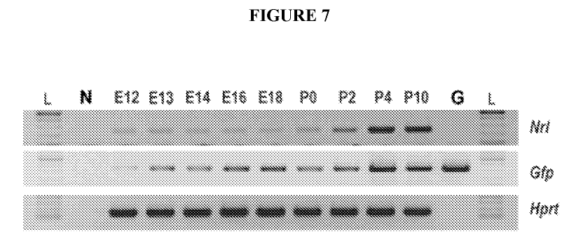

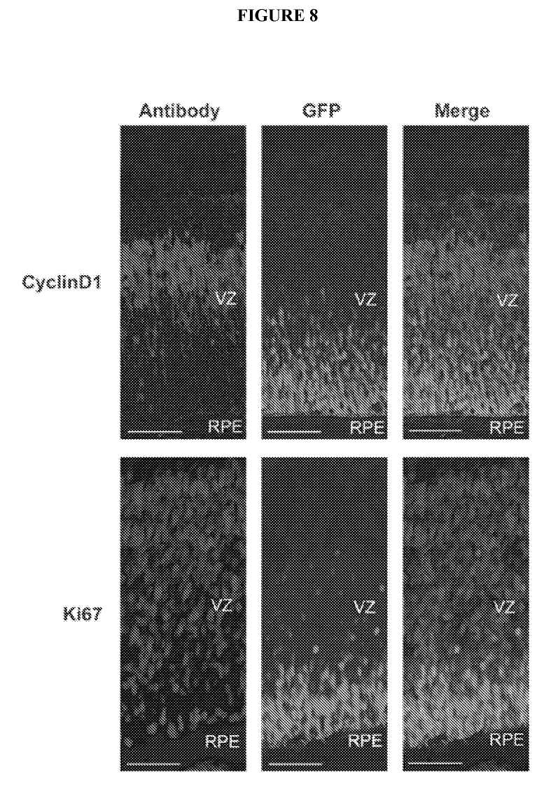

In rodents, rods are born over an extended developmental period (embryonic day 12 (E12) to postnatal day 10 (P10)) overlapping with the birth of all neuronal subtypes in the retina (See FIG. 2; and See, e.g., Carter-Dawson, L. D. & LaVail, M. M. (1979) J. Comp. Neurol 188, 263-272; Young, R. W. (1985) Anat. Rec 212, 199-205; and Morrow et al., (1998) J. Neurosci 18, 3738-3748). Nrl transcripts are detected by RT-PCR as early as E12 in mouse retina, considerably earlier than rhodopsin, which is expressed postnatally (See FIG. 2a). To examine whether Nrl expression corresponded to rod genesis, GFP expression was characterized in developing retinas of the Nrl-L-EGFP mice (herein referred to as “wild-type (wt)-Gfp”). The timing and kinetics of GFP expression in transgenic retinas, as revealed by RT-PCR, were consistent with early detection of Nrl transcripts (See FIG. 7). GFP-positive cells, although few and scattered, were first observed at E12 (See FIGS. 2b and 2b′) and subsequently increased in abundance over time (See FIGS. 2c-2h). The spatial and temporal expression of GFP completely correlated with the timing and central-to-peripheral gradient of rod genesis (See FIG. 2i; Carter-Dawson, L. D. & LaVail, M. M. (1979) J. Comp. Neurol 188, 263-272; Young, R. W. (1985) Anat. Rec 212, 199-205). No overlap was observed between GFP and the cell cycle markers Cyclin D1 and Ki67, expressed by cycling cells from late G₁ to M phase, and phosphohistone H3, expressed during M phase (See FIG. 3 and FIG. 8).

### GFP Expression Was Detected in Rod Precursors Shortly After Terminal Mitosis.

To further determine the onset of GFP expression in relation to the cell cycle, short-term BrdUrd pulse-chase experiments were performed in E16 embryos. Whereas GFP was not detected in BrdUrd-positive (S-phase) cells 1 h after the injection, double-labeled cells were observed in embryos harvested at 4 and 6 h (See FIG. 3), and their abundance increased at longer intervals after BrdUrd exposure. The durations of S and G₂+M phases have been estimated to be 10 and 4 h, respectively, in the E16 mouse retina (See, e.g., Young, R. W. (1985) Brain Res 353, 229-239; Sinitsina, V. F. (1971) Arkh. Anat. Gistol. Embriol 61, 58-67). Thus, the present invention provides that Nrl is expressed shortly after terminal division by cells that are fated to become rod photoreceptors, thereby establishing Nrl as the earliest identifiable marker specific to rods. Additional support for this conclusion was obtained by fate-mapping studies using cre-recombinase driven by the Nrl promoter.

Enhanced S-Cones in the Nrl^{-/-} Retina Originate from Postmitotic Rod Precursors.

The abundant S-cones in Nrl^{-/-} mice are presumed to derive from rods that do not follow their appropriate developmental pathway due to the absence of Nrl (See, e.g., Mears et al., (2001) Nat. Genet 29, 447-452). To directly evaluate the origin of enhanced S-cones in the Nrl^{-/-} retina, wt-Gfp mice were crossed with the Nrl^{-/-} mice to generate Nrl-L-EGFP:Nrl^{-/-} mice (herein referred to as “Nrl-ko-Gfp”). As shown in FIG. 4, the GFP+ cells (rod precursors in the wt retina) are colabeled with S-opsin in the Nrl-ko-Gfp retinas and in dissociated retinal cells from embryos and adults. Given that the S-opsin-expressing photoreceptors in the Nrl^{-/-} retina are cones by morphological, molecular, and functional criteria (See, e.g., Daniele et al., (2005) Invest. Ophthalmol. Visual Sci 46, 2156-2167), the present invention provides that S-cones represent the “default fate” for photoreceptors (See, e.g., Cepko, C. (2000) Nat. Genet 24, 99-100; Szel et al., (2000) J. Opt. Soc. Am. A 17, 568-579), at least in mice. Thus, although an understanding of the mechanism is not necessary to practice the present invention and the present invention is not limited to any particular mechanism of action, in some embodiments, the present invention provides that Nrl determines rod fate within “bipotent” photoreceptor precursors by modulating gene networks that simultaneously activate rod- and suppress cone-specific genes.

Gene Profiling of Purified GFP+ Photoreceptors Reveals Specific Regulatory Molecules Associated with Terminal Differentiation.

In order to elucidate the genes and regulatory networks associated with differentiation of photoreceptors from committed postmitotic precursors, genome-wide expression profiling was performed with GFP+ cells purified from the retinas of wt-Gfp and Nrl-ko-Gfp mice at five distinct developmental time points (E16, P2, P6, P10, and P28) (See FIGS. 9 and 10). Given that rods are born over a relatively long period of retinal development (E13-P10), GFP+ cells from wt-Gfp retinas at any specific time represent rods at discrete stages of differentiation; nonetheless, profiles from GFP+ cells at E16 and P2 broadly reflect genes expressed in early- and late-born rods, respectively. The profiles of GFP+ cells purified at P10 and P28 were hypothesized as capable of revealing many genes involved in outer segment formation and phototransduction, respectively. From GENECHIP data, a bitmap of present/absent calls was generated for all probe sets at the five developmental stages from wt-Gfp mice (See FIG. 5a); this diagram indicated the proportion of genes found to fit in any one of 32 potential present/absent patterns and included gene signatures for each time point. Together with a similar bitmap for Nrl-ko-Gfp, these data revealed expression of ˜20,000 transcripts in photoreceptors, consistent with previous retinal transcriptome estimates (See, e.g., Swaroop, A. & Zack, D. J. (2002) Genome Biol 3, 1022). Independent ranked lists were then generated of the top 1,000 genes that were differentially expressed across developmental stages for both wt-Gfp and Nrl-ko-Gfp retinas; each of these genes had a false discovery rate confidence interval (FDR-CI) P value less than or equal to 0.15 and true fold change greater than or equal to 2 in at least one pair of time points. Significantly more genes were differentially expressed over time in these FACS-purified cells (See FIG. 5b) than were identified in comparable gene profiles of the whole retina (See, e.g., Yoshida et al., (2004) Hum. Mol. Genet 13, 1487-1503). Self-organizing map (SOM) clusters were then derived from wt-Gfp and Nrl-ko-Gfp gene profiles, as described (See, e.g., Reich et al., (2004) Bioinformatics 20, 1797-1798). Unexpectedly, similar clusters in the two profiles revealed major differences, which in large part corresponded to distinctions between rods and cones (See, e.g., FIGS. 5c and 5d). The clusters that included rhodopsin (cluster 4 in wt-Gfp, See FIG. 5c) or S-opsin (cluster 5 in Nrl-ko-Gfp, See FIG. 5d) exhibit a significant increase in expression at P10 and P28 (See FIGS. 11 and 12). Thus, the present invention provides genes and gene profiles that facilitate discovery of genetic defects in photoreceptor diseases (e.g., independently or when used together with a whole retina microarray, serial analysis of gene expression, and/or in situ hybridization studies described in, e.g., Yoshida et al., (2004) Hum. Mol. Genet 13, 1487-1503; Swaroop, A. & Zack, D. J. (2002) Genome Biol 3, 1022; Blackshaw et al., (2004) PLoS Biol 2, E247; Mu et al., (2001) Nucleic Acids Res 29, 4983-4993; and Dorrell et al., (2004) Invest. Ophthalmol. Visual Sci 45, 1009-1019.

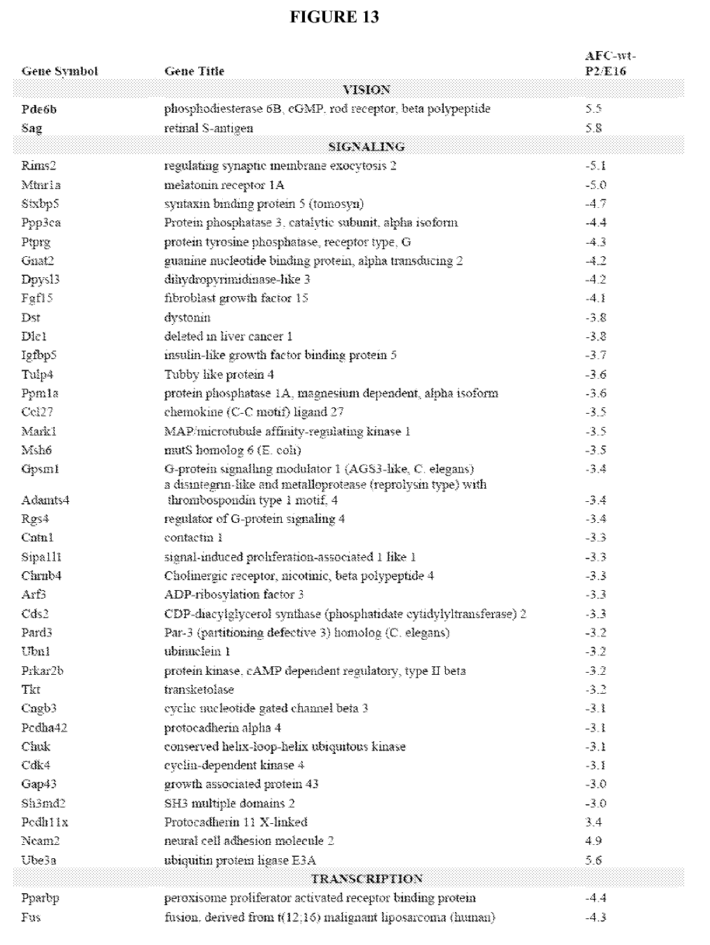

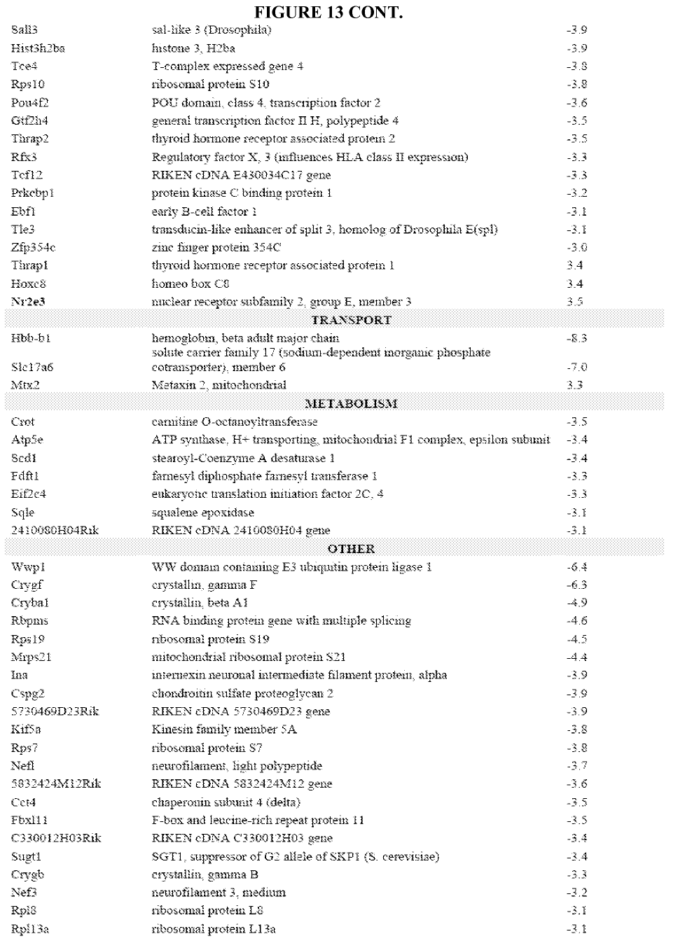

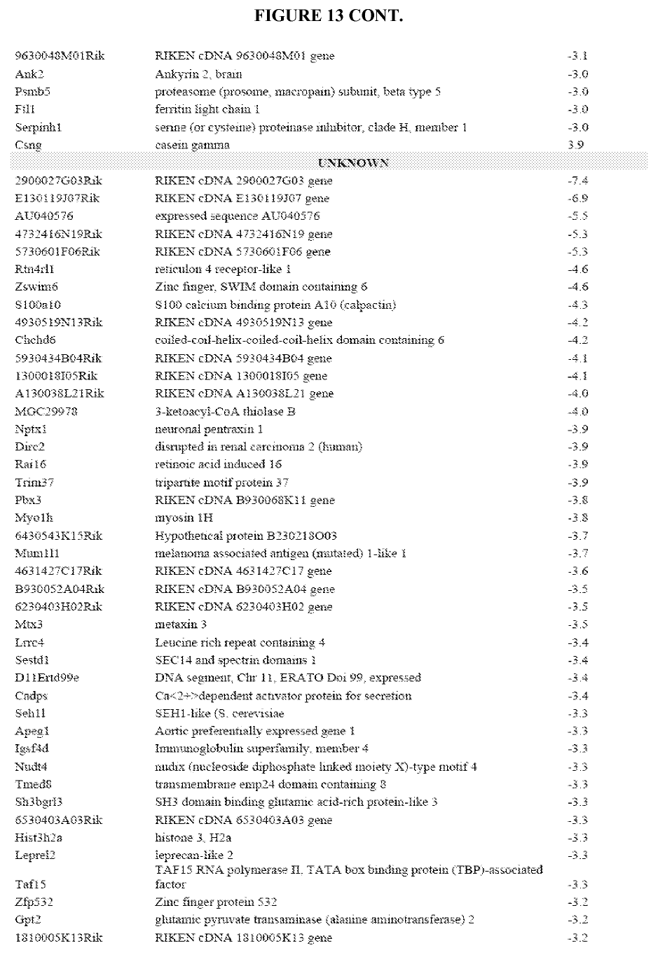

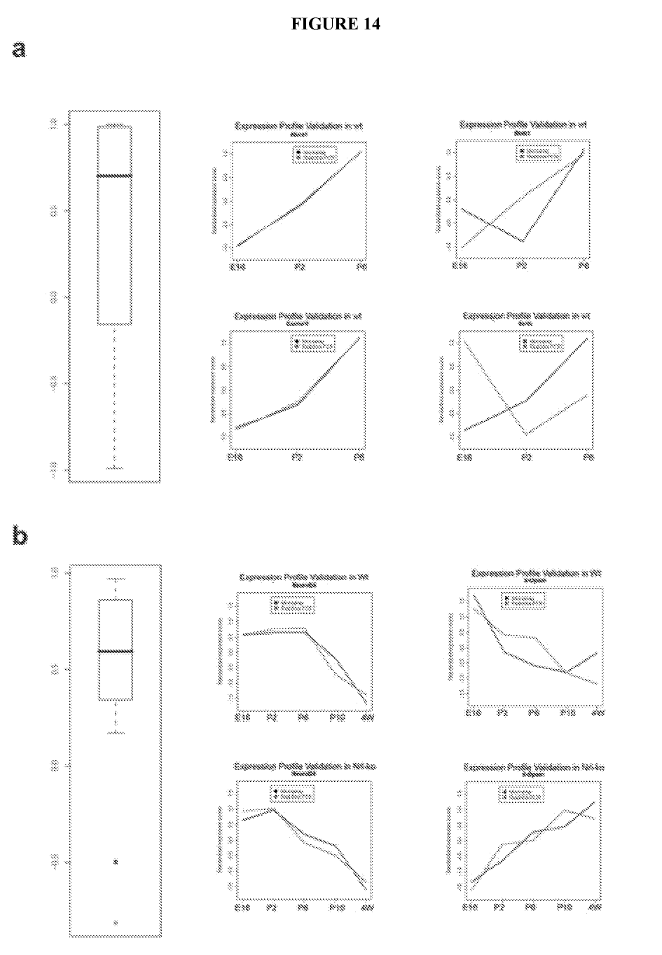

In order to characterize the delay (See, e.g., Cepko, C. (2000) Nat. Genet 24, 99-100; Morrow et al., (1998) J. Neurosci 18, 3738-3748) associated with the expression of phototransduction genes, the gene profiles of E16, P2, and P6 photoreceptors were compared (See FIG. 13); 25 of 34 differentially expressed genes were validated by real-time PCR (See FIG. 11a). At P6, high expression of genes involved in photoreceptor integrity and function (e.g., Rho, Pde6b, Rs1h, Rp1h, Rdh12, and Rpgr) were observed. A battery of regulatory factors were also observed at P6 when compared to the profiles at E16 or P2. Several of the genes displayed decreased expression as differentiation proceeded (e.g., anti-differentiation factors (e.g., Id2) or negative regulators (“the brake genes”) of rod maturation). Regulatory genes showing higher expression at P6 (e.g., Bteb1 and Jarid2) were identified as candidate coactivators of rod differentiation.

Cluster Analysis of Gene Profiles from the GFP-Tagged wt and Nrl^{-/-} Photoreceptors Identifies Expression Differences Between Rods and Cones.

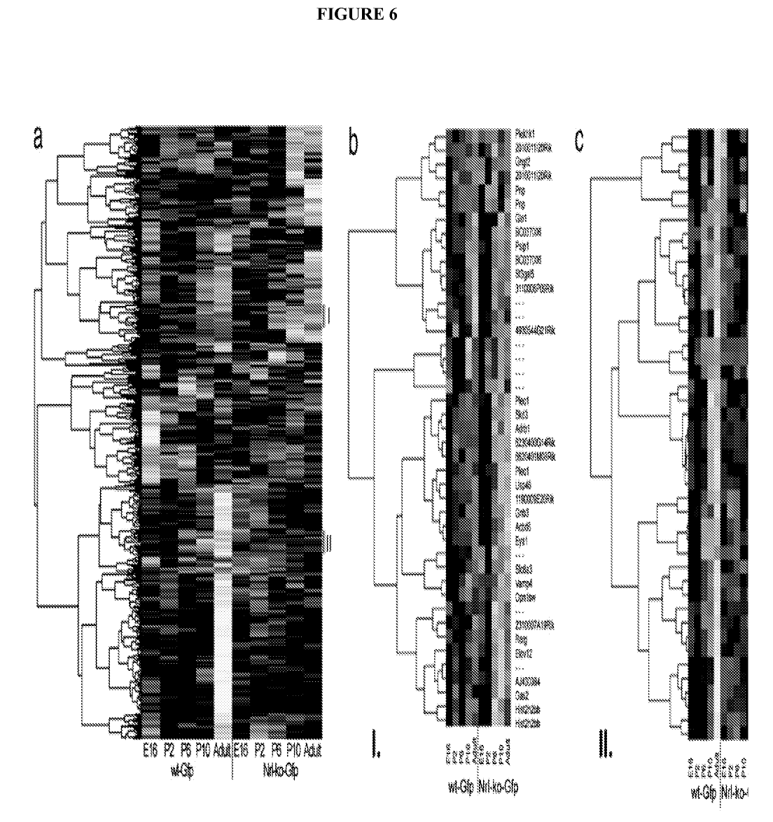

Wt-Gfp and Nrl-ko-Gfp data were then compared. Heat maps of the top 1,000 differentially expressed genes selected over five developmental stages revealed several expression clusters; two of the clusters revealed the genes whose expression increases (cluster I) or decreases (cluster II) with time in Nrl-ko-Gfp cells (See FIG. 6). Although cluster II included a number of rod-specific genes (such as Nrl, Nr2e3, Rho, and Pde6b), cluster I had several genes predicted to be involved in cone function. Real-time PCR analysis of 19 differentially expressed genes demonstrated complete to partial concordance with microarray data for 15 genes over five developmental stages in both wt-Gfp and Nrl-ko-Gfp cells (See FIG. 11). It is also possible that some expression changes in Nrl-ko-Gfp cells may be due, at least in part, to structural aberrations or stress response noted in these fate-switched photoreceptors (See, e.g., Mears et al., (2001) Nat. Genet 29, 447-452; Daniele et al., (2005) Invest. Ophthalmol. Visual Sci 46, 2156-2167; Strettoi, E., Mears, A. J. & Swaroop, A. (2004) J. Neurosci 24, 7576-7582). Thus, the present invention provides genes that are differentially expressed during development and between wt-Gfp and Nrl-ko-Gfp photoreceptors (e.g., that can be used as markers of photoreceptor development, for identification and characterization of candidate agents that alter photoreceptor development and function, or for identification and characterization of retinal dystrophies (See FIGS. 11, 12 and 13)).

### Example 2

### Retinal Repair Via Transplantation of Photoreceptor Precursor Cells

### Materials and Methods

Animals. Mice were maintained in the animal facility at University College London. All experiments have been conducted in accordance with the Policies on the Use of Animals and Humans in Neuroscience Research, revised and approved by the Society for Neuroscience in January 1995. Animal strains used included: Cba.gfp^{+/+}, Ck6.cfp (Jackson Laboratories), Nrl.gfp^{+/+}, rd, rds, rho^{-/-}. These have been described (See, e.g., Example 1; Okabe et al., FEBS Lett. 407, 313-319 (1997); Hadjantonakis et al., BMC. Biotechnol. 2, 11 (2002); Reuter, J. H. & Sanyal, Neurosci. Lett. 48, 231-237 (1984); Carter-Dawson et al., Invest Ophthalmol. Vis. Sci. 17, 489-498 (1978); Humphries et al., Nat. Genet. 15, 216-219 (1997)). Mice defined as “adult” were at least 6, but not more than 12, weeks old.

Dissociation of retinal cells and transplantation. Dissociated retinal cells were prepared from transgenic mice that were hemizygous for a ubiquitously expressed gfp transgene (See, e.g., Okabe et al., FEBS Lett. 407, 313-319 (1997)) or from Nrl.gfp^{+/+} transgenic mice (See, e.g., Example 1). Mice were sacrificed by cervical dislocation and neural retinas dissected free from surrounding tissues. Cells were dissociated using a papain-based kit (Worthington Biochemical, Lorne Laboratories UK) and diluted to a final concentration of ˜4×10⁵ cells/μl. Where appropriate, retinas from P1 Nrl.gfp^{+/+} mice were dissociated, as described above, before being sorted into Nrl.gfp-positive and Nrl.gfp-negative populations, using FACS. The final concentration of sorted Nrl.gfp-positive cells was ˜2×10⁵ cells/μl. Surgery was performed under direct ophthalmoscopy control through an operating microscope. Recipient mice were anaesthetised with a single intra-peritoneal injection of 0.15 ml of a mixture of Dormitor (1 mg/ml medetomidine hydrochloride, Pfizer Pharmaceuticals, Kent UK), ketamine (100 mg/ml, Fort Dodge Animal Health, Southampton, UK) and sterile water for injections in the ratio of 1:0.6:84 for P1 pups and 5:3:42 for adult mice. The tip of a 1.5 cm, 34-gauge hypodermic needle (Hamilton, Switzerland) was inserted through the sclera into the intravitreal space to reduce intraocular pressure. The needle was then withdrawn and loaded with cells before re-inserting tangentially through the sclera into the sub-retinal space, causing a self-sealing wound tunnel. Cell suspensions were injected (0.5 μl per eye for P1 recipients, 2 μl per eye for adults) slowly to produce a retinal detachment in the superior and/or inferior hemisphere around the injection sites. Mice were sacrificed at least 21 days after transplantation and eyes were fixed in 4% paraformaldehyde in phosphate-buffered saline (PBS). Retinal sections were prepared by cryoprotecting fixed eyes in 20% sucrose, before embedding in OCT (TissueTek) and frozen in isopentane cooled in liquid nitrogen. Cryosections (18 μm thick) were cut and affixed to poly-L-lysine coated slides. All sections were collected for analysis.

Histology and Immunohistochemistry. Retinal sections were pre-blocked in Tris-buffered saline (TBS) containing normal goat serum, bovine serum albumin and 0.1% Triton-X 100 for 1 h before being incubated with primary antibody overnight at 4° C. After rinsing 3×10 mins with TBS, sections were incubated with secondary antibody for 2 hrs at room temperature (RT), rinsed and counter-stained with Hoechst 33342. Negative controls omitted the primary antibody. The following antibodies were used: rabbit anti-peripherin-2, mouse anti-rhodopsin (Rho4D2), sheep antiphosducin (kind gift of V. Arshaysky), rabbit anti-bassoon (Stressgen) and rabbit anti-PKC (AbCam), with appropriate Cy3- (Jackson ImmunoResearch) or Alexa- (Molecular Probes, Invitrogen) tagged secondary antibodies.

BrdU labeling. Labelling dividing cells post-transplantation. P1 cells were prepared as described above. Recipient adult mice received intraperitoneal injections of bromodeoxyuridine, BrdU (100 ng/g body weight) immediately following transplant and every other day for the next 8 days.

Labelling donor cells prior to transplantation. P1 received×3 intra-peritoneal injections of BrdU (100 ng/g body weight) 4 hrs apart, in order to label the DNA of the nucleus of a cohort of donor cells. Cells were dissociated, as described above, and transplanted into adult wildtype recipients.

Immunohistochemistry for BrdU labeling. Retinal sections were washed in dH20 before incubating in 2M HCl for 2 hrs at 37° C., 0.1M Na-Borate for 20 mins at RT and 3×10 mins wash in TBS. Sections were then blocked in TBS containing normal goat serum, bovine serum albumin and 0.1% Triton-X 100 for 1 h at RT, prior to incubation with anti-BrdU (rat) primary antibody overnight at RT. Following 3×10 mins wash with TBS, sections were incubated with secondary antibody (goat anti-rat Cy3; Jackson ImmunoResearch) for 2 h at RT, washed in TBS and counter-stained with Hoechst 33342. Negative controls omitted the primary antibody.

Confocal microscopy. Histology/immunohistochemistry. Retinal sections were viewed on a confocal microscope (Leica SP2 or Zeiss LSM510). GFP-positive cells were located using epifluorescence illumination before taking a series of XY optical sections, approximately 0.2-0.4 μm apart, throughout the depth of the section. Individual XY scans were built into a stack to give an XY projection image. The fluorescence of Hoechst, GFP and Cy3/Alexa-546 were sequentially excited using the 350 nm line of a UV laser, the 488 nm line of an argon laser and the 543 nm line of a HeNe laser, respectively. In each case, projections of the XYZ stacks were generated, as described above. Unless otherwise stated, images show (i) show merged Nomarski and confocal fluorescence projection images of GFP (green) and the nuclear counter stain, Hoechst 33342 (blue) (or propidium iodide, in some instances), and immunolabelling where appropriate and (ii) the same region showing GFP signal only. For co-localisation assessments, single confocal sections were taken at the level of GFP signal from the integrated cell, in addition to the standard projection images. For simplicity, only the ONL is shown, unless otherwise stated.

To visualise GFP cells transplanted into CFP recipients, the fluorescence of GFP and CFP were excited sequentially. FP fluorescence was excited, as described above, and the emission collected at 505-550 nm, while that of CFP was excited using the 405 nm line of a blue diode laser and the emission collected at 450-485 nm. Separation of the fluorescence signals of the two proteins is complete when acquired at these wavelengths.

Calcium Imaging. Retinas transplanted with Nrl.gfp^{+/+} P1 cells were dissected free of all surrounding tissue. Whole-mount neural retinas were loaded with Fura Red-AM (15 μM, Molecular Probes) and the dispersant Cremophor-EL (0.03%, Sigma) for 1.5 hrs at 36° C. and then de-esterified in fresh DMEM-F12 (without phenol red) for 30 mins at 36° C. Retinas were transferred to the stage of an inverted microscope (SP2, Leica, UK) and held flat under a nylon-strung platinum wire ‘harp’. XY images were taken through the cell bodies in the inner region of the ONL, nearest the outer plexiform layer, where rod (as opposed to cone, which lie at the outer edge of the ONL) photoreceptor nuclei reside. Images were acquired at 3 sec intervals and analysed off-line. Cells were selected at random and the mean fluorescence of individual cells was calculated and normalised against the fluorescence at time 0s. Drugs were applied by micro-pipette injection into the bathing solution.

Drugs. DCPG ((S)-3,4-dicarboxyphenylglycine) (20 μM; final concentration in bath), CPPG ((RS)-alpha-cyclopropyl-4-phosphonophenylglycine) (100 μM) and NMDA (N-methyl-D-aspartate) (200 μM) were supplied by Tocris (UK).

Developmental window cell counts. To assess the integration potential of donor cells from a range of developmental stages, adult animals received a single 1 μl injection of 4×10⁵ cell/μl in each eye. Three weeks post-transplantation, animals were sacrificed and the eyes prepared for analysis as described above. Cells were considered integrated if the whole cell body was visible together with at least one of the following; spherule synapse and/or inner/outer segments. The average number of integrated cells per section was determined by counting all the integrated GFP-positive cells in every 1 in 4 serial sections through the site of injection in each eye. This was multiplied by the total number of sections that encompassed the injection site to give an estimate of the mean number of integrated cells per eye.

Assessment of light sensitivity. Pupillometry. Following dark-adaptation for at least 1 h during the light phase of their light/dark cycle, un-anaesthetized were manually held with the eye to be recorded perpendicular to an infrared sensitive camera fitted with a macro lens. Background illumination was provided by infrared LEDs throughout the experiment. Animals were subjected to a series of 10 second white light exposures of ascending irradiance controlled by neutral density filters provided by a fiber optic from a 100 W halogen lamp (Zeiss). At least 2 mins elapsed between exposures, during which time the animal was unrestrained. A complete intensity series was performed for one eye before retesting the other eye at identical intensities, with at least an hour of darkness between exposures of the 2 eyes. Subsequently, pupil area was determined from individual video frames captured 5 s after light exposure, at which time constriction was maximal. The effective intensity of each exposure was calculated by measuring the spectral irradiance (photons/s/m²/nm) incident on the cornea, at 1 nm intervals between 300-870 nm with a Ocean Optics USB2000 spectrometer fitted with a P-600-5-UV/Vis fiber optic and CC-3-UV irradiance collector (previously calibrated with reference to an Ocean Optics DH-2000-CAL calibration light) and weighting these data by the spectral sensitivity of the wildtype murine pupil response (See, e.g., Lucas et al., Nat. Neurosci. 4, 621-626 (2001)). To facilitate comparisons between individuals, pupil areas (ai) were expressed relative to the dilated area immediately prior to each exposure (a0). A 4 term sigmoid was fitted to the pupil area vs irradiance data for each eye and the irradiance required to give 50% of the dilated pupil area taken as a measure of that eye's sensitivity. Following pupil assessment, animals were sacrificed and eyes prepared for analysis as described above. The total number of integrated cells per eye was determined by counting all the GFP-positive cells in the ONL of every section. Slide identity was masked by an independent observer prior to assessment.

Extracellular Field Potential Recordings. Three weeks after transplantation, mice were dark-adapted for 1 h prior to sacrifice in the dark. Eyes were removed under infra-red light and the lens and vitreous were dissected away, but the RPE was left intact. Four small cuts were made to allow the retinal whole mount to lie flat. Preparations were mounted GCL side uppermost in a blacked-out interface recording chamber where they were continually perfused with oxygenated Krebs' solution (containing, in mM: NaCl, 124; KCl, 3; KH2PO4, 1.25; MgSO4, 1; CaCl2, 2; NaHCO3, 26 and glucose, 10), maintained at 34° C. Extracellular recordings were made approximately 30 mins after the retina was positioned in the chamber, from the GCL using glass microelectrodes (1-3 M-Ohm) filled with the same Krebs' medium as that used to maintain the slices in the recording chamber. Recordings were made in at least 8 independent regions around the optic nerve head. Light-evoked potentials were stimulated by flashes (100 ms duration, 0.5 s interval) of increasing intensity emitted by a green LED (562 nm peak wavelength) positioned 8 mm above the retina. Voltage responses, evoked by 10-20 flashes at each intensity, were recorded via an Axoprobe 1A amplifier (Axon Instruments), digitized via a CED1401 interface (Cambridge Electronic Design), and stored on a computer system running Spike2 software (Cambridge Electronic Design). Average responses (10-20 responses) were computed and average light intensity plots were drawn for each eye by determining the average voltage change from all regions of interest (ROIs) at each stimulus intensity. The stimulus threshold for a light-evoked response was determined as being the stimulus intensity that evoked a response magnitude that was 10% of the potential evoked by the maximum stimulus. Quantitative results are expressed as mean±SEM.

### Transplant Potential of Photoreceptor Progenitor Cells.

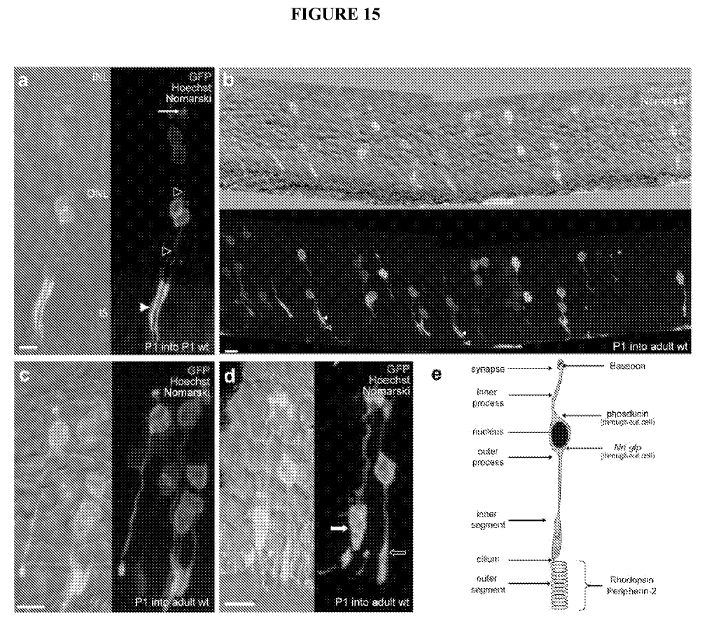

The transplantation potential of immature mouse retinal donor cells, taken from the early postnatal period at the peak of rod photoreceptor genesis (Postnatal day (P) 1) (See, e.g., Young, Anat. Rec. 212, 199-205 (1985)) was assessed. At this age, the retinal microenvironment is favourable to promote the differentiation and integration of transplanted cells within the ONL. Furthermore, transplanted cells have a higher probability of integration if recipient and donor retinas are at equivalent stages of development. Cell suspensions were prepared from P1 neural retinas of transgenic mice carrying a gfp reporter gene driven by a ubiquitously expressed promoter (Cba.gfp^{+/-}) (See, e.g., Okabe et al., FEBS Lett. 407, 313-319 (1997)) and ˜2×10⁵ cells were injected into the subretinal space of GFP-negative wildtype P1 littermates. Three weeks post-transplantation, a substantial number of cells (10-200 cells/eye) had migrated into the recipient neural retina. Most (>95%) of these were correctly orientated within the ONL and had morphological features typical of mature photoreceptors (See FIGS. 15a and 15e).

Since a population of cells within the P1 retina was able to integrate and differentiate into photoreceptors when transplanted in the immature retina, P1 cells (˜8×10⁵ cells/eye) were transplanted into the subretinal space of adult GFP-negative wildtype mice. In contrast to previous reports (See, e.g., Chacko et al., Biochem. Biophys. Res. Commun. 268, 842-846 (2000); Yang et al., J. Neurosci. Res. 69, 466-476 (2002), it was observed that transplanted cells did in fact migrate into the ONL of the adult recipient retina. The cells integrated into the ONL in proportionately similar numbers (300-1000 cells/eye), and had the morphological characteristics of mature photoreceptors (See FIGS. 1b-1e). Virtually all integrated cells were rod-like, a morphological characteristic of mature photoreceptors (See, e.g., Young, Anat. Rec. 212, 199-205 (1985); Carter-Dawson and LaVail, J. Comp Neurol. 188, 263-272 (1979)), although cone-like profiles were very occasionally observed (See FIG. 15d). The site of injection appeared important because on no occasion did intravitreal injections lead to integration within the ONL in either P1 or adult recipients.

### Plasticity of Photoreceptor Progenitor Cells.

Fusion between transplanted and host cells has been proposed as an explanation for the apparent plasticity of stem cells (See, e.g., Terada et al., Nature 416, 542-545 (2002); Ying et al., Nature 416, 545-548 (2002); Weimann et al., Nat. Cell Biol. 5, 959-966 (2003)). In order to further characterize photoreceptor precursor cells, dissociated P1 GFP-positive cells were transplanted into the subretinal space of adult transgenic mice ubiquitously expressing cyan fluorescent protein (Ck6.cfp^{+/+}) (See, e.g., Hadjantonakis et al., BMC. Biotechnol. 2, 11 (2002)). Confocal sections were examined through inner segments (the widest cytoplasmic part) of integrated GFP-positive cells, but co-localized GFP and CFP signals were not identified in any of the retinas studied (N=8) (See FIG. 16a). Other data indicates that cell fusion may result in multinuclear cells (See, e.g., Weimann et al., Nat. Cell Biol. 5, 959-966 (2003); Kashofer, K. & Bonnet, Gene Ther. 12, 1229-1234 (2005)). No more than a single nucleus was observed in any of the integrated cells. DNA labelling of P0 GFP-positive donor mice with intraperitoneal Bromo-deoxy-Uridine (BrdU) further confirmed that the single nuclei of integrated cells in the ONL originated from donors (See FIG. 16b), thereby ruling out occurrence of cell fusion.

Identification of Specific Photoreceptor Progenitor Cells that Integrated within the ONL.

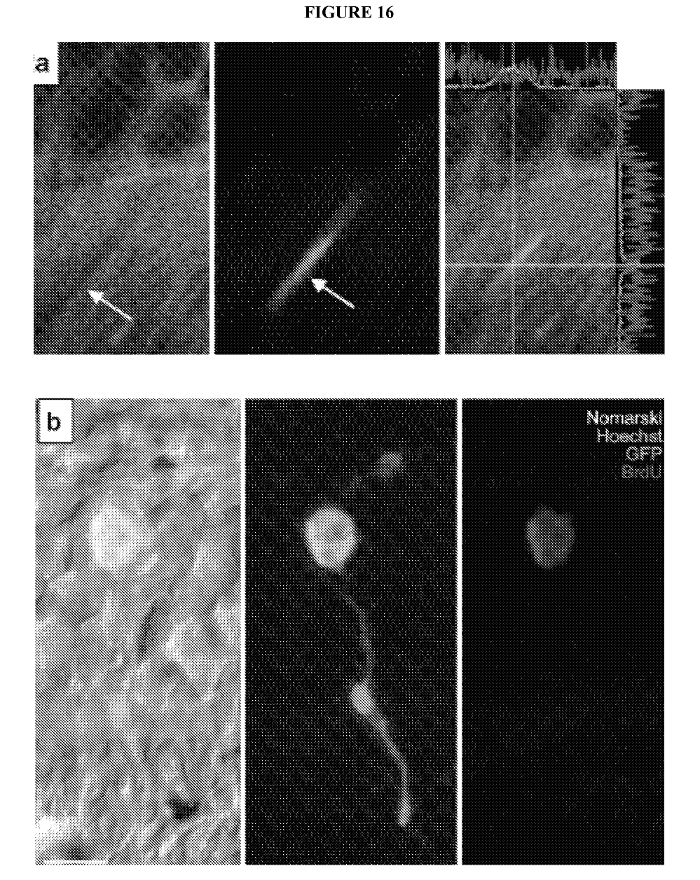

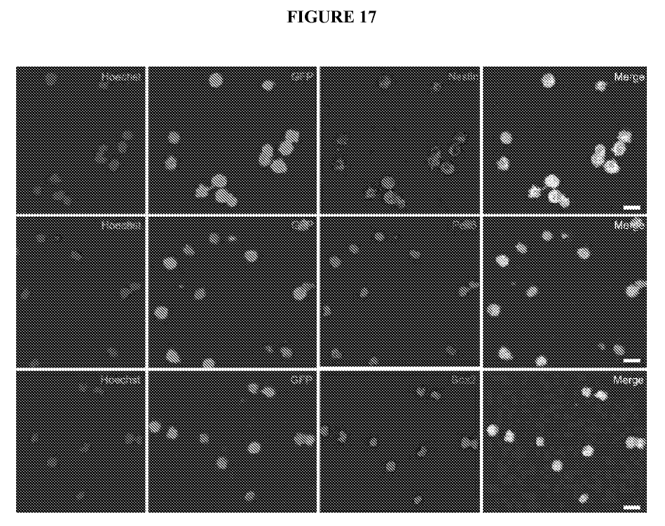

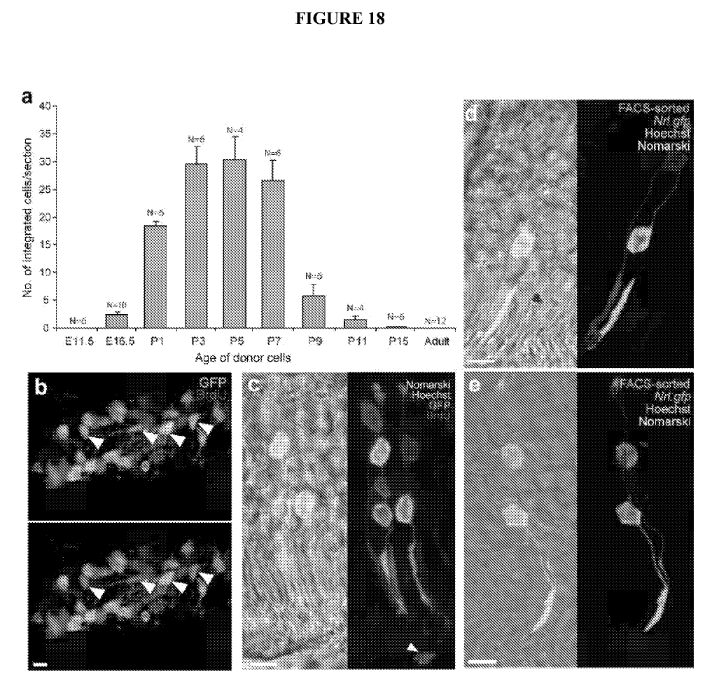

The population of cells derived from the P1 retina comprises a mixture of proliferating progenitors, post-mitotic precursors and differentiated cells that do not yet express the markers of mature photoreceptors (See, e.g., Young, Anat. Rec. 212, 199-205 (1985)). Thus, experiments were conducted during the development of the present invention to identify and characterize which of these cells integrated within the ONL. First, the developmental time window for obtaining donor cells that would successfully integrate following transplantation was determined. Dissociated cells were taken from embryonic day (E) 11.5, E16.5, P1-P15 or adult GFP-positive donors and transplanted by a single standardized injection into the subretinal space of adult wildtype recipients. Cells derived from E11.5 retinas, the latest stage that comprises almost entirely proliferating progenitors (See, e.g., Young, Anat. Rec. 212, 199-205 (1985); Carter-Dawson and LaVail, J. Comp Neurol. 188, 263-272 (1979); and See FIG. 17), survived in the subretinal space following transplantation, but in all cases failed to integrate (See FIG. 18a). Similarly, cells derived from adult retinas survived but consistently failed to integrate. In contrast, cells derived from P1-P7, that primarily include immature rod precursors, showed robust integration that was optimal when the donor cells originated from P3P5 donors, declining thereafter (See FIG. 18a). In all cases, a large mass of viable cells was found in the subretinal space at the time of sacrifice, indicating that lack of integration was not due to poor cell survival.

The failure of immature progenitors to integrate after transplantation was unexpected; nevertheless, it suggested a change in the properties of these cells at or after terminal mitosis. In order to test this, P1 cells were transplanted into the eyes of wildtype adult recipient mice (N=12), that concurrently received intraperitoneal injections of BrdU, and on every other day for 8 days. Thus, donor cells that undergo division after the transplantation are labelled with BrdU. Mitotic donor cells were found to survive and continue to divide in the subretinal space of the recipient eye (See FIG. 18b), but on no occasion were BrdU-labelled cells found to be integrated within the recipient retina (See FIG. 18c). Thus, the present invention provides that the cells capable of integrating into the recipient retina are not proliferating progenitors.

In order to further identify and characterize the nature of integrated cells, a transgenic mouse line that carries a gfp reporter gene driven by the Nrl promoter (Nrl.gfp^{+/+}, described in Example 1 above) was used. Nrl is a basic motif-leucine zipper transcription factor important for the differentiation (See, e.g., Example 1) and maintenance of rod photoreceptors (See, e.g., Bessant et al., Nat. Genet. 21, 355-356 (1999); Mears et al., Nat. Genet. 29, 447-452 (2001); and Swain et al., J. Biol. Chem. 276, 36824-36830 (2001)) and the gfp reporter gene in Nrl.gfp^{+/+} mice is a marker of new-born post-mitotic rod precursors (See Example 1). Fluorescence-activated cell sorting (FACS) was used to isolate GFP-positive post-mitotic rod precursors from dissociated P1 Nrl.gfp^{+/+} retinas, and these cells were transplanted into adult wildtype recipients. Donor cells derived from this sorted population routinely integrated within the ONL of recipient retinas (See FIGS. 18d and 18e). While the number of FACS-sorted cells per injection was ˜25% that of normal unsorted transplants, a similar number of cells (200-800 cells/eye; N=6) integrated, thereby providing that the optimal ontogenetic stage for donor cells for effective rod photoreceptor transplantation (e.g., integration and development) corresponds with Nrl expression (e.g., Nrl expression can be used as a photoreceptor progenitor cell marker (e.g., to identify specification of rod fate)).

The observation, made during development of the present invention, that Nrl.gfp-positive rod precursors, but not progenitor cells, integrate within the ONL of the adult retina, provides that the adult retina lacks developmental cues important for promoting the differentiation of a dividing progenitor cell through the multiple developmental steps required to generate new photoreceptors. By transplanting Nrl.gfp^{+/+} cells from E11.5 donors, a stage prior to the onset of Nrl expression, it was determined that these cells failed to integrate within the host retina. However, they were able to differentiate to a stage where both Nrl and rhodopsin were expressed, and formed organized rosettes structures within the subretinal space (See FIG. 19). Thus, the present invention provides that the adult retina is able to support the survival and differentiation of progenitor cells, whereas the integration and differentiation of rod photoreceptors can primarily be achieved when the cells are at the appropriate ontogenetic stage when transplanted (e.g., when the cells express Nrl).

### Characterization of Integrated Photoreceptor Progenitor Cells

Integrated cells had the morphological appearance of mature rod photoreceptors. In order to confirm their identity, two additional methods were used. First, as described above, sub-retinal injections of cells derived from the Nrl.gfp^{+/+} mouse led to their widespread integration into the ONL of adult recipients (See FIGS. 18d and 18e). These cells had a morphological appearance very similar to those derived from transgenic mice expressing GFP ubiquitously. The restriction of Nrl.gfp expression to rods (See, e.g., Example 1) provides direct genetic evidence that the majority of transplanted integrated cells within the ONL are rod photoreceptors. Second, retinal sections were stained with antibodies against a number of photoreceptor markers. At 3 weeks post-transplantation, numerous integrated cells were immunopositive for phosducin (See FIG. 19a) and the photopigment rhodopsin (See FIG. 21c), demonstrating that these cells differentiate to express elements of the phototransduction cascade. Importantly, integrated cells were also shown to express the ribbon synapse protein, bassoon (See FIG. 19b), indicating that these cells had assembled structural components of the spherule synapse (See, e.g., Tom et al., J. Cell Biol. 168, 825-836 (2005)), a requirement for these cells to communicate with the inner retina. Immunostaining for the rod bipolar cell marker, protein kinase C, further demonstrated that transplanted cells formed synapses with downstream targets in the recipient retina (See FIG. 19c). In addition, a pharmacological approach was used to assess the presence of a subtype of metabotropic glutamate receptor, mGluR8, that is rod-specific and localized exclusively to the rod spherule ribbon synapse (See FIGS. 19d-19f). See, e.g., Koulen et al., Proc. Natl. Acad. Sci. U.S.A 96, 9909-9914 (1999); Koulen and Brandstatter, Invest Ophthalmol. Vis. Sci. 43, 1933-1940 (2002)). Stimulation of rod mGluR8 receptors induces a decrease in intracellular calcium ([Ca²⁺]i), that can be measured using confocal microscopy. Application of either glutamate or the specific mGluR8 agonist DCPG consistently evoked changes in ([Ca²⁺]i) in both recipient and Nrl.gfp-positive integrated cells (See, e.g., FIGS. 19e and 19f), an effect that could be blocked by the metabotropic glutamate antagonist CPPG (See, e.g., FIGS. 19e and 19f). Conversely, agonists specific for a second glutamate receptor, the NMDA receptor, that is expressed by other retinal cell types but not photoreceptors (See, e.g., Koulen and Brandstatter, Invest Ophthalmol. Vis. Sci. 43, 1933-1940 (2002)), showed no effect (FIG. 19e). Thus, when taken together, the present invention provides the identity of transplanted cells that integrate into the ONL as rod photoreceptors (e.g., that express molecules essential for phototransduction). Furthermore, the present invention provides that these cells form synaptic connections with downstream targets and respond to specific, synapse-dependent stimuli, in a manner indistinguishable from endogenous photoreceptors in the recipient retina.

### Transplanted Cells Integrate and Survive in Degenerating Retinas and Resolve Visual Function.

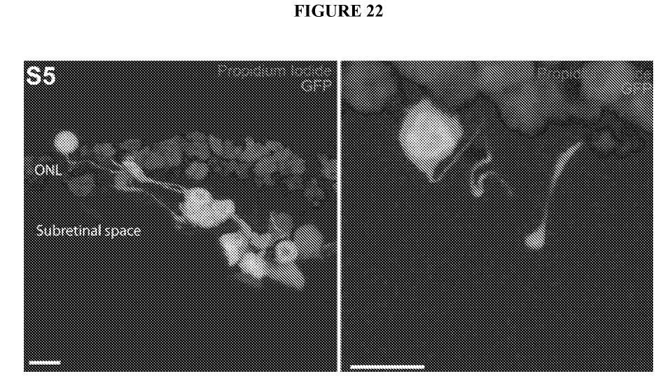

In order for cell transplantation to be a viable therapeutic strategy, donor cells must be able to integrate and survive in a degenerating retina and restore visual function. GFP-positive cells (unsorted) from P1 Nrl.gfp^{+/+} mice were transplanted into the sub-retinal space of three mouse models of inherited retinal degeneration; retinal degeneration slow (rds), retinal degeneration fast (rd) and a rhodopsin knockout (rho^{-/-}). Malfunction and degeneration of rods occurs in all of these strains and mutations in the corresponding human genes lead to various forms of retinal dystrophy (See, e.g., Wells et al., Nat. Genet. 3, 213-218 (1993); McLaughlin et al., Nat. Genet. 4, 130-134 (1993); and Rosenfeld et al., Nat. Genet. 1, 209-213 (1992)). The rds mouse has a mutation in the gene encoding peripherin-2, required for the generation of photoreceptor outer segment discs. The ONL starts to degenerate 2 weeks after birth, continuing slowly over the course of 12 months (See, e.g., Reuter, J. H. & Sanyal, Neurosci. Lett. 48, 231-237 (1984); Sanyal et al., Curr. Eye Res. 7, 1183-1190 (1988)). Nrl.gfp-positive donor cells integrated and differentiated as photoreceptors into the adult rds retina in numbers similar to that seen in wildtypes (See FIG. 21a), and remained viable for at least 10 weeks. Peripherin-2 staining was completely absent in recipient photoreceptors, but was seen in short outer segments emerging from transplanted cells (See FIGS. 21a and 21b) often connected by an identifiable GFP-positive cilium (See FIG. 21b). The rd mouse undergoes a rapid retinal degeneration, reducing the ONL to a single layer of predominantly cone cells by 3 weeks (See, e.g., Carter-Dawson et al., Invest Ophthalmol. Vis. Sci. 17, 489-498 (1978)). In contrast to host rods, P1 Nrl.gfp-positive cells transplanted into the P1 rd mouse retina survived, although with variable morphology due to the collapse of surrounding tissue (See FIG. 22)). In the rho^{-/-} mouse retinal degeneration is slower, but the ONL degenerates by 12 weeks (See, e.g., Humphries et al., Nat. Genet. 15, 216-219 (1997)). Thus, P1 Nrl.gfp-positive cells were transplanted into animals aged 4 weeks, and this again led to the integration of cells. Rhodopsin immunostaining was localized to the outer segments, in a pattern similar to that seen for peripherin-2 following transplantation into the rds mouse (See FIG. 21c).

In order to assess whether transplanted cells were light-responsive and making functional connections to downstream targets, two techniques were used; pupillometry, and extracellular field potential recordings from the ganglion cell layer. 7 week old rho^{-/-} mice, that have no functional rod photoreceptors and are thus insensitive to low light intensities (See, e.g., Toda et al., Vis. Neurosci. 16, 391-398 (1999); Lucas et al., Nat. Neurosci. 4, 621-626 (2001)), were recorded. These mice retain some cone function at early stages and are thus able to detect high intensity stimuli (>0.1 candelas/s/m²) (See, e.g., Toda et al., Vis. Neurosci. 16, 391-398 (1999)). Rho^{-/-} mice received P1 Nrl.gfp (rho^{+/+}) donor cells in one eye and a sham injection of P1 rho^{-/-} donor retinal cells in the other, three weeks prior to assessment.

Light-evoked extracellular field potentials recorded from the ganglion cell layer were used to examine the transfer of light information from the transplanted rod photoreceptors to inner retinal neurons. In uninjected rho^{-/-} mice, ganglion cell activity was absent at low light intensities (e.g., when rod responses would be elicited) with threshold responses of 10% of maximum being discernible only at stimulus intensities of 0.052 candelas/s/m² (See FIG. 21d). Such stimulus intensities fall within the range of cone stimulation in rho^{-/-} mice (See, e.g., Toda et al., Vis. Neurosci. 16, 391-398 (1999)). Similarly, no measurable response in sham (rho^{-/-} cells) injected eyes at low light intensities was observed. Again threshold responses were only observed at intensities of 0.052 candelas/s/m² (See FIGS. 21d and 21e). In contrast, threshold responses were elicited in the treated eyes (Nrl.gfp^{+/+}/rho^{+/+}) by stimuli as low as 5.7×10⁻³ candelas/s/m² (See FIGS. 21d and 21e, well within the rod photoreceptor range (See, e.g., Toda et al., Vis. Neurosci. 16, 391-398 (1999)). In uninjected wildtype mice, threshold responses were evoked at 4.1×10⁻³ candelas/s/m². Thus, the present invention provides that integrated cells are light responsive and make functional synaptic connections to downstream retinal targets.

Light-induced pupil constriction is a behavioral response that in mice requires photoreceptors to have functional connections with central brainstem targets. The pupil responses of both eyes of uninjected wildtype mice, and rho^{-/-} mice that had received Nrl.gfp/rho^{+/+} donor cells into one eye and sham injections (rho^{-/-}) into the other, were examined at various intensities (See FIGS. 21f-21i). Wildtype pupils were approximately 3.15 log units more sensitive than those of the sham injected eyes of rho^{-/-} mice (See FIGS. 21g and 21h). Sham-injected eyes in rho^{-/-} mice had no discernible pupil reflex at low light intensities (See FIG. 21h). However, eyes in 5 out of 9 rho^{-/-} mice injected with Nrl.gfp/rho^{+/+} cells were more sensitive than the sham-injected eye (See FIG. 21h). There was no difference between the two eyes in the remaining 4 animals. Following pupil assessment, the eyes were examined histologically for evidence of cell integration within the ONL. Across all 9 animals, the difference in pupil sensitivity compared with the control eye correlated with the number of integrated Nrl.gfp/rho^{+/+} cells counted in the host ONL (Pearson product moment correlation co-efficient R=0.87, P=0.0013; Spearman rank correlation coefficient r=0.783, P=0.010) (See FIG. 21i). Thus, the present invention provides that integrated cells are light responsive and make functional connections to the brain.

### Example 3

### Characterization of Transplanted Photoreceptor Precursor Cells in a Mouse Model of Retinal Degeneration

### Materials and Methods.

Experimental Animals. Experimental procedures strictly conformed to the Guidelines for Animal Experiments of Kyoto University. All animals were fed laboratory chow ad libitum with free access to water and kept on a 14/10-hour light-dark cycle.

Preparation of Donor Cells and Recipients. Donor cells were prepared from P0-P2 retinas of the neural retina leucine zipper (Nrl)-GFP transgenic mice (See Example 1). Nrl is a basic motif-leucine zipper transcription factor that is preferentially expressed in rod photoreceptors and required for rod differentiation (See, e.g., Swaroop et al., Proc Natl Acad Sci USA. 1992; 89:266-270; and Mears et al., Nat Genet. 2001; 29:447-452). The Nrl promoter directed expression of enhanced green fluorescent protein (EGFP) specifically to new-born rod precursors and mature rods in the Nrl-GFP transgenic mouse. Eyes were enucleated, and the neural retinas dissected and dissociated with a Papain-Protease-DNase solution. N-methyl-N-nitrosourea (MNU; Sigma, St. Louis, Mo.), an alkylating agent that induces photoreceptor degeneration by forming 7-methyldeoxyguanosine DNA adducts in the nuclei of photoreceptors, was administered at a dose of 60 mg/kg to adult C57B1/6 mice by intraperitoneal injection 7 days before transplantation (See, e.g., Doonan et al., J Neurosci. 2003; 23:5723-5731; Ogino et al., Toxicol Pathol. 1993; 21:21-25; Yuge et al., In Vivo. 1996; 10:483-488).

Transplantation Procedure. One μl of dissociated Nrl-GFP+ photoreceptor cell suspension (1.0×10⁵ cells/μl each) without or with chondroitinase ABC (ChABC) (0.025 U/μl, Wako, Tokyo, Japan) (Nrl group, Nrl/ChABC group, respectively) or 1 μl of PBS (sham group) was drawn into a tapered glass pipette connected to a modified tube and injected through the sclera into the subretinal space. The procedure was performed under surgical microscope.

Tissue Processing. Two or four weeks after surgery, the animals were perfused transcardially with 4% paraformaldehyde (Merck, Darmstadt, Germany) in 0.1 M phosphate buffer after sedation with ketamine (15 mg/kg). Eyes were removed and immersion fixed with 4% paraformaldehyde at 4° C. overnight and then in 25% sucrose-PBS to cryoprotect. The specimens were embedded in an optimal cutting temperature compound (Miles, Elkhart, Ind.) and consecutive 12-μm frozen sections were sliced on a cryostat.

Immunofluorescence. Sections were washed in PBS, preincubated with a blocking solution (containing 20% skim milk and 0.3% Triton X-100 in PBS) for 30 minutes, and then incubated overnight at 4° C. with primary antibodies diluted in a blocking solution (containing 5% skim milk and 0.3% Triton X-100 in PBS). The primary antibodies and working dilutions were as follows: mouse and rabbit anti-GFP (1:500, Molecular Probes, Eugene, Oreg.), mouse monoclonal CS-56 IgM antibody (1:200, Sigma) that reacts specifically with chondroitin sulfate containing proteoglycans, and anti-vesicular glutamate transporter 1 (VGluT1; 1:100, Chemicon, Hampshire, UK), a marker for active presynaptic formation (See, e.g., Fujiyama et al., J Comp Neurol. 2003; 465:234-249). Sections were incubated for 90 minutes with secondary antibodies diluted 1:500 in PBS containing 5% skim milk and 0.3% Triton X-100. The secondary antibodies used were as follows; goat anti-mouse IgG (H+L) antibodies (ALEXA FLUOR 488, ALEXA FLUOR 594, Molecular Probes) and goat anti-rabbit IgG (H+L) antibodies (ALEXA FLUOR 488, ALEXA FLUOR 594, Molecular Probes). Sections were counterstained with Cytox blue to reveal cell nuclei (1:500 in distilled water, Molecular Probes).

Images were collected with a laser-scanning confocal microscope (TCS SP2, Leica, Heidelberg, Germany). To verify the co-localization of GFP and VgluT1 obtained in the x-y plane, stained profiles appearing in serial optical sections were rescanned along the z-axis, producing two-dimensional cross-sectional images (x-z scan, y-z scan).

Analysis of Tissue Sections. Cells were counted using a 63× objective in every tenth section to sample across the entire retina. In each section, cells expressing GFP in each layer of the retina were counted. The GFP+ cells residing at the outer margin of MNU-treated host retina where the photoreceptor layer had originally existed were counted as residing within outer nuclear layer and/or outer plexiform layer. The percentage of GFP+ cells bearing neurites per GFP+ cells within the retina was also determined. To quantify the dendritic growth of transplanted cells, GFP+ cells with neurites that had extended into the host retina were counted and expressed as the percentage of GFP+ cells residing within the retina. Statistical significance was determined by Student's t-test. P<0.05 was considered to be statistically significant.

Electrophysiology. Electrophysiological recordings were performed as described (See, e.g., Ueda et al., Vision Res. 2005). Briefly, following overnight dark adaptation, each mouse was anesthetized by intraperitoneal injection of an anesthetic cocktail (150-200 μl) consisting of 0.04 ml/ml ketamine, 0.13 ml/ml xylazine, and 0.1 g/ml ethyl carbamate diluted in PBS. Pupils were dilated with 0.5% tropicamide. Animals were placed on a regulated heating pad under dim red illumination and electroretinograms (ERGs) were recorded with a gold loop electrode placed on the corneal surface maintained with 3% methylcellulose gel. A stainless steel reference electrode and ground electrode were each inserted subcutaneously on the head and in the tail of the mice. A strobe flash stimulus was performed to the dark-adapted, dilated eyes in a full-field stimulus dome (6.5 cm diameter Sanso). Responses were amplified, filtered, digitized and computer averaged at all intensities. The amplitude of the a-wave was measured from the prestimulus baseline to the a-wave trough. The amplitude of the b-wave was measured from the trough of the a-wave to the crest of the b-wave. Data were analyzed off-line using pClamp8 (Axon Instruments).

### Results

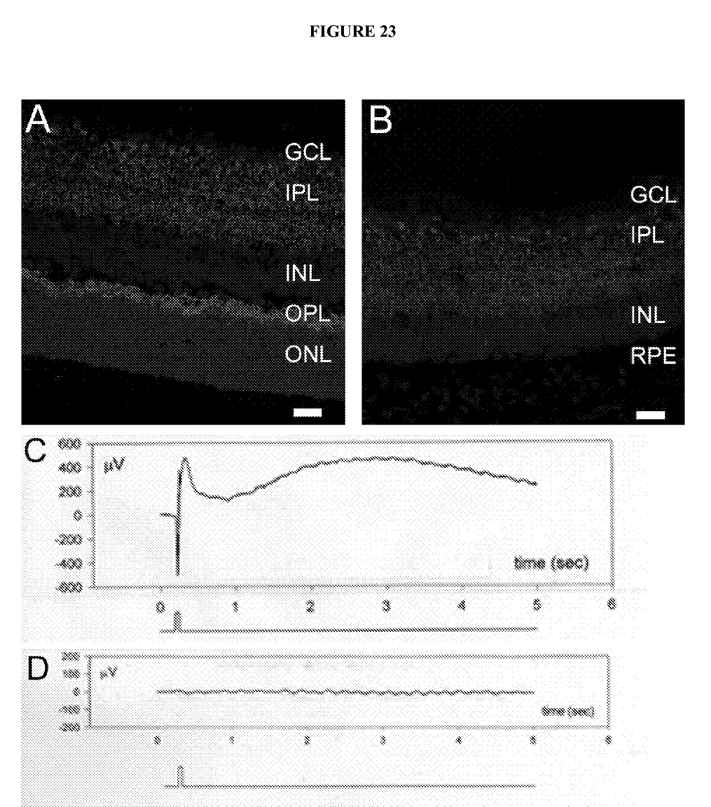

In order to induce apoptosis of photoreceptors, adult C57b1/6 mice were injected with a single intraperitoneal dose of MNU (60 mg/kg). This dose of MNU treatment initiates apoptosis in all photoreceptors within 1 week of injection (See, e.g., Yuge et al., In Vivo. 1996; 10:483-488). At 2 days after injection, TUNEL assays revealed nuclear labeling in the majority of the photoreceptor cells and invariably negative staining in the other cell layers. Immunostaining of retinal sections at this time-point with Cytox blue indicated that the thickness of outer nuclear layer (ONL) had decreased remarkably. At 1 week after injection, no TUNEL staining was observed, as reported previously (See, e.g., Doonan et al., J Neurosci. 2003; 23:5723-5731; Ogino et al., Toxicol Pathol. 1993; 21:21-25). Immunostaining against VgluT1 and counterstaining with Cytox blue revealed that ONL was essentially destroyed (See FIG. 23B). VgluT1 was present only in the IPL whereas intense VGIuT1 immunoreactivity was distributed in the inner plexiform layer (IPL) and outer plexiform layer (OPL) for age-matched controls (See FIG. 23A). To examine electrophysiological changes, electroretinograms (ERGs) were performed on these mice. ERG traces from MNU-treated mice demonstrated these animals were insensitive to visual stimulation as no responses were detectable (See FIGS. 23C and 23D), analogous to the data obtained by immunostaining.

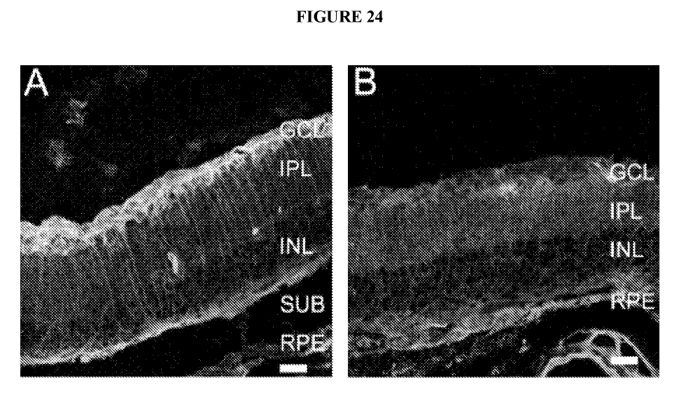

To determine the degree of glial scarring induced by the transplantation procedure, 1 μl of vehicle was transplanted into MNU-treated mice subretina, with examination of the retina 2 days after surgery. Two characteristics of glial scarring are the upregulation of CPSG and GFAP expression. Increased staining intensity was observed for both CS-56, an antibody that recognized CSPGs, and GFAP at the outer margin of host retina around the transplantation site (See FIG. 24A). Similar changes are observed at the lesion site elsewhere in the CNS (See, e.g., McKeon et al., J Neurosci. 1991; 11:3398-3411; Canning et al., Exp Neurol. 1993; 124:289-298; Dou & Levine, J Neurosci. 1994; 14:7616-7628; Smith-Thomas et al., J Cell Sci. 1994; 107 (Pt 6):1687-1695).

Next, to examine the effect of ChABC in vivo, 1 μl of cell suspension with or without vehicle containing ChABC was injected into the eyes of MNU-treated mice subretinally. The staining intensity of CS-56 at the outer margin of host retina was less in the chondroitinase-treated retinal section (See FIG. 24B) relative to the control without chondroitinase treatment (See FIG. 24A). Thus, the present invention provides that the ChABC treatment substantially, if not completely, degraded chondroitin sulfate proteoglycans (CSPG) in the extracellular matrix (ECM) of the glial scar at the injection site.

Next, photoreceptor precursor cells were transplanted into chemically induced photoreceptor degraded mice. For transplantation, MNU was injected intraperitoneally into Adult C57B1/6 mice (postnatal 6-7 weeks), photoreceptor precursor cells (GFP+) were transplanted transsclerally into the subretinal space 1 week later, and the fate of the GFP+ cells followed for different durations. The constitutive expression of GFP by the transplanted photoreceptors allowed, among other things, the ability to distinguish the grafted GFP+ cells from host retina and to determine graft-host connectivity (e.g., even after long survival times).

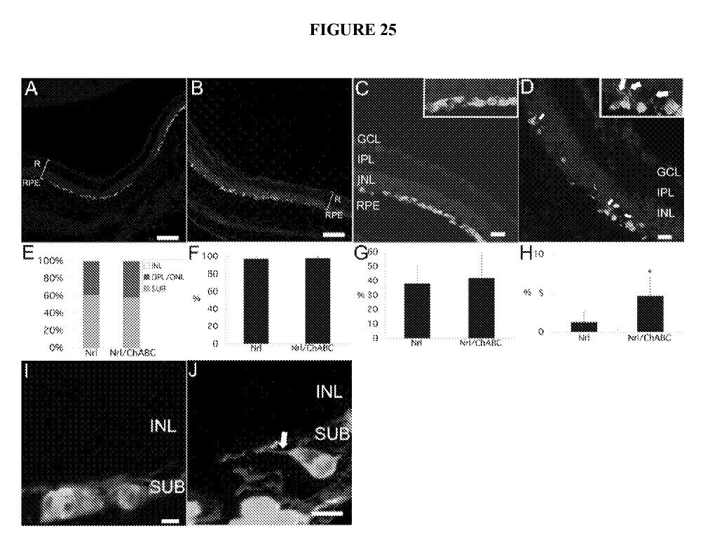

To determine the effect of ChABC, the outcome of transplantation using GFP+ photoreceptor cells with or without application of ChABC was compared. Although an understanding of the mechanism is not necessary to practice the present invention and the present invention is not limited to any particular mechanism of action, in some embodiments, degenerated retina can be repaired and retinal function rescued and/or recovered if the dysfunctional photoreceptors are replaced with new, healthy photoreceptors (e.g., that can make appropriate synaptic connections with the remaining functional circuitry within the retina). At 2 weeks after transplantation, grafted GFP+ photoreceptor cells in both groups were widespread at the outer margin of the host retina where the photoreceptor layer had originally resided. Morphologically, a portion of the GFP+ photoreceptor cells had extended neurites. The cell distribution and morphology were similar for both groups (See FIGS. 25A-D).

The relative distribution pattern of the transplanted cells from the ChABC-treated group was indistinguishable from untreated (See FIG. 25E). The majority of the grafted GFP+ photoreceptor cells were present at the outer margin of the host retina in both groups (99.63±0.52% in Nrl/ChABC group, 99.14±0.87% in Nrl group, P=0.31) (See FIG. 25F). The neurite outgrowth from the grafted cells of both groups was estimated by counting the number of GFP+ cells that extended neurites. In the Nrl/ChABC group, 33.61±9.68% of GFP+ cells within the retina sprouted neurites. Roughly the same percentage of cells with neurites were observed in the Nrl group (30.73±4.89%) (P=0.68) (See FIG. 25G). In contrast, 4.60±2.83% of the Nrl-GFP+ photoreceptors elaborated neurites toward the host retina in Nrl/ChABC group, while only 1.23±1.47% of neurons in the Nrl group extended neurites toward the host tissue. This difference is significant (P<0.05) (See FIG. 25H).

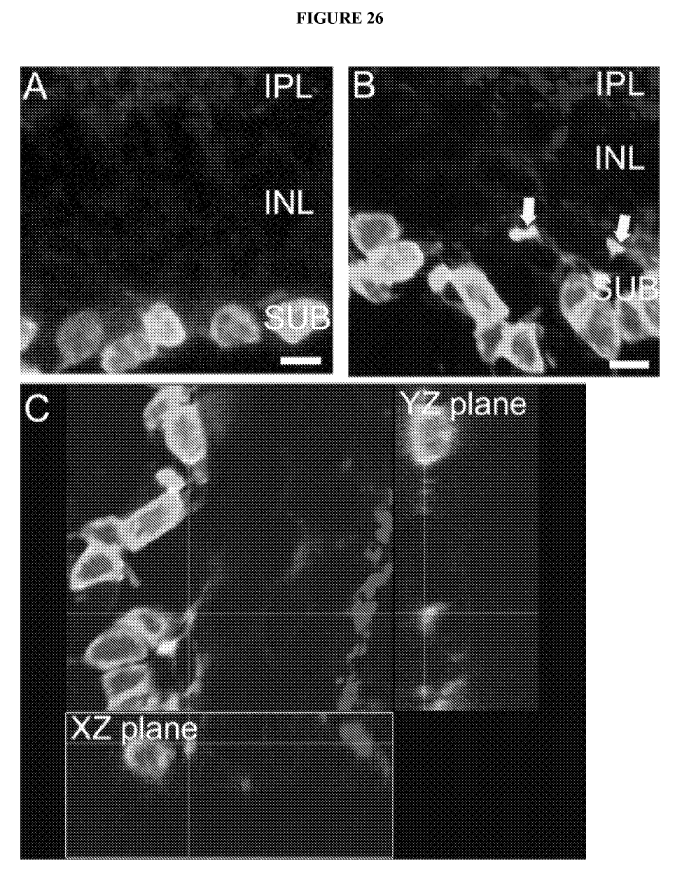

In order to examine the relationship between neurite formation by the grafted cells and glial scarring of host retina, immunofluorescent double staining for GFP and CS-56 or VgluT1 was performed. GFP+ neurites directed toward the host retina in the Nrl/ChABC group extended over the CSPG-rich ECM at the outer margin of the retina to contact neurons beyond this border (See FIG. 25J). In addition, these GFP+ neurites were immunopositive for VgluT1 (See FIG. 26B). Colocalization between GFP and VgluT1 was determined by three-dimensional analysis of a z-series of images collected with a confocal microscope (See FIG. 26C). Thus, the present invention identifies synaptic contacts between the grafted photoreceptor cells and the host retina (e.g., identified via colocalization of GFP and VgluT1 staining). Some transplanted neurons extended processes that resembled photoreceptor outer segments and established contact with the retinal pigment epithelium. In contrast, these morphologies were rarely observed in the Nrl group (See FIGS. 25I and 26A) although some neurites extended toward the host retina.

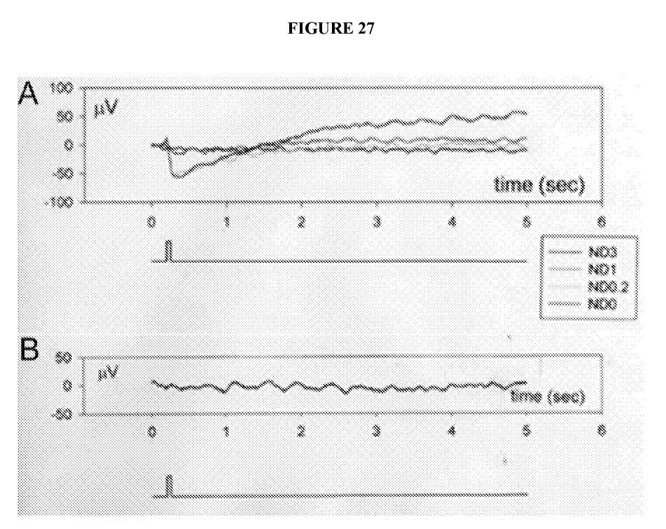

In order to evaluate whether these transplants could induce functional recovery, ERG recordings were performed 1 month after transplantation into the MNU-treated mice that had suffered complete retinal degeneration. Of 12 mice examined, one animal exhibited a-wave-like response in the treated eye but not the contralateral control eye (See FIG. 27). Moreover, the ERG amplitudes increased proportionally with light intensity (ND0-ND3). Thus, the present invention provides a functional recovery, in addition to morphological recovery, to chemically induced photorector degraded eyes via transplantation of photoreceptor precursor cells.

### Example 4

### Photoreceptor Precursor Cells Utilized to Identify Genes and Proteins Involved in Human Disease (e.g., Retinal Degeneration)

### Materials and Methods.

Animal studies. The mice were bred and maintained in standardized conditions at The Jackson Laboratory and Kellogg Eye Center. The use of mice was approved by the Institutional Animal Care and Use Committee. The rd16 mouse was discovered in strain BXD-24/Ty at about F140 generation and the mutation was fixed in this strain, but all BXD-24/Ty mice recovered from the embryo freezer at about F84 generation had normal retinas. Detailed methods for retinal examination, histology and electroretinography have been described (See, e.g., Pang et al., (2005) Mol. Vis., 11, 152-162). BXD-24/Ty-rd16 mice were mated with CAST/EiJ mice. The F1 mice, which exhibit no retinal abnormalities, were backcrossed (BC) to BXD-24/Ty-rd16 mice. DNAs from 165 BC offspring were genotyped using microsatellite markers to develop a structure map; detailed methods for mapping and mutation screening have been reported (See, e.g., 49 Pang et al., (2005) Mol. Vis., 11, 152-162).

DNA, RNA and protein analyses. DNA and RNA analysis methods have been described (See, e.g., Mears et al., (2001) Nat. Genet., 29, 447-452). Primer pairs for RT-PCR amplification of BC004690: were as follows: for Nucleotides 5118-5529: F1: 5′<TCATTCGTCTGGCCGAGATGG>3′ (SEQ ID NO. 1), R1: 5′<GCTGCTGTCATTTCCGACCGAAG>3′ (SEQ ID NO. 2); for Nucleotides 4242-6368 F2: 5′<CAATTGGCATGTGAAAATAGAAGAA>3′ (SEQ ID NO. 3), R2: 5′<AAAGACTGAGAATATTTCTCCTTTGAA>3′ (SEQ ID NO. 4), and for Primers used for generating probe for Southern Blot Nucleotides 4805 to 5072: F3: 5′<AAACTAAAAGAAAAAGAATCTGC>3′ (SEQ ID NO. 5) R3: 5′<CTCTCTGGCCTTCTCCAGAA>3′ (SEQ ID NO. 6).

Co-immunoprecipitaion (IP) experiments with retinal extracts were performed as described (See, e.g., Khanna et al., (2005) J. Biol. Chem., 280, 33580-33587). The rabbit polyclonal CEP290 peptide antibody was generated (Invitrogen) against the mouse sequence ⁵¹⁷SKRLKQQQYRAENQ⁵³⁰ (SEQ ID NO. 7) and ²⁴⁵⁷SEHSEDGESPHSFPIY²⁴⁷² (SEQ ID NO. 8). Rabbit polyclonal antibodies to RPGR, RPGRIP1 and NPHP5 have been described (See, e.g., Khanna et al., (2005) J. Biol. Chem., 280, 33580-33587; Otto et al., (2005) Nat. Genet., 37, 282-288). Antibodies against acetylated α-tubulin, γ-tubulin, p50-dynamitin, SMC1 and SMC3 were purchased from Chemicon (Temeculla, Calif.). Mouse anti-p150^{Glued} antibody was obtained from BD Transduction Labs (San Jose, Calif.); anti-KIF3A, anti-KAP3, anti-centrin and anti-pericentrin antibodies were obtained from Sigma and anti-ninein was from BioLegend (San Diego, Calif.). Anti-RP1 antibody was obtained from Dr Eric A. Pierce, anti-NPM obtained from Dr Alan F. Wright and anti-PCM1 obtained from Dr A. Merdes.

Cell culture and immunolocalization. Kidney m-IMCD-3 cells (American Type Culture Collection, Manassas, Va.; CRL 2123) were grown in six well plates and transfected with p50-dynamitin expression construct using FUGENE-6 reagent (Roche). Experimental details about immunocytochemistry and immunogold EM procedures are as described (See, e.g.; Khanna et al., (2005) J. Biol. Chem., 280, 33580-33587). Immunofluorescence microscopy of retinal sections for rhodopsin and arrestin was performed as described (See, e.g., Cheng et al., (2004) Hum. Mol. Genet., 13, 1563-1575). For immunolabeling of CEP290, eyes were fixed in methanol, and sections were labeled with 3G4, followed by goat anti-mouse conjugated to ALEXAFLUOR 488. Clinical and histological examination of the rd16 mouse.

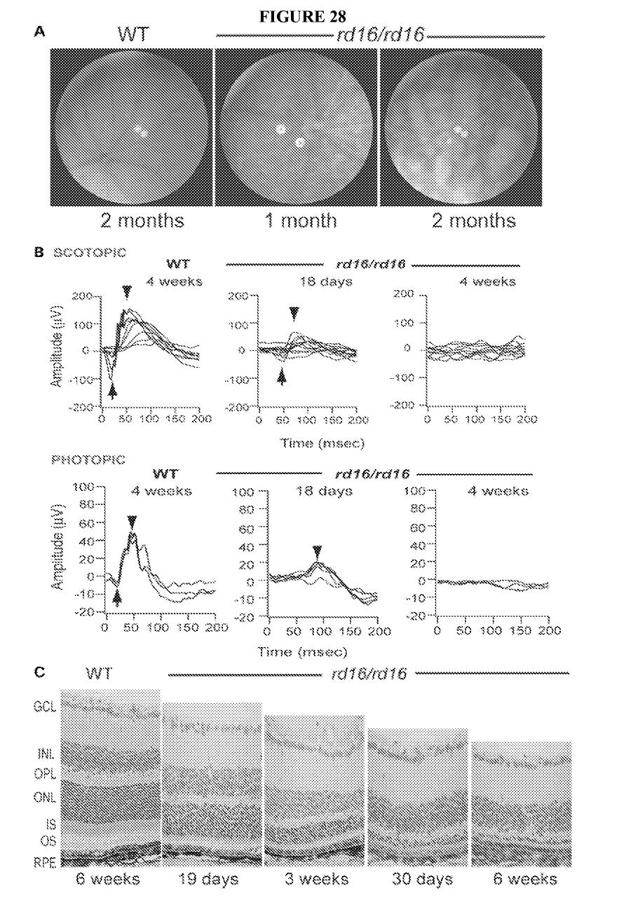

The phenotype of homozygote rd16 mice can be distinguished from wild-type (WT) animals by the appearance of white retinal vessels at 1 month and large pigment patches at 2 months of age (See FIG. 28A). Electroretinograms under dark- and light-adapted conditions indicate a considerable deterioration of rod and cone functions in the rd16 homozygotes compared with the WT as early as postnatal (P) day 18 (See FIG. 28B). Light microscopy of the rd16/rd16 retina shows degeneration of outer segments and reduction in the thickness of the outer nuclear layer as early as postnatal day 19 and progresses with age. Little or no change was observed in other cellular layers (See FIG. 28C).

Cep290 is Mutated in the rd16 Mouse

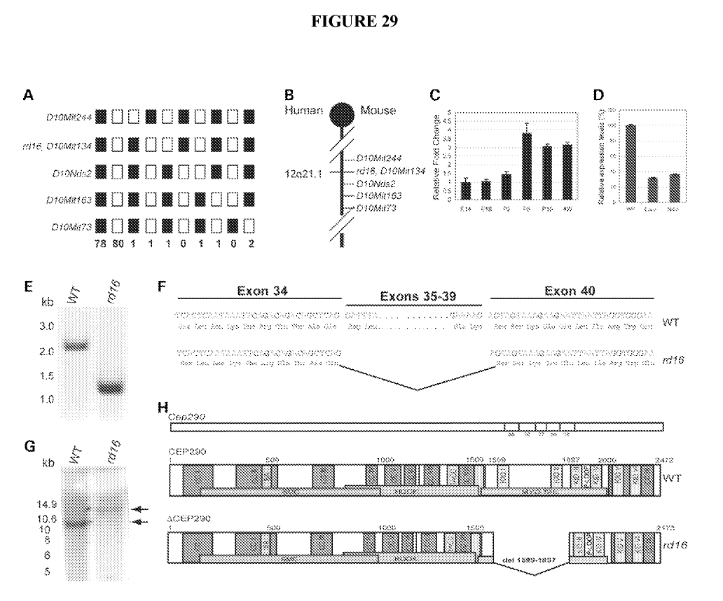

By linkage analysis of back-crossed mice, the causative gene defect in rd16 was mapped to chromosome 10 in the genomic region flanked by D10Mit244 (99.4 M) and D10Nds2 (105 M) (See FIGS. 29A and 29B). In silico analysis of the critical region revealed over 30 putative expressed sequences, which were then examined for differential expression in mouse photoreceptors using gene expression profiles (See, e.g., Example 1; Blackshaw et al., (2004) PLoS. Biol., 2, E247). The expression of one of the hypothetical genes, BC004690, was found to be increased nearly 3-fold during rod maturation (P2-P6). Its expression was dramatically reduced in the Crx^{-/-} mice in which photoreceptors fail to develop (See, e.g., Furukawa et al., (1999) Nat. Genet., 23, 466-4701) and in the rodless, cone-enriched retina of Nrl^{-/-} mice (See, e.g., Mears et al., (2001) Nat. Genet., 29, 447-452). Real-time PCR analysis using primer pair F1-R1 derived from BC004690 (See above) validated the gene-profiling data (See FIGS. 29C and 29D).

Further in silico analysis revealed that BC004690 is part of the mouse Cep290 gene (exons 27-48), that encodes a protein similar to human centrosomal protein CEP290 (See, e.g., Andersen et al., (2003) Nature, 426, 570-574). Given that mutations in certain centrosomal proteins may result in retinal degeneration owing to ciliary dysfunction in photoreceptors (See, e.g., Badano et al., (2005) Nat. Rev. Genet., 6, 194-205), the Cep290 gene was screened for possible mutations in the rd16 mouse. RT-PCR analysis using the F1-R1 primer pair did not amplify any product. However, another primer set (F2-R2; described above) encompassing the complete BC004690 sequence detected a 1.2 kb product with the rd16 retinal cDNA compared with an expected 2.1 kb product in WT cDNA (See FIG. 29E). Sequence analysis of the RT-PCR products identified an in-frame deletion of 897 by (5073-5969 by in cDNA), that corresponded to CEP290 amino acid residues 1599-1897 (See FIG. 29F showing the junction sequence). The truncated CEP290 protein was designated ΔCEP290. No other sequence alteration was detected. Southern analysis of the WT and rd16 homozygote genomic DNAs confirmed a deletion (from exon 35 to 39) within the Cep290 gene (See FIG. 29G).

### Domain Composition of CEP290.

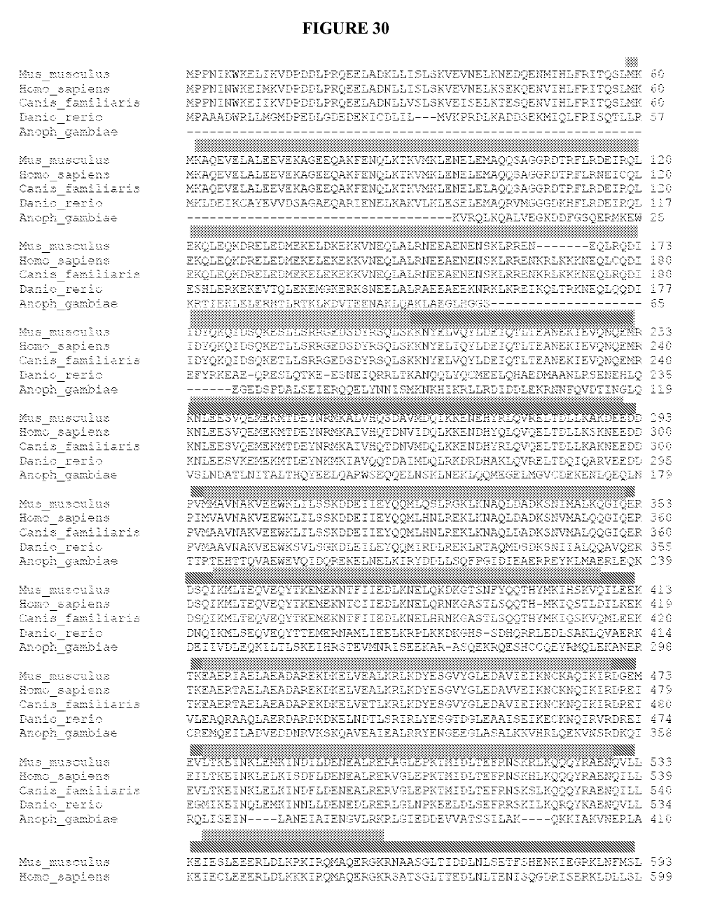

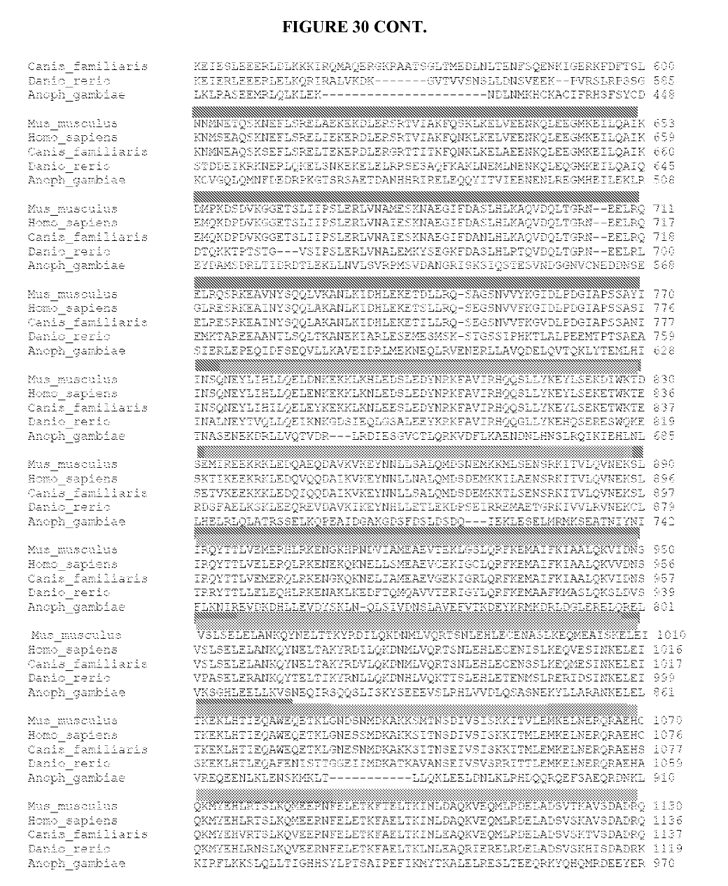

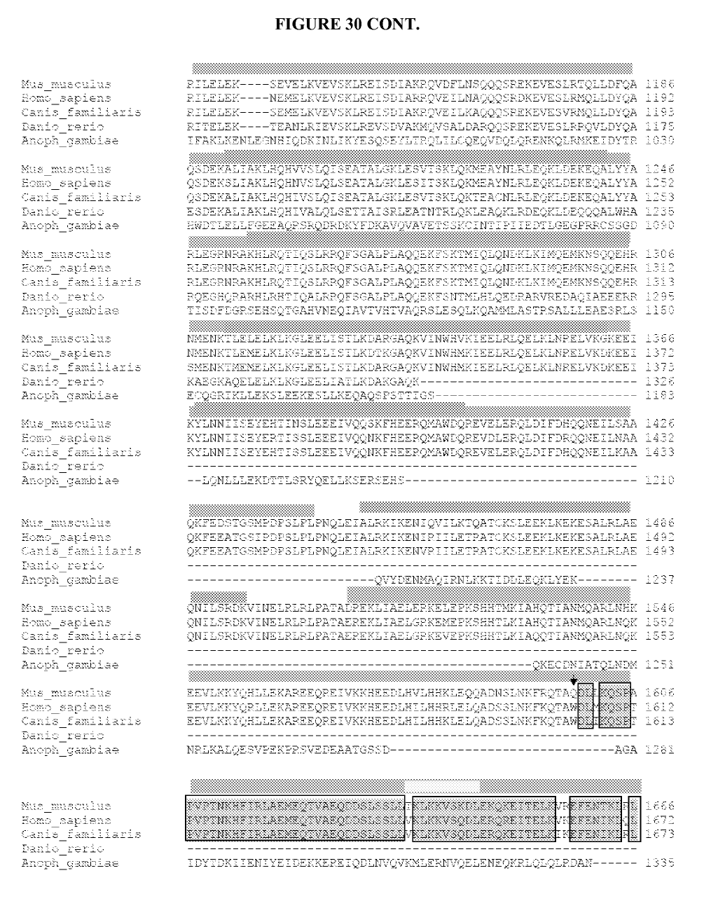

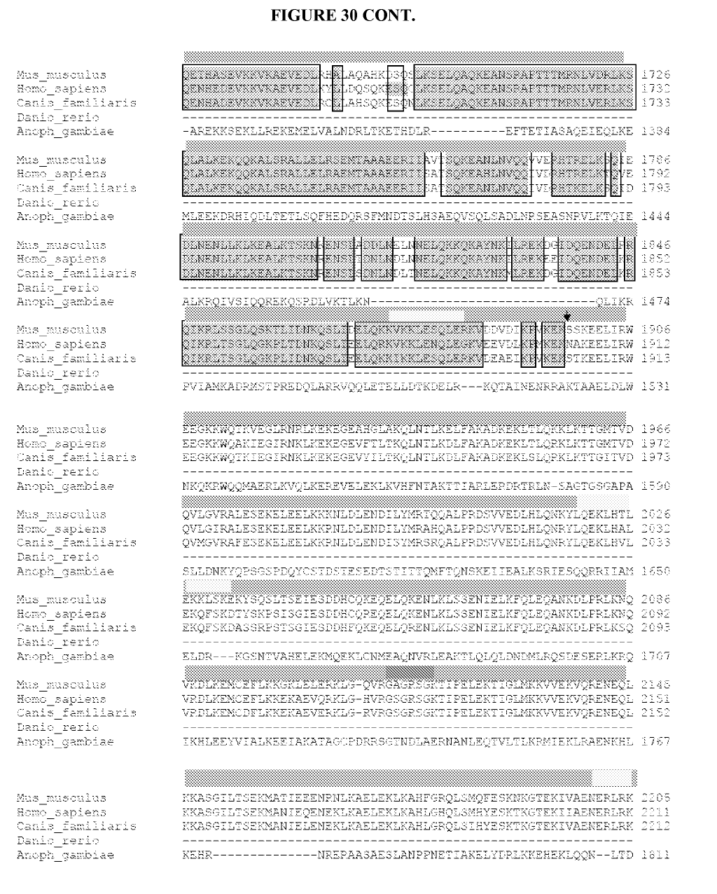

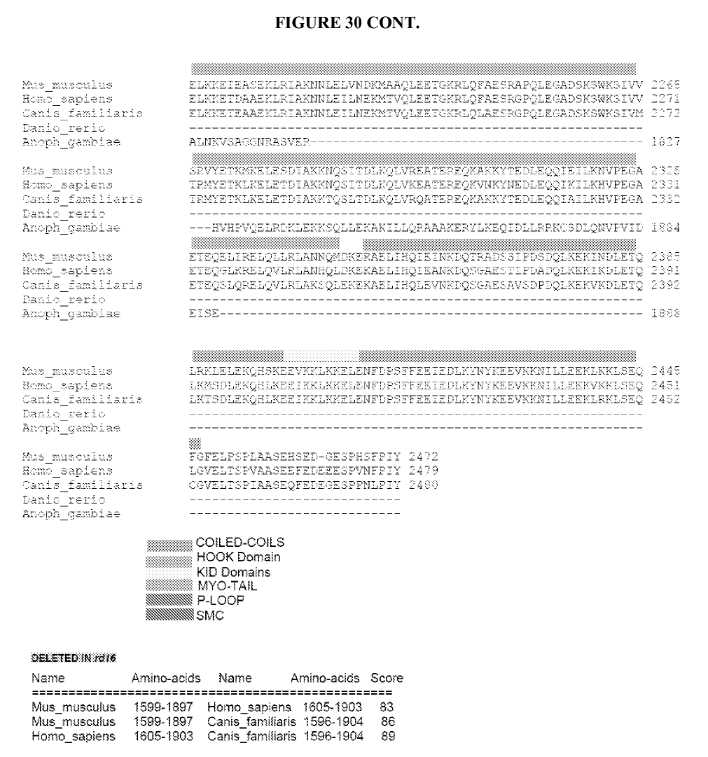

The Cep290 gene, spanning over 85 kb and 52 exons, encodes a putative protein of 2472 amino acids (apparent molecular weight 290 kDa). To investigate the domain structure of CEP290, the MotifScan and SMART protein databases (www.expasy.org) were scanned and at least nine coiled-coil domains and a C-terminal myosin-tail homology domain were identified, which provides a structural backbone to the myosin motor (See FIG. 29H). Moreover, CEP290 exhibits significant similarity to SMC (structural maintenance of chromosomes) chromosomal segregation ATPases (See, e.g., Nasmyth and Haering, (2005) Annu. Rev. Biochem., 74, 595 648), six RepA/Rep+ protein motifs KID, glycine-rich ATP/GTP-binding site motif (P-loop) involved in the binding of motor proteins to the nucleotides and the transforming acidic coiled-coil (TACC) domain involved in microtubule organization by centrosomal proteins. A majority of the myosin-tail homology region is deleted in rd16 mouse (See FIGS. 29H, and shaded amino acid sequence in FIG. 30). CLUSTALW analysis shows strong evolutionary conservation of the CEP290 protein, with orthologs in Danio rerio and Anopheles gambiae (See, e.g., FIG. 30).

### Expression and Localization of CEP290 in Mouse Retina

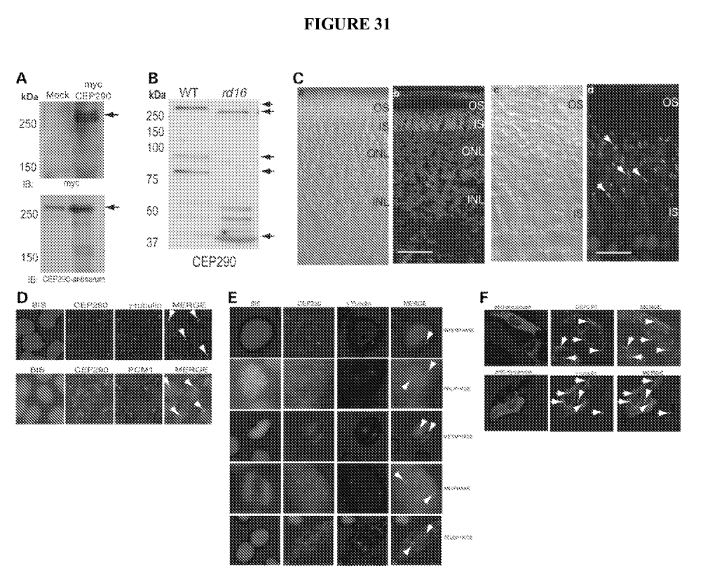

A monoclonal antibody, 3G4 (See, e.g., Guo et al., (2004) Biochem. Biophys. Res. Commun., 324, 922-930), against CEP290 recognized a band at ˜290 kDa in protein extracts from different tissues of WT mice as well as in COS1 cells transfected with a full-length myc-tagged CEP290 construct. A polyclonal antibody was also generated against two peptides corresponding to the mouse CEP290 protein; this antibody also recognized the CEP290 protein in transfected COS-1 cells (See FIG. 31A). Immunoblot analysis revealed a fainter band of faster mobility (ΔCEP290) in retinal extracts from the rd16 mouse compared with the 290 kDa band in WT (See FIG. 31B). Additional bands of low molecular mass were also observed in bovine retina extracts. On the basis of this and in silico analysis, the present invention provides that these bands represent alternately spliced isoforms of CEP290.

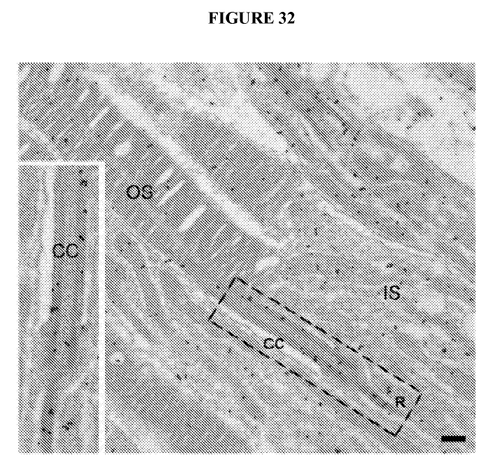

The localization of CEP290 in mouse retina was then characterized by immunofluorescence and immunogold microscopy. CEP290 is localized primarily to the connecting cilium of mouse photoreceptors, although some labeling is detected in the inner segments (See FIGS. 31 and 32). Connecting cilium staining of CEP290 was also observed in dissociated rod photoreceptors of mouse retina, as determined by co-localization with acetylated alpha-tubulin.

### CEP290 Localizes to Centrosomes in a Dynein-Independent Manner

Immunocytochemical analysis using the CEP290 antibody revealed that CEP290 co-localized with the centrosomal and pericentriolar matrix markers γ-tubulin and PCM1 (See, e.g., Doxsey, (2001) Nat. Rev. Mol. Cell. Biol., 2, 688-698) at the centrosomes of mouse kidney inner medullary collecting duct (IMCD-3) (See FIG. 31D). Co-localization with PCM1 is reminiscent of the staining pattern of BBS4, a ciliary/centrosomal protein involved in microtubule dynamics (See, e.g., Kim et al., (2004) Nat. Genet., 36, 462-470). Consistent co-labeling of CEP290 with γ-tubulin was detected through different stages of cell cycle (See FIG. 31E).

CEP290 recruitment and assembly at the centrosomes was analyzed next. Previous studies have shown that microtubule depolymerization using nocodazole does not alter centrosomal localization of CEP290 (See, e.g., Andersen et al., (2003) Nature, 426, 570-574). Given that a number of centrosomal proteins, including RPGR-ORF15 and PCM1, are anchored at the centrosomes via the functional dynein-dynactin molecular motor, whereas others such as γ-tubulin and BBS6 are not (See, e.g., Dammermann and Merdes, (2002) J. Cell. Biol., 159, 255-266; Kim et al., (2005) J. Cell. Sci., 118, 1007-1020), it was determined whether localization of CEP290 depends on dynein-dynactin motor by overexpressing the p50-dynamitin subunit of the dynactin complex (See, e.g., Vaughan and Vallee, (1995) J. Cell. Biol., 131, 1507-1516). Like γ-tubulin, the localization of CEP290 at centrosomes is not altered in cells overexpressing p50-dynamitin (See FIG. 31F). Although an understanding of the mechanism is not necessary to practice the present invention and the present invention is not limited to any particular mechanism of action, in some embodiments, the present invention provides that functional microtubule motor or polymerized microtubules are not necessary to maintain CEP290 at the centrosomes. In some embodiments, functional microtubule motor or polymerized microtubules are involved in the recruitment of newly synthesized CEP290 to the centrosomes.

CEP290 Associates with RPGR in Mammalian Retina

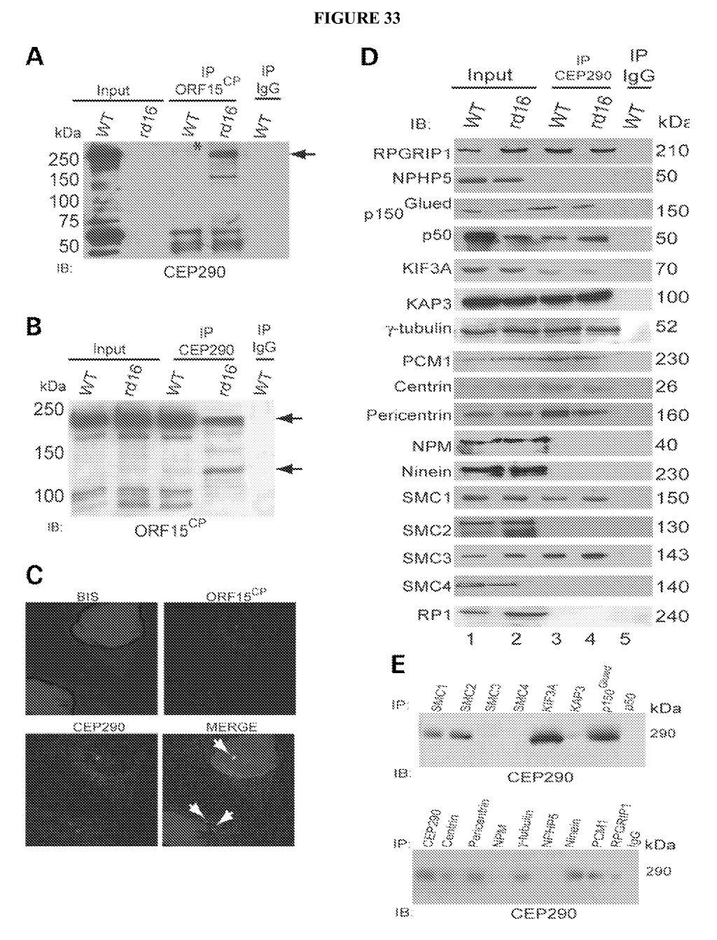

Given that RPGR, a ciliary/centrosomal protein (See, e.g., Hong et al., (2003) Ophthalmol. Vis. Sci., 44, 2413-2421; Shu et al., (2005) Hum. Mol. Genet., 14, 1183-1197; Khanna et al., (2005) J. Biol. Chem., 280, 33580-33587), mutations in which are detected in retinitis pigmentosa (See, e.g., Vervoort et al., (2000) Nat. Genet., 25, 462-466; Breuer et al., (2002) Am. J. Hum. Genet., 70, 1545-1554; Sharon et al., (2003) Am. J. Hum. Genet., 73, 1131-1146), interacts with centrosomal disease-associated proteins (See, e.g., Khanna et al., (2005) J. Biol. Chem., 280, 33580-33587; Dryja et al., (2001) Am. J. Hum. Genet., 68, 1295-1298; Hong et al., (2001) J. Biol. Chem., 276, 12091-12099), it was determined whether CEP290 may also associate with RPGR and its interacting proteins and participate in common functional pathways. The ORF15^{CP} antibody against the retina-enriched RPGR-ORF15 isoform(s) (See, e.g., Shu et al., (2005) Hum. Mol. Genet., 14, 1183-1197; Khanna et al., (2005) J. Biol. Chem., 280, 33580-33587; Otto et al., (2005) Nat. Genet., 37, 282-288) was able to precipitate low amounts of CEP290 from WT mouse retinal extracts (See FIG. 33A). Reverse co-immunoprecipitation using the 3G4 antibody detected RPGR-ORF15 upon immunoblotting (See FIG. 33B). Yeast two-hybrid experiments do not reveal a direct interaction of CEP290 with RPGR.

Co-immunoprecipitation experiments were performed using rd16 retinal extracts. RPGR-ORF15 recruited over 50 times higher levels of the ACEP290 protein from rd16 retina compared with the WT protein (See FIG. 33A). Reverse immunoprecipitation pulled down a few, but not all, RPGR-ORF15 isoforms from the rd16 retina (See FIG. 33B). Consistent with this, the endogenous CEP290 co-localized with RPGR-ORF15 in IMCD-3 cells (See FIG. 33C) and dissociated mouse rod photoreceptors.

### CEP290 is Part of Selected Centrosomal and Microtubule-Associated Protein Complex(es)

To evaluate whether CEP290 and ΔCEP290 are part of multi-protein complex(es) with other centrosomal and microtubule-associated motor assemblies, some of which may also overlap with RPGR-ORF15-containing complexes, additional co-immunoprecipitation experiments were conducted using mouse or bovine retinal extracts. Data accumulated indicated that CEP290 is present in complex with RPGR-interacting protein 1 (RPGRIP1), dynactin subunits p150^{Glued} and p50-dynamitin, kinesin subunit KIF3A, kinesin-associated protein (KAP3), γ-tubulin, PCMI, centrin, pericentrin and ninein, but not with nucleophosmin (NPM), or Nephrocystin-5 (NPHP5) (See FIGS. 33D and 33E). Dynein subunits are not detectable due to the low abundance or instability of the dynein-dynactin interaction. As RPGR-ORF15, CEP290 also interacts with SMC1 and SMC3. Varying degree of association with SMC proteins and p50-dynamitin may be due to relative abundance of the proteins. CEP290 is not associated with RP1, another ciliary protein mutated in retinopathies (See, e.g., Liu et al., (2004). J. Neurosci., 24, 6427-6436) (See FIG. 33D). Similar results were obtained with rd16 as well as bovine retinal extracts. No immunoreactive bands were detected when normal IgG was used for IP. Notably, RPGR-ORF15 interacts with NPM (See, e.g., Shu et al., (2005) Hum. Mol. Genet., 14, 1183-1197) and NPHP5 (See, e.g., Otto et al., (2005) Nat. Genet., 37, 282-288) but not with centrin and pericentrin (See, e.g., Khanna et al., (2005) J. Biol. Chem., 280, 33580-33587). Thus, the present invention provides that CEP290 and RPGR perform multiple overlapping yet distinct microtubule-based transport functions in the retina.

Perturbed Localization of RPGR and Opsin in the rd16 Retina

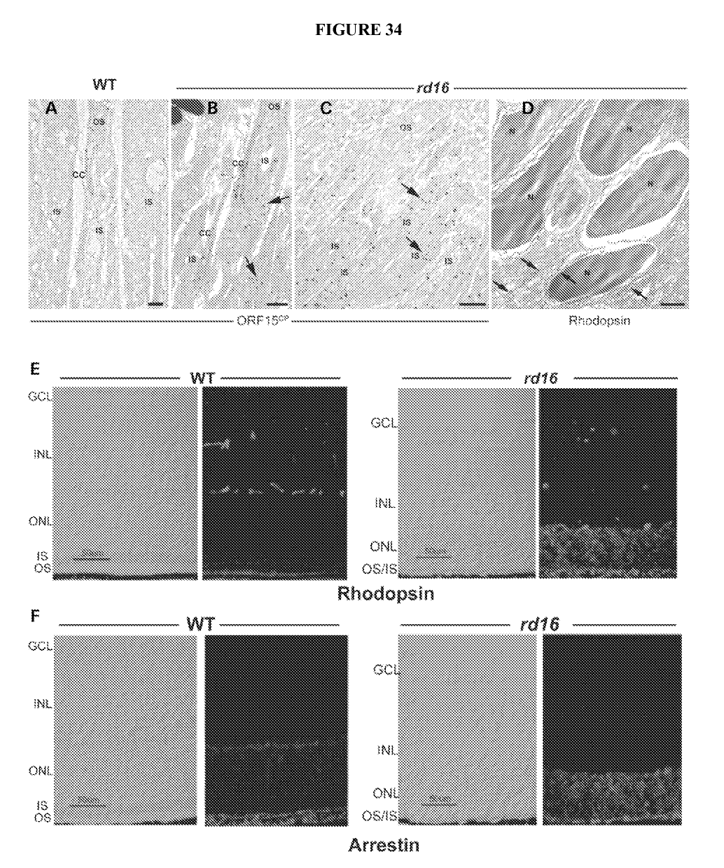

It was next determined whether increased association of ΔCEP290 affected the localization of RPGR-ORF15 in the rd16 retina. Immunoelectron microscopy (ImmunoEM) experiments revealed that RPGR-ORF15 aggregates were present in the inner segments of P12 rd16 retina, indicating a trafficking defect, whereas, as shown elsewhere (See, e.g., Khanna et al., (2005) J. Biol. Chem., 280, 33580-33587), the axoneme and basal bodies in photoreceptors of normal retinas are strongly labeled with the ORF15^{CP} antibody (See FIGS. 34A-C). However, obvious structural defects were not observed in the connecting cilium of the rd16 retina.

Given the involvement of RPGR-ORF15 in regulating intracellular trafficking in photoreceptors (See, e.g., Khanna et al., (2005) J. Biol. Chem., 280, 33580-33587; Hong et al., (2000) Proc. Natl Acad. Sci. USA, 97, 3649-3654), it was determined whether CEP290 mutation and/or RPGR mislocalization would have an effect on the trafficking of phototransduction proteins in the retina. Immunogold EM and immunofluorescence analyses revealed redistribution of rhodopsin and arrestin throughout the plasma membrane of rd16 photoreceptors when compared with the normal outer segment localization in WT photoreceptors (See FIG. 34D-F).

### Example 5

### NR2E3 Establishes Photoreceptor Identity During Mammalian Retinal Development

### Materials and Methods.

Transgenic mice. A 2.3 kb mouse Crx promoter DNA (from 22286 to p72, GenBank accession nos AF335248 and AF301006; (55) and the Nr2e3-coding region (GenBank accession no. NM013708) with an additional Kozak sequence (indicated as underlined letters) was amplified as a BglII-NotI (restriction enzyme sites are indicated as bold letters) fragment by PCR (forward primer: GACAGATCTGCCACCATGAGCTCTA CAGTGGCT (SEQ ID NO.: 9); reverse primer: CACTTGGCGCGGCCGCC TAGTTTTTGAACATGT (SEQ ID NO.: 10)) from mouse retina cDNA and cloned into BamHI-NotI sites of pcDNA4/HisMaxC (Invitrogen). Then the KpnI-NotI fragment was cloned into a modified promoter-less pCl (pCIpl) vector (See, e.g., Akimoto et al., (2004) Invest. Ophthalmol. Vis. Sci., 45, 42-47) as shown (FIG. 1A). The 4.2 kb Crx::Nr2e3 fragment was purified and injected into fertilized

Nrl^{-/-} (mix background of 129X1/SvJ and C57BL/6J) mouse oocytes (UM transgenic core facility). Transgenic founder mice and their progeny were identified by PCR, and then confirmed by Southern blot analysis of tail DNA. Transgenic founders were bred to the Nrl2/mice to generate F1 progeny. The transgenic progeny was also mated to C57BL/6J or Nrl^{-/-}/Crx2/mice to generate Crx::Nr2e3/2/2WT or Crx::Nr2e3/Nrl^{-/-}/Crxmice, respectively. The S-opsin::Nr2e3 transgenic mice were generated in a similar manner, except that a 520 by mouse S-opin promoter DNA (from 2870 to 2346, Genbank accession no. L27831) (49) was used.

All studies involving mice were performed in accordance with institutional and federal guidelines and approved by the University Committee on Use and Care of Animals at the University of Michigan.

DNA, RNA and protein analysis. Standard protocols were used for Southern analysis, PCR, qPCR, immunoblotting and immunofluorescence experiments (See, e.g., Mears et al., (2001) Nat. Genet., 29, 447-452; Akimoto et al., (2006) Proc. Natl Acad. Sci. USA, 103, 3890-3895.). The primary antibodies used in this study were: rabbit anti-NR2E3 antibody (See, e.g., Cheng et al., (2004) Hum. Mol. Genet., 13, 1563-1575), rabbit anti S-opsin, M-opsin or mouse cone arrestin polyclonal antibodies (gifts from C. Craft), mouse anti-rhodopsin (4D2) monoclonal antibody (gift from R. Molday), mouse anti-g tubulin monoclonal antibody (Sigma) and rat anti-BrdU monoclonal antibody (BU1/75, Harlan Sera-Lab, Loughborough, UK). Fluorescent detection was performed using AlexaFluor-488, 546 or 633 (Molecular Probes) and Texas Red (Jackson ImmunoResearch, West Grove, Pa., USA) conjugated secondary antibodies. Sections were visualized under a conventional fluorescent microscope or FV500 Confocal microscope and digitized.

BrdU labeling. Timed-pregnant females or pups received a single intraperitoneal injection of BrdU (BrdU, Sigma; 0.1 mg/g body weight). The eyes were fixed in 4% paraformaldehyde and cryosectioned at 3 weeks of age. THC and BrdU staining were performed as described in Example 1.

Transmission electron microscopy. Mice were perfusion-fixed with 2.5% glutaraldehyde in 0.1 M Sorensen's buffer, pH 7.4. Eye cups were excised, fixed, dehydrated and then embedded in Epon epoxy resin following the standard protocol. Semi-thin sections were stained with toluidine blue for tissue orientation. Central part of the dorsal retina was ultra-thin sectioned (70 nm in thickness) and stained with uranyl acetate and lead citrate. The sections were examined using a Philips CM100 electron microscope at 60 kV. Images were recorded digitally using a Hamatsu ORCA-HR digital camera system operated using AMT software (Advanced Microscopy Techniques Corp., Danvers, Mass., USA).

FACS enrichment and microarray analysis. Methods for microarray analysis have been described (See, e.g., Example 1, and Yoshida et al., (2004) Hum. Mol. Genet., 13, 1487-1503; Zareparsi et al., (2004). Invest. Ophthalmol. Vis. Sci., 45, 2457-2462). Mouse retinas were dissected at 4 week. GFP+ photoreceptors were enriched by FACS (FACSARIA, BD Biosciences, Franklin Lakes, N.J., USA). RNA was extracted from 1˜5×10⁵ flow-sorted cells using Trizol (Invitrogen). Total RNA (40-60 ng) was used for linear amplification with OVATION Biotin labeling system (Nugen), and 2.75 μg of biotin-labeled fragmented cDNA was hybridized to mouse GENECHIPS MOE430.2.0 (Affymetrix) having 45 101 probesets (corresponding to over 39 000 transcripts and 34 000 annotated mouse genes). Four independent samples were used for each time point. Normalized data were subjected to two-stage analysis based on false discovery rate with confidence interval (FDRCI) for screening differentially expressed genes (See, e.g., Chen et al., (1997) Neuron, 19, 1017-1030; Swaroop et al., (1992) Proc. Natl Acad. Sci. USA, 89, 266-270) with a minimum fold change of 4.

Electroretinograms Dark-adapted (>6 h) ERGs in response to increasing intensities (−4.2 to 0.3 log scot-cd.s.m⁻²) of blue lights were recorded from anesthetized mice using a computer-based system as described (See, e.g., Aleman et al., (2001) Vision Res., 41, 2779-2797). The threshold intensity that evokes a criterion (20 μV) dark-adapted b-wave was determined by plotting its amplitude as a function of stimulus intensity and linearly interpolating the stimulus intensity value that corresponded to the criterion. Dark-adapted photoresponses were then elicited with a pair of flashes (white; 3.6 log scot-cd.s.m⁻²) presented 4 s apart and were fit with a model of phototransduction activation (See, e.g., Cideciyan, A. V. and Jacobson, S. G. (1996) Vision Res., 36, 2609-2621). A second computer-based system (Espion, Diagnosys LLC, Littleton, Mass., USA) was used to generate light-adapted (40 cd.m² white background) ERGs in response to a Xenon UV flash (360 nm peak, Hoya U-360 filter, Edmund Optics, Barrington, N.J., USA). The energy of this flash was adjusted to evoke responses matched in waveform to those elicited with green LEDs (510 nm peak; 0.87 log phot-cd.s.m⁻², 4 ms) stimulus in WT mice. These stimuli were presented in a Ganzfeld lined with aluminum foil (See, e.g., Lyubarsky et al., Jr. (1999) J. Neurosci., 19, 442-455).

### Crx Promoter Directs Ectopic Expression of NR2E3 to Photoreceptor Precursors.

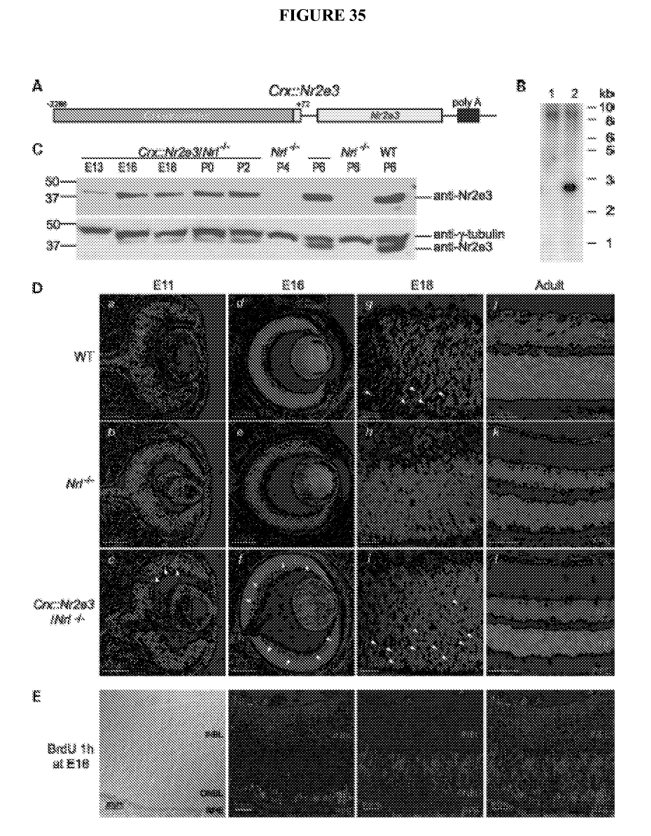

In order to investigate the function of NR2E3 in vivo, Nrl^{-/-} mice (rather than the rd7 mice) were utilized, since in the Nrl^{-/-} retina: (1) no endogenous NR2E3 transcript or protein is detectable; (2) rod-specific genes are not expressed; (3) the expression of cone genes is dramatically increased; and (4) the retinal phenotype is easy to distinguish with no rods and only functional cones (See, e.g., Mears et al., . (2001) Nat. Genet., 29, 447-452). In addition, the function of NR2E3 can be tested directly without interference from NRL, that can induce rod gene expression (See, e.g., Yoshida et al., (2004) Hum. Mol. Genet., 13, 1487-1503). Transgenic mice were generated in the Nrl^{-/-} background using Crx::Nr2e3 construct (See FIG. 35A), in which Nr2e3 transcription was driven by the Crx promoter resulting in its expression in all post-mitotic photoreceptor precursors. The endogenous Nr2e3 gene and the transgene can be discriminated as 9.0 and 2.8 kb bands, respectively, upon Southern blot analysis of the Crx::Nr2e3/Nrl^{-/-} mouse DNA (See FIG. 35B). The NR2E3 protein was detected in all six transgenic founders by immunoblot assays. The temporal expression of Nr2e3 transcripts was similar to that of Crx, and NR2E3 protein was detected even at embryonic day (E)13 in the transgenic mice (See FIG. 35C). By immunohistochemistry (IHC), NR2E3 protein was detected as early as E11 in the dorsal retina (See FIG. 35Dc), about 1 week earlier than wild-type (WT) (See FIG. 35Dg). At E16, NR2E3 was clearly detectable in the outer neuroblastic layer of the Crx::Nr2e3/Nrl^{-/-} transgenic retina but not in WT (See FIG. 35Dd-f). At E18, more NR2E3 positive cells were observed in the transgenic mice when compared with WT (See FIGS. 35Dg and i); however, at P6 and later stages, similar NR2E3 expression levels were detected in both Crx::Nr2e3/Nrl^{-/-} and WT retina (See FIG. 35C, Dj-l). A 1 h pulse labeling with (+)5-bromo-20-deoxyuridine (BrdU) did not reveal any BrdU-labeled cells in the E16 retina that also expressed NR2E3 (See FIG. 35E). Thus, temporal and spatial expression of NR2E3 in the transgenic mice reflects high fidelity of the 2.3 kb mouse Crx promoter.

### NR2E3 can Repress Cone-Specific Genes and Activate Rod Genes.

P21 retinas were examined from all six NR2E3-expressing Crx::Nr2e3/Nrl^{-/-} transgenic mouse lines by IHC using antibodies against a number of rod- and cone-specific proteins. In five transgenic lines, rhodopsin was detected in the entire outer nuclear layer

(ONL) with slightly stronger signal in the dorsal retina, whereas the Nrl^{-/-} retina showed no rhodopsin staining. Three of the transgenic lines had no S-opsin, M-opsin or cone arrestin labeling (See FIG. 36A-C), whereas two others displayed partial expression. The sixth transgenic line demonstrated patchy rhodopsin expression in the ONL, with no co-staining of cone-specific markers. These data provide a direct support of NR2E3's dual role in regulating rod and cone genes in vivo. The three transgenic lines with complete cone gene suppression were used in the following studies.

### NR2E3 can Partially Rescue Rod Morphology but not Function in the Nrl^{-/-} Retina.

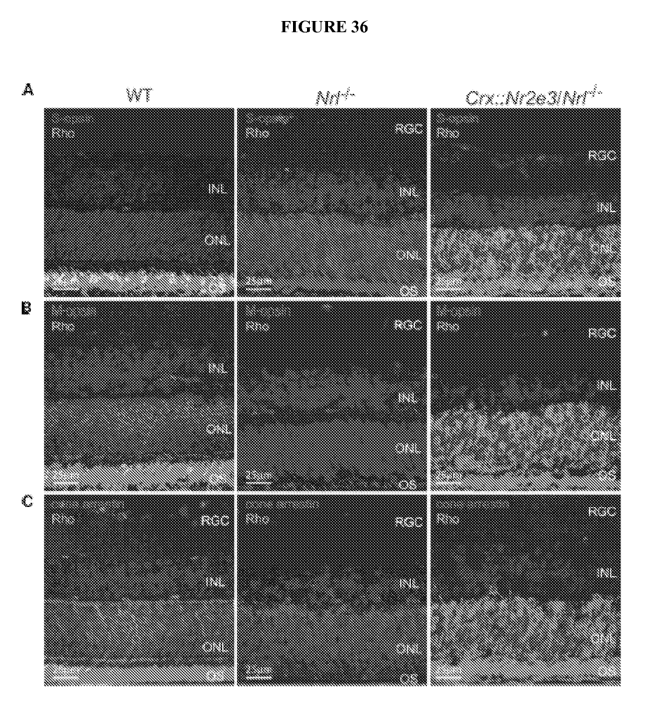

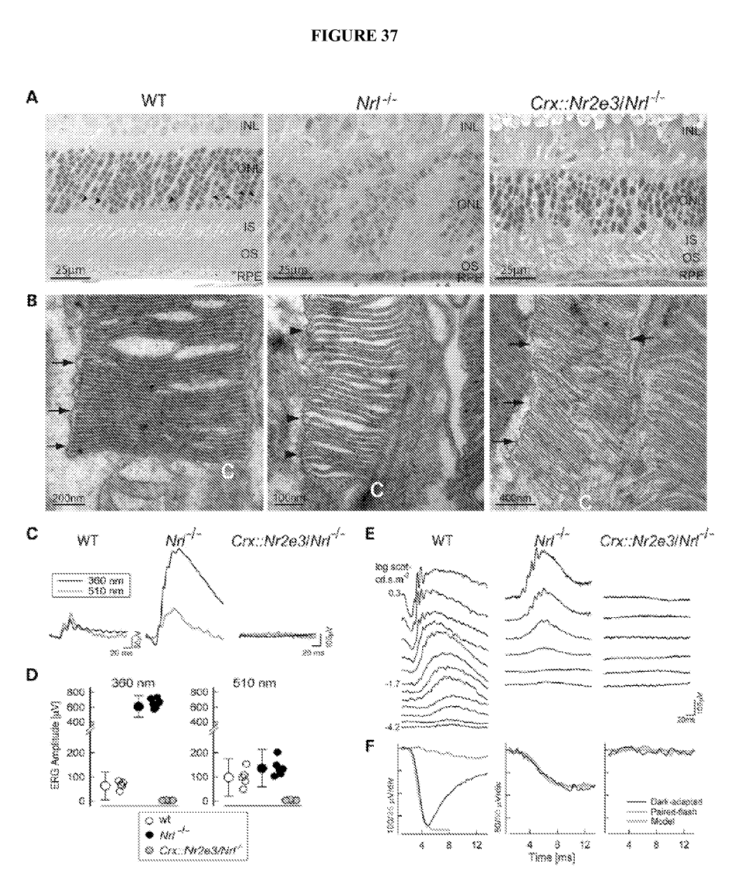

In the WT retina, cones have open outer segment (OS) discs, their cell bodies are located in the outermost rows of the ONL, and their nuclei display punctate staining of the heterochromatin. In the Nrl^{-/-} retina, all photoreceptors showed cone-like morphology with whorls and rosettes in the ONL (See, e.g., Daniele et al., (2005) Ophthalmol. Vis. Sci., 46, 2156-2167). Ectopic expression of NR2E3 in the Crx::Nr2e3/Nrl^{-/-} retina resulted in partial transformation from cone- to apparently rod-like photoreceptors in the ONL with no obvious whorls and rosettes. Although an understanding of the mechanism is not necessary to practice the present invention and the present invention is not limited to any particular mechanism of action, in some embodiments, this may be due to elongated OSs and dense nuclear chromatin (See FIG. 37A). Notably, oval whorls were still observed on the flat mount retina. The ONL was wavy and thinner when compared with the WT retina. Decreased number of cells in the ONL (20-40% less when compared with the WT) was due to increased apoptosis, as indicated by TUNEL staining. OS in the Crx::Nr2e3/Nrl^{-/-} retina were longer, but still misaligned and shorter than those of the WT (See FIG. 37A). The ultrastructure of the OS discs, revealed by transmission electron microscopy (TEM), showed rod-like closed discs in the Crx::Nr2e3/Nrl^{-/-} retina, although the length and orientation of the discs were not as organized as in the WT retina (See FIG. 37B). Ectopic expression of NR2E3 can therefore drive photoreceptor precursors towards the rod phenotype, even in the absence of NRL.

Retinal function of Crx::Nr2e3/Nrl^{-/-} mice was examined by electroretinography (ERG) (See FIG. 37C-F). The three transgenic lines with complete suppression of S- and M-opsin showed no detectable ERGs driven by bipolar cells post-synaptic to S- or M-cones. This is in contrast with Nrl^{-/-} mice where post-receptoral S-cone responses were nearly 10-fold greater in amplitude when compared with WT (See FIGS. 37C and D). Unexpectedly, even though there was high expression of rhodopsin (See FIG. 36), all animals from these transgenic lines showed no detectable ERGs when presented with stimuli known to activate rod photoreceptors (See FIGS. 37E and F). Under these dark-adapted conditions, activity of rod bipolar cells dominate ERG b-waves from −4 to −1 log scot-cd.s.m⁻² in WT mice; cone-derived function contributes increasingly at higher intensities as seen from the cone-only responses of Nrl^{-/-} mouse (See FIGS. 37E and F). ERG photoresponses directly originating from photoreceptor activity were also extinguished (See FIGS. 37E and F). With the paired high-intensity photoresponses used, rod activity normally dominates the first flash response (See FIG. 37F, black traces); and, cone activity dominates the second flash response. In the Nrl^{-/-} mice, photoresponses were smaller (68±18 versus 377±133 mV) and slower (1.93±0.35 versus 3.33±0.13 log scot-cd⁻¹.m².s⁻³) than those driven by WT rods, but they were larger than those driven by WT cones (See FIG. 37F).

The two Crx::Nr2e3/Nrl^{-/-} lines with incomplete cone suppression showed recordable ERGs with abnormal b-wave amplitudes and threshold elevations similar to the Nrl^{-/-} mice but with smaller amplitudes. In these lines, there was also no evidence of rod function, but there was detectable cone function, which was enriched in S-cone activity. ERG responses to the short wavelength stimulus in these lines were three to four times larger than those evoked by the longer wavelength flash; this ratio was three to six times in the Nrl^{-/-} mice. The transgenic line with minor cone-opsin suppression revealed ERGs similar to those of the Nrl^{-/-} mice.

Lack of Rod Function in the Crx::Nr2e3/Nrl^{-/-} Retina is Associated with Reduced or No Expression of Several Rod Phototransduction Genes.

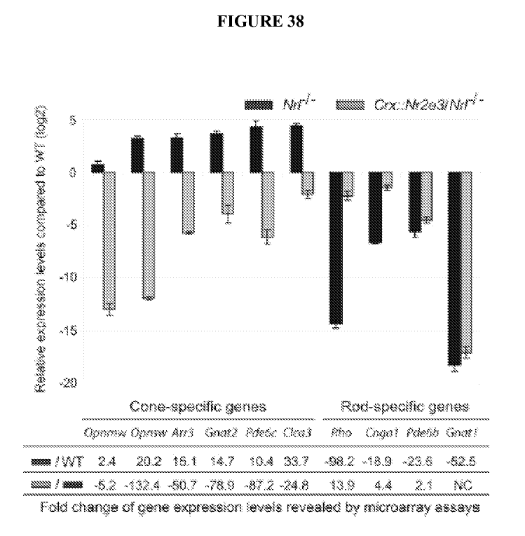

In order to investigate the underlying cause of the apparent lack of rod activity, despite the existence of rod-like cells with high rhodopsin expression, quantitative RT-PCR (qPCR) analysis of phototransduction genes was performed using total RNA from the WT, Nrl^{-/-} and Crx::Nr2e3/Nrl^{-/-} retina. Dramatically lower expression of genes encoding cone phototransduction proteins (such as S-opsin, M-opsin, Gnat2, Pde6c and Arr3) was observed in the Crx::Nr2e3/Nrl^{-/-} retina when compared with Nrl^{-/-}; however, among the rod genes tested by qPCR only rhodopsin transcripts were dramatically increased and almost reached the level of the WT (See FIG. 38). While a few of the rod phototransduction genes, such as Pde6b and Cnga1, exhibited higher yet variable level of expression, the transcripts for alpha subunit of rod transducin, Gnat1, were undetectable as in the Nrl^{-/-} mouse (See FIG. 38). Although an understanding of the mechanism is not necessary to practice the present invention and the present invention is not limited to any particular mechanism of action, in some embodiments, NR2E3 fails or is deficient in directing the expression of the full complement of rod-specific genes when NRL is not present.

### Potential Downstream Targets of NR2E3 Identified by Gene Profiling of FACS-Purified Photoreceptors.

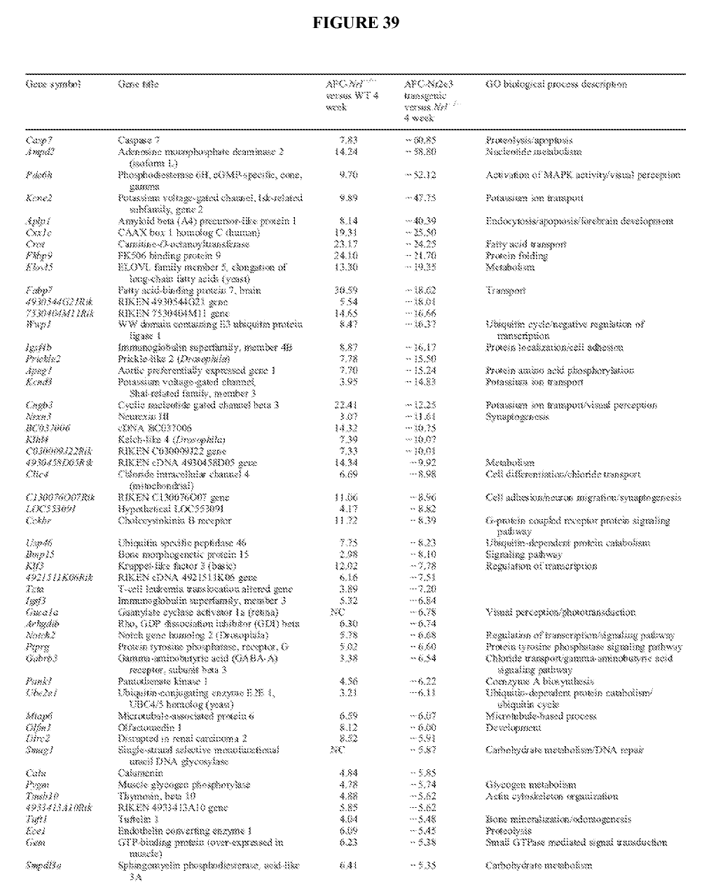

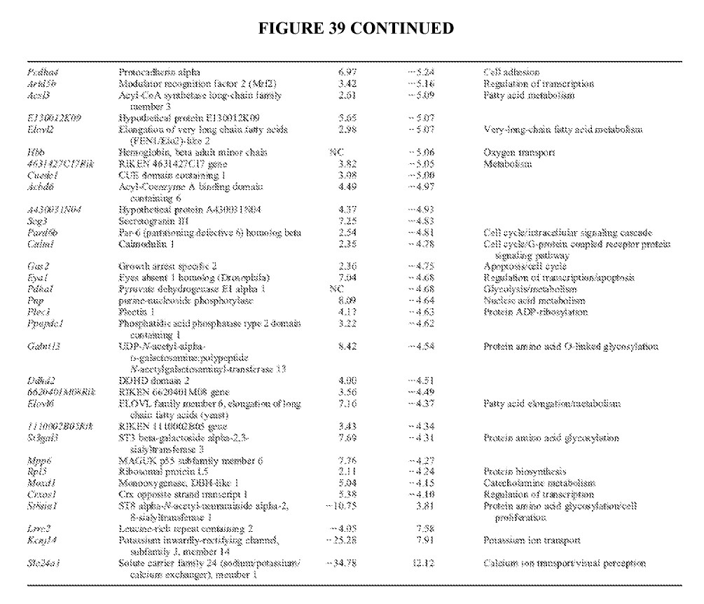

To validate qPCR results and explore additional possible downstream targets of NR2E3, the transgenic mice were mated with the Nrl::GFP transgenic mice, in which the expression of GFP is driven by an Nrl promoter (See, e.g., Example 1). In the resulting Nrl::GFP/Crx::Nr2e3/Nrl^{-/-} mice, all rod photoreceptors are specifically tagged with GFP and can therefore be purified by fluorescence-activated cell sorting (FACS). Expression profiling of FACS-purified GFP+ cells from Nrl::GFP/Crx::Nr2e3/Nrl^{-/-} mice was performed at 4 weeks. The comparison of gene profiles to those of GFP+ cells from Nrl::GFP/Nrl^{-/-} and Nrl::GFP/WT mice revealed that ectopic expression of NR2E3 suppressed a large number of genes, which were up-regulated in the Nrl::GFP/Nrl^{-/-} retina (See FIG. 39). Several of these genes are known to be preferentially expressed in cone photoreceptors (See FIG. 38). A set of genes was upregulated upon expression of NR2E3 in the Nrl^{-/-} retina; whereas rhodopsin was among the genes induced by NR2E3, several rod phototransduction genes showed only marginal or no increase in expression when compared with the Nrl^{-/-} retina (See FIG. 39). Although an understanding of the mechanism is not necessary to practice the present invention and the present invention is not limited to any particular mechanism of action, in some embodiments, the differentially expressed genes in the Crx::Nr2e3/Nrl^{-/-} retina, compared with Nrl^{-/-} retina, are direct downstream targets of NR2E3 (e.g., they are directly regulated by NR2E3 expression and/or activity).

### CRX is not Necessary for NR2E3-Mediated Gene Regulation.

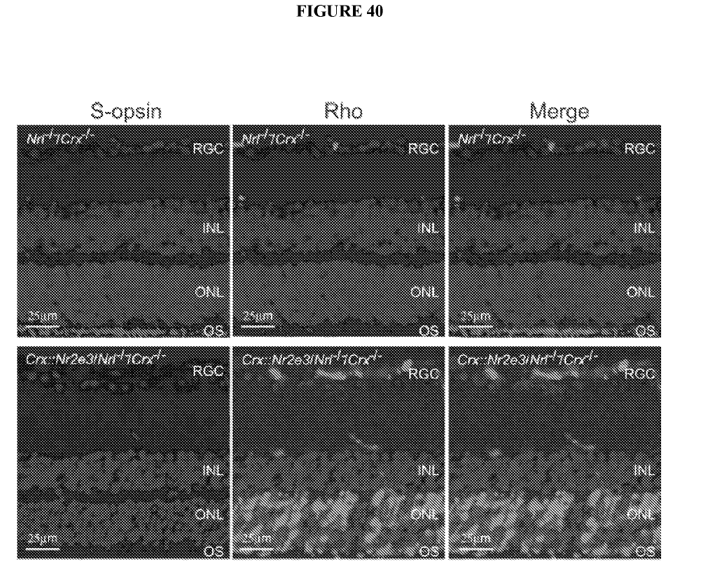

To evaluate the hypothesis that CRX is required for NR2E3-mediated transcriptional regulation (See, e.g., Peng et al., (2005) Hum. Mol. Genet., 14, 747-764), Crx::Nr2e3/Nrl^{-/-} mice were mated with the Nrl and Crx double knockout (Nrl^{-/-}/Crx^{-/-}) mice. In the Nrl^{-/-}/Crx^{-/-} retina, M-opsin is barely detectable because of the Crx2/2 background (See, e.g., Furukawa et al., (1999) Nat. Genet., 23, 466-470); however, S-opsin and cone arrestin are enriched and rhodopsin is undetectable because of the absence of NRL (See FIG. 40). In the Crx::Nr2e3/Nrl^{-/-}/Crx^{-/-} retina, ectopic expression of NR2E3 results in complete suppression of S-opsin and cone arrestin, whereas rhodopsin staining is observed in the ONL (See FIG. 40). A few rhodopsin positive cells are found even in the inner nuclear layer (INL) of the Crx::Nr2e3/Nrl^{-/-}/Crx^{-/-} retina (e.g., in some embodiments, reflecting migration defects). Thus, the present invention provides that NR2E3 can directly modulate rod and cone specification even in the absence of CRX and/or NRL.

### NR2E3 Transforms Cone Precursors to Rod-Like Cells in the WT Retina.

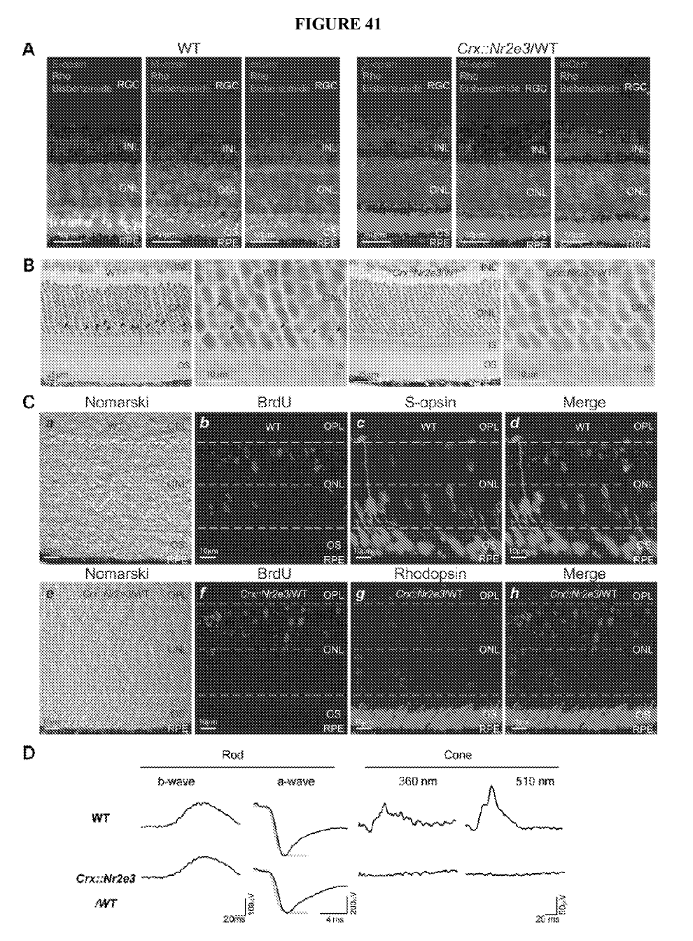

To further examine NR2E3 function, the Crx::Nr2e3 transgene was transferred to the WT background. Expression of rhodopsin in the Crx::Nr2e3/WT retina was similar to WT; however, no cone-specific markers were detected (See FIG. 41A). The retinal histology was apparently normal in the transgenic mice, except that cone-like nuclei were not observed (See FIG. 41B). To determine the fate of cone precursors in the Crx::Nr2e3/WT retina, a single dose of BrdU was injected in the pregnant mice at day 14 after fertilization (note that E13-E14 represents the peak of cone genesis) and the retinas were analyzed at P21. The number of strongly BrdU-labeled cells in the ONL near the optic nerve was not altered in transgenic retinas when compared with WT retinas; however, there was a difference in the location of these cells. In the WT retina, strongly BrdU-labeled cells were observed in both the inner and outer halves of the ONL, and most cells in the outer half co-expressed cone markers, such as S-opsin (See FIG. 41Ca-d). In the transgenic retina, almost all strongly BrdU-labeled cells were located in the inner part of the ONL (See FIG. 41Ce-f). TUNEL staining at E16, P2, P6, P10 and 4 weeks did not reveal any obvious differences between the WT and transgenic retinas. Although an understanding of the mechanism is not necessary to practice the present invention and the present invention is not limited to any particular mechanism of action, in some embodiments, the present invention provides that NR2E3 expression forces the early-born cone precursors to adopt the rod-like phenotype (e.g., these cells stay in the inner part of the ONL with other early-horn rods and do not migrate to the outer part of the ONL as WT cones). ERGs from the Crx::Nr2e3/WT transgenic mice show normal rod responses but undetectable S- or M-cone responses (See FIG. 41D). Thus, these retinas contain only rod photoreceptors.

Ectopic Expression of NR2E3 Transforms Differentiating S-Cones into Rod-Like Cells.

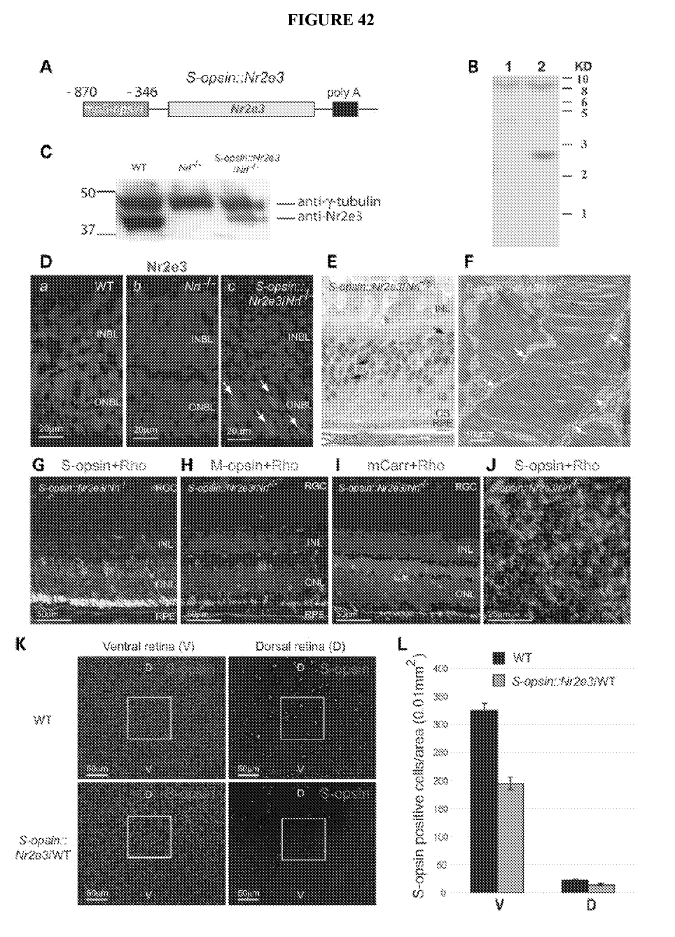

Experiments were then conducted to determine whether ectopic expression of NR2E3 can also suppress phototransduction genes in differentiating cones. NR2E3 was expressed under the control of S-opsin promoter (See, e.g., Akimoto et al., (2004) Invest. Ophthalmol. Vis. Sci., 45, 42-47) in both Nrl^{-/-} and WT genetic backgrounds (See FIG. 42). In the S-opsin::Nr2e3/Nrl^{-/-} retina, the temporal expression of Nr2e3 transcripts was similar to S-opsin in the early developmental stages but decreased after 3 weeks, and the protein amounts appeared considerably lower than the WT (See FIGS. 42C and D). Rhodopsin was detected in the ONL and OSs (See FIG. 42G-J) and was predominantly distributed in the dorsal retina. In retinal sections and whole mounts, rhodopsin and cone proteins did not colocalize (See FIGS. 42G and J). A few of the nuclei in the ONL of the S-opsin::Nr2e3/Nrl^{-/-} retina showed rod-like morphology and the OSs were rod-like (closed discs and long) but were distorted when compared with the Nrl^{-/-} retina (See FIGS. 42E and F). ERG studies showed no differences in visual function between the transgenic and the Nrl^{-/-} mice. qPCR analysis revealed the absence of Gnat1 transcripts in the S-opsin::Nr2e3/Nrl^{-/-} retina although rhodopsin expression could be detected. Although an understanding of the mechanism is not necessary to practice the present invention and the present invention is not limited to any particular mechanism of action, in some embodiments, a less dramatic phenotype in the S-opsin::Nr2e3 retina when compared with the Crx::Nr2e3 mice is due to the expression time and levels of NR2E3 in developing cones. Although an understanding of the mechanism is not necessary to practice the present invention and the present invention is not limited to any particular mechanism of action, in some embodiments, the reduced level of NR2E3 in S-opsin::Nr2e3 retina reflects an equilibrium between the NR2E3 expression driven by the S-opsin promoter and its subsequent repression by NR2E3 itself. In the S-opsin::Nr2e3/WT mice, retinal morphology and ERGs showed no obvious difference from WT. Although the dorsal-ventral pattern of S-opsin gradient was not altered in the S-opsin::Nr2e3/WT retina, the number of S-opsin positive cells was decreased in retinal flat mounts (See FIGS. 42K and L) and sections. Cone arrestin positive cells were also reduced but not the M-opsin positive cells.

### Example 6

### Transformation of Cone Precursors to Functional Rod Photoreceptors by Transcription Factor NRL

### Materials and Methods.

Plasmid Constructs and Generation of Transgenic Mice. A 2.3-kb mouse Crx promoter DNA (from −2286 to −72, GenBank accession nos. AF335248 and AF301006) and the Nrl coding region (GenBank accession no. NM008736) with an additional Kozak sequence were amplified and cloned into a modified promoterless pCl (pCIpl) vector (See, e.g., Akimoto et al., (2004) Invest Ophthalmol Vis Sci 45:42-47). The 3.7-kb Crxp-Nrl insert was purified and injected into fertilized Nrl^{-/-} (mixed background of 129×1/SvJ and C57BL/6J) mouse oocytes (University of Michigan transgenic core facility). Transgenic founders were bred to the Nrl^{-/-} mice to generate F1 progeny. The progeny was also mated to C57BL/6J to generate Crxp-Nrl/WT mice. The BPp-Nrl transgenic mice were generated in a similar manner, except that a 520-bp mouse S-opsin promoter DNA (See, e.g., Akimoto et al., (2004) Invest Ophthalmol Vis Sci 45:42-47) was used. All studies involving mice were performed in accordance with institutional and federal guidelines and approved by the University Committee on Use and Care of Animals at the University of Michigan.

Immunohistochemistry and Confocal Analysis. Retinal sections and dissociated cells were prepared as described (See, e.g., Cheng et al., (2004) Hum Mol Genet 13:1563-1575; Strettoi et al., (2002) J Neurosci 22:5492-5504) and probed with specific antibodies. Antibodies used for immunohistochemistry were as follows: rabbit anti S-opsin, Mopsin, and cone arrestin antibodies (gifts from C. Craft), mouse anti-rhodopsin (1D4) (gift from R. Molday), rabbit β-galactosidase (Cappel), rat anti-(galactosidase (gift from T. Glaser) rabbit anti-Cre (Covance), mouse anti-Cre (Chemicon), rabbit and mouse anti-Protein Kinase C-α (Sigma); rabbit anti-mGluR6 (Neuromics); rabbit anti-calbindin D-28k (Swant); mouse anti-G0α (Chemicon); mouse anti-Neurofilament 200 kD (clone N52, Sigma); mouse anti-Glutamine Synthetase (Chemicon); mouse anti-NK3-receptor (Abcam, Novus Biological Inc); rabbit anti-Disabled 3 (from Dr. Brian Howell); mouse anti-bassoon (Stressgen); mouse anti-kinesin 2 (Covance); mouse anti-synaptophysin (Boehringer); mouse anti-PSD95 (Abcam); goat anti-Choline Acetyl Transferase (ChAT; Chemicon); rabbit anti-Tyrosine Hydroxylase (Chemicon). Fluorescent detection was performed using AlexaFluor-488, 546 or 633 (Molecular Probes) conjugated secondary antibodies. Sections were visualized under an Olympus FLUOVIEW laser scanning confocal microscope (Olympus, Melville, N.Y.) or a Leica TSC NT confocal microscope (Leica, Bannockburn, Ill.), equipped with an argon-krypton laser. Images were digitized by using FLUOVIEW software version 5.0 or METAMORPH 3.2 software.

ChIP. Mouse retinas from different developmental stages were subjected to ChIP analysis using a CHIP-IT kit (Active Motif, Carlsbad, Calif.). IP was performed by using anti-NRL or normal rabbit Ig (IgG). PCR primers, derived from the Thrb and S-opsin promoter region (GenBank accession nos. NT_{—}039340.6 and NT_{—}039595.6, respectively) spanning the putative NRE, were used for amplification (from nucleotides 26331250 to 26331458 and 13773280 to 13773502, respectively) by using immunoprecipitated DNA as template. The albumin PCR primers were 5′-GGACACAAGACTTCTGAAAGTCCTC-3′ (SEQ ID NO.: 11)and 5′-TTCCTACCCCATTACAAAATCATA-3′ (SEQ ID NO.: 12).

EMSA. Oligonucleotides spanning the putative NRE were radiolabeled by using [γ-³²]P-ATP (Amersham Biosciences, Piscataway, N.J.) and incubated in binding buffer (20 mM Hepes, pH 7.5/60 mM KCl/0.5 mM DTT/1 mM MgCl2/12% glycerol) with bovine retinal nuclear extract (RNE; (See, e.g., Mitton et al., (2003) Hum Mol Genet 12:365-373)) (20 μg) and 50 μg/ml poly(dI-dC) for 30 min at room temperature, as described (See, e.g., Khanna et al., (2006) J Biol Chem 281:27327-27334). For competition experiments, nonradiolabeled oligonucleotides were used in molar excess of the labeled oligonucleotides. In some experiments, antibodies were added after the incubation of ³²P-labeled oligonucleotides with RNE. Samples were analyzed by 7.5% nondenaturing PAGE.

Electroretinography. ERGs were recorded as described (See, e.g., Mears et al., (2001) Nat Genet 29:447-452).

### Overexpression of Nrl in Photoreceptor Precursors Drives Rod Differentiation at the Expense of Cones.

It was hypothesized that if cones develop from a unique pool of competent cells, early cone precursors would not be responsive to NRL. On the other hand, transformation of cone precursors to rods by NRL would indicate an intrinsic capacity to give rise to both rods and cones. To directly test this, transgenic mouse lines were generated, (Crxp-Nrl/WT), expressing Nrl under the control of a previously characterized 2.5 kb proximal promoter of the Crx gene (Crxp-Nrl), which is specifically expressed in postmitotic cells that can develop into either cone or rod photoreceptors (See, e.g., Furukawa et al., (2002) J Neurosci 22:1640-1647; Cheng et al., (2006) Hum Mol Genet 15:2588-2602).

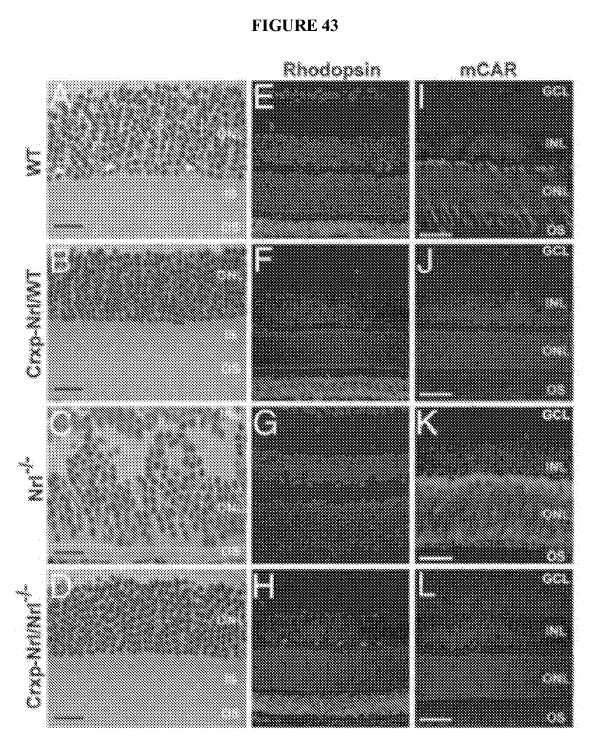

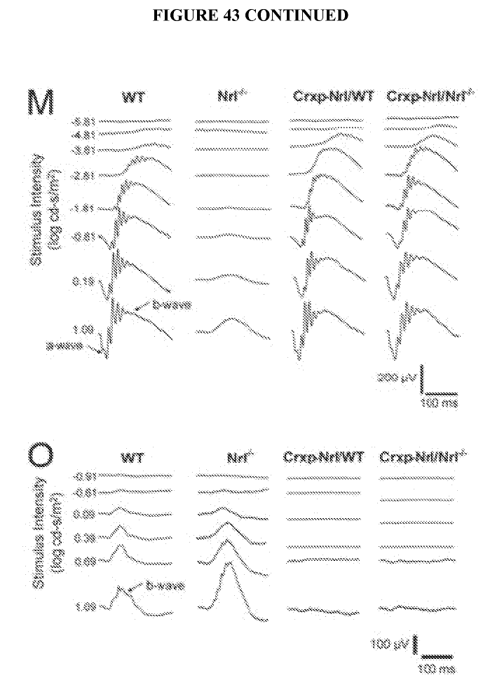

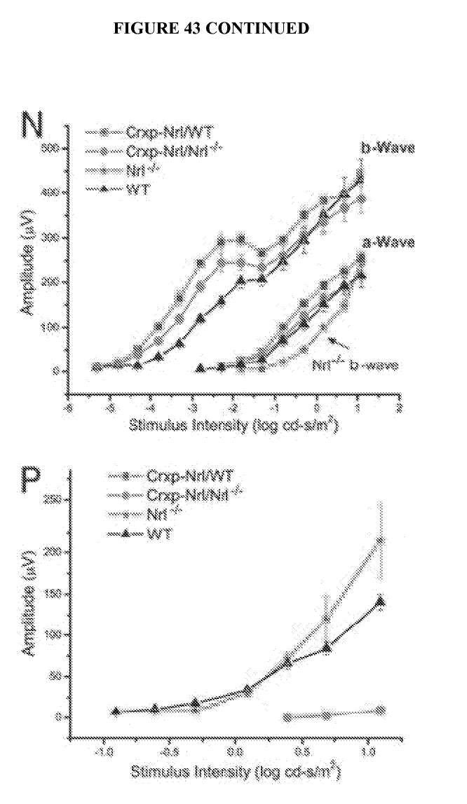

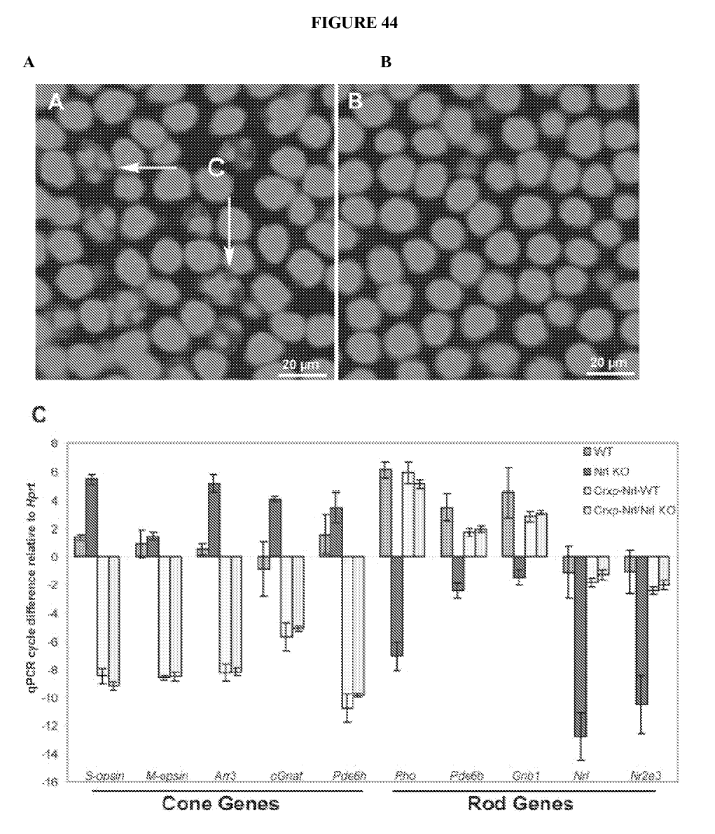

Light micrographs of semithin (plastic) sections of Crxp-Nrl/WT mouse retina showed normal laminar organization (FIGS. 1A and B). Immunofluorescence studies demonstrated comparable rhodopsin expression relative to WT and Nrl^{-/-} mice (See FIG. 43E-G); however, staining of cone-specific markers (cone arrestin, peanut agglutinin (PNA), S-opsin, and M-opsin) was undetectable in cryosections and flat-mount preparations from transgenic retinas (See FIG. 43I-K). Confocal examination of the outer nuclear layer revealed only the photoreceptor nuclei with dense chromatin (See FIGS. 44A and B) that are characteristics of rods in the WT retina (See, e.g., Carter-Dawson L D, LaVail M M (1979) J Comp Neurol 188:245-262). Dark-adapted corneal flash electroretinograms (ERGs) from Crxp-Nrl/WT mice revealed normal rod function even at 6 mo (FIGS. 43M and N), whereas the cone-derived photopic ERG response was absent at all ages (FIGS. 43O and P). These data provide a complete absence of cone functional pathway in the Crxp-Nrl/WT mice. Consistent with these results, quantitative RT-PCR analysis demonstrated no expression of cone phototransduction genes in the Crxp-Nrl/WT retina, with little or no change in rod-specific genes (See FIG. 44C).

The Crxp-Nrl transgenic mice were then bred into the Nrl^{-/-} background (Crxp-Nrl/Nrl^{-/-}) to test whether Nrl expression in a cone-only retina could convert a retina composed solely of cones to rods as seen in the Crxp-Nrl/WT mice. Analysis of retinal morphology uncovered a remarkable transformation of a dysmorphic retina with whorls and rosettes in the Nrl^{-/-} mice (See, e.g., Mears et al., (2001) Nat Genet 29:447-452) to a WT-like appearance (See FIGS. 43C and D). Images from toluidine blue-stained retinal sections revealed clear extended outer segments and a highly organized laminar structure (See FIG. 43D). Similar to the WT (See, e.g., Carter-Dawson L D, LaVail M M (1979) J Comp Neurol 188:245-262), and unlike the all-cone retina in Nrl^{-/-} mice (See, e.g., Mears et al., (2001) Nat Genet 29:447-452), the outer nuclear layer of Crxp-Nrl/Nrl^{-/-} retina had rod-like nuclei with dense chromatin. Immunolabeling of adult Crxp-Nrl/Nrl^{-/-} retinal sections demonstrated a complete absence of cone proteins (cone arrestin data are shown in FIG. 43L). In contrast to the Nrl^{-/-} retinas (See FIG. 43G), Crxp-Nrl/Nrl^{-/-} mice displayed normal levels of rhodopsin (See FIG. 43H). No photoreceptor degeneration was evident by histology or ERG at least up to 6 mo (See FIG. 43).

### Retinal Synaptic Architecture is Modified in the Absence of Cones.

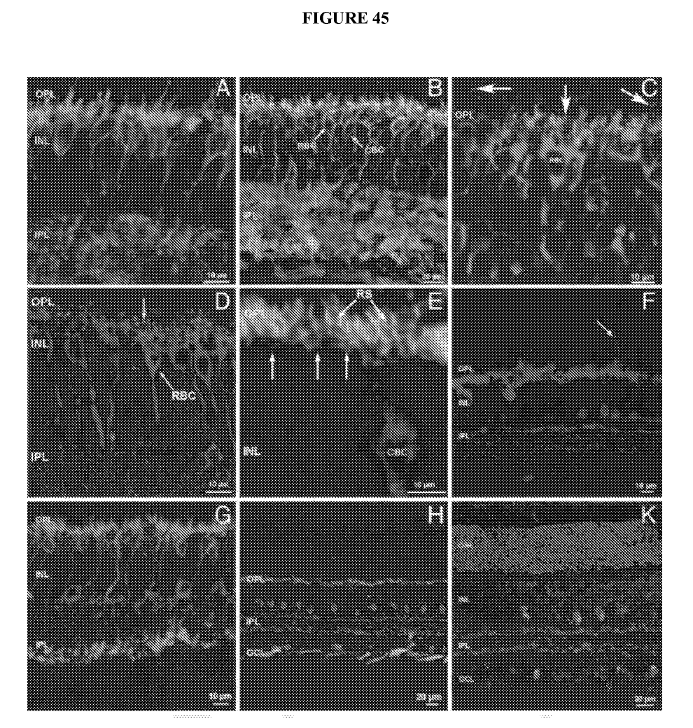

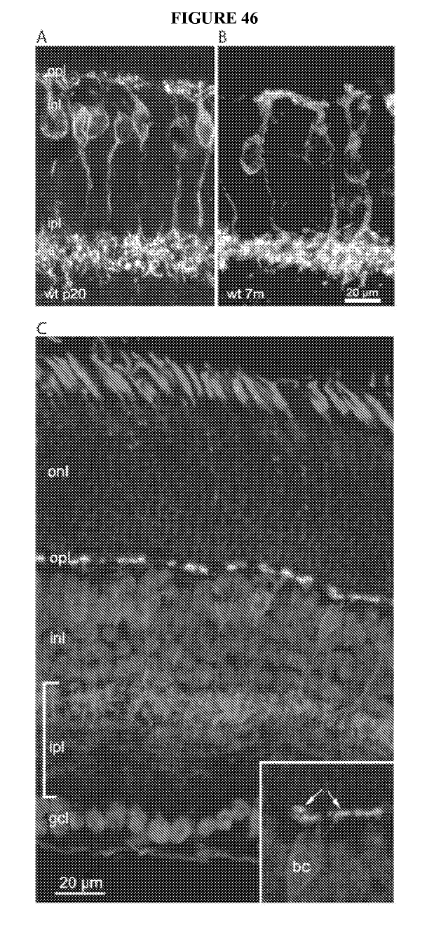

Given that a complete rod-only retina did not reveal gross changes in retinal morphology, it was contemplated whether cones are essential for proper development and lamination of cone connected neurons. Cones are presynaptic to dendrites originating from the cell bodies of horizontal cells and to at least nine different types of cone bipolar neurons (See, e.g., Ghosh et al., (2004) J Comp Neurol 469:70-82; Pignatelli V, Strettoi E (2004) J Comp Neurol 476:254-266). Immunostaining of Crxp-Nrl/WT retinas with a panel of cell-type-specific antibodies (See, e.g., Strettoi et al., (2002) J Neurosci 22:5492-5504) did not reveal any major difference in the distribution of the marker proteins for horizontal, bipolar, amacrine, and glial cells (See FIG. 45). Despite the absence of cones, it was apparent that both the ON and OFF subtypes of cone bipolar cells were retained (See FIGS. 45A, B, and E). All ON bipolar neurons (both rod and cone bipolar cells) carried metabotropic glutamate receptors on their dendritic tips (mGluR6), and thus they were postsynaptic to rod spherules. It was unclear whether cone bipolar cells belonging to the OFF functional type received synapses from rod photoreceptors. The dendrites of one type of OFF cone bipolar cells, marked with Neurokinin receptor 3 (NK3-R), form basal (or flat) junctions with cone pedicles in the outer plexiform layer (See FIG. 46). Although confocal microscopy does not reach the necessary resolution to detect such putative contacts, it is apparent from the preparations that not all of the dendrites of NK3-R-positive cone bipolar cells come in close apposition to the rod spherules and that basal junctions are therefore unlikely (See FIG. 45E).

To study the morphology of horizontal cells, Crxp-Nrl/WT retinas were stained with a calbindin antibody (See FIG. 45F). Although no gross changes were observed, rare ectopic sprouts were noticed emerging from the outer plexiform layer and extending into the outer nuclear layer. Other examined markers also revealed a normal distribution throughout the retina (See FIG. 45G-I). All amacrine neurons exhibited their peculiar bistratified morphology (See FIG. 45G). Cholinergic amacrine cells (See FIGS. 45H and I) showed a typical distribution in two mirror-symmetric populations. Dopaminergic amacrines and Muller glial cells also showed normal organization. Thus, besides the likely reconnections of ON cone bipolar and horizontal cells to rods, the retina from Crxp-Nrl/WT mice was indistinguishable from WT.

### Ectopic Expression of NRL Can Suppress Cone Function and Induce Rod Characteristics in a Subset of Photoreceptors Expressing S-Opsin.

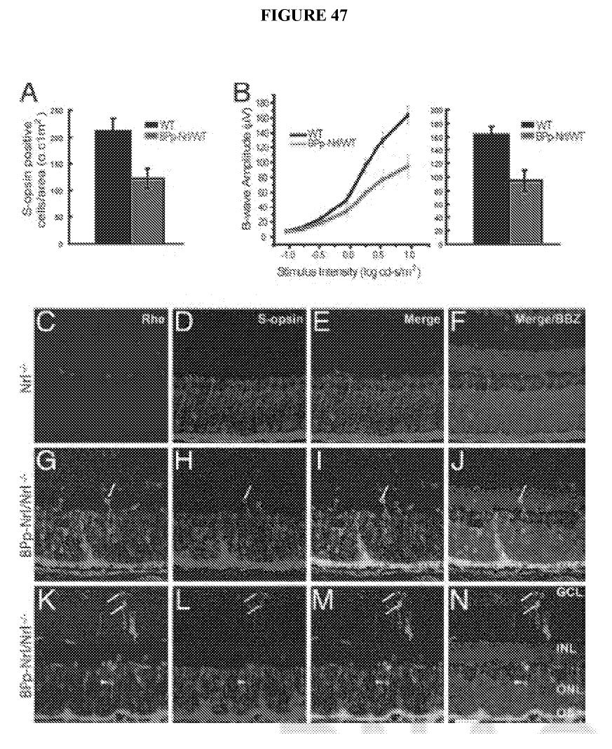

The onset of S-opsin expression begins at E16-E18 in rodents (See, e.g., Szel et al., (1993) J Comp Neurol 331:564-577; Chiu M I, Nathans J (1994) Vis Neurosci 11:773-780). To further delineate NRL's role in cell fate determination, transgenic mouse lines (BPp-Nrl/WT) were generated expressing NRL under the control of a previously characterized S-opsin promoter (See, e.g., Akimoto et al., (2004) Invest Ophthalmol Vis Sci 45:42-47). Immunostaining revealed a significant decrease of S-opsin-positive cells in the inferior region of flat-mounted retinas (See FIG. 47A). Consistent with histological and immunohistochemical analysis, ERGs from the BPp-Nrl/WT mice showed a 50% reduction in the photopic b-wave amplitude compared with the WT (See FIG. 47B); however, scotopic ERG a- and b-wave amplitudes were largely unaffected.

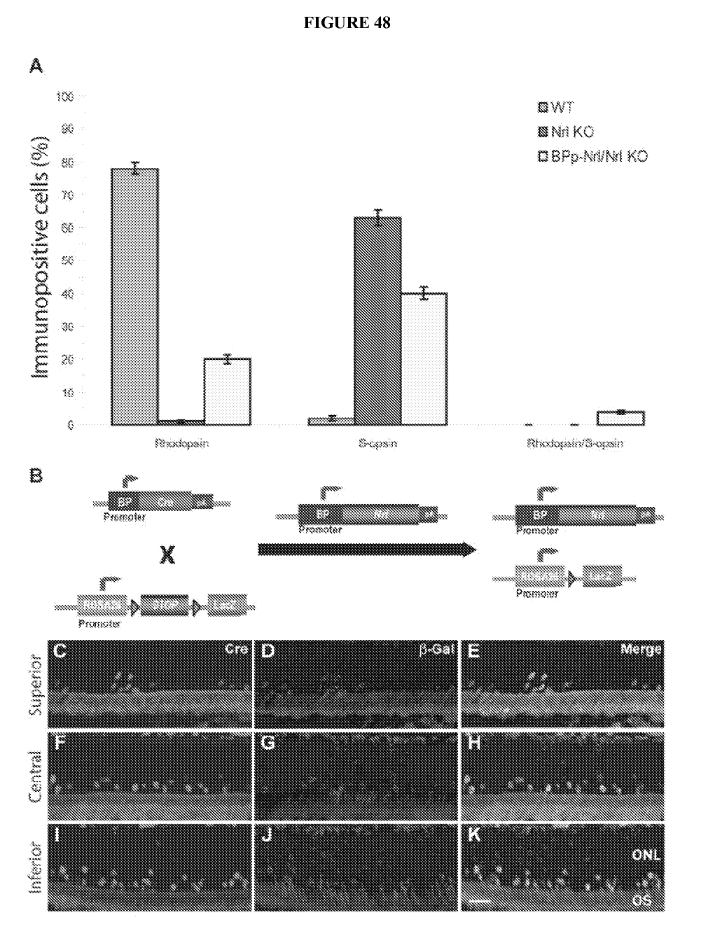

The BPp-Nrl transgene was then transferred to the Nrl^{-/-} background (BPp-Nrl/Nrl^{-/-}) mice. Ectopic expression of Nrl in the all-cone Nrl^{-/-} retina, even at this stage (i.e., under the control of S-opsin promoter), resulted in rhodopsin staining in the ONL; however, as in the Nrl^{-/-} mice (See FIG. 47C-F) the outer and inner segments remained stunted (See FIG. 47G-N). The BPp-Nrl/Nrl^{-/-} retina also revealed hybrid cells that expressed both S-opsin and rhodopsin in ONL, INL, and ganglion cell layer (See FIG. 47G-N and FIG. 48A). ERG data showed that, although the phototopic b-wave (cone-derived) was somewhat reduced, the scotopic b-wave amplitude was still undetectable in BPp-Nrl/Nrl^{-/-} mice.

In order to examine the fate of S-opsin-expressing cells, we mated the BP-Cre transgenic mice (that expresses Cre-recombinase under the control of the same S-opsin promoter; See, e.g., Akimoto et al., (2004) Invest Ophthalmol Vis Sci 45:42-47) were mated with the R26R reporter line and the BPp-Nrl/WT line (See FIG. 48B-K). A large number of Cre-negative cells were labeled with β-galactosidase in the BP-Cre; R26R; BPp-Nrl/WT background (See FIG. 48B-K). Approximately 40% of β-galactosidase-positive cells did not colocalize with S-opsin. Their position in the ONL and the lack of S-opsin staining indicate that these are rod photoreceptors, providing a possible fate switch in response to ectopic NRL expression. However, staining with the rod marker rhodopsin was inconclusive. TUNEL staining of sections from E18 retina did not detect obvious differences between WT and BPp-Nrl/WT mice.

NRL can Associate with Cone-Specific Promoter Elements.

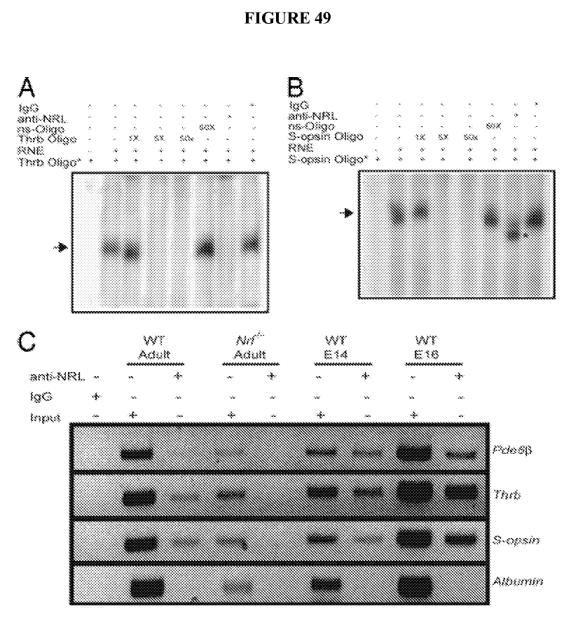

In order to examine whether NRL could directly modulate cone-specific promoters, 3 kb of 5′ upstream promoter regions of the two cone-expressed genes, Thrb (encoding Trβ2 that is involved in M-cone differentiation, See, e.g., Ng et al., (2001) Nat Genet 27:94-98) and S-opsin, were screened for the presence of Nrl or Maf response element (NRE/MARE) (See, e.g., Rehemtulla et al., (1996) Proc Natl Acad Sci USA 93:191-195). Oligonucleotides spanning the single putative NRE sites, identified within the Thrb and S-opsin promoters, were used for EMSA with bovine retinal nuclear extracts. A shifted band was detected that could be specifically competed by the addition of 50-fold molar excess of unlabeled NRE-oligonucleotide but not a random oligonucleotide (See FIGS. 49A and B). The addition of anti-NRL antibody abolished the shifted band for the Trβ2 oligonucleotide (See FIG. 49A), whereas S-opsin promoter-protein complex demonstrated an increased mobility in the native polyacrylamide gel (See FIG. 49B). Notably, disappearance of the shifted band may occur because of the dynamic nature of some DNA-protein interactions, whereas the net charge-to-mass (e/m) ratio of the ternary complex determines their rate of mobility in a native polyacrylamide gel (See, e.g., Sambrook J, Russell D (2001) Molecular Cloning (Cold Spring Harbor Lab Press, Cold Spring Harbor, N.Y.). Similar results were obtained when the radiolabeled oligonucleotides were incubated with anti-NRL antibody simultaneously with the retinal nuclear extract or with the nuclear extract preincubated with the anti-NRL antibody for 15 min. No effect on the gel-shift was observed in the presence of control rabbit IgG.

In order to further evaluate the association of NRL with Thrb and S-opsin promoter elements in vivo, ChIP assays was performed using WT embryonic and adult mouse retinas. PCR primer sets spanning the Thrb and S-opsin NRE-amplified specific products with DNA immunoprecipitated with the anti-NRL antibody but not with the rabbit IgG (See FIG. 49C). ChIP experiments using the Nrl−/− mouse retina (negative control) did not reveal specific amplified products (See FIG. 49C).

### Example 7

### Characterization of Nrl Phosphorylation and Transcriptional Activity

### Materials and Methods.

Cell culture and transfection. COS-1 and HEK293 cells were cultured in Dulbecco's modified Eagle's medium containing 10% fetal bovine serum and transfected using FUGENE 6 (Roche Applied Science, Indianapolis, Ind.), at 80% confluency, with plasmid DNA, as described (See, e.g., Nishiguchi et al., 2004; Proc Natl Acad Sci USA 101:17819-17824).

Plasmid construction and mutagenesis. The wild-type (WT) human NRL cDNA (GenBank #NM_{—}006177) was subcloned at the EcoRI-NotI sites in the pcDNA4 His/Max C vector (Invitrogen, Carlsbad, Calif.). The QUICKCHANGE XL site-directed mutagenesis kit (Stratagene, La Jolla, Calif.) was used, as described (See, e.g., Nishiguchi et al., 2004 Proc Natl Acad Sci USA 101:17819-17824), to generate mutants from the NRL expression construct. Constructs were sequenceverified before use.

Immunoblot analysis. Transfected COS-1 whole cell extracts were solubilized in 2×SDS sample buffer by heating to 100° C. for 5 min and separated by 15% SDS-PAGE. Proteins were transferred to nitrocellulose by electroblotting, and immunoblot analysis was performed using a mouse monoclonal ANTI-XPRESS antibody (Invitrogen) according to standard protocols (See, e.g., Ausubel et al., 1989, Current Protocols in Molecular Biology. New York: John Wiley and Sons. 10.8.1-10.8.7).

³²P metabolic labeling and immunoprecipitation (IP). Transfected COS-1 cells were metabolically labeled using 0.5 μCi/ml [γ-32P]ATP (GE Healthcare, Piscataway, N.J.) as described (Ausubel et al., 1989, Current Protocols in Molecular Biology. New York: John Wiley and Sons. 10.8.1-10.8.7). After 1 hr, labeled cells were harvested in PBS containing protease inhibitors, and sonicated. After cell extracts were preabsorbed with Protein-G beads (Invitrogen), the cell extracts were incubated with anti-XPRESS antibody and Protein-G agarose beads overnight at 4° C. with gentle shaking. The beads were washed with PBS containing 1% Triton X-100. The proteins were suspended in 2×SDS sample buffer and then analyzed by SDS-PAGE.

Phosphatase treatment. Transfected COS-1 cells were harvested with phosphatase buffer containing 0.1 mM PMSF and 1×complete proteinase inhibitor (Roche Applied Science), and treated for 1 hr at 30° C. with 80 units of λ-phosphatase (New England Biolabs, Beverly, Mass.). The reaction was terminated by heating to 100° C. for 5 min in 5×SDS sample buffer, and the samples were subjected to SDSPAGE.

Immunocytochemistry. Transfected COS-1 cells were washed with PBS, fixed using 4% paraformaldehyde/PBS for 10 min, and washed again in PBS. Cells were permeabilized using 0.05% Triton X-100/PBS for 10 min. After washing, a 5% BSA/PBS solution was applied and the cells were blocked for 30 min. The cells were incubated for 1 hr with an ANTI-XPRESS antibody (1:400 dilution) in 1% BSA/PBS, and with a secondary anti-mouse IgG Alexa fluor 488 (Molecular Probes, Eugene, Oreg.) (1:400 dilution). Nuclei were counterstained with bisbenzimide, and cells were examined by fluorescent microscopy.

Electrophoretic mobility shift assays (EMSA). Gel shift assays were performed essentially as described (See, e.g., Rehemtulla et al., 1996, Proc Natl Acad Sci USA 93:191-195), with minor modifications. Nuclear extracts from transfected COS-1 cells were prepared using a commercial kit (Active motif, Carlsbad, Calif.), and expression of mutant NRL protein was normalized by immunoblot analysis. Nuclear extracts were pre-incubated for 30 min on ice in binding buffer containing 20 mM HEPES (pH 7.9), 1 mM EDTA, 50 mM NaCl, 1 mM DTT, 10% Glycerol), 2.5 μg/ml poly(dI-dC). Radiolabeled DNA probes containing the rhodopsin-NRE site (NRE-F 5′-CTCCGAGGTGCTGATTCAGCCGGGA-3′ (SEQ ID NO.: 13); NRE-R 5′-TCCCGGCTGAATCAGCACCTCGGAG-3′ (SEQ ID NO.: 14)) were added and extracts were incubated another 30 min at room temperature. The non-specific oligonucleotides were NS-F 5′-GAGGGAGATATGCTTCATAAGGGCT-3′ (SEQ ID NO.: 15); and NS-R 5′-AGCCCTTATGAAGCATATCTCCCTC-3′ (SEQ ID NO.: 16). DNA-protein complexes were analyzed on 4% non-denaturing polyacrylamide gels in 0.5×TBE.

Luciferase assays. The luciferase reporter experiments were performed using HEK293 cells, and contained pGL2 with the bovine rhodopsin promoter driving a luciferase cDNA sequence (pBR130-luc), and expression constructs carrying the CRX cDNA (pcDNA4-CRX) and/or NR2E3 cDNA (pcDNA4-NR2E3), as described (See Bessant et al., 1999, Nat Genet 21:355-356; Nishiguchi et al., 2004, Proc Natl Acad Sci USA 101:17819-17824), with minor modifications. Increasing amount (0.01, 0.03, and 0.09, 0.3 μg) of a NRL expression construct containing either WT or NRL mutant/variant was also co-transfected with pBR130-luc (0.3 μg per well), and pcDNA4-CRX and/or pcDNA4-NR2E3 (0.5 μg per well), as indicated for individual experiments. Empty pcDNA4 expression vector and cytomegalovirus-β-gal (0.1 μg per well) were included to normalize for the amount of transfected DNA and transfection efficiency, respectively.

### Evolutionary Conservation of NRL Variants Identified in Retinopathy Patients.

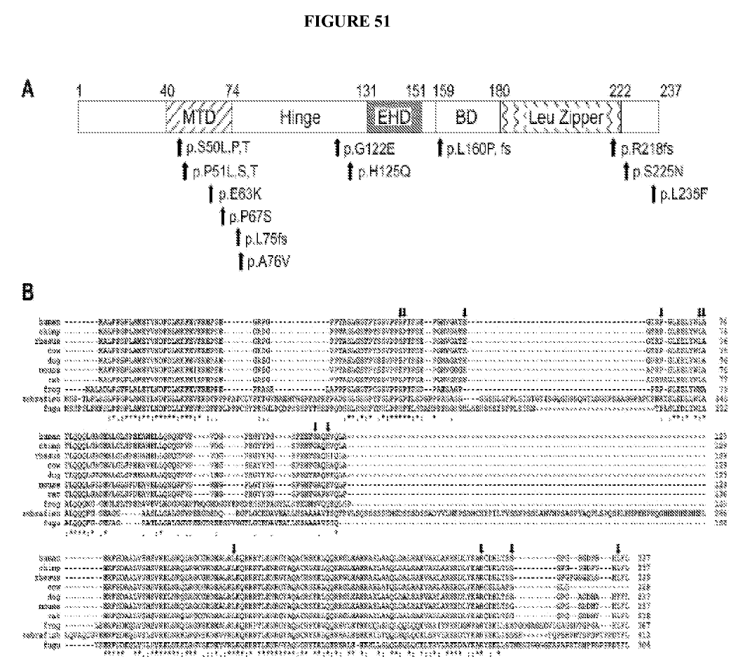

Evolutionary conservation of amino acid residues can provide significant insights into NRL function. NRL orthologs have been identified in many vertebrates with the exception of chicken (See, e.g., Coolen et al., 2005, Dev Genes Evol 215:327-339; Whitaker and Knox 2004, J Biol Chem 279:49010-49018). To date, 17 mutations and/or variations in the NRL gene have been detected (See, e.g., Bessant et al., 1999, Nat Genet 21:355-356; DeAngelis et al., 2002, Arch Ophthalmol 120:369-375; Martinez-Gimeno et al., 2001, Hum Mutat 17:520; Nishiguchi et al., 2004, Proc Natl Acad Sci USA 101:17819-17824; Wright et al., 2004, Hum Mutat 24:439; Ziviello et al., 2005, J Med Genet 42:e47); these include fourteen missense and three frameshift mutations (See FIG. 51A). All changes have been identified in twelve amino acids; three of these (p.S50, p.P51, p.L160) show more than one alteration. Five (p.S50, p.P51, p.A76, p.L160 and p.R218) of the twelve residues are conserved in all known orthologs of NRL from human to fugu (See FIG. 51B). Residues p.P67 and p.L75 are conserved in all orthologs, except zebrafish and frog, respectively (See FIG. 51B).

### Effect of NRL Mutations/Variants on Protein Stability and Phosphorylation Status.

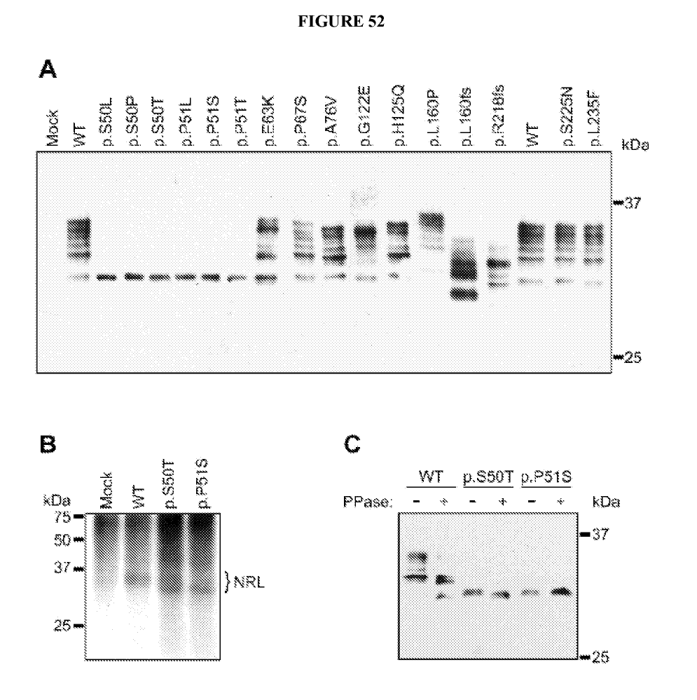

Previously, NRL isoforms from human retina extract showed a pattern similar to that of transfected COS-1 cells (See, e.g., Swain et al., 2001, J Biol Chem 276:36824-36830) or HEK293 cells, implying that modifications of NRL are congruous among retina and these cell types. Thus, WT and mutant NRL proteins were expressed in COS-1 cells to examine their effect on NRL stability and phosphorylation status. In contrast with at least six 30-35 kDa isoforms (including 4 kDa XPRESS epitope) of WT-NRL, all p.S50 and p.P51 mutants showed significant reduction of isoforms, with the appearance of a major 30 kDa band (See FIG. 52A). The p.P67S, p.H125Q and p.S225N proteins displayed patterns equivalent to that of WT-NRL, suggesting that these changes do not affect protein stability or phosphorylation (See FIG. 52A). Mutants p.E63K, p.A76V, p.G122E and p.L160P contained a different isoform pattern. The p.E63K's band sizes were in the WT range, while that of p.L160P were of higher molecular mass. p.A76V and p.G122E mutants were each missing the highest molecular mass band. p.L235F migrated slightly below WT, but had no change in pattern. The number of isoforms in the p.L160fs and p.R218fs mutants were decreased by three and migrated at lower molecular mass. The p.L75fs mutant could not be detected perhaps due to lower levels or unstable protein. WT and mutant NRL constructs were transfected into human Y79 retinoblastoma cells as well. However, transfected NRL isoforms (carrying XPRESS tag) could not be detected by immunoblot analysis because of low transfection efficiency.

To directly test NRL phosphorylation, metabolic labeling was performed using [γ-³² P]ATP and immunoprecipitation using anti-XPRESS antibody. WT, p.S50T and p.P51S mutants were phosphorylated, with the mutant proteins showing only the lower isoform(s) (See FIG. 52B). Phosphatase treatment of the WT-NRL-transfected COS-1 cell extracts demonstrated a reduction in NRL isoforms, while the treated mutant proteins migrated slightly below the untreated (See FIG. 52C). This is consistent with previous studies showing a reduction in NRL isoforms upon phosphatase treatment of human and bovine retina extracts (See, e.g., Swain et al., 2001, J Biol Chem 276:36824-36830).

### Effect of NRL Mutations/Variants on Nuclear Localization

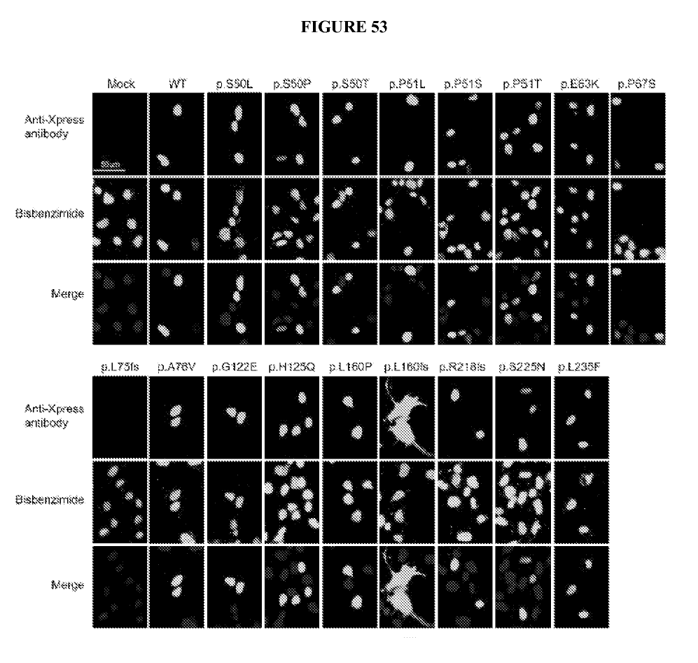

The subcellular distribution of mutant NRL proteins was next examined in COS-1 cells. All except two of the NRL mutant proteins (p.L75fs, p.L160fs) localized to the nucleus (See FIG. 53). Both of these mutations would be predicted to lose their bZIP domain and mislocalize to the cytoplasm. The p.L75fs mutant was essentially undetectable at exposure times equivalent to the other samples (See FIG. 53). At higher exposure, p.L75fs had very weak expression in a pattern similar to p.L160fs.

### Effect of NRL Mutations/Variants on DNA Binding

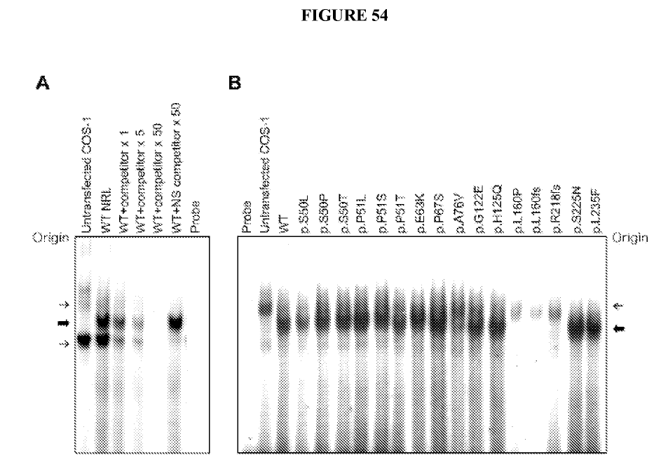

NRL is bound to NRE in the rhodopsin promoter (rhodopsin-NRE) (See, e.g., Rehemtulla et al., 1996, Proc Natl Acad Sci USA 93:191-195). COS-1 transfected NRL protein could also bind to the rhodopsin-NRE (See FIG. 54A). The intensity of the shifted bands was dramatically decreased by unlabeled rhodopsin-NRE in a concentration dependent manner; however, no change in intensity was detected with the non-specific (NS) control oligonucleotide, and in fact the NS probe reduced the non-specific oligonucleotide shifts (See FIG. 54A). Subsequent EMSA experiments were performed to investigate whether mutant NRL protein(s) affect rhodopsin-NRE binding. All variations except for p.L160P, p.L160fs and p.R218fs bound to the rhodopsin-NRE (See FIG. 54B). The p.A76V alteration appeared to have lower than WT binding.

### Effect of NRL Mutations/Variants on Transactivation of Rhodopsin Promoter.

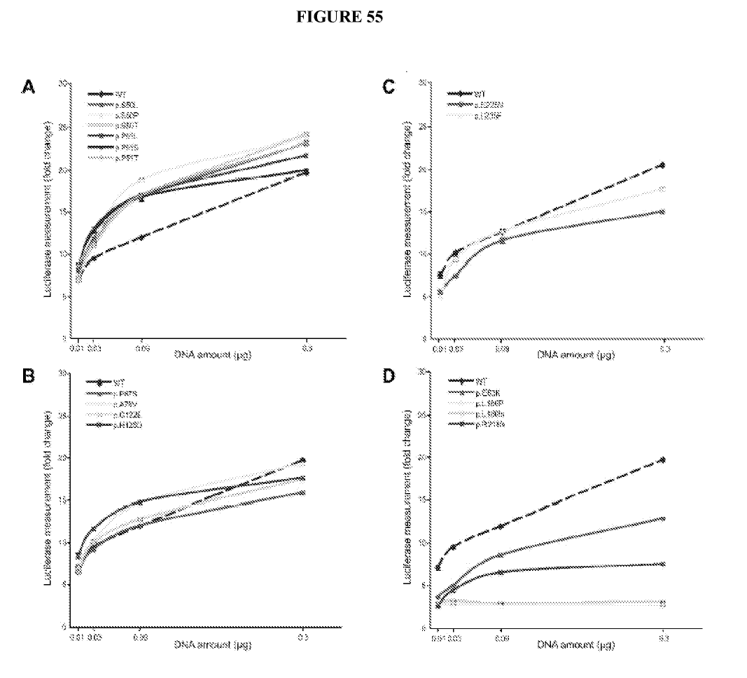

The effect of mutations in NRL on their ability to transactivate luciferase reporter activity driven by the bovine rhodopsin promoter in the presence of CRX was tested (See, e.g., Rehemtulla et al., 1996, Proc Natl Acad Sci USA 93:191-195). All p.S50 and p.P51 mutants showed a statistically significant increase (ANOVA with a post hoc test p<0.05) in transactivating the rhodopsin promoter when compared to WT-NRL at three of the four DNA concentrations tested (See FIG. 55A). The p.P67S, p.A76V and p.G122E alterations had no change from WT, while p.H125Q gave inconsistent results being significantly higher than WT using 0.03 μg or 0.09 μg DNA and lower with 0.3 μg NRL DNA (See FIG. 55B). Mutations exhibiting lower than WT transactivation were: p.E63K, p.L160P, p.L160fs, p.R218fs, and p.S225N (p<0.05, See FIGS. 55C, D). The p.L235F was significantly lower than WT at only two DNA concentrations (0.01 μg and 0.3 μg, See FIG. 55C).

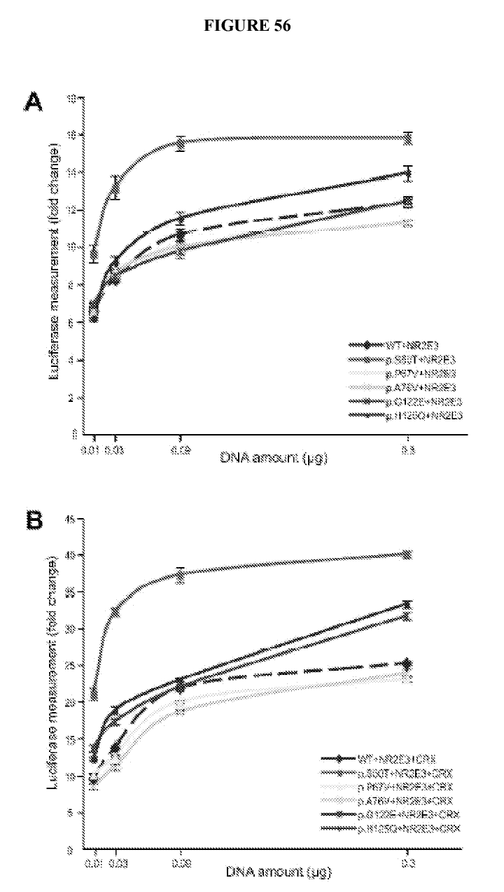

It was next determined whether mutant NRL proteins demonstrate altered transactivation of the rhodopsin promoter in the presence of NR2E3, which also acts as co-activator of rod genes with NRL and/or CRX (See, e.g., Cheng, et al., 2004, Hum Mol Genet 13:1563-1575). The p.S50T exhibited enhanced activation of the rhodopsin promoter when co-transfected with NR2E3 and/or CRX (See FIGS. 56A, B). The p.P67V and p.A76V did not show significant differences from WT in both experiments, whereas p.G122E and p.H125Q showed higher activities than WT when both NR2E3 and CRX were present (p<0.05, in at least three of four DNA concentrations tested, See FIG. 56B). The p.S50T and p.P51S mutants activated the rhodopsin promoter at higher levels than WT in the absence of CRX and NR2E3 and did not affect NRL's interaction with CRX or NR2E3, as revealed by co-IP experiments.

### Example 8

### Modulation of Nrl Expression/Activity: Retinoic Acid (RA) Influences Photoreceptor Differentiation and Rod-Specific Gene Expression

### Materials and Methods.

Reagents. Tissue culture media and serum were obtained from Invitrogen (Carlsbad, Calif.). Retinoic acids, growth factors, and other reagents were procured from Sigma. Stock solutions of RA and growth factors were prepared in 1% ethanol and/or dimethyl sulfoxide.

Cell Culture. Y79 human retinoblastoma cells (ATCC HTB 18) and HEK293 (ATCC CRL-1573) were maintained in RPMI 1640 and Dulbecco's modified Eagle's medium, respectively, under standard conditions with 15% (v/v) fetal bovine serum (FBS), penicillin G (100 units/ml), and streptomycin (100 μg/ml) at 37° C. and 5% CO₂. For serum starvation and RA treatment experiments, Y79 cells (5×10⁴) were cultured in the presence or absence of the serum (same batch of serum was used in all the experiments), at RA, 9-cis-RA, cycloheximide (CHX), and 4-(E-2-(5,6,7,8-tetrahydro-5,5,8,8-tetramethyl-2-naphthalenl)-1-propenyl) benzoic acid (TTNPB) at indicated concentrations. Me₂SO or ethanol was added to Y79 cells in lieu of the soluble factors as negative control.

For protein synthesis inhibition experiments, Y79 cells were serum-starved for 24 h, and then simultaneously treated with RA and CHX for 8 or 24 h. NRL expression was analyzed by immunoblotting. In another set of experiments, serum-starved Y79 cells were first incubated with RA alone for 8 or 24 h and then CHX was added. Cell extracts were then analyzed 24 h later for examining NRL expression by immunoblotting.

Primary cultures of new-born rat retinal cells and enriched adult porcine photoreceptors were prepared as described (See Traverso et al., (2003) Investig. Ophthalmol. Vis. Sci. 44, 4550-4558). For newborn rat retinal cultures, rat pups were anesthetized and decapitated, the retinas dissected into CO₂-independent Dulbecco's modified Eagle's medium and chopped into small fragments. The fragments were washed twice in Ca/Mg-free PBS and then digested in PBS containing 0.1% papain for 25 min at 37° C. Tissue was dissociated by repeated passage through flame polished Pasteur pipettes, then seeded into tissue culture plates precoated with laminin, in Neurobasal A medium (Invitrogen) containing 2% FBS. After 48 h, medium was changed to a chemically defined formula (Neurobasal A supplemented with B27) for a further 48 h, and then treated.

For pig photoreceptor cultures, eyes were obtained from freshly slaughtered adult pigs, the retinas removed and dissected under sterile conditions. Tissue was minced, digested with papain, and dissociated by mild mechanical trituration. Cells obtained from the first two supernatants were pooled and seeded at 5×10⁵/cm² into 6×35 well tissue culture plates as above. Cells were cultured as outlined above (48 h Neurobasal A/2% FBS, then 48 h Neurobasal A with B27).

Experimental Treatments and Immunochemistry. After the 4-day culture period, both primary cell models were treated as follows. RA was added to test wells (1, 5, 10, 20, and 40 μM, stock solution prepared in Me₂SO, 10 μl/well). Negative control wells received Me₂SO alone, and positive control wells were treated with Neurobasal containing 2% FBS. For immunoblotting, the medium was removed after 24 h; cells were rinsed in PBS and processed as indicated.

For immunocytochemical studies, medium was removed after 24 h, and cells were fixed in 4% paraformaldehyde in PBS for 15 min. Cells were permeabilized for 5 min using 0.1% Triton X-100, then preincubated in blocking buffer (PBS containing 0.1% bovine serum albumin, 0.1% Tween 20 and 0.1% sodium azide) for 30 min. Cells were incubated overnight in affinity-purified anti-NRL antiserum (1:1000 dilution), and monoclonal anti-rhodopsin antibody rho-4D2 (45), rinsed thoroughly, and incubated with secondary antibodies (anti-rabbit IgG-Alexa594 and anti-mouse IgG-Alexa488) combined with 4,6-di-amino-phenyl-indolamine (DAPI) (all from Molecular Probes Inc., Eugene, Oreg.) for 2 h. Cells were washed, mounted in PBS/glycerol, and examined under a Nikon OPTIPHOT 2 fluorescence microscope. All images were captured using a CCD camera and transferred to a dedicated PC. The same capture parameters were used for each stain, and final panels were made using untreated images for direct comparison of staining intensities.

Protein Expression Analysis. Y79 and newborn rat retinal cells were sonicated in PBS and clarified supernatant was used for further analysis. Protein concentration was determined using Bio-Rad protein assay reagent. Equal amounts of proteins were analyzed by SDS-PAGE followed by immunoblotting. Proteins were detected using anti-NRL polyclonal antibody as described (See, e.g., Cheng et al., (2004) Hum. Mol. Genet. 13, 1563-1575; Swain et al., (2001) J. Biol. Chem. 276, 36824-36830). Immunoblots from three independent experiments for rat and pig retinal cultures were analyzed by densitometric scanning, and normalized to serum-supple-mented control levels in each case. Statistical analysis of data were performed using the one-tailed Student's t test, with p<0.05 accepted as level of significance.

Plasmid Constructs. DNA fragments of 2.5 kb (Nl), 1.2 kb (Nm), and 200 by (Ns) from the 5′-flanking region of the mouse Nrl promoter (GENBANK: AY526079; (See Akimoto et al., (2006) Proc. Natl. Acad. Sci. U.S.A. 103, 3890-3895) were amplified and cloned into pGL3-basic vector (Promega, Madison, Wis.) in-frame with the luciferase reporter gene. The following site-directed mutants of the Nrl promoter were generated from pGL3-Nl using the QUICKCHANGE site-directed mutagenesis kit (Stratagene, La Jolla, Calif.) and sequence-verified: pGL3-Nl-mutIII-1, pGL3-Nl-mutIII-2, and pGL3-Nl-mutII-1, containing deletion of the putative RAREs at positions −781 to −767, −709 to −700, and −453 to −443, respectively.

DNaseI Footprinting and Electrophoretic Mobility Shift Assays (EMSA)—Bovine retinal nuclear extract (RNE) was prepared as described (See Lahiri, D. K., and Ge, Y. (2000) Brain Res. Brain Res. Protoc. 5, 257-265). Solid phase DNaseI footprinting was performed as described (Sandaltzopoulos, R., and Becker, P. B. (1994) Nucleic Acids Res. 22, 1511-1512), using 100 μg of RNE, and various fragments from the upstream conserved regions of the mouse Nrl promoter were used as template. For EMSA, oligonucleotides containing the wild-type mouse Nrl promoter sequence(oligo III-2 nucleotides −726 to −686: 5′-ACGGG-GAAAAGGTGAGAGGAAGC-3′ (SEQ ID NO.: 17), oligo II-1 nucleotides −469 to −427: 5′-GCAGGGGCTGAAATGTGAGGA-3′ (SEQ ID NO.: 18)) or deletion of the putative RAREs (mt-Oligo III-2: 5′-CTGAGACACCGCACGGGGAGGAAGCTGAGGGC-3′ (SEQ ID NO.: 19); and mt-Oligo II-1: 5′-GGTGAAGGTAGGGCAGTGAG-GATGCTTGAAAA-3′ (SEQ ID NO.: 20)) were end-labeled using[γ-³²P]ATP (Amersham Biosciences) and incubated in binding buffer (20 mM HEPES pH 7.5, 60 mM KCl, 0.5 mM dithiothreitol, 1 mM MgCl₂, 12% glycerol) with RNE (20 μg) and poly(dI-dC) (50 μg/ml) for 30 min at room temperature. In competition experiments, a non-radiolabeled oligonucleotide was used in molar excess of the labeled oligonucleotide. In some gel-shift experiments, antibodies were added after the incubation of ³²P-labeled oligonucleotides with RNE. Samples were loaded on 7.5% non-denaturing polyacrylamide gel. After electrophoresis, the gels were dried and exposed to x-ray film.

Transient Transfection and Luciferase Assay. Transient transfection of Y79 cells was performed using FUGENE 6 reagent (Roche Diagnostics, Indianapolis, Ind.). Prior to transfection, cells were serum-starved 24 h in OPTI-MEM (Invitrogen), diluted to 1.5×10⁵ cells in 250 μl and seeded into 24-well plates. Transfection was performed with 0.5 μg of promoter-luciferase construct and 1.5 μl of FUGENE 6. One hour after transfection, 10 μMRA or 1% ethanol was added to each well. Transfected cells were cultured for additional 24 h and harvested. Luciferase activity was measured using the Luciferase Assay System (Promega, Madison, Wis.). Experiments were repeated at least three times, and the luciferase activity was calculated as a fold change from the base line luciferase activity obtained in the presence of vector only.

Transient transfection of HEK293 (ATCC CRL-1573) cells was performed using LIPOFECTAMINE (Invitrogen) according to the manufacturer's instructions. The wild type and mutant Nrl promoter-luciferase constructs, and pCMV-β-gal were added to the cells at a concentration of 0.1 μg and 0.05 μg, respectively. After 3 h, 100 μl of Dulbecco's modified Eagle's medium with 0 or 500 nM at RA was added to each well. Cells were harvested after 24 h in 100 μl of GLO lysis buffer (Promega), and luciferase activity was measured.

### Serum-Deprivation of Y79 Cells.

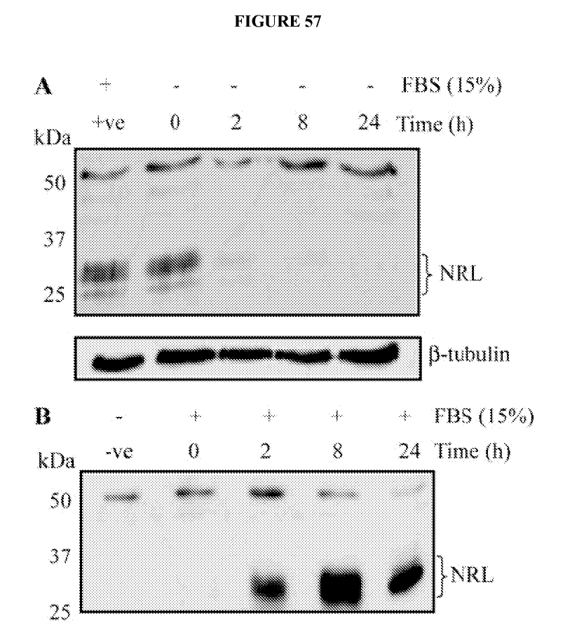

NRL is expressed in Y79 cells but not in other tested cell lines (See, e.g., Swaroop et al., (1992) Proc. Natl. Acad. Sci. U.S.A. 89, 266-270). To generate an efficient in vitro model system to study regulation of NRL expression, serum deprivation of Y79 cells was carried out. Northern blot analysis and RT-PCR failed to detect NRL transcripts within 24 h after serum deprivation. Immunoblot analysis showed that NRL expression in Y79 cells decreased 8 h after serum depletion and was undetectable by 24 h (See FIG. 57A). No cell death was detected because of serum deprivation within the time span of the experiments. When serum was supplied to these cells, NRL expression was detectable in 2 h and completely restored within 8 h (See FIG. 57B). Multiple immunoreactive bands in 29-35 kDa range represent different phosphorylated isoforms of NRL that are detected by affinity-purified anti-NRL antibody (See Swain et al., (2001) J. Biol. Chem. 276, 36824-36830). Additional bands observed in immunoblots may represent unrelated cross-reactive proteins, and their levels did not change after serum deprivation.

### RA Effect on NRL Expression.

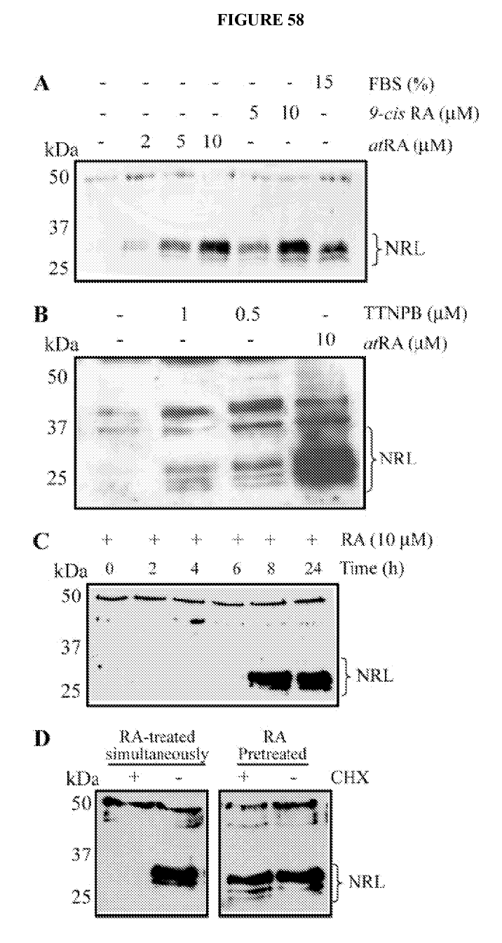

To identify possible activators in serum, the effect of a number of soluble factors on NRL expression were tested. A dose-dependent increase in NRL expression was observed following incubation with at RA and its isomer, 9-cis RA (See FIG. 58A). The effect of RA was mimicked by a RAR-specific agonist, TTNPB (See FIG. 58B). Northern blot analysis of RNA from the treated cells also showed RA induction of NRL transcripts.

The time course of NRL induction by RA was then analyzed. An increase in NRL protein was observed in serum-starved Y79 cells after 8 h of incubation with at RA (See FIG. 58C). A similar effect was observed with 9-cis RA. Treatment of cells with at RA and CHX (20 μg/ml), an inhibitor of protein synthesis (See, e.g., Vazquez, D. (1979) Mol. Biol. Biochem. Biophys. 30, i-x, 1-312), blocked NRL induction when both were added simultaneously (See FIG. 58D). This suggests that intermediate protein synthesis is necessary for RA-mediated induction of NRL expression. However, when cells were pretreated with RA for 8 or 24 h, CHX had no detectable effect on NRL expression (See FIG. 58D). Thus, the present invention provides that synthesis of intermediary factors necessary for NRL induction occurs within 8 hours of RA treatment.

### RA Stimulation of NRL Expression in Rat and Porcine Photoreceptors.

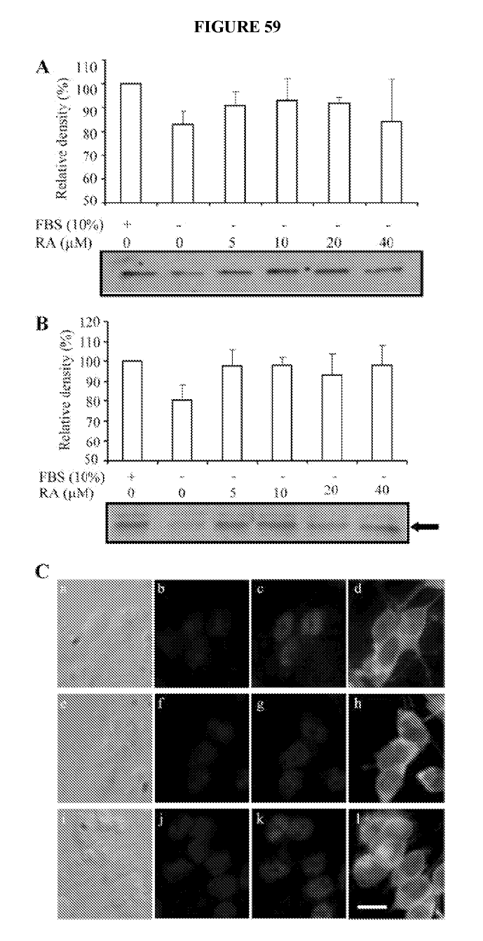

To investigate the effect of RA on the expression of NRL in photoreceptors in vitro, two different culture models were utilized. Immunoblotting of proteins isolated from monolayer cultures of newborn rat retina revealed that maintenance of cells in chemically defined conditions for 24 h led to moderate but reproducible decreases in NRL expression levels, and that either re-addition of serum or increasing doses of RA increased the NRL band intensity (See FIG. 59A). Only a single NRL-immunoreactive band was visible using the newborn rat retinal cells (See FIG. 59A). Similar induction in NRL expression was observed using highly enriched photoreceptor cultures prepared from adult pig retina, which however showed two NRL-immunoreactive bands (See FIG. 59B). In both rat and pig cultures, maximal effects were observed with 5-20 μM RA, and higher doses led to some toxicity especially in cells from new-born rat retina. Immunocytochemical studies of pig photoreceptor cultures revealed that NRL was confined to rod nuclei in all cases, and that signal was relatively strong in serum-or RA-supplemented conditions. The serum-free photoreceptor culture displayed a modest but reproducible decrease in NRL-specific signal in the nuclei, as seen in immunoblots (See FIG. 59C). Expression levels in newborn rat retinal cultures were too low to be detected by immunocytochemistry.

### Role of RA Receptors.

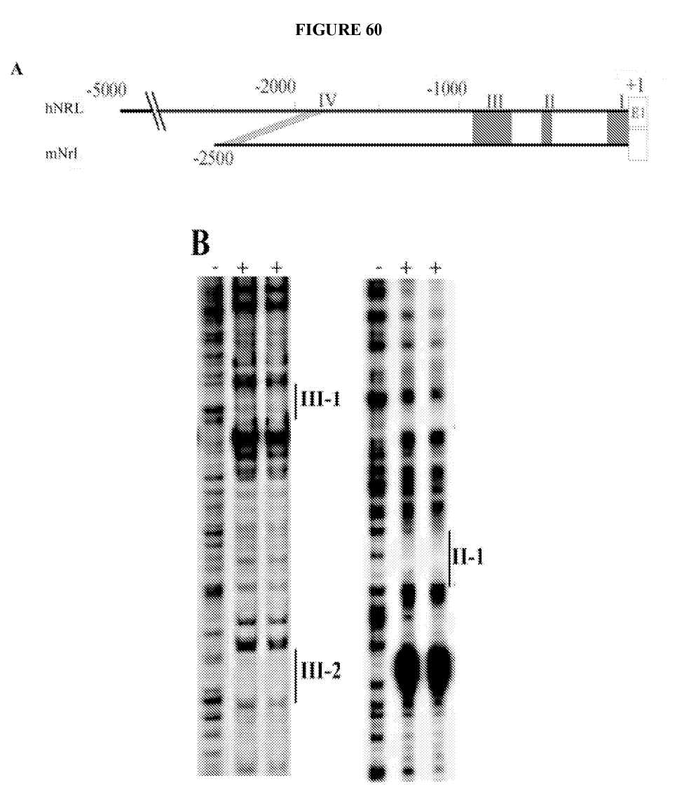

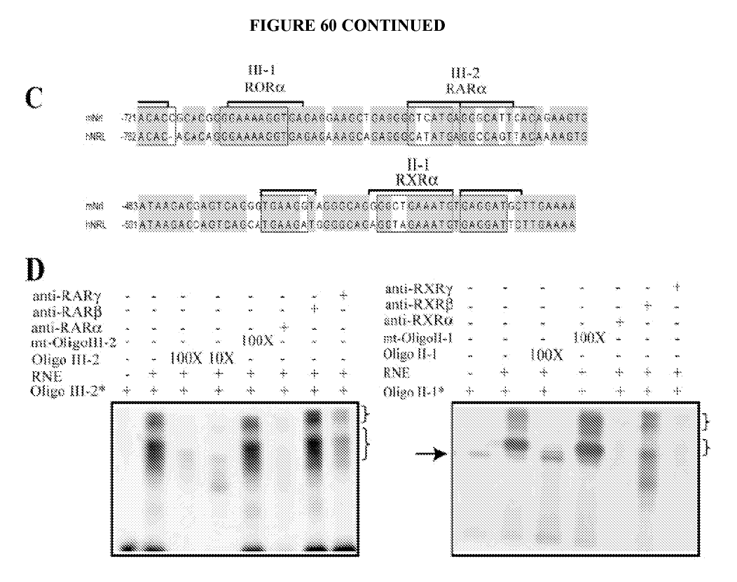

It was next determined whether RA acts directly on the Nrl promoter. DNaseI footprinting analysis of conserved sequences upstream of the transcription start site of the mouse Nrl gene identified putative RAREs (regions III-1, III-2, and II-1), in addition to other transcription factor binding elements (See, e.g., FIGS. 60, A and B). Oligonucleotides encompassing these protected sequences were radiolabeled and used for EMSA analysis (See FIG. 60C). Mobility shift was observed of the radiolabeled oligonucleotides in the presence of bovine retinal nuclear extracts (See FIG. 60D). The intensity of the shifted bands was reduced or eliminated by molar excess of the same non-radiolabeled oligonucleotide, but not by a mutant oligonucleotide carrying a deletion of the putative RAREs. The shifted bands were also diminished when anti-RARα, anti-RXRα, or anti-RXRγ but not RARβ, RARγ, or RXRβ-specific antibodies were added (See FIG. 60D).

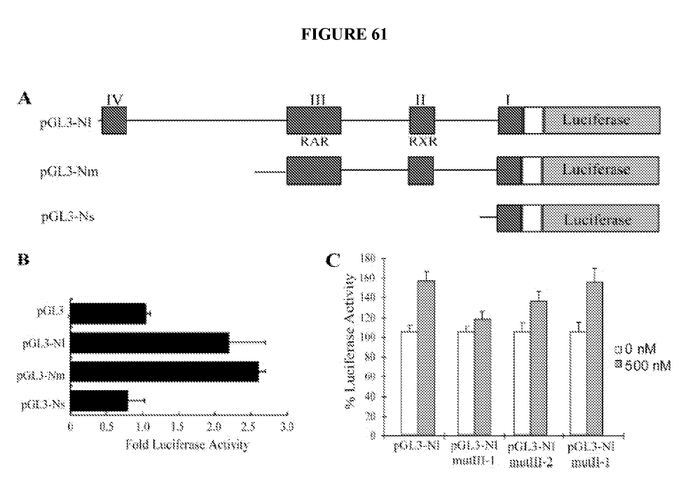

To investigate the functional relevance of the binding of RA receptors to the Nrl promoter, transient transfection experiments in serum-deprived Y79 cells were performed using Nrl promoter-luciferase constructs containing the 2.5-kb fragment (pGL3-Nl) as well as deletion variants encompassing the footprinted regions III and II (pGL3-Nm and pGL3-Ns) (See FIG. 61A). Addition of at RA showed over a 2-fold increase in luciferase activity with pGL3-NI and pGL3-Nm constructs, which included the putative RAREs (See FIG. 61B). The pGL3-Ns construct did not show a detectable increase in the reporter activity in the presence of RA. All three constructs induced luciferase reporter activity when transiently transfected into Y79 cells in the presence of serum.

To further ascertain the involvement of putative RAREs in RA-mediated up-regulation of Nrl promoter activity, site-directed mutagenesis was performed to delete the putative RAREs from the pGL3-Nl promoter-luciferase construct. The pGL3-Nl construct showed a dose-dependent response to RA treatment in HEK293 cells with maximum effect in the presence of 500 nM at RA (See FIG. 61C). However, deletions encompassing the region III-1 (pGL3-Nl-mutIII-1 and pGL3-Nl-mutIII-2) resulted in a reduction in luciferase activity in the presence of 500 nM at RA (See FIG. 61C). Although binding of RXRα and RXRγ on Nrl promoter was observed, deletion of the putative RXR binding site (pGL3-Nl-mutII-1) did not have any appreciable effect on the luciferase activity. Although an understanding of the mechanism is not necessary to practice the present invention and the present invention is not limited to any particular mechanism of action, in some embodiments, this might reflect heterodimerization between RARs and RXRs at other sites (e.g., footprint III-2) on the promoter (e.g., thereby compensating for the lack of binding of RXRs to footprint II-1).

### Example 9

### NRL Activates the Expression of Nuclear Receptor NR2E3 to Suppress the Development of Cone Photoreceptors

Materials and methods.

Transgenic mice. Crxp-Nrl/WT and Crxp-Nr2e3/WT mice were generated as described in Examples 5 and 6 above. Crxp-Nrl/WT mice were mated with rd7 mice (procured from Jackson Laboratory) to generate Crxp-Nrl/rd7 mice. The mice were in a mixed background of 129X1/SvJ and C57BL/6J. PCR primers for genotyping the Crxp-Nrl/WT allele were: F: 5′-AGCCAATGTCACCTCCTGTT-3′ (SEQ ID NO. 21) and R: 5′-GGGCTCCCTGAATAGTAGCC-3′ (SEQ ID NO. 22). PCR primers for genotyping the rd7 allele were as described (See Haider et al., Hum Mol Genet 10 (2001) 1619-1626). All studies involving mice were performed in accordance with institutional and federal guidelines and approved by the University Committee on Use and Care of Animals at the University of Michigan.

Gene Profiling. Microarray analysis was conducted as described (See, e.g., Yoshida et al., Hum Mol Genet 13 (2004) 1487-1503; Yu et al., J Biol Chem 279 (2004) 42211-42220; Zhu et al., J Comput Biol 12 (2005) 1029-1045). Briefly, total RNA (Trizol, INVITROGEN) from P28 retinas was used to generate double-stranded cDNA for hybridization to mouse GeneChips MOE430.2.0, per guidelines (AFFYMETRIX). Total retinal RNA from four independent samples was used for each evaluation. Normalized data were subjected to two-stage analysis based on false discovery rate with confidence interval (FDRCI) for identifying differentially expressed genes (See, e.g., Zhu et al., J Comput Biol 12 (2005) 1029-1045).

Immunohistochemistry. Retinal whole mounts and 10 μm sections were probed with the following antibodies: rabbit S-opsin, rabbit M-opsin, and rabbit cone-arrestin (from C. Craft, University of Southern California, Los Angeles, Calif., and CHEMICON), mouse anti-rhodopsin (1D4 and 4D2; from R. Molday, University of British Columbia, Vancouver, Canada). The secondary antibodies for fluorescent detection were ALEXAFLUOR488 and 546 (Molecular probes, INVITROGEN). Sections were visualized using an OLYMPUS FLUOVIEW 500 laser scanning confocal microscope. Images were subsequently digitized using FLUOVIEW software version 5.0. EMSA. The electrophoretic mobility shift assays were performed using established methods (See, e.g., Hao, et al., Blood 101 (2003) 4551-4560), with minor modifications. Nuclear protein extracts from transfected COS-1 cells were prepared using a commercial kit (ACTIVE MOTIF, Carlsbad, Calif.), and expression of NRL protein was confirmed by SDS-PAGE followed by immunoblotting. Nuclear extracts were incubated with 1 μg poly (dIdC) at 4° C. for 15 min in the binding buffer (12 mM HEPES (N-2-hydroxyethylpiperazine-N′-2-ethanesulfonic acid), pH 7.9; 60 mM KCl; 4 mM MgCl2; 1 mM EDTA (ethylenediaminetetra acetic acid); 12% glycerol; 1 mM dithiothreitol; and 0.5 mM phenylmethylsulfonyl fluoride (PMSF)). Then, ³²P-labeled doublestranded oligonucleotide (40,000 cpm) was added and the reaction was incubated at 4° C. for 20 min. The DNA probe (−2820 nt to −2786 nt: NRE F5′-TGGCCTCTGGTGGCTTTGTCAGCAGTTCCAAGGCT-3′ (SEQ ID NO. 23), NRE R 5′-AGCCTTGGAACTGCTGACAAAGCCACCAGAGGCCA-3′) (SEQ ID NO. 24) contains a putative NRL-response element (NRE) (underlined) that is predicted by GENOMATIX. In competition studies, nuclear extracts were pre-incubated with 50 or 100× unlabeled oligonucleotide for 30 min at room temperature and incubated with labeled probe at room temperature for 20 min. A mutant oligonucleotide (F: 5′-TGGCCTCTGGTGGCTT TATTTGCAGTTCCAAGGCT-3′ (SEQ ID NO. 25), R: 5′-AGCCTTGGAACTGCAAATAAAGC CACCAGAGGCCA-3′) (SEQ ID NO. 26) with three nucleotide change in the NRE site was also used to compete for the protein binding to the probe. In order to immunologically identify the components in protein-DNA complexes, nuclear extracts were incubated with 2.0 μg of the anti-Nrl antibody or normal rabbit IgG for 30 min at room temperature, followed by the addition of labeled probe and a further incubation for 20 min at room temperature. The reaction mixtures were electrophoresed on 6% polyacrylamide gels at 175 volts for 2.5 hr and subjected to autoradiography.

ChIP. Chromatin immunoprecipitation assays were performed using a commercial kit (ACTIVE MOTIF, Carlsbad, Calif.). Briefly, four snap-frozen retinas from wild type C57BL/6J mice were cross-linked for 15 min at room temperature with 1% formaldehyde in

PBS containing protease inhibitors (See, e.g., Oh et al., Proc Natl Acad Sci USA 104 (2007) 1679-1684). The reaction was stopped by adding glycine (125 mM), followed by 5 min incubation at room temperature. The remaining steps were performed according to the manufacturer's instructions, using anti-NRL polyclonal antibody or normal rabbit IgG. ChIP DNAs were used for PCR amplification of a 248-bp fragment (−2989 nt to −2742 nt), containing a putative NRE (as determined by GENOMATIX), with primers 5′-GCATGCACTGTTCAAACACC-3′ (SEQ ID NO. 27) and 5′-GATAGGCTGTGCAGGGGTTA-3′ (SEQ ID NO. 28). PCR with another pair of primers (5′-TGTCCTGAGTCTCC CTGCTT-3′ (SEQ ID NO. 29) and 5′-TAAGGCTGGCCAT AAAGTGG -3′) (SEQ ID NO. 30) that amplify a 209-bp fragment (1230 nt to 1438 nt) located about 4 kb downstream from the NRE site, served as a negative control.

ERG. Electroretinography recordings were performed on 2-3 month old adult mice, as described (See, e.g., Mears et al., Nat Genet 29 (2001) 447-452).

### Results.

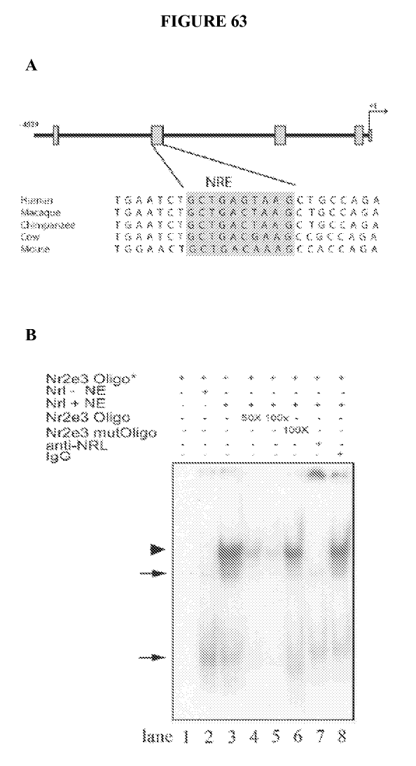

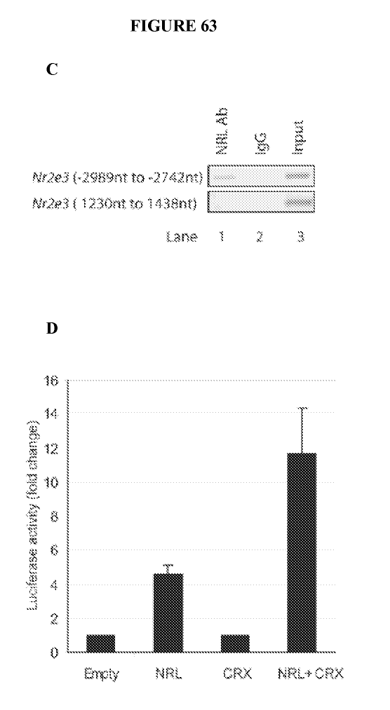

NRL directly binds to the Nr2e3 promoter. To examine whether NRL can modulate NR2E3 expression, the promoter of the Nr2e3 gene was first analyzed and four sequence regions were identified that are conserved in mammals (See FIG. 63A). In silico analysis revealed a putative NRL response element (NRE) in one of the conserved regions (See FIG. 63A). This NRE sequence could bind to COS-1 cell expressed NRL protein in electrophoretic mobility shift assays (EMSA) (See FIG. 63B). The specificity of Nr2e3-NRE element for NRL binding is substantiated by competition with an excess of unlabeled oligonucleotide spanning NRE but not with a mutant sequence. To determine whether NRL could bind the Nr2e3 promoter in the context of native chromatin, chromatin immunoprecipitation (ChIP) experiments were performed. Cross-linked protein-DNA complexes from adult wild-type retinas were immunoprecipitated with an anti-NRL antibody, and purified ChIP DNA was used for PCR with primers flanking the NRE site. Strong enrichment of the Nr2e3-NRE promoter fragment was observed with anti-NRL antibody compared to a nonspecific antibody (rabbit IgG) (See FIG. 63C). Additionally, no significant enrichment was detected for another sequence in the Nr2e3 gene (used as a negative control) under similar conditions (See FIG. 63C).

NRL induces the Nr2e3 promoter activity in transfected cells. Next, it was determined whether NRL could activate a 4.5 kb Nr2e3 promoter sequence encompassing the conserved NRE sequence (See FIG. 63A). Transfection of HEK-293 cells with NRL (but not CRX) expression plasmid activated the luciferase reporter gene driven by the Nr2e3 promoter (See FIG. 63D). Co-transfection of CRX with NRL resulted in further increase in the Nr2e3 promoter activity (See FIG. 63D).

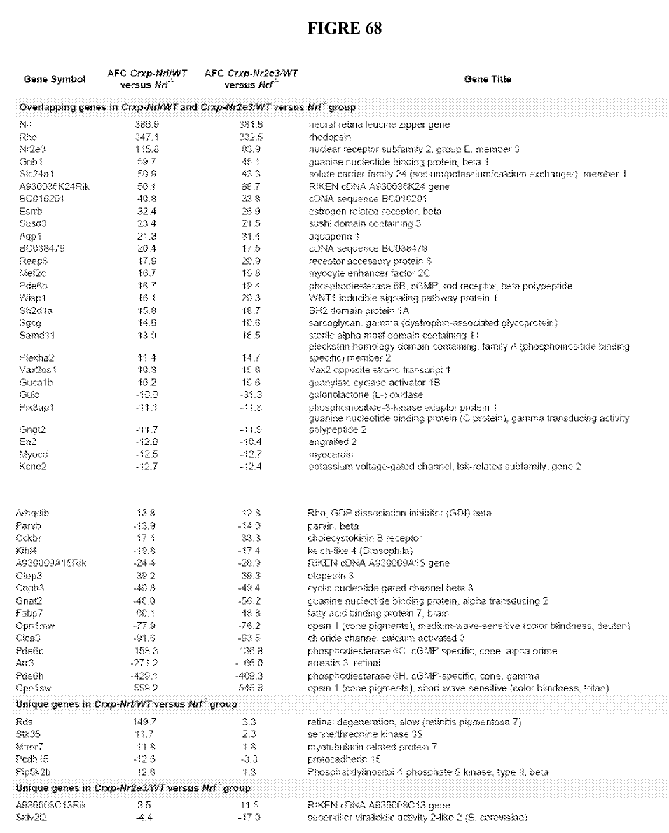

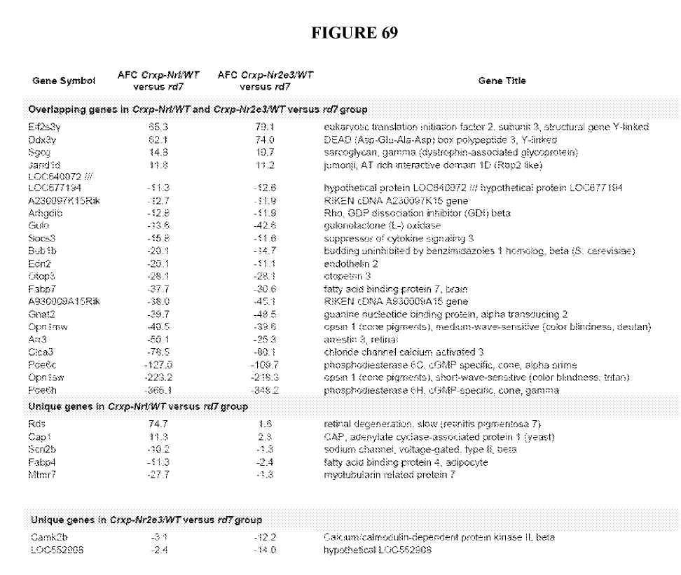

Overlapping yet distinct gene profiles are generated by NRL and NR2E3. In order to dissect the transcriptional activity of NRL versus NR2E3, two transgenic mouse models that do not have cone photoreceptors, Crxp-Nrl/WT and Crxp-Nr2e3/WT were utilized. In the Crxp-Nrl/WT retinas, NRL and consequently NR2E3 (See FIG. 1) are ectopically expressed in cone precursors (See FIG. 63 and Example 6); while only NR2E3 is ectopically expressed in cone precursors of the Crxp-Nr2e3/WT retina. Gene profiling of retinas from Crxp-Nrl/WT and Crxp-Nr2e3/WT mice can therefore reveal expression changes induced by NRL+NR2E3 or NR2E3 alone, respectively. Retinal RNA from adult mice (28 days post-natal) was hybridized to AFFYMETRIX MOE430.2.0 GENECHIPS, which contain 45,101 probesets for mouse transcripts. A comparative analysis of gene clusters from Crxp-Nrl/WT and Crxp-Nr2e3/WT retinas to WT samples revealed genes involved in diverse signaling pathways and transcriptional regulation; FIG. 67 shows the genes with FDRCI P value of <0.1 and a fold change >4. In some embodiments, the present invention provides that these unique genes represent downstream targets that may be exclusively cone-enriched. Crxp-Nrl/WT and Crxp-Nr2e3/WT gene profiles were then compared to Nrl−/− (cone-only) and rd7 (1.5-2 fold more S-cones) profiles. Many cone phototransduction genes that are upregulated in the Nrl−/− (cone-only, FIG. 68) and rd7 (1.5-2 fold more S-cones, FIG. 69) retinas are also significantly repressed in the Crxp-Nrl/WT and Crxp- Nr2e3/WT samples. Gene expression changes showing FDRCI P-value <0.1 and a fold change >10 are listed in FIG. 68 and FIG. 69.

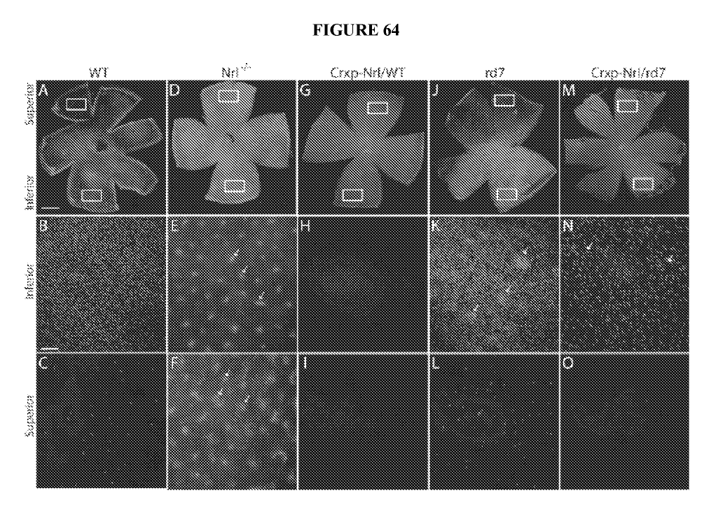

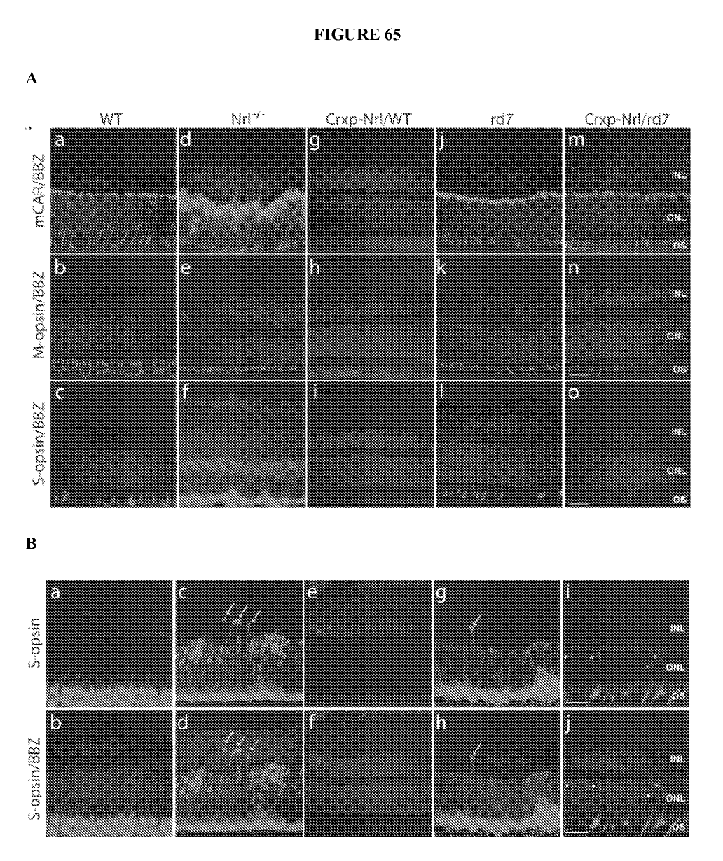

Expression of NRL can only suppress a subset of S-cones in the absence of NR2E3. Similarities in gene profiles of Crxp-Nrl/WT and Crxp-Nr2e3/WT retinas raise the question whether NRL can suppress cone gene expression and differentiation in the absence of NR2E3. In order to evaluate this, Crxp-Nrl/WT mice were mated to rd7 mice to generate a transgenic mouse line (Crxp-Nrl/rd7) that expresses NRL in both cone and rod precursors but not NR2E3. Cone markers were analyzed, such as S- and M-opsin, in retinal whole mounts. An inferior to superior gradient of S opsin expression was observed (See FIG. 64A-C; Applebury et al., Neuron 27 (2000) 513-523) and a superior to inferior gradient of M-opsin in the WT mice was observed. S-opsin was detected throughout in the Nrl−/− retinal whole mounts (See FIG. 64D-F) and increased S-opsin staining was observed in the rd7 retinas (See FIG. 64J-L); however, both S-opsin and M-opsin could not be detected in Crxp-Nrl/WT retinas (See FIG. 64G-I). In both Nrl−/− and rd7 mice, whorls are detected in the whole mount preparations (See FIGS. 64D-F and J-K). In Crxp-Nrl/rd7 retinal whole mounts, a large absence of S-opsin staining in the superior domain was observed (See FIGS. 64M, O) yet a small population of S-opsin positive cells in the inferior retina (See FIGS. 64M, N) was detected. The expression of M-opsin was unaltered, and whorls could be detected throughout the retinas (See FIG. 64M-O). The number of cone arrestin and S-opsin positive cells in retinal cross-sections from Nrl−/− and rd7 retinas were increased compared to WT, and there is an absence of cone-specific markers in Crxp-Nrl/WT mice (See FIG. 65A: a-o). In Crxp-Nrl/rd7 sections, normal cone arrestin and M-opsin staining was observed but there was an absence of S-opsin in the superior domain (See FIG. 65A: m-o). In the inferior domain, a few S-opsin positive cones and many S-opsin positive cell bodies were identified at the inner portion of the ONL (See FIG. 65B: i, j). This was in contrast to S-opsin positive cells distributed throughout the ONL and INL in Nrl−/− and rd7 retinas (See FIG. 65B: c-d and g-h). Thus, in some embodiments, the present invention provides (e.g., based on the presence of cone arrestin and M-opsin expression in the Crxp-Nrl/rd7 mice (harboring the Crxp-Nrl transgene in rd7 background with no NR2E3 function) but not in the Crxp-Nrl/WT mice (harboring the Crxp-Nrl transgene in wild-type background)) that NR2E3 is a primary suppressor of cone gene expression and cone differentiation.

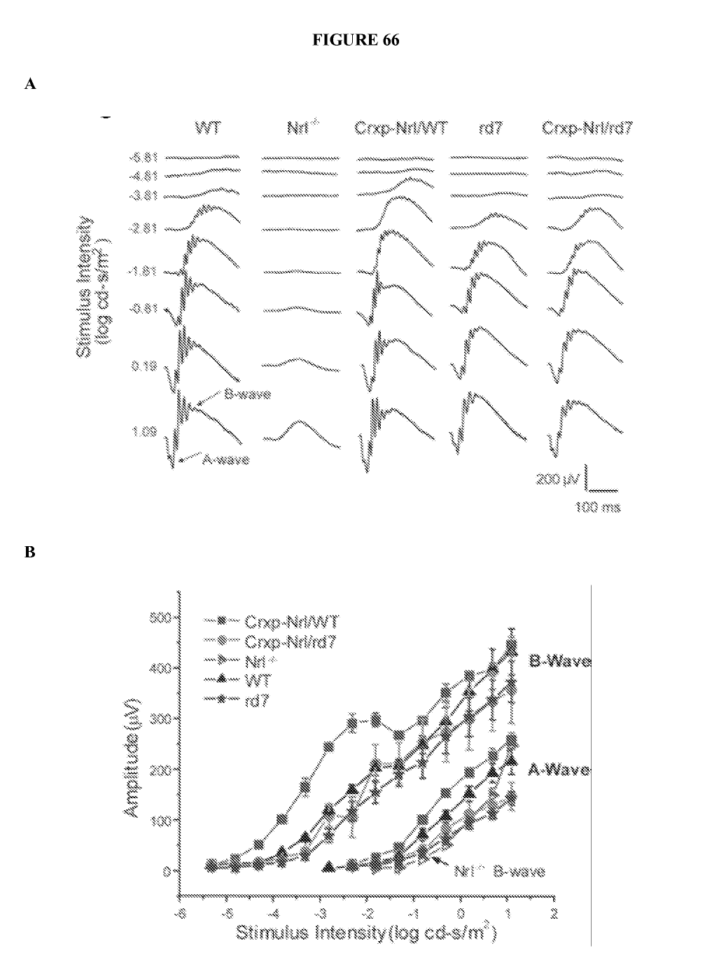

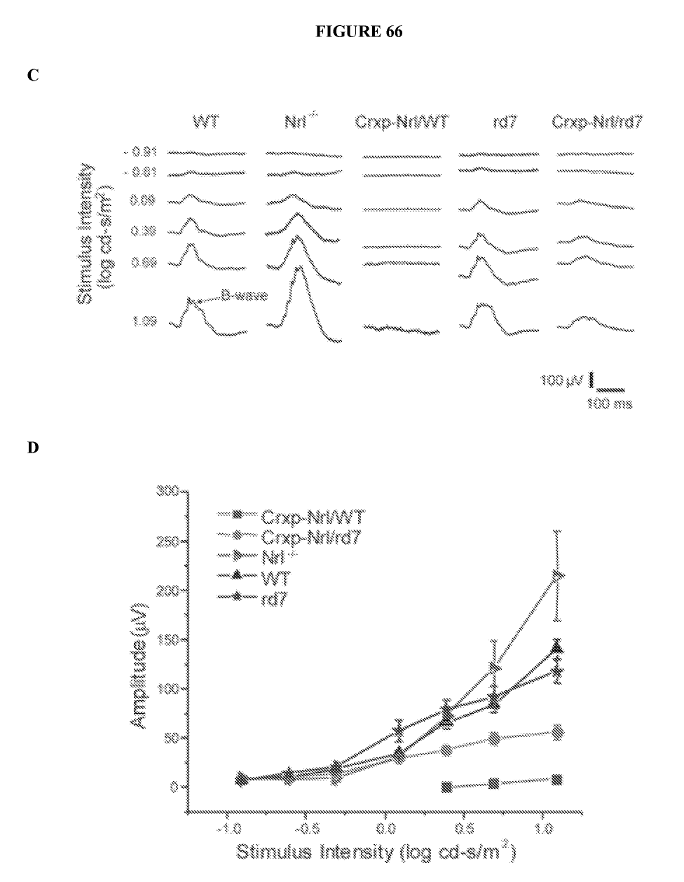

Cone function is detected but reduced in the Crxp-Nrl/rd7 mice. Electroretinography (ERG) recordings was performed to measure the massed-field potential across the retina in the different transgenic lines. The ectopic expression of NRL in cone precursors (Crxp-Nrl/WT) resulted in an absence of cone-driven responses, whereas rod-driven components were preserved (See FIG. 66). In order to characterize the functionality of conedriven neurons in the absence of NR2E3, the photopic response from Crxp-Nrl/rd7 mice was analyzed (See FIGS. 66C, D). In response to brief flashes of white light, a cone-driven b-wave was first detected at 0.09 log cd-s/m². At the higher flash intensity of 1.09 log cd-s/m² the maximum b-wave amplitude was about 40% of the WT response amplitude (See FIG. 66).

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1-53. (canceled)

54. A composition comprising a purified photoreceptor precursor cell.

55. The composition of claim 54, wherein said cell expresses Nrl.

56. The composition of claim 55, wherein expression of Nrl identifies said cell as a rod photoreceptor precursor cell.

57. The composition of claim 54, wherein said cell comprises heterologous nucleic acid sequence encoding a Nrl promoter operatively linked to green fluorescent protein.

58. The composition of claim 54, wherein said cell is able to survive and differentiate when placed within a retina.

59. The composition of claim 54, wherein said cell is purified from an embryonic mouse or a post-natal mouse.

60. The composition of claim 54, wherein said cell integrates within the outer nuclear layer of a retina when injected into the subretinal space of said retina.

61. The composition of claim 54, wherein the integrated cell forms synaptic connections with downstream targets in said retina.

62. The composition of claim 57, wherein the integrated cell responds to a light stimulus.

63. A method screening test compounds comprising:
a) providing a photoreceptor cell comprising a heterologous nucleic acid sequence encoding a Nrl promoter operatively linked to green fluorescent protein;
b) exposing said cell to one or more test compounds; and
c) detecting a change in photoreceptor cell function.

64. The method of claim 63, wherein said photoreceptor cell is present within a transgenic, non-human animal whose genome comprises a heterologous nucleic acid sequence encoding a Nrl promoter operatively linked to green fluorescent protein.

65. The method of claim 63, wherein said detecting a change in photoreceptor cell function comprises detecting a change in expression of green fluorescent protein.

66. The method of claim 63, wherein said detecting a change in photoreceptor cell function comprises detecting a change in expression of one or more biomarkers selected from the group consisting of a gene described in FIG. 11, a gene described in FIG. 12, and a gene described in FIG. 13.

67. The method of claim 63, wherein said detecting a change in photoreceptor cell function comprises characterizing the ability of said photoreceptor cell to make synaptic connections with downstream targets in a retina.

68. The method of claim 63, wherein said detecting a change in photoreceptor cell function comprises characterizing the ability of said photoreceptor cell to integrate within a retina.

69. The method of claim 63, wherein said detecting a change in photoreceptor cell function comprises characterizing the ability of said photoreceptor cell to respond to a synapse-dependent stimulus.

70. A method of converting a non-rod cell to a rod photoreceptor cell comprising altering Nrl expression and/or activity in said non-rod cell.

71. The method of claim 70, wherein altering Nrl expression and/or activity comprises inducing Nrl expression with a small molecule.

72. The method of claim 70, wherein altering Nrl expression and/or activity comprises altering the post-translational modification of Nrl.

73. The method of claim 72, wherein altering Nrl expression and/or activity alters the expression of one or more gene targets of Nrl.
